(19) United States
(12) Patent Application Publication
(10) Pub. No.: US 2009/0304697 A1
(43) Pub. Date: Dec. 10, 2009

(54) Identification and use of prognostic and predictive markers in cancer treatment

(75) Inventors: Soonmyung Paik, Pittsburgh, PA (US); Chungyeul Kim, Wexford, PA (US)

Correspondence Address: VINSON & ELKINS L.L.P., First City Tower, 1001 Fannin Street, Suite 2300, HOUSTON, TX, 77002-6760, US

(73) Assignee: NSABP Foundation, Inc., Pittsburgh, PA (US)

(21) Appl. No.: 12/455,551

(22) Filed: Jun. 2, 2009

Related U.S. Application Data
(60) Provisional application No. 61/130,678, filed on Jun. 2, 2008.

(51) Int. Cl. A61K 39/395; C12Q 1/68

(52) U.S. Cl. 424/138.1; 435/6

(57) ABSTRACT

The present invention provides a method of screening for markers useful in predicting the efficacy of a specified cancer that includes: (a) constructing a tissue microarray from a tissue bank comprising multiple tissue samples that are annotated with clinical follow up data; (b) labeling polynucleic acid probes specific for oncogenes or cancer associated genes known to be potential amplicons; (c) performing fluorescent in situ hybridization analysis on the tissue microarray; and (d) correlating the result of the fluorescent in situ hybridization with the clinical follow up data. The present disclosure also provides methods of treating breast cancer that include screening a breast cancer patient for amplification of one or more of the genes disclosed herein, and treating a patient having amplification of one or more of these genes with a therapeutically effective amount of a compound that interferes with HER2 signaling.

### CROSS-REFERENCE TO RELATED APPLICATIONS

This application claims the benefit of U.S. Provisional Patent Application Ser. No. 61/130,678, filed on Jun. 2, 2008, which is incorporated herein by reference in its entirety.

### STATEMENT REGARDING FEDERALLY SPONSORED RESEARCH OR DEVELOPMENT

N/A

### THE NAMES OF PARTIES TO A JOINT RESEARCH AGREEMENT

N/A

### INCORPORATION-BY-REFERENCE OF MATERIAL SUBMITTED ON A COMPACT DISC

N/A

### BACKGROUND OF THE INVENTION

Breast cancer is a heterogeneous disease with respect to clinical behavior and response to therapy. This variability is a result of the differing molecular make up of cancer cells within each subtype of breast cancer. However, only two molecular characteristics are currently being exploited as therapeutic targets. These are estrogen receptor and HER2, which are targets of antiestrogens (tamoxifen and aromatase inhibitors) and HERCEPTIN® (trastuzumab), respectively. Efforts to target these two molecules have proven to be extremely productive. Nevertheless, those tumors that do not have these two targets are often treated with chemotherapy, which generally targets proliferating cells. Since some important normal cells are also proliferating, they are damaged by chemotherapy at the same time. Therefore, chemotherapy is associated with severe toxicity. Identification of molecular targets in tumors in addition to ER or HER2 is critical in the development of new anticancer therapy.

Recent studies using a combination of cDNA array based expression profiling and comparative genomic hybridization (“CGH”) have elucidated the role of gene amplification in the transcriptional program of breast cancer. In one study, copy number alteration and expression levels across 6691 mapped human genes were examined in 44 locally advanced breast cancer and 10 breast cancer cell lines (Pollack, et al., Proc. Natl. Acad. Sci. USA 99:12963-12968, 2002). The data from this study suggests that at least 12% of all the variation in gene expression among breast cancer is directly attributable to underlying variation in gene copy numbers. The total number of genomic alterations (gains and losses) correlated significantly with high grade (p=0.008), negative ER (P=0.04), and p53 mutation (p=0.0006). Of 117 high level amplifications (representing 91 different genes), 62% (representing 54 genes) were found to be associated with at least moderately elevated mRNA levels, and 42% (representing 36 different genes) with highly elevated mRNA levels. In another study, the correlation between copy number changes and expression levels was examined in 14 breast cancer cell lines using a cDNA microarray of 13,824 genes (Hyman, et al., Cancer Res. 62:6240-6245, 2002). The study found 44% of highly amplified genes resulted in overexpression, with 10.5% of overexpressed genes being amplified. Together these results indicate a profound role of gene amplification in transcriptional control of gene expression in breast cancer, and provide rationale for pursuing amplified genes as a preferred target for developing therapeutics and diagnostics.

Unfortunately, no study has correlated clinical outcome with a comprehensive list of amplified genes in breast cancer although amplification of a handful of genes has been identified by array CGH and have been examined by fluorescence in situ hybridization (“FISH”) and found to be prognostic. The biggest barrier for the screening of amplification pattern is the cost and need for high quality DNA for array CGH assays.

On the other hand, FISH is a stable method that works with formalin-fixed paraffin-embedded sections in a routine clinical setting. FISH probes for HER2 have been approved by the United States Food and Drug Administration (“FDA”) as a predictive test for response to HERCEPTIN®. Due to the stability of DNA in the paraffin-embedded sections, it is more reliable than RNA-based or immunohistochemistry-based clinical assays. However, FISH probes for potentially important amplified genes have not been comprehensively developed. In fact, there is only one vendor (Vysis, Incorporated, Downers Grove, Ill.) that supplies an array of probes, but most of these probes have not been clinically validated as prognostic factors. These probes are also very expensive (cost about $300 per case) and of limited variety, barely scratching the repertoire of potentially important amplicons in solid tumors such as breast and colon cancer.

In a recent survey of five Vysis supplied commercial FISH probes (HER2, MDM2, MYC, CCND1, EGFR) for potentially presumed important amplicons in breast cancer in 1100 cases, some but not all the five gene amplifications correlate with survival outcome in a poorly defined clinical cohort with no treatment information (Al-Kuraya, et al., Cancer Res. 64:8534-8540, 2004). Nevertheless, 60% of the cases did not have any amplification of the five genes examined. In addition, a gene amplification dosage effect was found in which survival rate was in the following order; no amplification>1 amplified>2 amplified>3 amplified. This data supports the so called “amplificatory” phenotype with an increased level of genomic instability and high likelihood for amplification development, and therefore supports the need for a comprehensive clinical correlation of amplicons in breast cancer.

Approximately 15 to 20% of all breast cancer has overexpression of HER2 protein on its cell surface (Paik, et al., J. Clin. Oncol. 8:103-112, 1990). Such tumors are known to have a worse prognosis than those without HER2 protein overexpression (Paik, et al., supra). Overexpression of HER2 protein is almost invariably due to amplification or increased copy number of the gene encoding HER2.

Multiple drugs have been developed to target HER2 signaling as means to stop growth of cancer cells that have overexpression of HER2 protein on its surface. One of these drugs is HERCEPTIN® (trastuzumab), developed by Genentech. HERCEPTIN® has recently been shown to be effective in prolonging survival in patients diagnosed with advanced breast cancer with HER2 overexpression (Slamon, et al., N. Engl. J. Med. 344:783-792, 2001). Recently HERCEPTIN® has also been shown to reduce recurrences and death in patients with early stage breast cancer which have HER2 protein overexpression or HER2 gene amplification (Romond, et al., N. Engl. J. Med. 353:1673-1684, 2005). The overall reduction in recurrence rate is about 50% with HERCEPTIN® when compared to chemotherapy alone in an adjuvant setting (Romond, et al., supra). Not all patients seem to gain benefit from this expensive treatment, which also has potential serious cardiotoxicity. A method to identify those patients who will benefit most from HERCEPTIN® or other HER2-targeting drugs is required (Slamon, et al., supra, Goldman, J. Natl. Cancer Inst. 95:1744-1746, 2003). Many laboratories have been pursuing abnormalities in components of the HER2 signaling pathway, such as PTEN, as predictors of response to HERCEPTIN®, with the hypothesis that such abnormalities will render tumor cells resistant to HERCEPTIN® even in the presence of HER2 protein overexpression (Crowder, et al., Cancer Cell 6:103-104, 2004, Nagata, et al., Cancer Cell 6:117-127, 2004). Such studies have concentrated only on molecules that may have a direct role in the HER2 signaling pathway>However, none have been substantiated in clinical studies, and there is no marker used for the prediction of response to HERCEPTIN® in clinical practice.

There are many genes that are amplified in breast cancer, as demonstrated by CGH studies. As noted above, about 10% of genes overexpressed in breast cancer are due to gene amplification (Pollack, et al., supra). One of the frequently amplified genes in human cancers is cMYC, which is located on chromosome 8. In normal cells, cMYC is expressed in a highly regulated manner driving cells from G1 to S phase. Perhaps due to its important role in normal cell proliferation, efforts to block cMYC has not been a major focus of the pharmaceutical industry, with only one company (Cylene Pharmaceuticals) currently having a drug undergoing clinical testing. Studies have suggested that cMYC has an important role as a molecular switch that determines the fate of the cell to go through programmed cell death or cell proliferation (Pelengaris, et al., Nat. Rev. Cancer 2:764-776, 2002, Pelengaris, et al., Cell 109:321-334, 2002). When cMYC is overexpressed, cells go into uncontrolled cell proliferation and become susceptible to programmed cell death in the absence of a survival signal. cMYC induces apoptosis by regulating many components of the programmed cell death pathway, but the main effector seems to be Bax (Pelengaris, et al., Nat. Rev. Cancer supra).

Eventually cells with cMYC overexpression will go through mass suicide due to the exhaustion of locally available survival factors. At the same time, cMYC overexpression has been shown to cause genomic instability. This could cause amplification of other oncogenes, such as HER2 (Fest, et al., Oncogene 21:2981-2990, 2002). Amplification of other genes could generate anti-apoptotic signals and therefore lead to the inhibition of the apoptotic pathway. For example, in the case of HER2 amplification, studies have demonstrated that HER2 induces Bcl-2, an anti-apoptotic protein that inhibits Bax (Milella, et al., Clin. Cancer Res. 10:7747-7756, 2004).

Therefore, a need remains to identify markers/genes that provide prognostic indicators of therapy efficacy.

### SUMMARY OF THE INVENTION

The present disclosure describes a number of genes that are predictors of response to HERCEPTIN® (trastuzumab) in an adjuvant setting. The disclosed genes thus predict the degree of benefit from trastuzumab added to adjuvant chemotherapy in cancer, and particularly breast cancer.

Furthermore, cMYC, is a predictor of response to HERCEPTIN®, in such a way that for patients with cMYC amplification together with HER2 amplification/overexpression, there is a 75% reduction in cancer recurrence rate when HERCEPTIN® is added to chemotherapy, compared to only 45% reduction in recurrence rate for those patients without cMYC amplification. cMYC is amplified in approximately 30% of the breast cancer patients with HER2 amplification or overexpression. Inhibition of HER2 signaling by Trastuzumab apparently changes the cMYC role from proliferation switch to pro-apoptotic switch. The invention has the following clinical applications: optimization of methods for patient selection and determining treatments using Trastuzumab and other drugs that target a HER2 signaling pathway: optimization of methods for patient selection for future clinical studies that test the addition of other drugs or targeted therapies, such as Bevacizumab (Avastin) that targets angiogenesis, by allowing identification of patients who are at high risk of relapse even after Trastuzumab or HER2 targeted therapy: PCR-based assay that will detect the gene amplification status of both HER2 and cMYC in a single tube assay for prognostication and prediction of response in breast cancer patients: and rational development of cMYC targeted therapy through indirect modulation of its pro-apoptotic activity by inhibiting anti-apoptotic signal from other activated oncogenes.

The present disclosure also describes a new prognostic and therapeutic target, the HTPAP gene, which when amplified confers poor prognosis in breast cancer patients even after treatment with standard chemotherapy containing doxorubicin, cyclophosphamide, and paclitaxel. HTPAP amplification is an independent prognosticator of tumor size, treatment, number of positive axillary lymph nodes, age and hormone receptor status, HER2 amplification, and cMYC amplification.

The present disclosure also provides methods for treating a patient having breast cancer, comprising measuring the expression level or amplification of one or more of the genes listed in Tables 5-14 in a patient having breast cancer, and providing a patient having increased expression level or amplification of one or more of the genes listed in Tables 5-14 with a therapeutically effective amount of a combination of one or more adjuvant chemotherapeutic compounds and at least one compound that inhibits the activity, amount, or signaling of HER2. In Tables 5-14, genes are referenced by a number of different methods, including gene symbol, genomic coordinates, and GenBank accession numbers. It is well-known to those of skill in the art that any of these identification methods is sufficient to identify a specific gene, and thus these identification methods are considered to be interchangable. Therefore, a skilled artisan provided with a GenBank accession number can readily determine the identity of the referenced gene utilizing the publicly available web site of the National Center for Biotechnology Information (“NCBI”).

Although increased expression or amplification of any of the genes listed in Tables 5-14 is predictive of the benefit of administration of a compound that inhibits the activity, amount, or signaling of HER2, in certain embodiments it may be useful to identify increased expression or amplification of a plurality of genes listed in Tables 5-14. Thus, in certain aspects, increased expression or amplification of two, three, four, five, six, seven, eight, nine, ten eleven, twelve, thirteen, fourteen, fifteen, sixteen, seventeen, eighteen, nineteen, twenty, twenty-five, thirty, thirty-five, forty, forty-five, or fifty genes or more listed in Tables 5-14 is indicative of a patient that would benefit from the administration of a compound that inhibits the activity, amount, or signaling of HER2 in an adjuvant chemotherapeutic setting. Thus, the present disclosure also provides assays that can be used to identify patients that would benefit from administration of a compound that inhibits the activity, amount, or signaling of HER2.

As disclosed herein, gene expression levels can be measured by any of the numerous methods known to those of skill in the art, including, but not limited to, an enzyme-linked immunosorbent assay, a radioimmunoassay, flow cytometry, or real time quantitative polymerase chain reaction assay. Furthermore, any method of measuring or determining gene amplification can be used with the disclosed methods, including, but not limited to, fluorescent in situ hybridization.

In certain embodiments, the compound that inhibits the activity, amount, or signaling of HER2 is an anti-HER2 antibody, a HER2 antisense molecule, a HER2 small inhibitory RNA molecule (“siRNA”), or a small molecule inhibitor of HER2. In embodiments wherein the compound that inhibits the activity, amount, or signaling of HER2 is an anti-HER2 antibody, the anti-HER2 antibody may be an anti-HER2 monoclonal antibody, including, but not limited to, trastuzumab.

The presently disclosed treatment methods can be utilized in conjunction with a single adjuvant chemotherapeutic compound, or a plurality of adjuvant chemotherapeutic compounds. Chemotherapeutic compounds that can be used in conjunction with the disclosed methods include, but are not limited to, doxorubicin, cyclophosphamide, and paclitaxel, and combinations thereof.

As disclosed herein, treatment of cancer patients with a combination of adjuvant chemotherapy and a compound that inhibits the activity, amount, or signaling of HER2 provides a significant benefit even to patients that do not have increased expression or amplification of the HER2 gene. However, in certain situations the patient can be screened for increased expression or amplification of the HER2 gene. However, it will be understood that the presently disclosed treatment methods will provide a benefit to patients that have increased expression or amplification of the HER2 gene, as well as patients that do not have increased expression or amplification of the HER2 gene.

The present disclosure also provides methods of identifying or diagnosing a patient with breast cancer undergoing adjuvant therapy that would benefit from administration of a compound that inhibits the activity, amount, or signaling of HER2, comprising measuring the expression level or amplification of one or more of the genes listed in Tables 5-14 in the patient, wherein an increased expression level or amplification of one or more of the genes listed in Tables 5-14 is indicative of a patient with breast cancer undergoing adjuvant therapy that would benefit from administration of a compound that inhibits the activity, amount, or signaling of HER2. Once again, although such patients can also be screened for increased expression or amplification of the HER2 gene, administration of a compound that inhibits the activity, amount, or signaling of HER2 will benefit patients that have increased expression or amplification of the HER2 gene, as well as patients that do not have increased expression or amplification of the HER2 gene.

As used herein, and unless otherwise indicated, the terms “treat,” “treating,” and “treatment” contemplate an action that occurs while a patient is suffering from a disease or disorder, that reduces the severity of one or more symptoms or effects of the disease or disorder, or a related disease or disorder. As used herein, and unless otherwise indicated, the terms “manage,” “managing,” and “management” encompass preventing, delaying, or reducing the severity of a recurrence of a disease or disorder in a patient who has already suffered from the disease or disorder. The terms encompass modulating the threshold, development, and/or duration of the disease or disorder, or changing the way that a patient responds to the disease or disorder.

As used herein, and unless otherwise specified, a “therapeutically effective amount” of a compound is an amount sufficient to provide any therapeutic benefit in the treatment or management of a disease or disorder, or to delay or minimize one or more symptoms associated with a disease or disorder. A therapeutically effective amount of a compound means an amount of the compound, alone or in combination with one or more other therapy and/or therapeutic agent, which provides any therapeutic benefit in the treatment or management of a disease or disorder, or related diseases or disorders. The term “therapeutically effective amount” can encompass an amount that cures a disease or disorder, improves or reduces a disease or disorder, reduces or avoids symptoms or causes of a disease or disorder, improves overall therapy, or enhances the therapeutic efficacy of another therapeutic agent.

### DESCRIPTION OF THE INVENTION

One reason for the high cost of commercially available FISH probes is the cost and difficulty of directly fluorescence labeling bacterial artificial clones (“BAC”) representing the probes. This disclosure provides a method for fluorescently labeling BAC clones representing known amplicons efficiently by combining a series of whole genome amplification methods and an efficient FISH method for paraffin embedded tissue which has been archived more than 10 years. Briefly, the literature and array CGH data is reviewed, and candidate amplicons (˜50) are selected. BAC clones from public sources that correspond to the candidate amplicons are obtained and labeled for FISH analysis of tissue microarrays (“TMAs”) constructed from a tissue bank containing over 30,000 samples created from National Surgical Adjuvant Breast and Bowel Project (“NSABP”) trials. The data is used for clinical correlation and model building, and validated using an independent data set TMA from NSABP.

This labeling and FISH method is a log order less expensive as compared to commercially available probes. Using paraffin block tissue samples for over 30,000 breast and colon cancer cases that are all annotated with clinical follow-up information and treatment received provided a unique source for clinical correlative science studies. Combining the FISH method with tissue microarrays allows screening of more than 100 cases using a single microscopic section, making screening of multiple amplicons in thousands of cases a reality. One of ordinary skill in the art will readily recognize that any number of methods well-known in the art can be used to label probes for FISH applications. Furthermore, because FISH is used to determine amplification, numerous other quantitative or semi-quantitative methods may be used, including, but not limited to, antibody based assays (such as ELISA (enzyme-linked immunosorbent assay)) and qtPCR.

### Pilot Project:

In a pilot demonstration project, more than 987 cases from the NSABP trial B-28 were screened comparing 4 cycles of arimycin (doxorubicin) plus cyclophosphamide (“AC”) versus 4 cycles of AC followed by four cycles of TAXOL® (paclitaxel) (“ACT”). In this study, tissue microarrays were constructed and FISH assays performed for 10 different in-house developed probes based on array CGH data (two sets are very close to each other: HER2 and MLN64; and APPBP2 and PPM1D). The amplicons and their chromosomal locations are shown in Table 1.

TABLE 1

| Amplicon | Chromosomal Location |
|---|---|
| SEB4D | 20q13.32 |
| ZNF217 | 20q13.2 |
| APPBP2 | 17q23.2 |
| TPD52 | 8q21 |
| MLN64 | 17q11-q12 |
| PPM1D | 17q23.2 |
| HER2 | 17q21.1 |
| CYCLIND1 | 11q13 |
| MAL2 | 8q23 |
| C-MYC | 8q24.12-q24.13 |

After hybridization of individual probes, cases were scored as either amplified (if signal more than 3 copies per nuclei) or not-amplified (2 copies or less). In order to find the natural class of amplification patterns of these 10 amplicons, non-supervised hierarchical clustering was performed. The results of the pilot study are shown in Table 2.

TABLE 2

| Amplicon 1 | Amplicon 2 | Correlation Score |
|---|---|---|
| PPM1D | APPBP2 | 1.0 |
| MLN64 | HER2 | 0.947 |
| cMYC | MAL2 | 0.522 |
| SEB4D | ZNF217 | 0.505 |
| TPD52 | MAL2 | 0.401 |
| cMYC | TPD52 | 0.378 |
| MLN64 | APPBP2 | 0.333 |
| HER2 | APPBP2 | 0.333 |
| cMYC | HER2 | 0.262 |
| cMYC | MLN64 | 0.260 |
| Cyclin D1 | APPBP2 | 0.228 |

What is notable in the result is the close correlation of the amplification status of PPM1D and APPBP2, and HER2 and MLN64, as expected based on their very close proximity in their chromosomal location. This data shows that the disclosed methods for BAC labeling leads to highly reproducible results.

In addition, there are cases with no amplification of any of the 10 amplicons. While the proportion of such cases will decrease as more amplicons are screened, it is likely that such subgroups do exist that are relatively resistant to amplification.

The prognostic value of non-amplification versus any amplification in B-28 according to treatment was examined. Recurrence-free survival of those patients with no amplification of any of the 10 amplicons were significantly better than those with amplification of any of the genes, while as expected from the nature of the genes in the 10 selected amplicons in this pilot there was no interaction with the benefit from adding taxol to AC based on amplification phenotype in general in this protocol.

Additionally, 27 candidate amplicons that are associated with overexpression were systematically screened using a univariate COX proportional hazard model for each amplicon. Also included is the presence of any amplification and number of amplification. The results are shown in Table 3.

TABLE 3

| | | | ChiSq | | Hazard | Lower | Upper |
|---|---|---|---|---|---|---|---|
| Amplicons | Estimate | StdErr | score | p value | Ratio | CL | CL |
| any_amplicons | 0.62392 | 0.09873 | 41.234 | <0.0001 | 1.866 | 1.538 | 2.265 |
| HER2 | 0.54108 | 0.11464 | 22.8233 | <0.0001 | 1.718 | 1.372 | 2.151 |
| MLN64 | 0.55419 | 0.11773 | 22.7323 | <0.0001 | 1.741 | 1.382 | 2.192 |
| number_amplicons | 0.07627 | 0.01613 | 22.4652 | <0.0001 | 1.079 | 1.046 | 1.114 |
| cMYC | 0.5156 | 0.12165 | 18.3631 | <0.0001 | 1.675 | 1.319 | 2.126 |
| HTPAP | 0.48668 | 0.14443 | 11.5791 | 0.0007 | 1.627 | 1.226 | 2.159 |
| TPD52 | 0.55161 | 0.22118 | 6.3773 | 0.0116 | 1.736 | 1.125 | 2.678 |
| MAL2 | 0.44767 | 0.1837 | 6.0382 | 0.014 | 1.565 | 1.092 | 2.243 |
| ZNF217 | 0.37832 | 0.15995 | 5.6609 | 0.0173 | 1.46 | 1.067 | 1.197 |
| NCOA3 | 0.45526 | 0.23643 | 3.7721 | 0.0521 | 1.577 | 0.992 | 2.506 |
| ZHX1 | 0.39753 | 0.21037 | 3.6178 | 0.0572 | 1.488 | 0.985 | 2.248 |
| BM_009 | 0.30628 | 0.16418 | 3.4934 | 0.0616 | 1.358 | 0.985 | 1.874 |
| BMP7 | 0.32718 | 0.18117 | 3.2904 | 0.0697 | 1.387 | .0972 | 1.978 |
| STK6 | 0.4405 | 0.26498 | 2.8085 | 0.0938 | 1.553 | 0.924 | 2.611 |
| SEB4D | 0.29955 | 0.19145 | 2.4536 | 0.1173 | 1.349 | 0.927 | 1.964 |
| DECR1 | 0.49841 | 0.32199 | 2.4457 | 0.1179 | 1.646 | 0.876 | 3.094 |
| CACNB1 | 0.27966 | 0.18937 | 2.1866 | 0.1392 | 1.323 | 0.913 | 1.917 |
| TRAF4 | 0.34353 | 0.23789 | 2.1059 | 0.1467 | 1.41 | 0.885 | 2.247 |
| HBS1L | 0.47545 | 0.38277 | 1.572 | 0.2099 | 1.609 | 0.76 | 3.406 |
| GRB2 | 0.2947 | 0.26481 | 1.2395 | 0.2656 | 1.343 | 0.799 | 2.256 |
| PPM1D | 0.13435 | 0.16327 | 0.6779 | 0.4103 | 1.144 | 0.831 | 1.575 |
| SIP2-28 | −0.25338 | 0.38169 | 0.4442 | 0.5051 | 0.776 | 0.367 | 1.64 |
| APPBP2 | 0.10275 | 0.16151 | 0.405 | 0.5245 | 1.108 | 0.808 | 1.521 |
| MRPS7 | −0.10766 | 0.29402 | 0.1342 | 0.7141 | 0.898 | 0.505 | 1.598 |
| GRO1 | −0.14334 | 0.451 | 0.1012 | 0.7503 | 0.866 | 0.358 | 2.097 |
| RPS6KB1 | 0.04841 | 0.17472 | 0.0768 | 0.7817 | 1.05 | 0.745 | 1.478 |
| FLJ10783 | 0.05525 | 0.2567 | 0.0463 | 0.8296 | 1.057 | 0.639 | 1.748 |
| CHK | −0.05045 | 0.23673 | 0.0454 | 0.8312 | 0.951 | 0.598 | 1.512 |
| Cyclin D1 | 0.0266 | 0.13602 | 0.0376 | 0.8463 | 1.027 | 0.787 | 1.341 |

The results show that three amplicons (HER2, cMYC, and HTPAP, which is also known as PPAPDC1B) were identified that are independently prognostic in node-positive breast cancer treated with standard chemotherapy when they are tested in multivariate analysis including other prognostic variables. These three amplicons were identified using the following BACs: HER2-PathVysion HER2 Assay (Vysis, Incorporated); cMYC-LSI C-MYC (Vysis, Incorporated); and HTPAP-RP11-513D5. Nevertheless, one of ordinary skill in the art would readily recognize multiple other probe sources for the same genes can be utilized with this invention. One of ordinary skill in the art would readily recognize multiple other method of labeling any probe sources for the same genes can be utilized with this invention. These could include both fluorogenic and chromogenic probe labeling methods.

These 27 amplicons were screened by FISH on TMA constructed from a NSABP trial B-28, in which auxiliary node-positive breast cancer patients were randomly assigned to receive 4 cycles of AC or 4 cycles of AC followed by 4 cycles of TAXOL® (paclitaxel) (N=1901). This means that approximately 51,327 FISH assays were performed (27×1901). Selection of the 27 amplicons was based on the following criteria: 1) selected amplicons had all been shown to be associated with moderate to high level of gene expression of the coded genes when amplified in breast cancer tumors or cell lines in studies mentioned previously (Pollack, et al., supra, and Hyman, et al., supra); 2) the public genome sequence map was examined and FISH-validated BAC clones were selected that corresponded best with the selected amplicons; and 3) some amplicons, such as MLN64, which were located very close to HER2, were included as an internal control for reproducibility and validity of the assay (that is, HER2 and MLN64 amplification were expected to correlate extremely tightly due to their close proximity in chromosome location).

Amplification status was categorized as either amplified or non-amplified, with gene amplification defined as having more than 4 signals (4 dots per single tumor cell nucleus) from in situ hybridization. Correlation with clinical outcome using univariate Cox proportional hazard model showed that HER2, MLN64 (which is very close to HER2 and highly correlated), cMYC, HTPAP, TPD52, MAL2, and ZNF217 are significantly correlated with clinical outcome of patients entered into the B-28 trial (Table 3). In addition, the presence of any amplification and number of amplifications also showed significant correlation with outcome.

Multivariate analysis including conventional prognostic markers (tumor size, number of positive nodes, hormone receptor status, and age) was performed. Three amplicons remained significant: HER2; cMYC; and HTPAP (Table 4).

TABLE 4

| Variable | Chi Square | P Value | Hazard Ratio | Lower CL | Upper CL |
|---|---|---|---|---|---|
| Treatment (AC vs. ACT) | 2.7084 | 0.0998 | 0.854 | 0.708 | 1.031 |
| Positive Node (4-9) | 33.6884 | <0.0001 | 1.844 | 1.500 | 2.268 |
| Positive Node (≧10) | 34.2087 | <0.0001 | 2.852 | 2.008 | 4.053 |
| Medium Tumor Size | 2.2142 | 0.1367 | 1.169 | 0.952 | 1.436 |
| Large Tumor Size | 14.8289 | 0.0001 | 1.761 | 1.320 | 2.348 |
| Young_ER_Negative | 30.8256 | <0.0001 | 2.131 | 1.631 | 2.783 |
| Old_ER_Positive | 3.8946 | 0.0484 | 0.775 | 0.602 | 0.998 |
| Old_ER_Negative | 7.5734 | 0.0059 | 1.499 | 1.124 | 2.000 |
| Intermediate_Grade | 5.8421 | 0.0156 | 1.898 | 1.129 | 3.192 |
| Poor_Grade | 8.532 | 0.0035 | 2.161 | 1.289 | 3.624 |
| Missing_Grade | 2.0411 | 0.1531 | 1.653 | 0.830 | 3.293 |
| HER2 | 3.4508 | 0.0663 | 1.245 | 0.988 | 1.569 |
| cMYC | 4.7202 | 0.0298 | 1.307 | 1.027 | 1.665 |
| HTPAP | 14.2542 | 0.0002 | 1.726 | 1.300 | 2.292 |

The results of this study showed that HER2, cMYC, and HTPAP are three independent amplified genes that confer a worse prognosis, even after standard combination taxane-containing adjuvant chemotherapy. Furthermore, cases that had co-amplification of HER2 and cMYC had a much worse prognosis than cases with amplification of either one of the genes alone.

### HTPAP:

HTPAP is a novel gene that translates into a protein with a phosphatidic acid phosphatase homology domain and a 5′ transmembrane domain, as well as a signal peptide that indicates that the protein product is secreted. The BAC clone used for generation of FISH probe for HTPAP (clone RP11-513D5) has only three genes in it: HTPAP; WHSC1L1; and DDHD2. Of these, other studies correlating gene amplification with expression in breast cancer cell lines have shown that HTPAP is the one that is overexpressed when this region is amplified (Pollack, et al., supra, Hyman, et al., supra, Ray, et al., Cancer Res. 64:40-47, 2004). In a review of data from microarray analysis of gene expression in breast cancer, it was reported that HTPAP overexpression is associated with poor prognosis of patients with breast cancer, together with 94 other genes (Jenssen, et al., Hum. Genet. 111:411-420, 2002). These results demonstrate that amplification of the HTPAP gene is an independent prognosticator for breast cancer even after treatment with standard chemotherapy.

While amplification and overexpression of HTPAP in a limited number of breast cancers with 8p11-12 amplification has been described by other investigators, these studies have not pinpointed HTPAP as the main driver gene in those amplifications, since there are other genes that are overexpressed from the region of amplification. By taking advantage of the use of relatively small FISH probes containing only three genes in which HTPAP is the only overexpressed gene, and screening of large number of cases with defined treatment from a single prospective clinical trial, this disclosure is the first to demonstrate the role of HTPAP as a prognostic factor independent of other prognosticators in breast cancer. Since it is amplified and correlated with poor prognosis even after standard chemotherapy, HTPAP is also an important therapeutic target for breast cancer.

The following characteristics of HTPAP make it an ideal therapeutic and diagnostic target in breast cancer: 1) HTPAP is amplified, and stable clinical diagnostic assay using FISH or PCR can be used to detect the amplification status; 2) it is an independent prognostic factor in heavily treated patients; 3) it is a transmembrane protein with enzyme activity; and 4) it is also secreted. The amplification of HTPAP being highly correlated with poor prognosis indicates that blocking of these activities will have beneficial therapeutic effects (as exemplified by the HER2 gene, which has similar characteristics of being amplified, a prognostic factor, and a cell surface receptor).

Certain embodiments of the present invention include monoclonal antibodies or series of monoclonal antibodies with specificity for the extracellular domain of the HTPAP protein. These antibodies can be used either alone or in combination with chemotherapeutic drugs or antibodies to other targets. The generation of such antibodies can be performed via any number of methods for monoclonal production which are well known in the art.

In certain embodiments of the present invention, these anti-HTPAP antibodies are used to detect HTPAP protein secreted in the serum, plasma, or other body fluid (such as nipple aspirate from the patients), and compared to normal levels in the diagnosis or monitoring of disease during therapy. Detection may be accomplished by any number of methods well known in the art, including, but not limited to, radioimmunoassay, flow cytometry, ELISA, or other colormetric assays.

Phosphatidic acid phosphatase domains typically act as an important signaling molecule in cancer cells. Certain embodiments of the present invention include the use of these domains of the HTPAP gene in targeting and development of small molecules that interfere or modulate such activity. Furthermore, the use of antibodies to HTPAP can be used to identify signaling molecules downstream to HTPAP, which can be subsequently targeted by small molecule therapeutics. Certain other embodiments include blocking HTPAP gene activity using siRNA, antisense oligonucleotide, or ribozyme approaches, which are well known in the art.

Thus, the present disclosure provides methods of treating breast cancer that include measuring the expression levels or amplification of HTPAP in a patient having breast cancer and then providing a patient having increased levels of HTPAP expression or HTPAP amplification with therapeutic quantities of at least one compound that interferes with the phosphatidic acid phosphatase activity of HTPAP.

Other genes found to be of marginal prognostic power in this study cohort of AC- or ACT-treated node positive breast cancer may have significant prognostic power in untreated or node negative patients. These include TPD52, MAL2, ZNF217, NCOA3, ZHX1, BM_{—}009, BMP7, and STK6, and thus these genes may also provide attractive target for therapeutic development. In certain embodiments of the present invention, three prognostic amplified genes (HER2, cMYC, and HTPAP) can be utilized to create a prognostic index to guide treatment decision making for breast cancer patients. Certain other embodiments include HER2, cMYC, and HTPAP together with clinical variables to generate a prognostic index to guide treatment decision making.

cMYC Predictor:

Cells primed for malignant transformation by cMYC amplification seem to be able to escape the fate of apoptosis with the help of HER2 amplification, however, it is believed that this also makes them dependent on HER2 signaling to survive. Therefore inhibition of the HER2 signal by trastuzumab could trigger the pro-apoptotic function of cMYC in such cancer cells. This was verified in a retrospective analysis of tumor specimens collected as part of the NSABP trial B-31, in which patients diagnosed with HER2 overexpressing tumors were randomized to receive chemotherapy or chemotherapy plus HERCEPTIN® (trastuzumab). The results of this analysis clearly demonstrated that tumors with co-amplification of both the HER2 and cMYC genes are sensitive to trastuzumab.

The status of cMYC in 1344 patients enrolled in the NSABP B-31 trial were examined to test the potential benefits of addition of trastuzumab to chemotherapy in the treatment of patients diagnosed with early stage breast cancer with HER2 gene amplification/overexpression. FISH was used to enumerate the cMYC gene copy number using a commercially available DNA probe (Vysis, Incorporated). Any tumor with more than 10% of cells showing more than 4 copies of cMYC gene was classified as cMYC gene amplified in this analysis. Out of a total of 1344 cases studied, 399 cases were classified as cMYC amplified. Tumors with cMYC amplification were believed to be sensitive to inhibition of HER2 signaling due to its activation of a pro-apoptotic signal when the HER2 signal is inhibited by trastuzumab, and that this would translate into a much more significant reduction in recurrence rate in cMYC amplified cohort in comparison to patients with no amplification of cMYC.

Recurrence-free survival of patients from the B-31 trial according to cMYC amplification status was investigated. In patients with no amplification of the cMYC gene (N=945), there was a 34% reduction in recurrence rate when trastuzumab was added to chemotherapy (p=0.02). On the other hand, in patients with cMYC amplification (N=399), there was a 74% reduction in recurrence rate when trastuzumab was added to chemotherapy (p<0.0001). The P-value for the interaction test (to determine if the difference between the two cohorts is statistically meaningful) was 0.014, thus verifying the cMYC by trastuzumab interaction. In spite of starting with a very poor prognosis (as detailed above, cases that had co-amplification of HER2 and cMYC had a much worse prognosis than cases with amplification of either one of the genes alone), patients with tumors that have co-amplification of HER2 and cMYC end up enjoying near cure of their disease with trastuzumab plus chemotherapy.

Although trastuzumab does not cure all HER2 overexpressing tumors, strategies to add other targeted therapies such as an inhibitor of angiogenesis may be useful. However, such an approach is highly toxic and very expensive. Patients undergoing chemotherapy having amplification of cMYC should not need additional therapy (other than trastuzumab), due to their sensitivity to trastuzumab. Therefore, one invention of the present disclosure is the screening of patients for approaches that add other targeted therapies to trastuzumab. Furthermore, the present disclosure includes a method of determining the cMYC and HER2 amplification status of cancer patients. The present disclosure is also applicable to other HER2-targeted therapies, since the effect is an indirect one through activation of the pro-apoptotic role of cMYC. In other words, the invention disclosed herein includes methods of determining treatments and treating patients with trastuzumab and other materials based on the status of cMYC and HER2 in a patient.

In other embodiments, the present invention can be applied in exploiting the pro-apoptotic function of cMYC in cMYC-amplified tumors without HER2 amplification. Instead of directly inhibiting cMYC activity, indirect approaches inhibiting survival signals will likely make such tumors go through programmed cell death by activation of the pro-apoptotic function of cMYC.

The test for cMYC in the present disclosure can be either in the format of FISH, quantitative polymerase chain reaction, immunohistochemistry, or other immunological detection method in homogenized tumor tissue, including a single tube, “real-time” quantitative polymerase chain reaction (“qtPCR”) assay that includes HER2, cMYC, HTPAP, and a reference gene to simultaneously detect the presence of amplification of these three genes and provide both prognostic information as well as prediction of response to trastuzumab or other HER2-targeted therapies, as well as assays and methods of treating a patient based on the results of such an assay.

Discovery and Refinement of Genes that Predict the Degree of Benefit from Trastuzumab Added to Adjuvant Chemotherapy in Breast Cancer:

The NSABP B-31 trial compared the benefit of adding trastuzumab to standard adjuvant ACT chemotherapy (Romond, et al., supra). While all of the originally intended study population was supposed to be positive for HER2 gene amplification or HER2 protein overexpression, about 10% of them were HER2 negative. However, the HER2 negative population still gained significant clinical benefit from adding trastuzumab to standard adjuvant ACT chemotherapy. Thus, HER2 gene copy number was not predictive of the degree of benefit from trastuzumab. This data suggested that there should be other molecular markers that dictate the response to trastuzumab.

Tumor blocks were submitted for 1829 of the 2043 patients enrolled in the NSABP B-31 trial, all of whom had provided informed consent. Among these, 1795 had available information on clinical follow-up, number of positive nodes, and estrogen receptor status of the tumors. In order to further elucidate the mechanism of action of trastuzumab in an adjuvant setting and to develop a predictive algorithm, microarray gene expression analyses of the formalin fixed paraffin embedded tumor blocks are being carried out in a three step effort: 1) discover predictive genes; 2) refine the gene list in an independent cohort; and 3) final prospective testing of a predictive model in yet another independent cohort.

From the 1795 cases with tumor blocks, two cohorts of 400 cases each were randomly selected to perform microarray gene expression analyses using Affymetrix GeneChip U133 plus 2.0 and Agilent 4×44 human genome expression arrays. In the first cohort, microarray expression analyses were performed with both Affymetrix and Agilent arrays, and in the second cohort only the Agilent array was used.

Total RNA extracted from paraffin blocks were amplified and hybridized to Agilent 4×44 arrays. A commercial kit was used for RNA extraction (Ambion) and RNA was amplified with Transplex whole transcriptome amplification kit (Rubicon Genomics). The resulting amplified cDNA was labeled with either biotin or cy-3 fluorescence dye before hybridizing to an Affymetrix or Agilent microarray. The hybridization signals were obtained by scanning with scanners from Affymetrix and Agilent respectively. The raw data was processed and normalized with commercially available software Partek Genomic Suite (Partek). Expression levels for each gene probe were dichotomized using median cut, hazard ratios for treatment arms were calculated, and interaction tests were performed.

The normalized expression data for each gene was correlated with clinical outcome to test for prediction of degree of benefit from adding trastuzumab to chemotherapy using Cox proportional hazard model. The measure of prediction was expressed in “p-value for interaction.” Any gene that showed p-value for interaction below 0.05 was regarded as significantly predictive of the degree of benefit from trastuzumab. Since studies with a sample size of 400 could be underpowered to detect interaction at a significance level of 0.05 even if there is true interaction, when looking for genes that are predictive in both cohorts, genes that are significant in one cohort at 0.05 level and at 0.1 level in the other cohorts were noted. Genes that are significant at 0.05 level in all three experiments provided the set of genes with the most confidence.

The results of the analyses are listed in Tables 5-11. Table 5 shows the results from the Agilent Second Cohort.

TABLE 5

| | | | p-value for |
|---|---|---|---|
| ProbeID | Gene Symbol | Genomic Coordinates | interaction |
| A_23_P208090 | MBD1 | chr18: 46049374-46049315 | 0.000004 |
| A_24_P43876 | C9orf40 | chr9: 76752674-76752615 | 0.000023 |
| A_32_P201521 | TMEM97 | chr17: 23677865-23677924 | 0.000267 |
| A_23_P354929 | CHD9 | chr16: 51837253-51838754 | 0.000268 |
| A_23_P14798 | NEO1 | chr15: 71382673-71382732 | 0.000382 |
| A_23_P257924 | ETS2 | chr21: 39117433-39117492 | 0.000467 |
| A_24_P33552 | LOC442260 | chr6: 133360475-133360416 | 0.000493 |
| A_32_P142149 | | chr5: 37322394-37322335 | 0.000529 |
| A_24_P854964 | | chr17: 24899003-24899062 | 0.000565 |
| A_32_P34149 | HNRPH1 | chr5: 178974309-178974250 | 0.000598 |
| A_24_P244100 | DNAJB12 | chr10: 73762891-73762832 | 0.000621 |
| A_24_P78862 | SLC43A2 | chr17: 1440886-1436077 | 0.000670 |
| A_32_P45894 | DKFZP434A0131 | chr7: 74834197-74834256 | 0.000686 |
| A_24_P152635 | TXNDC14 | chr11: 57264370-57264429 | 0.000728 |
| A_24_P401013 | | chr3: 1612721-1612662 | 0.000734 |
| A_24_P317762 | LY6E | chr8: 144174191-144174382 | 0.000800 |
| A_23_P4353 | WSB1 | chr17: 22663795-22663854 | 0.000831 |
| A_23_P71014 | WBSCR22 | chr7: 72745670-72746260 | 0.000833 |
| A_23_P12784 | FRAT2 | chr10: 99082454-99082395 | 0.000835 |
| A_23_P130089 | IFT20 | chr17: 23681677-23681618 | 0.001008 |
| A_24_P418878 | ZNF174 | chr16: 3394877-3394937 | 0.001097 |
| A_23_P68717 | DSCR2 | chr21: 39469330-39469271 | 0.001099 |
| A_23_P66698 | SLC46A1 | chr17: 23749924-23749865 | 0.001112 |
| A_23_P137143 | DKC1 | chrX: 153658781-153658840 | 0.001329 |
| A_23_P78122 | MYO18A | chr17: 24425005-24424946 | 0.001359 |
| A_23_P6481 | TNRC6B | chr22: 39049678-39049737 | 0.001606 |
| A_23_P127231 | FRAT1 | chr10: 99070217-99070277 | 0.002029 |
| A_24_P336754 | MCL1 | chr1: 148813911-148813852 | 0.002030 |
| A_23_P30024 | NFKB1 | chr4: 103756902-103756961 | 0.002050 |
| A_24_P683011 | | chr8: 92040415-92040356 | 0.002074 |
| A_23_P380857 | APOL4 | chr22: 34915888-34915829 | 0.002120 |
| A_32_P52046 | | chr10: 97918034-97918093 | 0.002265 |
| A_23_P55706 | RELB | chr19: 50233225-50233284 | 0.002353 |
| A_24_P392436 | | chr16: 002912909-002912850 | 0.002370 |
| A_24_P317637 | ZKSCAN5 | chr7: 98968033-98968092 | 0.002376 |
| A_24_P171873 | FBXO4 | chr5: 41970094-41970153 | 0.002549 |
| A_24_P392351 | | chr7: 007748327-007748268 | 0.002595 |
| A_24_P911973 | | | 0.002600 |
| A_24_P273635 | HEL308 | chr4: 84593538-84593479 | 0.002630 |
| A_23_P49842 | UNC119 | chr17: 23898991-23898932 | 0.002651 |
| A_24_P917344 | | chr17: 23753487-23753428 | 0.002760 |
| A_23_P365719 | TAPBP | chr6: 33375755-33375696 | 0.002768 |
| A_24_P276853 | GON4L | chr1: 153987058-153986999 | 0.002799 |
| A_23_P126735 | IL10 | chr1: 205008076-205008017 | 0.002840 |
| A_32_P191696 | | chr14: 049122502-049122796 | 0.002989 |
| A_24_P383076 | AASDH | chr4: 56910276-56910217 | 0.003047 |
| A_32_P128097 | | chr20: 030909905-030909846 | 0.003094 |
| A_23_P49327 | ZNF174 | chr16: 3398990-3399049 | 0.003166 |
| A_32_P38806 | | chr19: 041822728-041822787 | 0.003175 |
| A_32_P855872 | | chr11: 109731385-109731444 | 0.003184 |
| A_24_P936671 | CCT8 | chr21: 29366812-29366753 | 0.003203 |
| A_32_P101031 | LYPD1 | chr2: 133118960-133118901 | 0.003214 |
| A_23_P200203 | ECHDC2 | chr1: 53134552-53134493 | 0.003307 |
| A_24_P671875 | | chr20: 29716636-29716577 | 0.003346 |
| A_23_P60225 | GRHPR | chr9: 37419806-37420539 | 0.003372 |
| A_23_P1280 | ASCC1 | chr10: 73527067-73527008 | 0.003425 |
| A_23_P160881 | SMPDL3B | chr1: 28154961-28155104 | 0.003442 |
| A_23_P62361 | UBL4A | chrX: 153367124-153367065 | 0.003464 |
| A_32_P78681 | GLP2R | chr17: 9736065-9736124 | 0.003480 |
| A_23_P251855 | SLC12A7 | chr5: 1105147-1105088 | 0.003504 |
| A_23_P311740 | PARC | chr6: 43300223-43300282 | 0.003512 |
| A_23_P89283 | PITPNA | chr17: 1370471-1370412 | 0.003680 |
| A_32_P108544 | RGPD1 | chr2: 87078358-87093171 | 0.003703 |
| A_24_P67699 | RPL23A | chr17: 24074717-24074776 | 0.003732 |
| A_24_P401521 | | chr7: 006724586-006724534 | 0.003760 |
| A_23_P501183 | NSUN5C | chr7: 72056825-72056766 | 0.003774 |
| A_24_P456468 | | chr17: 42755764-42755705 | 0.003777 |
| A_23_P202100 | | | 0.003845 |
| A_23_P103787 | HIPK1 | chr1: 114317812-114317871 | 0.003918 |
| A_32_P20582 | VPS13D | chr1: 12491788-12491847 | 0.003921 |
| A_23_P102471 | MSH2 | chr2: 47563665-47563724 | 0.003951 |
| A_23_P47058 | CUZD1 | chr10: 124581865-124581806 | 0.003961 |
| A_23_P69877 | ZFP62 | chr5_random: 12747-12688 | 0.003977 |
| A_32_P179676 | TOB2 | chr22: 40159750-40159691 | 0.003977 |
| A_24_P383130 | hCG_1642354 | chr12: 2761565-2761624 | 0.004045 |
| A_24_P557019 | | chr19: 18339451-18339392 | 0.004086 |
| A_23_P31372 | MDH2 | chr7: 75532132-75532191 | 0.004119 |
| A_24_P392863 | | chr7: 99874256-99874315 | 0.004188 |
| A_32_P158536 | | chr17: 24137598-24137539 | 0.004229 |
| A_24_P115443 | BIN3 | chr8: 22537399-22534994 | 0.004241 |
| A_24_P107142 | ANXA11 | chr10: 81904945-81904886 | 0.004242 |
| A_24_P341176 | | chr10: 108300056-108300115 | 0.004259 |
| A_23_P150267 | BBS1 | chr11: 66057430-66057489 | 0.004274 |
| A_23_P161125 | MOV10 | chr1: 113042606-113042901 | 0.004309 |
| A_32_P129660 | GCC2 | chr2: 108482597-108490461 | 0.004371 |
| A_24_P121171 | MIZF | chr11: 118510354-118510413 | 0.004398 |
| A_23_P15654 | TRAF4 | chr17: 24100665-24100724 | 0.004440 |
| A_23_P141555 | TBX21 | chr17: 43178388-43178447 | 0.004484 |
| A_24_P295330 | C9orf97 | chr9: 99406918-99406859 | 0.004525 |
| A_23_P27460 | | | 0.004551 |
| A_32_P178842 | | chr1: 036310024-036310083 | 0.004568 |
| A_24_P938433 | | chr3: 144313823-144313882 | 0.004603 |
| A_24_P336551 | BGLAP | chr1: 154479679-154479736 | 0.004643 |
| A_23_P123265 | SUMF2 | chr7: 56115173-56115232 | 0.004656 |
| A_32_P73217 | C1orf119 | chr1: 109440762-109440821 | 0.004658 |
| A_23_P111929 | OTUD6B | chr8: 92159866-92159925 | 0.004681 |
| A_24_P353732 | SPEN | chr1: 16136930-16136989 | 0.004784 |
| A_23_P120784 | HTF9C | chr22: 18479703-18479644 | 0.004787 |
| A_24_P59239 | | chrX: 014762751-014762686 | 0.004819 |
| A_24_P322215 | DHRS13 | chr17: 24249769-24249710 | 0.004860 |
| A_24_P135748 | GRTP1 | chr13: 113047218-113028388 | 0.004905 |
| A_24_P700052 | | chr17: 23968439-23968498 | 0.004916 |
| A_23_P70007 | HMMR | chr5: 162851415-162851474 | 0.004931 |
| A_23_P111698 | MAFK | chr7: 1546815-1546874 | 0.004976 |
| A_24_P48014 | SOCS1 | chr16: 11256503-11256444 | 0.004992 |
| A_23_P141606 | BLMH | chr17: 25599533-25599474 | 0.005032 |
| A_24_P185205 | TP53I13 | chr17: 24923471-24923530 | 0.005064 |
| A_32_P72652 | | chr15: 49183054-49182995 | 0.005193 |
| A_23_P432512 | PPIL2 | chr22: 20381823-20381882 | 0.005201 |
| A_23_P98092 | OAT | chr10: 126076306-126076247 | 0.005255 |
| A_23_P208393 | | chr19: 46518286-46518345 | 0.005298 |
| A_24_P632160 | | chr5: 178357888-178357830 | 0.005301 |
| A_24_P93425 | LOC730663 | chr1: 210549183-210549124 | 0.005349 |
| A_23_P202408 | HERC4 | chr10: 69384422-69384363 | 0.005362 |
| A_23_P154025 | SSB | chr2: 170376560-170376619 | 0.005403 |
| A_32_P60076 | | chr17: 24071726-24071785 | 0.005420 |
| A_24_P936556 | ADARB1 | chr21: 45390178-45390237 | 0.005446 |
| A_23_P143178 | LBP | chr20: 36426783-36428848 | 0.005447 |
| A_24_P99639 | NEK8 | chr17: 24092129-24092280 | 0.005460 |
| A_23_P72117 | SMPDL3A | chr6: 123172304-123172363 | 0.005504 |
| A_23_P169766 | | chr7: 138907963-138907904 | 0.005534 |
| A_23_P120062 | C2orf3 | chr2: 75745114-75745055 | 0.005536 |
| A_32_P76811 | RP3-377H14.5 | chr6: 29802573-29802514 | 0.005622 |
| A_23_P69868 | SERF1B | chr5: 69374401-69374460 | 0.005645 |
| A_23_P105747 | APPL2 | chr12: 104092132-104092073 | 0.005713 |
| A_24_P227831 | ABCC1 | chr16: 16143525-16143584 | 0.005774 |
| A_24_P333106 | | chr14: 34025181-34025240 | 0.005791 |
| A_23_P79591 | APOB | chr2: 21078063-21078004 | 0.005792 |
| A_23_P109655 | TMEM22 | chr3: 138056971-138057030 | 0.005808 |
| A_24_P219474 | MGAT5B | chr17: 72457940-72457999 | 0.005850 |
| A_23_P100912 | GIT1 | chr17: 24925338-24925279 | 0.005879 |
| A_23_P18818 | CNOT6 | chr5: 179928861-179928920 | 0.005917 |
| A_23_P126844 | TNFRSF25 | chr1: 6443891-6443832 | 0.005946 |
| A_24_P131697 | ACTR8 | chr3: 53879154-53879095 | 0.006045 |
| A_24_P170496 | | chr21: 39421677-39421736 | 0.006097 |
| A_23_P158537 | | | 0.006115 |
| A_23_P306148 | PML | chr15: 72112803-72122383 | 0.006162 |
| A_24_P91140 | RPL23A | chr17: 24075215-24075274 | 0.006197 |
| A_24_P355267 | SLC25A25 | chr9: 129911256-129911315 | 0.006237 |
| A_32_P92489 | PKD1L2 | chr16: 79692374-79692315 | 0.006239 |
| A_24_P255253 | MGC70863 | chr22: 49584135-49584194 | 0.006302 |
| A_24_P151920 | TMEM97 | chr17: 23677691-23677750 | 0.006337 |
| A_32_P101420 | | chr9: 138814991-138814932 | 0.006342 |
| A_24_P75093 | | chr1: 152012469-152012410 | 0.006500 |
| A_23_P90419 | PBX4 | chr19: 19533597-19533538 | 0.006514 |
| A_23_P89710 | CEP192 | chr18: 13114695-13114754 | 0.006523 |
| A_24_P322908 | | chrX: 049533337-049533396 | 0.006579 |
| A_23_P41588 | HARS2 | chr5: 140058675-140058734 | 0.006617 |
| A_23_P373568 | FUZ | chr19: 55002175-55002116 | 0.006617 |
| A_23_P29747 | RPL23A | chr17: 24074765-24075050 | 0.006631 |
| A_32_P118461 | | chr17: 24075059-24074774 | 0.006686 |
| A_23_P151307 | RAPGEF3 | chr12: 46419194-46418747 | 0.006702 |
| A_23_P30411 | | chr5: 001122135-001122076 | 0.006722 |
| A_32_P121737 | | chr11: 22763336-22763277 | 0.006789 |
| A_24_P162911 | PRKD3 | chr2: 37337552-37337493 | 0.006815 |
| A_32_P163458 | tcag7.1017 | chr7: 99756467-99756526 | 0.006863 |
| A_23_P415633 | FLJ25006 | chr17: 23962353-23962294 | 0.006941 |
| A_24_P466102 | | chr2: 153055200-153055259 | 0.006976 |
| A_32_P219620 | EYA4 | chr6: 133894789-133894848 | 0.006989 |
| A_24_P238525 | | chr2: 106390376-106390435 | 0.007033 |
| A_23_P46903 | CAMK2G | chr10: 75246815-75244981 | 0.007047 |
| A_32_P7521 | | chr21: 32564709-32569068 | 0.007147 |
| A_23_P207614 | KIAA0100 | chr17: 23966120-23966061 | 0.007210 |
| A_24_P41662 | | chr1: 041337197-041337256 | 0.007217 |
| A_24_P12065 | | chr4: 78309441-78309500 | 0.007222 |
| A_24_P734892 | | chr5: 001386540-001386486 | 0.007252 |
| A_32_P155043 | | chr19: 042670572-042670631 | 0.007293 |
| A_23_P32279 | BARX1 | chr9: 95754167-95754108 | 0.007349 |
| A_32_P15898 | | chr16: 15734095-15734154 | 0.007363 |
| A_32_P128023 | | chr11: 62098727-62098786 | 0.007374 |
| A_24_P272993 | MEG3 | chr14: 100396938-100396997 | 0.007416 |
| A_23_P382045 | TULP4 | chr6: 158852650-158852709 | 0.007451 |
| A_23_P52373 | NDST2 | chr10: 75232068-75232009 | 0.007486 |
| A_23_P75283 | RBP4 | chr10: 95341718-95341659 | 0.007515 |
| A_23_P109974 | RAB6B | chr3: 135030253-135030194 | 0.007521 |
| A_23_P99452 | BRCA2 | chr13: 31870996-31871055 | 0.007576 |
| A_23_P38446 | TNFAIP1 | chr17: 23698035-23698094 | 0.007589 |
| A_32_P216193 | | chr17: 23720929-23720988 | 0.007600 |
| A_24_P342272 | PCDHGA5 | chr5: 140725250-140725309 | 0.007623 |
| A_23_P333951 | | chr1: 223221800-223221859 | 0.007754 |
| A_23_P79130 | PLEKHF1 | chr19: 34858097-34858156 | 0.007769 |
| A_24_P88554 | PEX11B | chr1: 144234960-144235019 | 0.007847 |
| A_32_P76992 | | chr15: 38405933-38405992 | 0.007894 |
| A_32_P174999 | | chr10: 103448055-103448114 | 0.007896 |
| A_24_P341897 | ACVR1B | chr12: 50674874-50674933 | 0.007905 |
| A_24_P943000 | POP1 | chr8: 99240271-99240330 | 0.007922 |
| A_24_P628618 | | chr2: 101256978-101257037 | 0.007985 |
| A_24_P711477 | | chr1: 26674467-26674408 | 0.008004 |
| A_23_P40039 | AMMECR1L | chr2: 128335869-128335810 | 0.008008 |
| A_23_P47941 | HOXC11 | chr12: 52655517-52655576 | 0.008063 |
| A_23_P49878 | FAM64A | chr17: 6294966-6295025 | 0.008072 |
| A_23_P166 | MOBKL2C | chr1: 46846412-46846353 | 0.008075 |
| A_23_P72157 | MFSD7 | chr4: 665759-665700 | 0.008106 |
| A_23_P32036 | C9orf95 | chr9: 76866199-76866140 | 0.008156 |
| A_23_P143083 | FLJ20254 | chr2: 27115533-27116168 | 0.008170 |
| A_32_P20492 | | chr1: 22291591-22291532 | 0.008252 |
| A_23_P71981 | ERAL1 | chr17: 24211933-24211992 | 0.008253 |
| A_23_P24384 | CCDC88 | chr11: 63881511-63881570 | 0.008315 |
| A_24_P269315 | SERPINB1 | chr6: 2777714-2777655 | 0.008321 |
| A_23_P133807 | TBN | chr6: 42152896-42152955 | 0.008344 |
| A_24_P381838 | MAN2A2 | chr15: 89264774-89264833 | 0.008398 |
| A_32_P91773 | | chr6: 160020489-160020548 | 0.008425 |
| A_23_P216282 | ARHGEF10 | chr8: 1894121-1894180 | 0.008531 |
| A_23_P28886 | PCNA | chr20: 5044102-5043957 | 0.008538 |
| A_23_P86330 | IER5 | chr1: 179326458-179326517 | 0.008595 |
| A_24_P300841 | CKAP5 | chr11: 46728512-46728453 | 0.008606 |
| A_23_P49878 | FAM64A | chr17: 6294966-6295025 | 0.008609 |
| A_23_P213069 | TMEM33 | chr4: 41651018-41651077 | 0.008674 |
| A_24_P496683 | | chr6: 99827973-99828032 | 0.008709 |
| A_32_P156549 | | chr17: 025095197-025095138 | 0.008722 |
| A_32_P135767 | | chr2: 224949580-224949521 | 0.008802 |
| A_32_P190568 | | chr3: 162629838-162629897 | 0.008807 |
| A_23_P335069 | SLC46A1 | chr17: 23747041-23746982 | 0.008824 |
| A_23_P331235 | C5orf38 | chr5: 2805741-2805800 | 0.008829 |
| A_23_P48807 | GALK2 | chr15: 47407593-47407652 | 0.008855 |
| A_23_P357794 | MTBP | chr8: 121604115-121604752 | 0.008883 |
| A_32_P116105 | | chr19: 54691848-54691907 | 0.008946 |
| A_23_P49878 | FAM64A | chr17: 6294966-6295025 | 0.008950 |
| A_24_P942589 | | chr4: 115818239-115818298 | 0.008986 |
| A_23_P167417 | CNOT6 | chr5: 179936201-179936260 | 0.009017 |
| A_32_P76842 | | chr17: 24071812-24071871 | 0.009019 |
| A_24_P110967 | PCMTD1 | chr8: 52935997-52920857 | 0.009051 |
| A_24_P121642 | LOC441743 | chr16: 376840-376899 | 0.009066 |
| A_24_P12932 | MRPS16 | chr10: 74680738-74680679 | 0.009085 |
| A_24_P142442 | POLDIP2 | chr17: 23698515-23698456 | 0.009094 |
| A_32_P308779 | ZNF620 | chr3: 40532619-40532678 | 0.009117 |
| A_23_P110266 | C4orf31 | chr4: 122176925-122176866 | 0.009206 |
| A_23_P215132 | HSPC049 | chr7: 134521164-134521105 | 0.009208 |
| A_24_P118874 | | chr8: 006744105-006744046 | 0.009299 |
| A_24_P940125 | CNOT6 | chr5: 179937504-179937563 | 0.009321 |
| A_32_P28634 | | chr4: 39134543-39135649 | 0.009348 |
| A_23_P37347 | SNW1 | chr14: 77253944-77253885 | 0.009385 |
| A_32_P38859 | | chr8: 49756619-49756678 | 0.009428 |
| A_23_P148737 | MYBPH | chr1: 201403642-201403583 | 0.009477 |
| A_23_P138631 | SMC3 | chr10: 112352641-112352700 | 0.009485 |
| A_32_P932089 | PCDHB19P | chr5: 140603623-140603682 | 0.009546 |
| A_23_P25019 | PRIM1 | chr12: 55419378-55418551 | 0.009568 |
| A_23_P203344 | ZFP91 | chr11: 58144444-58144503 | 0.009795 |
| A_24_P409728 | KIAA0913 | chr10: 75215741-75215800 | 0.009835 |
| A_24_P95007 | GTF21 | chr7: 73804407-73805389 | 0.009836 |
| A_24_P379649 | RPL23AP13 | chr2: 54610232-54610291 | 0.009871 |
| A_23_P427703 | MT1L | chr16: 055210081-055210140 | 0.009897 |
| A_23_P50990 | CENPO | chr2: 24894076-24894135 | 0.009946 |
| A_32_P42075 | C7orf28B | chr7: 6805308-6805249 | 0.009962 |
| A_24_P102293 | SLITRK5 | chr13: 87129587-87129646 | 0.009969 |
| A_24_P267293 | SARM1 | chr17: 23751868-23751927 | 0.009970 |
| A_23_P60933 | MT1G | chr16: 55258306-55258247 | 0.009999 |
| A_24_P932168 | | chr6: 086441770-086441829 | 0.010028 |
| A_24_P925100 | | chr7: 72136405-72136464 | 0.010130 |
| A_24_P934434 | | chr5: 053538519-053538578 | 0.010157 |
| A_23_P71537 | CSPP1 | chr8: 68270186-68270245 | 0.010231 |
| A_23_P20045 | PEX1 | chr7: 91956971-91956622 | 0.010281 |
| A_24_P515866 | | chr3: 49990116-49990057 | 0.010315 |
| A_24_P50328 | | chr6: 090581784-090581843 | 0.010334 |
| A_23_P141484 | C17orf63 | chr17: 24107968-24107909 | 0.010337 |
| A_32_P122639 | | chr11: 107167288-107167347 | 0.010356 |
| A_23_P312610 | DNAJC4 | chr11: 63755978-63757966 | 0.010361 |
| A_23_P12514 | RHOC | chr1: 113045421-113045362 | 0.010373 |
| A_23_P18465 | RFC1 | chr4: 38966529-38966470 | 0.010381 |
| A_23_P79591 | APOB | chr2: 21078063-21078004 | 0.010441 |
| A_24_P195693 | MGC70863 | chr22: 49584169-49584228 | 0.010459 |
| A_23_P134484 | CALCR | chr7: 92892064-92892005 | 0.010483 |
| A_23_P342641 | SLC44A5 | chr1: 75452000-75449789 | 0.010526 |
| A_23_P145895 | TP53AP1 | chr7: 86808744-86808685 | 0.010574 |
| A_23_P60793 | CXYorf2 | chrY: 1491698-1491757 | 0.010580 |
| A_24_P332532 | LOC731749 | chr11: 66285652-66285622 | 0.010612 |
| A_23_P250380 | MAN2B2 | chr4: 6674181-6674240 | 0.010649 |
| A_23_P30848 | HLA-E | chr6: 30566046-30566105 | 0.010690 |
| A_32_P32043 | | chr9: 15461724-15461665 | 0.010734 |
| A_24_P808579 | | chr5: 040825818-040825877 | 0.010851 |
| A_24_P930613 | | chr1: 146872718-146872659 | 0.010898 |
| A_23_P106844 | MT2A | chr16: 55200833-55200892 | 0.010901 |
| A_24_P342316 | FRYL | chr4: 48195358-48195299 | 0.010913 |
| A_32_P201603 | | chr2: 108669318-108669259 | 0.010935 |
| A_23_P13344 | EEF1G | chr11: 62084136-62083846 | 0.010971 |
| A_24_P84508 | LOC648294 | chr9: 6629362-6629423 | 0.011000 |
| A_23_P28318 | PRO1853 | chr2: 37328980-37329039 | 0.011017 |
| A_32_P65943 | EEF1D | chr8: 144740043-144739546 | 0.011098 |
| A_24_P849628 | RDBP | chr6: 32028030-32027971 | 0.011144 |
| A_23_P85941 | ALX3 | chr1: 110404693-110404634 | 0.011161 |
| A_24_P81841 | CDKN1B | chr12: 12766207-12766266 | 0.011227 |
| A_24_P126873 | SUPT6H | chr17: 24050992-24051330 | 0.011249 |
| A_23_P252413 | MT2A | chr16: 55200838-55200897 | 0.011307 |
| A_23_P169756 | HIPK2 | chr7: 138907983-138907924 | 0.011338 |
| A_24_P920758 | | chr5: 167775773-167775832 | 0.011376 |
| A_24_P25326 | ZMYM6 | chr1: 35225535-35225476 | 0.011411 |
| A_23_P62188 | KIAA1166 | chrX: 64053180-64053121 | 0.011504 |
| A_23_P82941 | ARFGEF1 | chr8: 68272807-68272748 | 0.011510 |
| A_23_P41159 | PPP4R2 | chr3: 73197352-73197411 | 0.011586 |
| A_24_P611114 | hCG_1730474 | chr11: 57270216-57270275 | 0.011617 |
| A_32_P50187 | | chr18: 32020811-32020794 | 0.011618 |
| A_23_P164179 | TOB1 | chr17: 46295556-46295497 | 0.011625 |
| A_24_P324577 | KIAA1466 | chr7: 134516865-134516924 | 0.011639 |
| A_23_P256059 | MGC70863 | chr22: 49584398-49584457 | 0.011729 |
| A_24_P282309 | FER1L3 | chr10: 95152091-95152032 | 0.011735 |
| A_23_P18119 | IMPG2 | chr3: 102432616-102432557 | 0.011828 |
| A_24_P81841 | CDKN1B | chr12: 12766207-12766266 | 0.011851 |
| A_23_P350074 | C19orf46 | chr19: 41186113-41186054 | 0.012009 |
| A_32_P43415 | | chr15: 070350601-070350660 | 0.012025 |
| A_23_P103765 | FCER1A | chr1: 157542658-157544219 | 0.012057 |
| A_24_P56887 | CRLF3 | chr17: 26133905-26133846 | 0.012116 |
| A_24_P921213 | | chr16: 017325340-017325399 | 0.012136 |
| A_24_P216654 | SOAT1 | chr1: 177590947-177591006 | 0.012146 |
| A_23_P121686 | SCYE1 | chr4: 107477549-107477608 | 0.012151 |
| A_32_P9382 | RP11-11C5.2 | chr13: 72180694-72180635 | 0.012187 |
| A_24_P36745 | CXorf38 | chrX: 40374259-40374200 | 0.012261 |
| A_24_P134727 | TFAM | chr10: 59825178-59825237 | 0.012358 |
| A_24_P256863 | THRAP3 | chr1: 36532055-36532114 | 0.012403 |
| A_23_P387722 | SFXN4 | chr10: 120895802-120895743 | 0.012443 |
| A_23_P97871 | ARID5B | chr10: 63523177-63523236 | 0.012478 |
| A_32_P41136 | | chr5: 058951903-058951962 | 0.012602 |
| A_23_P371777 | ACE | chr17: 58951414-58952448 | 0.012642 |
| A_32_P380365 | | chr13: 82392074-82392015 | 0.012718 |
| A_23_P417261 | EFHB | chr3: 19896036-19895977 | 0.012725 |
| A_24_P935400 | | chr3: 184959678-184959737 | 0.012804 |
| A_23_P93818 | DKFZP434A0131 | chr7: 74857450-74857509 | 0.012865 |
| A_23_P130241 | KRTAP1-3 | chr17: 36443992-36443933 | 0.012892 |
| A_23_P327777 | GATS | chr7: 99636814-99636755 | 0.012915 |
| A_23_P70888 | FBXO24 | chr7: 100036532-100036591 | 0.012942 |
| A_23_P103256 | CFHR3 | chr1: 195015043-195015102 | 0.012953 |
| A_32_P229311 | | chr10: 121321551-121321610 | 0.012993 |
| A_23_P51217 | CLCA1 | chr1: 86738403-86738462 | 0.013044 |
| A_24_P328872 | CCT5 | chr5: 10318930-10318989 | 0.013068 |
| A_24_P76546 | WASF2 | chr1: 27605574-27605515 | 0.013129 |
| A_24_P310009 | USP43 | chr17: 9572967-9573026 | 0.013208 |
| A_24_P896188 | | chr2: 45016034-45015975 | 0.013220 |
| A_32_P113110 | | chr8: 128310422-128310363 | 0.013397 |
| A_24_P932646 | | chr2: 10061255-10061196 | 0.013400 |
| A_23_P202964 | TMEM123 | chr11: 101777616-101777557 | 0.013423 |
| A_23_P39215 | NUDT19 | chr19: 37892091-37894509 | 0.013464 |
| A_23_P80635 | IRAK2 | chr3: 10236456-10239361 | 0.013521 |
| A_23_P42168 | MDFI | chr6: 41729620-41729679 | 0.013534 |
| A_24_P311771 | ZFR | chr5: 32421443-32421384 | 0.013547 |
| A_32_P82119 | LOC652549 | chr1: 78988445-78988503 | 0.013596 |
| A_23_P502808 | PRIMA1 | chr14: 93273403-93273344 | 0.013597 |
| A_24_P570378 | | chr10: 52233288-52233229 | 0.013643 |
| A_23_P134573 | RCP9 | chr7: 65255342-65255401 | 0.013661 |
| A_32_P8551 | NRN1L | chr16: 66477712-66477771 | 0.013670 |
| A_24_P81841 | CDKN1B | chr12: 12766207-12766266 | 0.013683 |
| A_32_P56584 | | chr8: 101234700-101233264 | 0.013763 |
| A_24_P264106 | RPL23A | chr17: 24073905-24073964 | 0.013860 |
| A_23_P206806 | ITGAL | chr16: 30441409-30441468 | 0.013914 |
| A_24_P244356 | NLRX1 | chr11: 118559866-118559925 | 0.013998 |
| A_32_P232327 | | chr10: 73704560-73704501 | 0.014097 |
| A_23_P23171 | EIF2C4 | chr1: 36093007-36093066 | 0.014109 |
| A_24_P785293 | SNHG1 | chr11: 62376208-62376149 | 0.014129 |
| A_24_P81841 | CDKN1B | chr12: 12766207-12766266 | 0.014175 |
| A_23_P373687 | FLJ32312 | chr2: 61022986-61022927 | 0.014231 |
| A_23_P360079 | NAP5 | chr2: 133199757-133199698 | 0.014248 |
| A_32_P99055 | WNK3 | chrX: 54240317-54240258 | 0.014347 |
| A_23_P259632 | TMPIT | chr7: 75454498-75454439 | 0.014409 |
| A_24_P346277 | SRF | chr6: 43256969-43257028 | 0.014437 |
| A_23_P144704 | | chr5: 43164083-43164141 | 0.014451 |
| A_32_P208713 | | chr19: 023311797-023311856 | 0.014473 |
| A_24_P926566 | ARHGEF6 | chrX: 135691363-135691304 | 0.014524 |
| A_23_P76918 | TIMM9 | chr14: 57945278-57945219 | 0.014594 |
| A_24_P121956 | TMEM86B | chr19: 60430207-60430148 | 0.014598 |
| A_24_P298027 | AXIN2 | chr17: 60984909-60984850 | 0.014602 |
| A_24_P3627 | | chr5: 042589834-042589893 | 0.014610 |
| A_24_P10214 | STXBP6 | chr14: 24396107-24395123 | 0.014754 |
| A_23_P138435 | ZMIZ1 | chr10: 80746042-80746101 | 0.014782 |
| A_23_P137573 | LEFTY2 | chr1: 224191756-224191697 | 0.014816 |
| A_24_P16678 | C8orf34 | chr8: 69866272-69866331 | 0.014879 |
| A_32_P105217 | | chr2: 206339983-206339924 | 0.014902 |
| A_24_P202527 | SMAD2 | chr18: 43625804-43625745 | 0.014915 |
| A_24_P260582 | TRIM52 | chr5: 180615030-180614971 | 0.014935 |
| A_24_P134568 | RP2 | chrX: 46598362-46598421 | 0.014964 |
| A_23_P77430 | PRMT7 | chr16: 66948563-66948622 | 0.014985 |
| A_24_P932614 | | chr3: 99784790-99784731 | 0.015025 |
| A_24_P403928 | PAPLN | chr14: 72803230-72803289 | 0.015084 |
| A_24_P359856 | HDAC4 | chr2: 239639395-239639336 | 0.015087 |
| A_24_P381494 | SLC11A2 | chr12: 49666625-49666566 | 0.015135 |
| A_23_P16834 | FNDC4 | chr2: 27568826-27568767 | 0.015164 |
| A_24_P36229 | DTNA | chr18: 30685882-30692297 | 0.015182 |
| A_24_P336113 | CABIN1 | chr22: 22904422-22904481 | 0.015215 |
| A_24_P924202 | PCNXL3 | chr11: 65161147-65161206 | 0.015218 |
| A_23_P5938 | C20orf117 | chr20: 34853488-34853429 | 0.015268 |
| A_23_P202374 | COX15 | chr10: 101464276-101464217 | 0.015358 |
| A_23_P63067 | DAP3 | chr1: 153974781-153974840 | 0.015363 |
| A_23_P350658 | C9orf93 | chr9: 15875906-15875965 | 0.015417 |
| A_23_P53137 | HBG1 | chr11: 5226169-5226110 | 0.015474 |
| A_23_P138985 | CD3D | chr11: 117716348-117715821 | 0.015513 |
| A_23_P122493 | TRIM31 | chr6: 30186429-30186370 | 0.015572 |
| A_23_P169154 | KIAA1815 | chr9: 5775753-5775694 | 0.015693 |
| A_24_P924462 | PRKCZ | chr1: 2022121-2022180 | 0.015758 |
| A_23_P61580 | NSUN6 | chr10: 18874789-18874730 | 0.015815 |
| A_24_P112110 | GTF2I | chr7: 73751245-73751304 | 0.015848 |
| A_23_P138271 | ARL8A | chr1: 200369355-200369296 | 0.015940 |
| A_23_P431591 | C5orf39 | chr5: 43075280-43075221 | 0.015951 |
| A_23_P29023 | C21orf119 | chr21: 32688056-32688115 | 0.015969 |
| A_23_P111005 | YIPF3 | chr6: 43587823-43587764 | 0.016072 |
| A_24_P942151 | | chr1: 115049977-115050036 | 0.016081 |
| A_23_P29081 | PFKL | chr21: 44567292-44567351 | 0.016095 |
| A_23_P202918 | KCNJ1 | chr11: 128214612-128214553 | 0.016112 |
| A_23_P328074 | SALL1 | chr16: 49728135-49728076 | 0.016143 |
| A_24_P60381 | KNG1 | chr3: 187918143-187918202 | 0.016146 |
| A_24_P531884 | | chr3: 127654657-127654716 | 0.016176 |
| A_23_P50455 | POLD1 | chr19: 55613018-55613077 | 0.016200 |
| A_23_P213810 | CCDC69 | chr5: 150542731-150542672 | 0.016214 |
| A_24_P922056 | FLJ39378 | chr12: 122522560-122522619 | 0.016224 |
| A_23_P252155 | STRBP | chr9: 124927732-124927673 | 0.016265 |
| A_24_P732181 | | chr17: 22589073-22589132 | 0.016306 |
| A_23_P29866 | | chr4: 39375382-39375323 | 0.016350 |
| A_24_P934826 | | chr8: 143279551-143279610 | 0.016352 |
| A_23_P70328 | CENPQ | chr6: 49564899-49564958 | 0.016377 |
| A_23_P38457 | TAOK1 | chr17: 24895395-24895454 | 0.016392 |
| A_23_P378288 | IKZF4 | chr12: 54718398-54718457 | 0.016405 |
| A_32_P32722 | | chr5: 180189647-180189588 | 0.016416 |
| A_24_P56894 | CRLF3 | chr17: 26134846-26134787 | 0.016419 |
| A_24_P75808 | | chr12: 61684594-61684655 | 0.016500 |
| A_32_P170800 | | chr10: 43202412-43202471 | 0.016524 |
| A_23_P8834 | EPHX2 | chr8: 27458032-27458091 | 0.016592 |
| A_23_P123478 | PDE7A | chr8: 66799575-66799091 | 0.016594 |
| A_23_P114649 | RP5-1077B9.4 | chr1: 12011908-12012450 | 0.016608 |
| A_23_P201979 | CREM | chr10: 35541248-35541307 | 0.016635 |
| A_23_P116387 | INCENP | chr11: 61676318-61676377 | 0.016647 |
| A_24_P76635 | ARP11 | chr7: 149575359-149575300 | 0.016722 |
| A_24_P252020 | TRIM41 | chr5: 180594222-180594281 | 0.016738 |
| A_24_P843020 | LOC729111 | chr19: 46698829-46698770 | 0.016748 |
| A_23_P200976 | HYI | chr1: 43689740-43689681 | 0.016784 |
| A_24_P190168 | TMEM97 | chr17: 23679051-23679110 | 0.016790 |
| A_24_P927537 | TFAM | chr10: 59828761-59828820 | 0.016804 |
| A_23_P256716 | SMARCA5 | chr4: 144688632-144688691 | 0.016808 |
| A_23_P203475 | PRKCDBP | chr11: 6296845-6296786 | 0.016835 |
| A_23_P347623 | ERO1LB | chr1: 234451894-234450877 | 0.016844 |
| A_24_P316257 | FLJ36208 | chr16: 559160-559219 | 0.016933 |
| A_24_P5550 | PHF23 | chr17: 7080585-7080526 | 0.016959 |
| A_24_P274640 | GTF2H2 | chr5: 70373168-70373109 | 0.016966 |
| A_23_P95292 | KIAA0652 | chr11: 46650404-46650463 | 0.016976 |
| A_24_P296508 | SLC43A2 | chr17: 1424511-1424452 | 0.016987 |
| A_24_P353740 | CTTNBP2NL | chr1: 112801146-112801205 | 0.017058 |
| A_32_P150449 | | chr12: 114960595-114960536 | 0.017125 |
| A_24_P244096 | DNAJB12 | chr10: 73762682-73762623 | 0.017148 |
| A_23_P250825 | RABGEF1 | chr7: 65911718-65911777 | 0.017283 |
| A_23_P352619 | TIFA | chr4: 113417400-113417341 | 0.017303 |
| A_23_P218918 | FGF2 | chr4: 124038472-124038531 | 0.017304 |
| A_24_P41170 | PLEKHA5 | chr12: 19350939-19350998 | 0.017312 |
| A_32_P116989 | | chrX: 103246735-103246794 | 0.017324 |
| A_24_P24905 | DIRAS2 | chr9: 92414206-92414147 | 0.017364 |
| A_32_P186185 | | chr1: 28968624-28968565 | 0.017399 |
| A_24_P113341 | C19orf12 | chr19: 34883981-34883922 | 0.017416 |
| A_23_P138881 | ACTN3 | chr11: 66086174-66086321 | 0.017464 |
| A_23_P67072 | ZNF615 | chr19: 57187881-57187822 | 0.017479 |
| A_32_P16204 | LOC375295 | chr2: 177203078-177203019 | 0.017577 |
| A_24_P923102 | KLHL23 | chr2: 170316449-170316508 | 0.017590 |
| A_24_P108301 | RTTN | chr18: 65822143-65822084 | 0.017607 |
| A_23_P399146 | ZNF549 | chr19: 62743465-62743524 | 0.017644 |
| A_23_P416732 | TCTE3 | chr6: 169882269-169882210 | 0.017677 |
| A_23_P124327 | NSD1 | chr5: 176655180-176655239 | 0.017681 |
| A_23_P31903 | VPS28 | chr8: 145619881-145619822 | 0.017744 |
| A_24_P393389 | L3MBTL | chr20: 41611824-41611883 | 0.017771 |
| A_32_P234459 | HLA-H | chr6: 29964292-29964351 | 0.017790 |
| A_32_P6535 | PLGLB1 | chr2: 87091639-87091580 | 0.017793 |
| A_32_P211765 | | chr12: 8400853-8400826 | 0.017897 |
| A_24_P454931 | | chr4: 109119338-109051109 | 0.017897 |
| A_23_P66719 | DHRS13 | chr17: 24249234-24249175 | 0.017969 |
| A_24_P682285 | HSPA1A | chr6: 31892373-31892432 | 0.017979 |
| A_23_P13885 | ATN1 | chr12: 6921516-6921575 | 0.018015 |
| A_23_P74993 | | chr1: 142509545-142509486 | 0.018054 |
| A_24_P255654 | LOC400039 | chr12: 51834250-51834310 | 0.018159 |
| A_23_P118705 | SDF2 | chr17: 23999899-23999840 | 0.018282 |
| A_23_P334123 | ITFG1 | chr16: 45750391-45750332 | 0.018298 |
| A_23_P26674 | KIAA0430 | chr16: 15595868-15595809 | 0.018309 |
| A_23_P374288 | CASC3 | chr17: 35581538-35581597 | 0.018409 |
| A_24_P348925 | CCNK | chr14: 99029615-99029674 | 0.018413 |
| A_23_P255805 | ZNF7 | chr8: 146039070-146039129 | 0.018459 |
| A_32_P197026 | | chr8: 023480243-023480184 | 0.018478 |
| A_23_P123315 | | chr8: 92149966-92149907 | 0.018489 |
| A_32_P27917 | KIF26A | chr14: 103716594-103716653 | 0.018494 |
| A_24_P339560 | SIGLEC11 | chr19: 55145070-55145011 | 0.018536 |
| A_23_P205098 | APRIN | chr13: 32249525-32249584 | 0.018576 |
| A_23_P35035 | EDG7 | chr1: 85051761-85051702 | 0.018583 |
| A_32_P178339 | | chr7: 065837605-065837546 | 0.018629 |
| A_24_P153399 | RUTBC3 | chr22: 39135890-39135949 | 0.018674 |
| A_23_P217487 | FOXO4 | chrX: 70238432-70238491 | 0.018704 |
| A_24_P273203 | | chr3: 75756931-75756872 | 0.018801 |
| A_23_P98830 | UBQLN3 | chr11: 5485170-5485111 | 0.018811 |
| A_24_P91272 | SOCS6 | chr18: 66145104-66145163 | 0.018863 |
| A_23_P43484 | CDKN2A | chr9: 21957839-21957780 | 0.018887 |
| A_23_P165937 | DSN1 | chr20: 34814228-34814169 | 0.018932 |
| A_23_P136478 | IRF2 | chr4: 185546312-185546253 | 0.018946 |
| A_24_P350245 | DOCK5 | chr8: 25157129-25157188 | 0.018954 |
| A_23_P135184 | RALGDS | chr9: 134963092-134963033 | 0.019007 |
| A_23_P30870 | BAT2 | chr6: 31712020-31712079 | 0.019141 |
| A_23_P48237 | MCRS1 | chr12: 48238769-48238710 | 0.019143 |
| A_24_P375205 | | chr16: 14242019-14242078 | 0.019212 |
| A_23_P75038 | DCLRE1A | chr10: 115584658-115584599 | 0.019267 |
| A_24_P105191 | HS6ST2 | chrX: 131587828-131587769 | 0.019305 |
| A_32_P41526 | CTGLF1 | chr10: 45642218-45642159 | 0.019312 |
| A_24_P27977 | TRPM2 | chr21: 44686639-44686698 | 0.019355 |
| A_23_P22224 | EIF4EBP1 | chr8: 38036875-38036934 | 0.019370 |
| A_24_P81841 | CDKN1B | chr12: 12766207-12766266 | 0.019384 |
| A_23_P164210 | TBC1D3 | chr17: 33369239-33369298 | 0.019386 |
| A_23_P69339 | ACAA1 | chr3: 38139419-38139360 | 0.019409 |
| A_23_P152876 | RAB34 | chr17: 24065535-24065476 | 0.019455 |
| A_23_P138480 | C10orf95 | chr10: 104199994-104199935 | 0.019533 |
| A_32_P72351 | | chr7: 295672-295731 | 0.019547 |
| A_23_P54488 | ACSBG1 | chr15: 76252997-76250875 | 0.019559 |
| A_32_P300427 | APCDD1L | chr20: 56467974-56467915 | 0.019589 |
| A_23_P307346 | CA5B | chrX: 15702387-15703294 | 0.019617 |
| A_32_P199736 | | chr15: 39361278-39361219 | 0.019624 |
| A_23_P103405 | | chr1: 222713795-222713854 | 0.019710 |
| A_23_P86623 | ENTPD7 | chr10: 101455180-101455239 | 0.019755 |
| A_32_P133244 | PARN | chr16: 14437452-14437393 | 0.019776 |
| A_32_P129310 | | chr17: 25082166-25082107 | 0.019844 |
| A_24_P81841 | CDKN1B | chr12: 12766207-12766266 | 0.019861 |
| A_23_P203332 | FAM111A | chr11: 58679008-58679067 | 0.019925 |
| A_24_P169462 | GOSR1 | chr17: 25832371-25835380 | 0.019957 |
| A_24_P158298 | | chr9: 70789905-70789964 | 0.019987 |
| A_24_P159548 | SLC46A1 | chr17: 23755998-23755939 | 0.020070 |
| A_24_P117803 | | chrX: 114339373-114339432 | 0.020120 |
| A_23_P85441 | IGSF9 | chr1: 158163619-158163560 | 0.020150 |
| A_23_P110851 | TERT | chr5: 1306364-1306305 | 0.020183 |
| A_32_P202042 | ANAPC5 | chr12: 120269135-120269990 | 0.020184 |
| A_23_P24275 | C10orf110 | chr10: 1079998-1080057 | 0.020201 |
| A_23_P201636 | LAMC2 | chr1: 181480233-181480292 | 0.020239 |
| A_32_P169406 | LOC400043 | chr12: 52812598-52812657 | 0.020249 |
| A_23_P118246 | GINS2 | chr16: 84269214-84269155 | 0.020313 |
| A_23_P8561 | RHBDD2 | chr7: 75355618-75355677 | 0.020373 |
| A_24_P134074 | RPS19 | chr19: 47057071-47064949 | 0.020407 |
| A_24_P153043 | RPL23AP7 | chr2: 114085525-114085498 | 0.020453 |
| A_23_P51936 | TNFRSF9 | chr1: 7903369-7903310 | 0.020474 |
| A_24_P75344 | SMYD4 | chr17: 1633119-1631465 | 0.020477 |
| A_24_P938572 | gm127 | chr1: 225951989-225951930 | 0.020527 |
| A_32_P189324 | | chr2: 179018566-179018625 | 0.020656 |
| A_24_P919876 | SHMT1 | chr17: 18192546-18192488 | 0.020757 |
| A_23_P398040 | YTHDF3 | chr8: 64285048-64285107 | 0.020820 |
| A_23_P77761 | ACOX1 | chr17: 71456064-71456005 | 0.020824 |
| A_32_P86705 | | chr4: 174325731-174324501 | 0.020836 |
| A_23_P1248 | C10orf26 | chr10: 104563853-104563912 | 0.020846 |
| A_32_P63284 | | | 0.020918 |
| A_32_P852607 | DYRK1A | chr21: 37661549-37661608 | 0.020922 |
| A_23_P59807 | WNT2 | chr7: 116704680-116704621 | 0.020985 |
| A_23_P371168 | TRIM60 | chr4: 166181963-166182022 | 0.020999 |
| A_23_P38041 | NAGPA | chr16: 5015070-5015011 | 0.021004 |
| A_24_P381224 | SYVN1 | chr11: 64651634-64651575 | 0.021005 |
| A_23_P133438 | FAM105A | chr5: 14663510-14663569 | 0.021078 |
| A_24_P93901 | SIN3B | chr19: 16826094-16826153 | 0.021112 |
| A_24_P377489 | C7orf28A | chr7: 5930024-5930083 | 0.021128 |
| A_23_P41476 | LOC152573 | chr4: 42098938-42098997 | 0.021190 |
| A_24_P125353 | CASC3 | chr17: 35578184-35579149 | 0.021301 |
| A_23_P134814 | THAP1 | chr8: 42811213-42811154 | 0.021302 |
| A_32_P26483 | | chr2: 202960491-202960550 | 0.021314 |
| A_23_P6335 | SERPIND1 | chr22: 19471720-19471779 | 0.021325 |
| A_23_P160869 | LRIG2 | chr1: 113468578-113468637 | 0.021330 |
| A_24_P203244 | C9orf79 | chr9: 89693548-89693607 | 0.021333 |
| A_32_P165993 | | chr21: 44004985-44004926 | 0.021343 |
| A_23_P50535 | DMPK | chr19: 50964920-50964861 | 0.021348 |
| A_23_P416536 | | chr10: 118582578-118582637 | 0.021444 |
| A_24_P929974 | | | 0.021449 |
| A_23_P344194 | LOC387856 | chr12: 46865151-46865210 | 0.021453 |
| A_32_P94660 | LRRC37B | chr17: 27401135-27401194 | 0.021473 |
| A_24_P937489 | | chr2: 170186426-170186367 | 0.021508 |
| A_24_P69538 | TLR4 | chr9: 119516677-119516736 | 0.021522 |
| A_23_P33207 | | | 0.021529 |
| A_23_P115762 | ECD | chr10: 74564386-74564327 | 0.021545 |
| A_23_P130285 | VPS53 | chr17: 382818-382759 | 0.021624 |
| A_23_P59950 | SLC39A14 | chr8: 22335781-22335840 | 0.021699 |
| A_23_P139471 | RPS26 | chr12: 54722006-54722065 | 0.021742 |
| A_32_P91902 | TSPAN14 | chr10: 82271838-82271897 | 0.021909 |
| A_23_P99811 | MDP-1 | chr14: 23753166-23753107 | 0.021914 |
| A_23_P18672 | GBA3 | chr4: 22430188-22430247 | 0.021916 |
| A_24_P298723 | TTTY20 | chrY: 9232439-9232380 | 0.021931 |
| A_23_P207400 | BRCA1 | chr17: 38450007-38449948 | 0.021986 |
| A_24_P6790 | ATXN7L1 | chr7: 105038285-105038226 | 0.021999 |
| A_32_P127105 | | chr17: 24902793-24902852 | 0.022021 |
| A_23_P52531 | FAM24B | chr10: 124599980-124598937 | 0.022092 |
| A_23_P133755 | PREP | chr6: 105832578-105832519 | 0.022117 |
| A_23_P356070 | FLT4 | chr5: 179969561-179968651 | 0.022119 |
| A_23_P8541 | POM121 | chr7: 72056827-72056886 | 0.022203 |
| A_24_P921823 | TCF7L2 | chr10: 114917255-114917314 | 0.022220 |
| A_32_P43349 | LOC400456 | chr15: 93632599-93632540 | 0.022226 |
| A_23_P202156 | NFKB2 | chr10: 104151043-104151219 | 0.022268 |
| A_23_P368067 | TAP2 | chr6: 32897841-32897782 | 0.022282 |
| A_24_P361896 | MT2A | chr16: 55200782-55200841 | 0.022332 |
| A_23_P254612 | DBF4 | chr7: 87375257-87375316 | 0.022345 |
| A_23_P53137 | HBG1 | chr11: 5226169-5226110 | 0.022463 |
| A_24_P406132 | MAPK13 | chr6: 36215293-36215352 | 0.022476 |
| A_23_P64499 | HCCA2 | chr11: 1447795-1447736 | 0.022489 |
| A_23_P40952 | RAF1 | chr3: 12600670-12600611 | 0.022491 |
| A_23_P23194 | PINK1 | chr1: 20849912-20849971 | 0.022535 |
| A_23_P69206 | SLC6A6 | chr3: 14488791-14493737 | 0.022599 |
| A_23_P33791 | SSBP2 | chr5: 80752083-80752024 | 0.022602 |
| A_24_P312790 | SCN3B | chr11: 123014233-123014174 | 0.022620 |
| A_24_P136619 | CRISPLD2 | chr16: 83437096-83437155 | 0.022653 |
| A_32_P88349 | | chr1: 142535707-142535763 | 0.022702 |
| A_23_P259692 | PSAT1 | chr9: 80134598-80134657 | 0.022748 |
| A_24_P627678 | | chr1: 026674404-026674455 | 0.022765 |
| A_24_P940385 | | chr17: 004876500-004876559 | 0.022893 |
| A_24_P637226 | | chr1: 110714912-110714853 | 0.022895 |
| A_24_P278273 | MGC14425 | chr10: 64895612-64895671 | 0.022898 |
| A_23_P341938 | NOG | chr17: 52027218-52027277 | 0.022934 |
| A_32_P164971 | | chr16: 10944015-10944074 | 0.022964 |
| A_24_P143138 | FGD1 | chrX: 54490530-54490471 | 0.023000 |
| A_32_P20686 | | chr8: 93799805-93781337 | 0.023034 |
| A_23_P78888 | FBL | chr19: 45017032-45016973 | 0.023064 |
| A_32_P80587 | | chr13: 31998075-31998134 | 0.023073 |
| A_24_P592871 | | chr8: 96063101-96063160 | 0.023091 |
| A_23_P4190 | FLJ20920 | chr17: 45906354-45906607 | 0.023098 |
| A_23_P153797 | NCAN | chr19: 19223667-19223726 | 0.023098 |
| A_23_P377245 | | chr1: 35213975-35213916 | 0.023099 |
| A_23_P427014 | CLDN8 | chr21: 30508896-30508837 | 0.023134 |
| A_23_P423331 | NTNG2 | chr9: 134107927-134107986 | 0.023154 |
| A_24_P415168 | URM1 | chr9: 130192598-130192653 | 0.023197 |
| A_24_P676999 | | chr8: 146073197-146073256 | 0.023198 |
| A_24_P71280 | GPR157 | chr1: 9109912-9109853 | 0.023234 |
| A_24_P192978 | FLJ33790 | chr11: 74817137-74815275 | 0.023261 |
| A_23_P212554 | TBL1XR1 | chr3: 178222180-178222121 | 0.023262 |
| A_23_P100478 | MYST1 | chr16: 31046907-31048843 | 0.023264 |
| A_24_P246787 | REST | chr4: 57490974-57491033 | 0.023279 |
| A_24_P122891 | ZFAND2B | chr2: 219782544-219782603 | 0.023297 |
| A_23_P165408 | KLHL23 | chr2: 170314544-170314603 | 0.023350 |
| A_23_P166023 | PFDN4 | chr20: 52265292-52265351 | 0.023361 |
| A_24_P490911 | | chr19: 43849831-43849890 | 0.023377 |
| A_24_P399622 | COPE | chr19: 18891033-18884798 | 0.023408 |
| A_23_P110851 | TERT | chr5: 1306364-1306305 | 0.023412 |
| A_32_P126485 | | chr1: 143052296-143052237 | 0.023414 |
| A_24_P506893 | | chr8: 75891150-75891091 | 0.023416 |
| A_24_P593036 | LOC284072 | chr17: 68099946-68100005 | 0.023462 |
| A_32_P203559 | C11orf39 | chr11: 131037370-131037311 | 0.023480 |
| A_32_P111019 | | chr11: 47552096-47552155 | 0.023577 |
| A_23_P254512 | EFNA1 | chr1: 153373832-153373891 | 0.023655 |
| A_23_P89509 | SPAG5 | chr17: 23929224-23929165 | 0.023699 |
| A_23_P97250 | GON4L | chr1: 153986200-153986141 | 0.023709 |
| A_24_P25872 | DEPDC1 | chr1: 68715058-68714999 | 0.023724 |
| A_23_P11926 | FLJ21272 | chr1: 143702189-143702248 | 0.023734 |
| A_32_P113173 | | chr7: 88265154-88265213 | 0.023874 |
| A_24_P923553 | | chr13: 046681440-046681381 | 0.023878 |
| A_23_P9209 | NIPSNAP3B | chr9: 106568541-106568600 | 0.023921 |
| A_24_P823708 | FAM91A2 | chr1: 143012161-143012102 | 0.023931 |
| A_24_P336983 | CAPN13 | chr2: 30807740-30807124 | 0.023938 |
| A_24_P363548 | HIP1 | chr7: 75002247-75002188 | 0.024093 |
| A_24_P226008 | MGLL | chr3: 128896522-128893812 | 0.024107 |
| A_24_P363953 | RAB11FIP2 | chr10: 119788860-119788801 | 0.024188 |
| A_23_P4014 | SMG6 | chr17: 1909922-1909883 | 0.024247 |
| A_23_P168470 | BAIAP2L1 | chr7: 97779483-97779424 | 0.024291 |
| A_32_P24140 | GAS2 | chr11: 22790770-22790829 | 0.024438 |
| A_24_P201064 | LOC286044 | chr8: 9031282-9031223 | 0.024505 |
| A_23_P209625 | CYP1B1 | chr2: 38149007-38148948 | 0.024516 |
| A_23_P7957 | GNMT | chr6: 43038841-43038900 | 0.024528 |
| A_23_P163942 | SUPT4H1 | chr17: 53778348-53778289 | 0.024567 |
| A_24_P347946 | GIT1 | chr17: 24925955-24925896 | 0.024629 |
| A_23_P91590 | RANBP1 | chr22: 18494529-18494588 | 0.024634 |
| A_23_P157715 | PPP1R16A | chr8: 145698252-145698311 | 0.024670 |
| A_32_P68408 | LOC161635 | chr15: 34897398-34897339 | 0.024732 |
| A_23_P206068 | CHD2 | chr15: 91366152-91366211 | 0.024738 |
| A_23_P45409 | UPF3B | chrX: 118856395-118856021 | 0.024893 |
| A_23_P200984 | PTPRF | chr1: 43859152-43859211 | 0.024918 |
| A_24_P160401 | CDCP1 | chr3: 45128604-45127304 | 0.024938 |
| A_23_P1638 | RBM4B | chr11: 66189244-66189185 | 0.024965 |
| A_32_P2177 | | chr3: 9932945-9932886 | 0.024994 |
| A_23_P97889 | POLL | chr10: 103329471-103329412 | 0.024996 |
| A_23_P167789 | FAM53C | chr5: 137712897-137712956 | 0.025001 |
| A_23_P4561 | SERPINB8 | chr18: 59805517-59805576 | 0.025009 |
| A_23_P112113 | KIAA0196 | chr8: 126106049-126105990 | 0.025014 |
| A_23_P105423 | | chr12: 53630405-53630346 | 0.025034 |
| A_32_P211799 | USF2 | chr19: 40462164-40462105 | 0.025074 |
| A_24_P125871 | RIPK4 | chr21: 42032761-42032702 | 0.025085 |
| A_24_P782102 | | chr11: 062379527-062379586 | 0.025099 |
| A_24_P913546 | CTGLF9P | chr10: 46164006-46163947 | 0.025185 |
| A_23_P140362 | C14orf115 | chr14: 73895658-73895717 | 0.025203 |
| A_32_P16736 | | chr19: 41862924-41862865 | 0.025234 |
| A_24_P15856 | KIAA1875 | chr8: 145244136-145244195 | 0.025256 |
| A_24_P590164 | | chr16: 82517108-82517167 | 0.025355 |
| A_24_P931443 | GPR68 | chr14: 90768741-90768682 | 0.025385 |
| A_23_P111712 | MTERF | chr7: 91340721-91340662 | 0.025455 |
| A_32_P166653 | | chr1: 218150652-218150593 | 0.025456 |
| A_23_P28625 | WDR12 | chr2: 203453889-203453830 | 0.025466 |
| A_23_P362183 | ANKS6 | chr9: 100534184-100534125 | 0.025504 |
| A_23_P110005 | NICN1 | chr3: 49435653-49435594 | 0.025518 |
| A_23_P27810 | ZNF607 | chr19: 42879688-42879629 | 0.025521 |
| A_23_P55319 | FLOT2 | chr17: 24230908-24230849 | 0.025526 |
| A_24_P359050 | | chr8: 27261915-27261974 | 0.025537 |
| A_23_P82738 | RAD54B | chr8: 95453591-95453532 | 0.025549 |
| A_24_P926719 | | chr21: 35658691-35658750 | 0.025571 |
| A_23_P38219 | PRPF8 | chr17: 1501288-1501229 | 0.025648 |
| A_23_P7412 | BTNL8 | chr5: 180310378-180310437 | 0.025652 |
| A_23_P145247 | ZNF187 | chr6: 28353058-28353117 | 0.025657 |
| A_23_P128503 | IL26 | chr12: 66881570-66881511 | 0.025658 |
| A_24_P321093 | SPOCK2 | chr10: 73489746-73489687 | 0.025712 |
| A_24_P548866 | | chr3: 042799466-042799407 | 0.025722 |
| A_23_P110725 | PRKAA1 | chr5: 40798790-40798731 | 0.025736 |
| A_23_P48936 | SMAD3 | chr15: 65274445-65274504 | 0.025754 |
| A_23_P301328 | | chr21: 43982132-43982191 | 0.025759 |
| A_24_P919330 | FTH1 | chr11: 61491601-61491542 | 0.025780 |
| A_23_P210358 | LIMS1 | chr2: 108666947-108667006 | 0.025810 |
| A_23_P425880 | TRIO | chr5: 14561436-14561495 | 0.025837 |
| A_24_P11980 | | chr4: 026090159-026090100 | 0.025841 |
| A_23_P33768 | ZFYVE9 | chr1: 52584661-52584720 | 0.025849 |
| A_23_P325700 | C10orf126 | chr10: 29209683-29209742 | 0.025881 |
| A_23_P23542 | C1orf82 | chr1: 92574497-92574556 | 0.025926 |
| A_24_P409420 | | chr3: 032275758-032275821 | 0.025945 |
| A_23_P70213 | APC | chr5: 112208721-112208780 | 0.025946 |
| A_23_P390116 | SPATA13 | chr13: 23775575-23775634 | 0.025948 |
| A_24_P931932 | | chr9: 115283220-115283161 | 0.025955 |
| A_32_P10960 | FAM21C | chr10: 45604791-45604850 | 0.025984 |
| A_23_P322043 | SMG5 | chr1: 154485978-154485919 | 0.026219 |
| A_32_P20691 | CLK3 | chr15: 72709440-72709499 | 0.026283 |
| A_23_P43484 | CDKN2A | chr9: 21957839-21957780 | 0.026369 |
| A_24_P918131 | | chr1: 033551302-033551361 | 0.026373 |
| A_32_P154956 | | chr1: 142516996-142516937 | 0.026389 |
| A_23_P52885 | C11orf61 | chr11: 124142449-124142390 | 0.026570 |
| A_23_P27186 | KRT26 | chr17: 36177419-36176441 | 0.026598 |
| A_23_P31399 | PON2 | chr7: 94872495-94872436 | 0.026654 |
| A_24_P925966 | | chr2: 059522732-059522782 | 0.026697 |
| A_23_P49878 | FAM64A | chr17: 6294966-6295025 | 0.026715 |
| A_23_P49878 | FAM64A | chr17: 6294966-6295025 | 0.026730 |
| A_32_P200076 | tcag7.1017 | chr7: 99756554-99756613 | 0.026817 |
| A_24_P494194 | | chr15: 49360032-49359973 | 0.026837 |
| A_24_P330773 | CALCOCO2 | chr17: 44296923-44296982 | 0.026887 |
| A_23_P410600 | IDI2 | chr10: 1058643-1056799 | 0.026897 |
| A_23_P58464 | PCDHB6 | chr5: 140512424-140512483 | 0.026938 |
| A_23_P56127 | C19orf61 | chr19: 48929379-48929320 | 0.026946 |
| A_23_P90220 | ZNF585A | chr19: 42334475-42334416 | 0.026955 |
| A_32_P8351 | LOC595101 | chr16: 29444051-29443992 | 0.026958 |
| A_24_P937677 | | chr19: 057111047-057110988 | 0.026981 |
| A_23_P251303 | KIAA0310 | chr9: 138455189-138455130 | 0.027005 |
| A_23_P150950 | CCDC131 | chr12: 70290122-70290063 | 0.027017 |
| A_23_P24157 | C10orf33 | chr10: 100136977-100134785 | 0.027128 |
| A_24_P678104 | STMN3 | chr20: 61742447-61742388 | 0.027160 |
| A_24_P249824 | DYRK2 | chr12: 66340287-66340346 | 0.027164 |
| A_24_P392414 | | chr14: 105987250-105987190 | 0.027178 |
| A_23_P127565 | LAYN | chr11: 110936670-110936729 | 0.027246 |
| A_23_P37514 | C15orf39 | chr15: 73291486-73291545 | 0.027264 |
| A_23_P29575 | MRPS36 | chr5: 68560792-68560851 | 0.027268 |
| A_24_P63563 | KIAA0141 | chr5: 141299710-141299769 | 0.027275 |
| A_24_P133822 | ELAC2 | chr17: 12837738-12837038 | 0.027285 |
| A_32_P114739 | | chr1: 33172324-33172265 | 0.027305 |
| A_23_P20303 | DPYS | chr8: 105461144-105461085 | 0.027330 |
| A_23_P43484 | CDKN2A | chr9: 21957839-21957780 | 0.027338 |
| A_24_P247749 | RAB21 | chr12: 70462089-70462632 | 0.027356 |
| A_23_P141770 | NAPG | chr18: 10530343-10536328 | 0.027389 |
| A_23_P113111 | AR | chrX: 66860758-66860817 | 0.027391 |
| A_24_P85283 | POLR3A | chr10: 79423004-79420916 | 0.027405 |
| A_23_P203406 | GANAB | chr11: 62149137-62149078 | 0.027411 |
| A_32_P142881 | SMG1 | chr16: 18778411-18777978 | 0.027441 |
| A_23_P319013 | ZNF383 | chr19: 42425844-42425903 | 0.027450 |
| A_23_P65741 | DIS3L | chr15: 64412633-64412692 | 0.027451 |
| A_23_P218158 | SMEK1 | chr14: 90994362-90994303 | 0.027453 |
| A_23_P257863 | CCT5 | chr5: 10317913-10317972 | 0.027473 |
| A_24_P476086 | KPNA5 | chr6: 117163325-117163384 | 0.027533 |
| A_23_P113111 | AR | chrX: 66860758-66860817 | 0.027538 |
| A_23_P371777 | ACE | chr17: 58951414-58952448 | 0.027566 |
| A_23_P6935 | CD47 | chr3: 109262312-109261053 | 0.027575 |
| A_32_P153456 | | chr5: 040870101-040870365 | 0.027622 |
| A_24_P306479 | | chr16: 994213-994272 | 0.027625 |
| A_24_P780709 | | chr2: 107811640-107811559 | 0.027677 |
| A_23_P259707 | RP11-35N6.1 | chr9: 103126731-103126790 | 0.027684 |
| A_23_P319792 | XRN1 | chr3: 143508361-143508302 | 0.027684 |
| A_32_P4018 | ROR1 | chr1: 64419380-64419439 | 0.027814 |
| A_23_P60210 | RLN1 | chr9: 5325232-5325173 | 0.027849 |
| A_24_P460887 | | chr5: 131729974-131730033 | 0.027864 |
| A_24_P20767 | | chr5: 115657420-115657479 | 0.027889 |
| A_32_P217401 | BTBD9 | chr6: 38247776-38247717 | 0.027906 |
| A_32_P15887 | | chr17: 77062852-77062911 | 0.028009 |
| A_32_P934840 | C8orf34 | chr8: 69893131-69893190 | 0.028012 |
| A_23_P15146 | IL32 | chr16: 3059309-3059368 | 0.028042 |
| A_24_P256219 | MAF | chr16: 78188920-78188860 | 0.028046 |
| A_24_P315291 | RNF32 | chr7: 156144828-156144887 | 0.028062 |
| A_32_P230398 | | chr9: 92907181-92907122 | 0.028063 |
| A_23_P110851 | TERT | chr5: 1306364-1306305 | 0.028071 |
| A_24_P182433 | MAPBPIP | chr1: 154291798-154294409 | 0.028082 |
| A_23_P31306 | HOXA13 | chr7: 27204479-27204420 | 0.028086 |
| A_24_P196665 | GNGT1 | chr7: 93378047-93378106 | 0.028149 |
| A_24_P599237 | | chr12: 020975831-020975890 | 0.028150 |
| A_23_P15450 | TMEM100 | chr17: 51152817-51152758 | 0.028150 |
| A_23_P118690 | ZNF207 | chr17: 27721069-27721128 | 0.028187 |
| A_24_P80135 | PTPN18 | chr2: 130848551-130848610 | 0.028211 |
| A_23_P143047 | ATP6V1E2 | chr2: 46592697-46592638 | 0.028331 |
| A_23_P20932 | PDHB | chr3: 58391428-58390951 | 0.028344 |
| A_24_P100613 | LAMA1 | chr18: 6948512-6946742 | 0.028360 |
| A_32_P20679 | | chr10: 76976989-76977048 | 0.028441 |
| A_23_P66637 | SGCA | chr17: 45608226-45608285 | 0.028448 |
| A_23_P28707 | OGFR | chr20: 60915658-60915717 | 0.028464 |
| A_23_P43484 | CDKN2A | chr9: 21957839-21957780 | 0.028470 |
| A_24_P924651 | | chr1: 14930188-14930247 | 0.028483 |
| A_24_P163405 | TRIM27 | chr6: 28980392-28980333 | 0.028487 |
| A_23_P9485 | ORM2 | chr9: 116133726-116133936 | 0.028495 |
| A_23_P336693 | CLDN18 | chr3: 139226224-139231355 | 0.028516 |
| A_23_P38408 | ZNF403 | chr17: 32020040-32020099 | 0.028544 |
| A_24_P238578 | KIAA1729 | chr4: 10053987-10053928 | 0.028567 |
| A_24_P75680 | SLC4A8 | chr12: 50151469-50151528 | 0.028580 |
| A_24_P63397 | SPAR | chr4: 148169340-148169399 | 0.028608 |
| A_24_P41306 | | chr1: 31675015-31674956 | 0.028616 |
| A_24_P921683 | FOXP2 | chr7: 113853834-113853893 | 0.028632 |
| A_24_P123155 | ZNF639 | chr3: 180534583-180534642 | 0.028721 |
| A_23_P216984 | RAB14 | chr9: 122983349-122983290 | 0.028774 |
| A_24_P112447 | ENTPD7 | chr10: 101455019-101455078 | 0.028777 |
| A_24_P265111 | MLLT6 | chr17: 34127638-34127697 | 0.028808 |
| A_24_P253818 | FLOT2 | chr17: 24233332-24233273 | 0.028808 |
| A_23_P50389 | NAT14 | chr19: 60690324-60690382 | 0.028892 |
| A_24_P372123 | METTL2B | chr7: 127925411-127928230 | 0.028915 |
| A_24_P374163 | APOL4 | chr22: 34923541-34923482 | 0.029054 |
| A_23_P14019 | APOBEC1 | chr12: 7696388-7696329 | 0.029141 |
| A_24_P930829 | | | 0.029145 |
| A_23_P417100 | KRTAP4-6 | chr17: 36549520-36549461 | 0.029157 |
| A_24_P636974 | | chr7: 056402763-056402824 | 0.029251 |
| A_23_P423482 | FAM81A | chr15: 57602093-57602152 | 0.029254 |
| A_24_P367249 | | chr4: 152167972-152168033 | 0.029275 |
| A_23_P250607 | PLS3 | chrX: 114790572-114790631 | 0.029289 |
| A_23_P202258 | TFAM | chr10: 59825211-59825270 | 0.029362 |
| A_32_P222450 | TMEM158 | chr3: 45242210-45242151 | 0.029400 |
| A_23_P161634 | MUS81 | chr11: 65390175-65390234 | 0.029415 |
| A_23_P215318 | VPS41 | chr7: 38730357-38730298 | 0.029418 |
| A_23_P37410 | CYP19A1 | chr15: 49289400-49289341 | 0.029433 |
| A_24_P464118 | | chr2: 208441735-208441676 | 0.029458 |
| A_32_P61132 | | chr3: 186679700-186679641 | 0.029472 |
| A_23_P411335 | SGOL2 | chr2: 201146758-201146817 | 0.029521 |
| A_23_P406227 | MGC23284 | chr16: 87281000-87281059 | 0.029568 |
| A_23_P359043 | PALM2-AKAP2 | chr9: 111973579-111973638 | 0.029581 |
| A_23_P337292 | | chr1: 109181485-109181426 | 0.029675 |
| A_23_P113111 | AR | chrX: 66860758-66860817 | 0.029733 |
| A_32_P221832 | | chr2: 216698844-216698837 | 0.029779 |
| A_24_P37665 | SLC26A1 | chr4: 972310-972251 | 0.029878 |
| A_24_P25252 | ANKRD12 | chr18: 9248575-9248634 | 0.029888 |
| A_24_P941787 | PRPF4B | chr6: 4010126-4010185 | 0.029902 |
| A_23_P217379 | COL4A6 | chrX: 107285765-107285706 | 0.030053 |
| A_23_P398073 | PPM1B | chr2: 44313967-44314026 | 0.030074 |
| A_32_P151152 | | chr2: 10484961-10484902 | 0.030097 |
| A_24_P920629 | | chr7: 100988521-100988462 | 0.030107 |
| A_23_P113111 | AR | chrX: 66860758-66860817 | 0.030110 |
| A_23_P370005 | RIPK1 | chr6: 3058940-3058999 | 0.030116 |
| A_24_P804267 | ZNF818 | chr19: 58411084-58411143 | 0.030120 |
| A_24_P77681 | PAIP1 | chr5: 43569607-43565690 | 0.030144 |
| A_32_P181008 | | chrX: 112509266-112509325 | 0.030175 |
| A_23_P20622 | CDC14B | chr9: 98304870-98304813 | 0.030192 |
| A_24_P184732 | PAPLN | chr14: 72781174-72782109 | 0.030194 |
| A_32_P205323 | tcag7.1017 | chr7: 99756547-99756606 | 0.030225 |
| A_32_P162880 | LOC285708 | chr5: 101972425-101972366 | 0.030239 |
| A_24_P324141 | MUC5B | chr11: 1225946-1226005 | 0.030305 |
| A_24_P401645 | AP4S1 | chr14: 30631995-30632054 | 0.030336 |
| A_23_P83149 | FAM108B1 | chr9: 73671564-73667314 | 0.030420 |
| A_23_P409489 | DNTT | chr10: 98088071-98088130 | 0.030537 |
| A_24_P144054 | | chrX: 081648630-081648569 | 0.030543 |
| A_24_P273583 | | chr1: 9253583-9253524 | 0.030563 |
| A_23_P207837 | CPD | chr17: 25817189-25817248 | 0.030602 |
| A_23_P211445 | LIMK2 | chr22: 30005654-30005713 | 0.030616 |
| A_23_P121773 | FRG1 | chr4: 191120055-191121274 | 0.030629 |
| A_23_P163428 | PPCDC | chr15: 73128887-73128946 | 0.030633 |
| A_23_P102471 | MSH2 | chr2: 47563665-47563724 | 0.030633 |
| A_23_P105227 | ME3 | chr11: 85876036-85853841 | 0.030636 |
| A_24_P928250 | | | 0.030644 |
| A_24_P628384 | ALKBH2 | chr12: 108015520-108015461 | 0.030646 |
| A_24_P581311 | | chrX: 064931026-064931085 | 0.030668 |
| A_23_P360379 | EGLN3 | chr14: 33464451-33464392 | 0.030674 |
| A_24_P891116 | | chr1: 070875960-070875901 | 0.030808 |
| A_24_P187119 | OR5T2 | chr17: 5275612-5275671 | 0.030830 |
| A_24_P935889 | ACOX1 | chr17: 71452190-71452131 | 0.030844 |
| A_23_P130585 | CIB3 | chr19: 16136614-16136555 | 0.030887 |
| A_23_P115861 | ZNF485 | chr10: 43432512-43432571 | 0.030922 |
| A_32_P148476 | LOC389842 | chrX: 25574255-25574196 | 0.030967 |
| A_32_P213330 | RGNEF | chr5: 73235947-73241012 | 0.030980 |
| A_32_P88605 | | chr12: 077929433-077929492 | 0.031068 |
| A_32_P1173 | C6orf150 | chr6: 74195247-74195188 | 0.031077 |
| A_23_P340251 | RAB2A | chr8: 61667019-61667078 | 0.031080 |
| A_23_P149470 | NDUFS2 | chr1: 159446761-159447021 | 0.031095 |
| A_24_P36898 | GBP2 | chr1: 89345065-89345006 | 0.031104 |
| A_23_P14458 | C14orf149 | chr14: 59012671-59012612 | 0.031159 |
| A_24_P916407 | | chr12: 47811440-47811499 | 0.031173 |
| A_32_P182968 | | chr6: 132836682-132836741 | 0.031178 |
| A_23_P43484 | CDKN2A | chr9: 21957839-21957780 | 0.031220 |
| A_24_P921832 | | | 0.031226 |
| A_23_P102471 | MSH2 | chr2: 47563665-47563724 | 0.031233 |
| A_23_P95640 | C1orf186 | chr1: 204405696-204405637 | 0.031338 |
| A_32_P171747 | KIAA1729 | chr4: 10062358-10062299 | 0.031356 |
| A_23_P212781 | | chr4: 78180187-78180246 | 0.031377 |
| A_23_P104451 | DDX50 | chr10: 70376457-70376516 | 0.031388 |
| A_24_P55391 | | chr7: 75000838-75000779 | 0.031401 |
| A_23_P3514 | C16orf80 | chr16: 56706220-56705382 | 0.031408 |
| A_32_P137926 | C6orf167 | chr6: 97817994-97817935 | 0.031466 |
| A_23_P98057 | ZNF32 | chr10: 43460158-43460099 | 0.031487 |
| A_24_P200854 | HOXA2 | chr7: 27106934-27106875 | 0.031517 |
| A_23_P69918 | GNL3L | chrX: 54604276-54604335 | 0.031530 |
| A_23_P128828 | PABPN1 | chr14: 22862531-22863071 | 0.031556 |
| A_23_P83736 | ING2 | chr4: 184668870-184668929 | 0.031562 |
| A_24_P276791 | LRRC42 | chr1: 54198663-54198722 | 0.031582 |
| A_24_P374962 | DKFZP434A0131 | chr7: 74834647-74834706 | 0.031587 |
| A_24_P187154 | | chr7: 042802054-042802119 | 0.031587 |
| A_23_P43484 | CDKN2A | chr9: 21957839-21957780 | 0.031594 |
| A_24_P407224 | RASSF8 | chr12: 26116524-26116583 | 0.031660 |
| A_24_P173790 | RP4-621O15.2 | chr1: 226850835-226850894 | 0.031663 |
| A_23_P25835 | FNTB | chr14: 64598767-64598826 | 0.031687 |
| A_24_P220485 | OLFML2A | chr9: 126616841-126616900 | 0.031710 |
| A_32_P226907 | LOC284112 | chr17: 6859924-6859865 | 0.031721 |
| A_24_P147849 | | | 0.031834 |
| A_23_P37856 | HBA1 | chr16: 167382-167441 | 0.031885 |
| A_23_P117424 | WDR23 | chr14: 23662651-23662710 | 0.031928 |
| A_23_P250506 | DPM3 | chr1: 153379167-153379108 | 0.031946 |
| A_23_P62868 | EXOSC10 | chr1: 11050694-11049376 | 0.031948 |
| A_23_P305877 | C9orf123 | chr9: 7787510-7787451 | 0.031977 |
| A_32_P213342 | | chr12: 1483044-1483103 | 0.032055 |
| A_24_P25498 | | chr2: 133404842-133404901 | 0.032110 |
| A_23_P397910 | CBLC | chr19: 49987504-49987563 | 0.032125 |
| A_24_P145249 | ATP6V1B1 | chr2: 71042684-71042743 | 0.032130 |
| A_23_P3849 | TRAP1 | chr16: 3661732-3656070 | 0.032144 |
| A_32_P115077 | | chr9: 130090921-130090862 | 0.032200 |
| A_32_P2605 | | chr1: 224254578-224254637 | 0.032261 |
| A_32_P34003 | | chr2: 164167751-164167692 | 0.032304 |
| A_32_P104746 | ZFYVE28 | chr4: 2241317-2241258 | 0.032324 |
| A_23_P100092 | ZSCAN29 | chr15: 41440680-41440621 | 0.032341 |
| A_23_P78585 | CYP2F1 | chr19: 46319875-46320622 | 0.032366 |
| A_24_P107208 | CAPRIN1 | chr11: 34068390-34068688 | 0.032398 |
| A_24_P456043 | | chr6: 017754043-017754101 | 0.032475 |
| A_24_P110799 | LSS | chr21: 46433786-46433727 | 0.032546 |
| A_32_P105200 | | chr2: 108755584-108755643 | 0.032551 |
| A_24_P52714 | RNASEH2C | chr11: 65244366-65244307 | 0.032556 |
| A_24_P336759 | MCL1 | chr1: 148814305-148814246 | 0.032559 |
| A_32_P220567 | | chr2: 206644805-206644746 | 0.032561 |
| A_23_P43484 | CDKN2A | chr9: 21957839-21957780 | 0.032588 |
| A_32_P79152 | | chr10: 13809705-13809764 | 0.032633 |
| A_32_P109933 | | chr7: 12591036-12590977 | 0.032645 |
| A_23_P250910 | UBQLN1 | chr9: 85466399-85466340 | 0.032666 |
| A_24_P733016 | LOC390427 | chr13: 113982309-113982250 | 0.032692 |
| A_24_P255845 | LOC400011 | chr12: 16443009-16442994 | 0.032697 |
| A_23_P44207 | ACOT12 | chr5: 80662097-80662038 | 0.032706 |
| A_32_P15838 | | chr5: 11652894-11652835 | 0.032766 |
| A_23_P136870 | MAGEA6 | chrX: 151620825-151620884 | 0.032767 |
| A_32_P182991 | | chr2: 242314087-242314028 | 0.032781 |
| A_23_P95050 | ARFIP1 | chr4: 154050802-154050861 | 0.032810 |
| A_32_P104694 | | chr14: 93917188-93918697 | 0.032821 |
| A_23_P319153 | | chr11: 70887238-70887297 | 0.032825 |
| A_24_P544230 | | chr5: 133557999-133558058 | 0.032847 |
| A_23_P305761 | PHF12 | chr17: 24263546-24263487 | 0.032867 |
| A_24_P232856 | RPL9 | chr4: 39136276-39136217 | 0.032900 |
| A_23_P21346 | PCGF3 | chr4: 749946-750005 | 0.032914 |
| A_32_P95757 | C6orf128 | chr6: 37294437-37294378 | 0.032942 |
| A_32_P224327 | LOC730091 | chr3: 157948010-157947951 | 0.032951 |
| A_23_P361569 | SLC35F5 | chr2: 114191841-114189225 | 0.033007 |
| A_24_P186944 | LOC389404 | chr6: 64383628-64383600 | 0.033024 |
| A_32_P1144 | | chr8: 17127026-17126967 | 0.033088 |
| A_23_P110851 | TERT | chr5: 1306364-1306305 | 0.033111 |
| A_23_P166431 | C22orf24 | chr22: 30659920-30659861 | 0.033125 |
| A_32_P212566 | | chr12: 031414130-031414189 | 0.033129 |
| A_23_P93690 | MCM7 | chr7: 99528515-99528456 | 0.033132 |
| A_24_P257539 | FBXW11 | chr5: 171228351-171228292 | 0.033159 |
| A_24_P731255 | | chr2: 69851041-69851100 | 0.033173 |
| A_32_P201118 | | chr8: 140997982-140998041 | 0.033200 |
| A_23_P407662 | KLRA1 | chr12: 10637717-10637658 | 0.033206 |
| A_23_P8571 | SRCRB4D | chr7: 75856644-75856585 | 0.033212 |
| A_23_P5831 | HPCAL1 | chr2: 10484431-10484490 | 0.033248 |
| A_24_P926580 | | chr2: 164267881-164267822 | 0.033268 |
| A_32_P466514 | C14orf4 | chr14: 76561745-76561686 | 0.033358 |
| A_23_P145846 | MET | chr7: 116223309-116223368 | 0.033375 |
| A_24_P225845 | | chr3: 154040850-154040909 | 0.033379 |
| A_23_P320290 | LOC152485 | chr4: 146905763-146905704 | 0.033462 |
| A_24_P557274 | | chr6: 11186240-11186299 | 0.033520 |
| A_23_P158484 | CYP2C19 | chr10: 96524956-96525184 | 0.033526 |
| A_23_P361336 | ADARB1 | chr21: 45467377-45467436 | 0.033570 |
| A_23_P125147 | RAB28 | chr4: 12980610-12979331 | 0.033622 |
| A_23_P110430 | MSX1 | chr4: 4915945-4916004 | 0.033641 |
| A_23_P323930 | TSPAN5 | chr4: 99612490-99612431 | 0.033647 |
| A_24_P919812 | | chr3: 186990813-186990872 | 0.033667 |
| A_24_P800683 | | chr4: 90933938-90933997 | 0.033680 |
| A_32_P51082 | | chr10: 75213008-75212949 | 0.033771 |
| A_23_P108948 | MREG | chr2: 216516404-216516345 | 0.033808 |
| A_32_P19460 | | chr10: 094199882-094199823 | 0.033834 |
| A_23_P137361 | WDR8 | chr1: 3538734-3538675 | 0.033839 |
| A_32_P203066 | | chrX: 115711174-115711115 | 0.033853 |
| A_23_P168651 | CDK6 | chr7: 92138707-92090309 | 0.033864 |
| A_23_P311834 | MLLT10 | chr10: 21915257-21924297 | 0.033865 |
| A_24_P205589 | ACOT7 | chr1: 6277570-6277511 | 0.033885 |
| A_32_P110575 | | chr2: 54051867-54051926 | 0.033909 |
| A_23_P210048 | HDAC4 | chr2: 239635672-239635613 | 0.033915 |
| A_24_P936363 | | chr16: 2662120-2662061 | 0.033961 |
| A_24_P336577 | C1orf183 | chr1: 112070774-112070715 | 0.033969 |
| A_23_P416036 | UCHL5IP | chrX: 152373052-152366509 | 0.033994 |
| A_23_P71067 | TWIST1 | chr7: 19122084-19122025 | 0.034006 |
| A_24_P460309 | | chr15: 93808469-93808410 | 0.034009 |
| A_24_P122403 | TCEB3 | chr1: 23960866-23960925 | 0.034010 |
| A_23_P83781 | PSCD1 | chr17: 74181792-74181733 | 0.034042 |
| A_23_P132486 | OXSM | chr3: 25810791-25810850 | 0.034095 |
| A_24_P923591 | | chr22: 20354316-20354375 | 0.034104 |
| A_24_P924667 | | chr1: 199261653-199261594 | 0.034122 |
| A_24_P696009 | | chr3: 132662342-132662401 | 0.034143 |
| A_23_P156880 | ENPP1 | chr6: 132253564-132253623 | 0.034163 |
| A_24_P119609 | MYO1D | chr17: 28116180-28116121 | 0.034217 |
| A_24_P401051 | | | 0.034232 |
| A_23_P251051 | NF2 | chr22: 28424507-28424566 | 0.034315 |
| A_24_P246091 | FLJ37201 | chr10: 91441562-91441503 | 0.034330 |
| A_23_P341471 | UBE2N | chr12: 92327538-92327479 | 0.034353 |
| A_23_P88234 | C14orf122 | chr14: 23678419-23678197 | 0.034382 |
| A_23_P13820 | SLCO1A2 | chr12: 21318754-21318695 | 0.034403 |
| A_23_P146354 | POMT1 | chr9: 133388947-133389006 | 0.034410 |
| A_23_P340922 | ZNF414 | chr19: 8483309-8482824 | 0.034430 |
| A_24_P50801 | NRP2 | chr2: 206316325-206316384 | 0.034504 |
| A_23_P113111 | AR | chrX: 66860758-66860817 | 0.034531 |
| A_32_P109516 | | chr2: 239988892-239988951 | 0.034582 |
| A_23_P38519 | ITGB3 | chr17: 42743726-42743785 | 0.034602 |
| A_23_P71989 | UPP1 | chr7: 48114681-48114740 | 0.034612 |
| A_24_P706953 | LOC155006 | chr7: 138140418-138140477 | 0.034612 |
| A_23_P25187 | KRT81 | chr12: 50966199-50966140 | 0.034628 |
| A_23_P144294 | SCC-112 | chr4: 39502433-39502374 | 0.034640 |
| A_24_P384573 | EWSR1 | chr22: 28013062-28013121 | 0.034754 |
| A_23_P163178 | CALM1 | chr14: 89941478-89941537 | 0.034757 |
| A_32_P149536 | SUMO2 | chr17: 70675569-70675510 | 0.034785 |
| A_24_P126417 | USP34 | chr2: 61268996-61268937 | 0.034793 |
| A_23_P336838 | | chr9: 97576280-97576339 | 0.034799 |
| A_23_P34473 | RPE65 | chr1: 68667538-68667479 | 0.034814 |
| A_23_P63668 | IFIT5 | chr10: 91168323-91168382 | 0.034822 |
| A_23_P29851 | LRPAP1 | chr4: 3496441-3491669 | 0.034835 |
| A_23_P317207 | ATXN7L2 | chr1: 109835411-109835470 | 0.034904 |
| A_24_P13032 | UBE2D1 | chr10: 59797707-59797766 | 0.034921 |
| A_24_P229638 | C9orf117 | chr9: 129516002-129516061 | 0.034946 |
| A_23_P412990 | HRH3 | chr20: 60223873-60223814 | 0.034954 |
| A_23_P35576 | ZNF518 | chr10: 97910005-97910064 | 0.034976 |
| A_23_P124164 | ISY1 | chr3: 130336440-130336381 | 0.034984 |
| A_23_P112859 | CST1 | chr20: 23679379-23679320 | 0.034995 |
| A_23_P14035 | AKAP3 | chr12: 4605980-4595320 | 0.035035 |
| A_23_P141917 | TYK2 | chr19: 10326213-10325877 | 0.035057 |
| A_23_P89506 | | chr17: 023715452-023715393 | 0.035105 |
| A_23_P113111 | AR | chrX: 66860758-66860817 | 0.035116 |
| A_24_P306921 | LOC124496 | chr16: 74300671-74300612 | 0.035165 |
| A_24_P923945 | ZUBR1 | chr1: 19311421-19311362 | 0.035323 |
| A_23_P501805 | LIPT1 | chr2: 99140458-99140517 | 0.035327 |
| A_23_P257155 | ATXN7 | chr3: 63956919-63956978 | 0.035354 |
| A_24_P933268 | | chr5: 112186922-112186981 | 0.035403 |
| A_23_P133506 | DOK3 | chr5: 176863597-176863538 | 0.035435 |
| A_23_P127367 | POLD4 | chr11: 66875773-66875714 | 0.035440 |
| A_24_P29641 | NSUN5C | chr7: 72058363-72057950 | 0.035447 |
| A_24_P197196 | ABCB6 | chr2: 219783388-219783247 | 0.035450 |
| A_23_P54709 | RAB26 | chr16: 2143477-2143536 | 0.035495 |
| A_23_P40548 | YPEL1 | chr22: 20385424-20385365 | 0.035507 |
| A_24_P294124 | SERTAD2 | chr2: 64712664-64712605 | 0.035526 |
| A_23_P82913 | TRMT12 | chr8: 125533948-125534007 | 0.035549 |
| A_23_P88362 | PPIL5 | chr14: 49144371-49144430 | 0.035617 |
| A_23_P210015 | | chr2: 130848938-130848997 | 0.035700 |
| A_23_P132115 | SNF1LK | chr21: 43659621-43659562 | 0.035731 |
| A_23_P171314 | FUNDC2 | chrX: 153914891-153914950 | 0.035781 |
| A_23_P30020 | PLA2G12A | chr4: 110855065-110855006 | 0.035884 |
| A_23_P316974 | SYNJ2 | chr6: 158439219-158439278 | 0.035888 |
| A_24_P135483 | | chr10: 82270494-82270553 | 0.035897 |
| A_24_P852601 | HNF4G | chr8: 76640553-76640612 | 0.035907 |
| A_32_P17133 | PPP1R2 | chr3: 196722492-196722479 | 0.035999 |
| A_23_P402787 | FAM100A | chr16: 4599052-4598993 | 0.036007 |
| A_23_P141479 | SUPT6H | chr17: 24053088-24053147 | 0.036008 |
| A_24_P940059 | PIP3-E | chr6: 154518125-154518066 | 0.036026 |
| A_24_P570930 | | chr16: 030961347-030961288 | 0.036030 |
| A_23_P104438 | MYPN | chr10: 69640999-69641058 | 0.036048 |
| A_24_P52882 | HRK | chr12: 115803412-115803353 | 0.036173 |
| A_32_P193378 | LOC283658 | chr15: 53618931-53618872 | 0.036217 |
| A_24_P1910 | SUPT4H1 | chr17: 53777730-53777671 | 0.036218 |
| A_23_P78108 | ALDOC | chr17: 23924609-23924550 | 0.036224 |
| A_32_P114215 | COMMD6 | chr13: 74998704-74998645 | 0.036239 |
| A_32_P57728 | | chr7: 99756589-99756530 | 0.036270 |
| A_23_P305977 | GRAMD2 | chr15: 70239939-70239880 | 0.036284 |
| A_23_P165450 | ATG9A | chr2: 219792746-219792687 | 0.036332 |
| A_24_P195037 | C1orf52 | chr1: 85496884-85496825 | 0.036386 |
| A_24_P191781 | DKFZP564O0823 | chr4: 76193776-76193835 | 0.036432 |
| A_23_P99386 | TNFSF11 | chr13: 42079387-42079446 | 0.036454 |
| A_23_P131375 | PQLC3 | chr2: 11236125-11236184 | 0.036464 |
| A_23_P371613 | CHCHD1 | chr10: 75212932-75212991 | 0.036482 |
| A_23_P133923 | BAT4 | chr6: 31737919-31737860 | 0.036483 |
| A_23_P397455 | ACVR1C | chr2: 158097461-158097402 | 0.036488 |
| A_24_P221858 | PIWIL3 | chr22: 23450872-23449182 | 0.036526 |
| A_23_P145463 | SLC35B3 | chr6: 8366051-8365992 | 0.036546 |
| A_23_P122481 | | chr6: 028129755-028129696 | 0.036584 |
| A_24_P332314 | FAM111B | chr11: 58648678-58648737 | 0.036609 |
| A_23_P145370 | C6orf107 | chr6: 34948465-34948524 | 0.036638 |
| A_24_P275723 | WDR90 | chr16: 653048-653107 | 0.036676 |
| A_23_P88781 | CIAPIN1 | chr16: 56020201-56020142 | 0.036717 |
| A_24_P80532 | CCNG2 | chr4: 78301734-78301883 | 0.036790 |
| A_32_P24338 | | chr3: 178377535-178377476 | 0.036842 |
| A_23_P97700 | TXNIP | chr1: 144153804-144153863 | 0.036861 |
| A_32_P154342 | SLCO4C1 | chr5: 101597933-101597874 | 0.036868 |
| A_32_P14881 | | chr21: 43603420-43603361 | 0.036927 |
| A_32_P226498 | HAR1A | chr20: 61205765-61205824 | 0.036930 |
| A_23_P26468 | RHBDL1 | chr16: 666856-666989 | 0.036933 |
| A_24_P53282 | CPD | chr17: 25819173-25819232 | 0.037003 |
| A_23_P131846 | SNAI1 | chr20: 48038577-48038636 | 0.037010 |
| A_32_P427222 | | chr2: 234405582-234405641 | 0.037012 |
| A_24_P53778 | ITLN2 | chr1: 159181651-159181592 | 0.037030 |
| A_24_P289043 | | chr9: 004935132-004935071 | 0.037045 |
| A_23_P74068 | MSH4 | chr1: 76137964-76151012 | 0.037067 |
| A_23_P74828 | GJA10 | chr1: 39112802-39112743 | 0.037068 |
| A_23_P116614 | ME3 | chr11: 85830001-85829942 | 0.037106 |
| A_24_P50696 | | chr2: 201182347-201185446 | 0.037151 |
| A_23_P19226 | DSE | chr6: 116865133-116865192 | 0.037312 |
| A_24_P76210 | | chrX: 053189643-053189584 | 0.037351 |
| A_23_P119418 | C19orf2 | chr19: 35195068-35195127 | 0.037363 |
| A_24_P937649 | HLA-DQB1 | chr6: 32734003-32733944 | 0.037463 |
| A_24_P360601 | NOL8 | chr9: 94108991-94107877 | 0.037504 |
| A_24_P842872 | | chr1: 110061966-110062025 | 0.037511 |
| A_32_P98776 | | chr16: 087848904-087848845 | 0.037545 |
| A_23_P160481 | IQWD1 | chr1: 166302292-166304243 | 0.037591 |
| A_23_P113111 | AR | chrX: 66860758-66860817 | 0.037619 |
| A_23_P106814 | | chr16: 002829368-002829427 | 0.037647 |
| A_23_P129157 | NEIL1 | chr15: 73434426-73434485 | 0.037653 |
| A_32_P225734 | | chr16: 29464072-29464131 | 0.037669 |
| A_23_P204947 | GJB2 | chr13: 19659974-19659915 | 0.037718 |
| A_23_P158933 | GPR125 | chr4: 21998981-21998922 | 0.037758 |
| A_23_P406986 | MOSPD2 | chrX: 14849256-14849315 | 0.037777 |
| A_24_P130962 | TOR3A | chr1: 177329884-177329943 | 0.037867 |
| A_24_P93703 | LOC440104 | chr12: 54516091-54516150 | 0.037889 |
| A_24_P803801 | GTF2I | chr7: 73805391-73806118 | 0.037912 |
| A_24_P196592 | MMP28 | chr17: 31130413-31130186 | 0.037918 |
| A_23_P424689 | RBMY2FP | chrY: 22870752-22870811 | 0.037933 |
| A_24_P67189 | | chr17: 7265313-7265371 | 0.038097 |
| A_23_P32474 | CDCP1 | chr3: 45102513-45102454 | 0.038137 |
| A_24_P124875 | OTUB2 | chr14: 93580116-93580705 | 0.038182 |
| A_24_P27644 | | chr17: 18813553-18813612 | 0.038210 |
| A_24_P148521 | TMBIM1 | chr2: 218852052-218851521 | 0.038242 |
| A_23_P27048 | C17orf32 | chr17: 23713044-23713103 | 0.038395 |
| A_32_P119096 | MBOAT2 | chr2: 8910591-8910532 | 0.038420 |
| A_23_P157736 | PPAPDC3 | chr9: 133174399-133174458 | 0.038515 |
| A_24_P784213 | | | 0.038530 |
| A_23_P160968 | LAMC2 | chr1: 181475924-181476076 | 0.038693 |
| A_24_P94054 | STK4 | chr20: 43049326-43057178 | 0.038739 |
| A_23_P502609 | PIGQ | chr16: 573578-573637 | 0.038788 |
| A_23_P48936 | SMAD3 | chr15: 65274445-65274504 | 0.038807 |
| A_23_P8513 | SNX10 | chr7: 26380261-26380320 | 0.038829 |
| A_23_P27285 | MPPE1 | chr18: 11874349-11874290 | 0.038907 |
| A_24_P22562 | 2-Apr | chr17: 39350372-39350431 | 0.038907 |
| A_23_P26878 | NDEL1 | chr17: 8311542-8311601 | 0.038959 |
| A_23_P33123 | SPOCK3 | chr4: 167895325-167895266 | 0.038985 |
| A_24_P201171 | STXBP1 | chr9: 129494013-129494072 | 0.039012 |
| A_23_P164068 | DNAH9 | chr17: 11813608-11813667 | 0.039035 |
| A_23_P150818 | MBD6 | chr12: 56209715-56210033 | 0.039070 |
| A_24_P230808 | BTBD7 | chr14: 92777623-92777564 | 0.039117 |
| A_32_P144342 | PARP4 | chr13: 23893222-23893163 | 0.039132 |
| A_24_P272561 | ZDHHC13 | chr11: 19124388-19125739 | 0.039145 |
| A_24_P868334 | | chr5: 88025226-88025167 | 0.039154 |
| A_23_P47073 | WDR37 | chr10: 1167673-1167732 | 0.039172 |
| A_23_P253701 | | chr3: 54888128-54888187 | 0.039243 |
| A_24_P760130 | | chr8: 98809847-98809906 | 0.039245 |
| A_23_P80473 | CHST13 | chr3: 127744440-127744499 | 0.039305 |
| A_24_P335092 | SAA1 | chr11: 18247444-18247886 | 0.039458 |
| A_23_P218047 | KRT5 | chr12: 51194948-51194889 | 0.039527 |
| A_23_P394323 | FAM98B | chr15: 36562353-36562412 | 0.039531 |
| A_32_P144018 | | chr12: 25999269-25999210 | 0.039575 |
| A_24_P330030 | ORAOV1 | chr11: 69191933-69191874 | 0.039605 |
| A_23_P49155 | CDH3 | chr16: 67290313-67290372 | 0.039633 |
| A_23_P162589 | VDR | chr12: 46521828-46521769 | 0.039652 |
| A_23_P215421 | WIPI2 | chr7: 5237711-5237770 | 0.039665 |
| A_23_P118306 | DNAJA3 | chr16: 4446393-4446452 | 0.039697 |
| A_23_P143694 | SOX10 | chr22: 36699094-36699035 | 0.039700 |
| A_32_P186038 | | chr15: 55635700-55635641 | 0.039742 |
| A_23_P7423 | NSUN2 | chr5: 6652588-6652529 | 0.039751 |
| A_32_P179854 | BRP44 | chr1: 166152876-166152817 | 0.039771 |
| A_32_P232883 | | chr11: 121404323-121404264 | 0.039792 |
| A_32_P203998 | | chr6: 122806787-122806846 | 0.039794 |
| A_24_P847772 | | chr20: 427309-427368 | 0.039803 |
| A_24_P919399 | ATP10D | chr4: 47209523-47209582 | 0.039860 |
| A_23_P34233 | QPRT | chr16: 29616171-29616230 | 0.039865 |
| A_23_P205236 | PPP2R5C | chr14: 101463324-101463383 | 0.039905 |
| A_24_P144254 | | chrX: 132632766-132632830 | 0.039924 |
| A_24_P331711 | THEM5 | chr1: 150085988-150085929 | 0.039969 |
| A_24_P168510 | C19orf50 | chr19: 18536774-18538943 | 0.040003 |
| A_24_P130865 | PHF8 | chrX: 54006075-54006016 | 0.040095 |
| A_23_P67847 | GALNT14 | chr2: 30986933-30986874 | 0.040117 |
| A_24_P923807 | LOC730038 | chr15: 26832933-26832928 | 0.040140 |
| A_23_P11922 | FLJ21272 | chr1: 143702517-143702576 | 0.040145 |
| A_23_P9707 | | | 0.040160 |
| A_23_P374284 | CASC3 | chr17: 35581863-35581922 | 0.040231 |
| A_24_P521544 | | chr3: 073009769-073009710 | 0.040233 |
| A_23_P106682 | EMP2 | chr16: 10534260-10534201 | 0.040308 |
| A_24_P296907 | THAP8 | chr19: 41218197-41218138 | 0.040360 |
| A_24_P139742 | NMNAT1 | chr1: 9965365-9965424 | 0.040419 |
| A_32_P155786 | RPS2 | chr16: 1953245-1954323 | 0.040521 |
| A_24_P136744 | | chr9: 83580909-83580968 | 0.040531 |
| A_24_P940288 | PGS1 | chr17: 73929219-73929278 | 0.040605 |
| A_23_P35399 | ANXA11 | chr10: 81907396-81905609 | 0.040606 |
| A_24_P914155 | USP54 | chr10: 74960069-74960010 | 0.040611 |
| A_23_P148798 | UCHL5 | chr1: 191252026-191251967 | 0.040639 |
| A_23_P9340 | BARHL1 | chr9: 134455236-134455295 | 0.040669 |
| A_24_P141332 | CAMK2G | chr10: 75242432-75242373 | 0.040687 |
| A_24_P342944 | CSPG5 | chr3: 47589218-47585701 | 0.040722 |
| A_24_P485105 | | chr9: 131737515-131737456 | 0.040729 |
| A_24_P120734 | MGC16075 | chr7: 47772344-47772403 | 0.040732 |
| A_23_P105012 | HRASLS2 | chr11: 63076959-63076900 | 0.040756 |
| A_23_P148600 | INE1 | chrX: 46949676-46949735 | 0.040768 |
| A_24_P286013 | HIP2 | chr4: 39456380-39456439 | 0.040838 |
| A_23_P325075 | RNGTT | chr6: 89377068-89377009 | 0.040947 |
| A_24_P45767 | AGBL5 | chr2: 27146934-27146993 | 0.040965 |
| A_23_P118313 | GABARAPL2 | chr16: 74169049-74169108 | 0.040977 |
| A_32_P808 | SLAIN2 | chr4: 48080467-48080526 | 0.041042 |
| A_32_P179572 | | chr16: 23490894-23490835 | 0.041047 |
| A_23_P126218 | CSF3R | chr1: 36704892-36704833 | 0.041057 |
| A_23_P29784 | ARMC8 | chr3: 139499640-139499699 | 0.041103 |
| A_23_P136870 | MAGEA6 | chrX: 151620825-151620884 | 0.041159 |
| A_32_P168053 | | chr3: 117591444-117591503 | 0.041169 |
| A_23_P133386 | RASGRF2 | chr5: 80557529-80557588 | 0.041187 |
| A_24_P918507 | | chr2: 87878752-87878811 | 0.041245 |
| A_23_P212160 | C3orf31 | chr3: 11824334-11807024 | 0.041297 |
| A_23_P256804 | SNUPN | chr15: 73677562-73677503 | 0.041308 |
| A_24_P214043 | | chr21: 42562366-42562425 | 0.041312 |
| A_24_P146603 | DDX21 | chr10: 70412865-70412924 | 0.041340 |
| A_23_P314755 | STC1 | chr8: 23756122-23756063 | 0.041354 |
| A_23_P208961 | MUM1 | chr19: 1329272-1329331 | 0.041366 |
| A_24_P943709 | | chr8: 127636198-127636257 | 0.041493 |
| A_32_P5205 | | chr8: 102542922-102542863 | 0.041495 |
| A_24_P919364 | | chr6: 043110054-043109995 | 0.041545 |
| A_24_P912994 | | | 0.041550 |
| A_24_P289709 | | chr11: 5227339-5227398 | 0.041615 |
| A_23_P111092 | OR2H1 | chr6: 29538001-29538060 | 0.041654 |
| A_23_P336342 | | chr12: 50195437-50195496 | 0.041673 |
| A_32_P92281 | | chr7: 44013845-44013786 | 0.041699 |
| A_24_P252310 | FAM131B | chr7: 142761088-142761029 | 0.041801 |
| A_23_P337849 | TNRC4 | chr1: 149941710-149941651 | 0.041823 |
| A_24_P229936 | CIITA | chr16: 10909524-10909585 | 0.041839 |
| A_32_P34365 | | chr1: 146387818-146387759 | 0.041931 |
| A_23_P342185 | WAPAL | chr10: 88187242-88187183 | 0.041959 |
| A_24_P317604 | SLC37A3 | chr7: 139681408-139681349 | 0.041962 |
| A_24_P715644 | | chr8: 144867528-144867587 | 0.041962 |
| A_24_P895687 | | chr12: 127001717-127001777 | 0.041965 |
| A_24_P318319 | MED6 | chr14: 70127847-70127788 | 0.041984 |
| A_23_P55544 | CCBE1 | chr18: 55253070-55253011 | 0.041988 |
| A_24_P330021 | SLC22A9 | chr11: 62906315-62930593 | 0.042020 |
| A_23_P165541 | | chr2: 102826424-102826483 | 0.042061 |
| A_24_P393548 | | chr7: 86944555-86944615 | 0.042062 |
| A_24_P919585 | | chr17: 024130120-024130179 | 0.042113 |
| A_32_P134167 | | chr11: 107384681-107384622 | 0.042122 |
| A_23_P160720 | SNFT | chr1: 210926710-210926651 | 0.042170 |
| A_24_P157156 | FLJ39660 | chr2: 197298980-197299039 | 0.042208 |
| A_23_P159125 | SLC16A5 | chr17: 70613744-70613803 | 0.042216 |
| A_23_P202540 | MAPK8 | chr10: 49314336-49314395 | 0.042217 |
| A_32_P5432 | | chr16: 80095079-80095138 | 0.042219 |
| A_23_P97021 | EIF2C3 | chr1: 36294139-36294198 | 0.042232 |
| A_23_P13094 | MMP10 | chr11: 102146674-102146615 | 0.042286 |
| A_24_P201089 | SLC25A37 | chr8: 23479714-23479773 | 0.042302 |
| A_23_P426989 | SH3BP5L | chr1: 247071517-247071458 | 0.042323 |
| A_23_P328034 | C20orf96 | chr20: 205767-205708 | 0.042327 |
| A_23_P90752 | NAG | chr2: 15244386-15244327 | 0.042361 |
| A_23_P50217 | ZNF671 | chr19: 62923107-62923048 | 0.042362 |
| A_32_P140211 | | chr1: 246928834-246928893 | 0.042429 |
| A_23_P40503 | ZNF295 | chr21: 42284860-42284801 | 0.042442 |
| A_24_P127748 | CLASP2 | chr3: 33628560-33628501 | 0.042510 |
| A_24_P930927 | | chr12: 100683862-100683803 | 0.042514 |
| A_23_P10647 | CYTL1 | chr4: 5067682-5067623 | 0.042526 |
| A_24_P405313 | SLC25A12 | chr2: 172378105-172374984 | 0.042534 |
| A_23_P110606 | MAML1 | chr5: 179136122-179136181 | 0.042549 |
| A_24_P356830 | | chr19: 3416248-3416307 | 0.042558 |
| A_23_P163251 | PAQR5 | chr15: 67483280-67483339 | 0.042614 |
| A_23_P51187 | PRKCZ | chr1: 2106563-2106622 | 0.042615 |
| A_32_P214054 | | chr8: 123902808-123902867 | 0.042621 |
| A_32_P220938 | RPE | chr2: 210592844-210592903 | 0.042651 |
| A_23_P432352 | RP3-452H17.2 | chrX: 115507053-115506994 | 0.042667 |
| A_24_P303524 | MICALL2 | chr7: 1440600-1440541 | 0.042672 |
| A_23_P410998 | RAB5B | chr12: 54674293-54674352 | 0.042677 |
| A_32_P493702 | | chr2: 171205814-171203206 | 0.042687 |
| A_23_P18246 | XCR1 | chr3: 46037989-46037930 | 0.042752 |
| A_24_P370702 | GBP3 | chr1: 89246100-89246041 | 0.042758 |
| A_24_P884376 | | chr7: 045821304-045821363 | 0.042774 |
| A_32_P136450 | | chr20: 14476013-14475954 | 0.042805 |
| A_24_P244410 | C11orf51 | chr11: 71498826-71498608 | 0.042805 |
| A_23_P216153 | MMP16 | chr8: 89123035-89122976 | 0.042846 |
| A_32_P235128 | RPL37 | chr5: 40870096-40868430 | 0.042856 |
| A_32_P172509 | | chr15: 39381165-39381106 | 0.042863 |
| A_32_P18251 | LOC388965 | chr2: 84372288-84372347 | 0.042893 |
| A_23_P390984 | KCNH6 | chr17: 58977750-58977809 | 0.042893 |
| A_23_P412059 | ZRANB3 | chr2: 135674003-135673944 | 0.042964 |
| A_23_P434807 | RND2 | chr17: 38431574-38432768 | 0.043028 |
| A_32_P224302 | ZNF135 | chr19: 63271999-63272058 | 0.043033 |
| A_24_P943283 | FLJ20054 | chr1: 195841336-195841277 | 0.043050 |
| A_23_P103942 | DNAJC11 | chr1: 6616847-6616818 | 0.043070 |
| A_23_P124202 | ING5 | chr2: 242313101-242313158 | 0.043145 |
| A_24_P59062 | GGTL4 | chr22: 21318821-21318880 | 0.043167 |
| A_32_P70420 | | chr12: 120323184-120323243 | 0.043207 |
| A_23_P303423 | | chr4: 48122180-48122239 | 0.043208 |
| A_32_P43320 | | chr1: 184916820-184916879 | 0.043221 |
| A_24_P63178 | CHL1 | chr3: 424779-424838 | 0.043233 |
| A_24_P933761 | | chr12: 093573280-093573221 | 0.043251 |
| A_23_P372386 | SCARA5 | chr8: 27879913-27879854 | 0.043261 |
| A_23_P207596 | NF1 | chr17: 26725374-26725433 | 0.043278 |
| A_23_P78734 | MYH14 | chr19: 55505129-55505188 | 0.043282 |
| A_24_P114255 | MBOAT2 | chr2: 8914684-8914625 | 0.043315 |
| A_23_P150619 | TRIM3 | chr11: 6428396-6427233 | 0.043332 |
| A_32_P203515 | SPECC1L | chr22: 23142928-23142987 | 0.043343 |
| A_24_P312845 | CEND1 | chr11: 778360-778301 | 0.043370 |
| A_24_P252973 | CLP1 | chr11: 57185218-57185277 | 0.043388 |
| A_24_P746650 | | chr1: 52077703-52077762 | 0.043415 |
| A_23_P124619 | S100A14 | chr1: 151853689-151853630 | 0.043483 |
| A_32_P120120 | | chr1: 115070389-115070448 | 0.043486 |
| A_23_P148584 | DOCK11 | chrX: 117703980-117704039 | 0.043511 |
| A_24_P272594 | MAPKBP1 | chr15: 39907279-39907338 | 0.043513 |
| A_23_P118633 | SPATA20 | chr17: 45987984-45988043 | 0.043559 |
| A_24_P508946 | | chrX: 048191408-048191349 | 0.043583 |
| A_24_P417460 | SFMBT2 | chr10: 7244787-7244728 | 0.043589 |
| A_23_P100203 | HSBP1 | chr16: 82403745-82403804 | 0.043619 |
| A_24_P910660 | SLC6A2 | chr16: 54296811-54296870 | 0.043638 |
| A_24_P634911 | | chr9: 133269084-133269143 | 0.043690 |
| A_32_P212802 | | chr7: 101781825-101781766 | 0.043696 |
| A_24_P913078 | | chr10: 006933025-006933084 | 0.043723 |
| A_23_P366312 | SP1 | chr12: 52092756-52092815 | 0.043729 |
| A_24_P476913 | | chr16: 029046066-029046125 | 0.043759 |
| A_23_P103698 | MAPBPIP | chr1: 154291810-154294421 | 0.043759 |
| A_32_P221452 | RABL3 | chr3: 121888708-121888649 | 0.043785 |
| A_32_P51524 | LOC595101 | chr16: 29444066-29444007 | 0.043819 |
| A_24_P366787 | FLJ42875 | chr1: 2973774-2970681 | 0.043837 |
| A_32_P87074 | | chr18: 57704630-57704571 | 0.043846 |
| A_23_P30122 | IL2 | chr4: 123594398-123594339 | 0.043948 |
| A_32_P65371 | | chr6: 086058925-086058984 | 0.043978 |
| A_23_P42935 | BRAF | chr7: 140080947-140080888 | 0.044046 |
| A_32_P228784 | PPP4R2 | chr3: 73197369-73197428 | 0.044052 |
| A_23_P94754 | TNFSF15 | chr9: 116592119-116592060 | 0.044059 |
| A_23_P61268 | C8orf30A | chr8: 145267451-145267510 | 0.044086 |
| A_23_P16476 | KLF16 | chr19: 1805273-1805215 | 0.044123 |
| A_24_P16071 | LOC442256 | chr6: 126620791-126620760 | 0.044124 |
| A_24_P919835 | | chr5: 75802731-75802672 | 0.044127 |
| A_24_P911442 | | chr1: 100426554-100426613 | 0.044134 |
| A_23_P366312 | SP1 | chr12: 52092756-52092815 | 0.044143 |
| A_23_P372151 | C19orf29 | chr19: 3563194-3563135 | 0.044151 |
| A_24_P894132 | LOC649290 | chr18: 74537325-74537266 | 0.044159 |
| A_23_P30972 | ASCC3 | chr6: 101422603-101422544 | 0.044191 |
| A_23_P140675 | EPB42 | chr15: 41276857-41276798 | 0.044202 |
| A_23_P43034 | ELP3 | chr8: 28103468-28103527 | 0.044270 |
| A_23_P73992 | | chr1: 55307087-55307028 | 0.044281 |
| A_32_P171984 | | chr7: 091601586-091601645 | 0.044353 |
| A_24_P935114 | | chr8: 134126135-134126194 | 0.044362 |
| A_24_P414952 | TMEM168 | chr7: 112193553-112193494 | 0.044439 |
| A_24_P131066 | IGSF2 | chr1: 117369655-117369714 | 0.044490 |
| A_23_P404045 | MTHFR | chr1: 11777062-11777008 | 0.044501 |
| A_24_P818010 | | chr1: 124783-124724 | 0.044565 |
| A_23_P207896 | CSNK1D | chr17: 77795992-77795933 | 0.044587 |
| A_23_P96965 | SYNC1 | chr1: 32918948-32918889 | 0.044614 |
| A_24_P941038 | C10orf72 | chr10: 49897287-49897228 | 0.044621 |
| A_24_P328446 | TLX2 | chr2: 74595605-74595664 | 0.044631 |
| A_24_P105164 | RP11-217H1.1 | chrX: 76983399-76972993 | 0.044646 |
| A_23_P101829 | EDG4 | chr19: 19596330-19596271 | 0.044653 |
| A_24_P113636 | C6orf35 | chr6_random: 1511883-1511824 | 0.044746 |
| A_23_P107963 | FUT1 | chr19: 53943137-53943080 | 0.044781 |
| A_24_P106591 | MUTED | chr6: 7959277-7959218 | 0.044804 |
| A_24_P913178 | | chr22: 36501865-36501806 | 0.044862 |
| A_23_P153320 | ICAM1 | chr19: 10257298-10257358 | 0.044881 |
| A_23_P13482 | C11orf16 | chr11: 8898277-8898218 | 0.044896 |
| A_23_P49878 | FAM64A | chr17: 6294966-6295025 | 0.044898 |
| A_23_P8240 | FAM50B | chr6: 3796192-3796251 | 0.044902 |
| A_24_P932016 | HUWE1 | chrX: 53628803-53628744 | 0.044913 |
| A_23_P352226 | MAPKBP1 | chr15: 39904920-39904979 | 0.045047 |
| A_24_P464489 | hCG_2041321 | chr1: 158257140-158257199 | 0.045122 |
| A_23_P154643 | BMP7 | chr20: 55177264-55177211 | 0.045123 |
| A_23_P23765 | ITGB3BP | chr1: 63693157-63692682 | 0.045141 |
| A_23_P64751 | ADMR | chr12: 55676187-55676246 | 0.045143 |
| A_24_P937528 | | chr9: 138740638-138740697 | 0.045216 |
| A_23_P207736 | | chr17: 74303782-74303723 | 0.045237 |
| A_23_P57306 | CHAF1B | chr21: 36710828-36710887 | 0.045243 |
| A_23_P257144 | PXDN | chr2: 1616225-1616166 | 0.045264 |
| A_23_P29199 | NF2 | chr22: 28387293-28391002 | 0.045300 |
| A_23_P254612 | DBF4 | chr7: 87375257-87375316 | 0.045302 |
| A_24_P419323 | | chr4: 1303169-1303228 | 0.045358 |
| A_24_P37712 | TEC | chr4: 47833326-47833267 | 0.045358 |
| A_23_P8363 | MGC16075 | chr7: 47772624-47772683 | 0.045395 |
| A_24_P743869 | LOC92482 | chr10: 112649060-112649001 | 0.045414 |
| A_23_P79066 | SPPL2B | chr19: 2304022-2304081 | 0.045421 |
| A_24_P739344 | NOX4 | chr11: 88699330-88699271 | 0.045454 |
| A_23_P88163 | C14orf103 | chr14: 95821310-95821251 | 0.045489 |
| A_24_P221883 | PARL | chr3: 185042872-185042813 | 0.045489 |
| A_23_P70213 | APC | chr5: 112208721-112208780 | 0.045555 |
| A_23_P319520 | TSNARE1 | chr8: 143308796-143291578 | 0.045582 |
| A_23_P166964 | TFDP2 | chr3: 143154042-143153983 | 0.045605 |
| A_23_P390097 | C9orf52 | chr9: 15162034-15161975 | 0.045626 |
| A_23_P35021 | B3GALT6 | chr1: 1158831-1158890 | 0.045635 |
| A_24_P161725 | | chr18: 14468365-14468306 | 0.045677 |
| A_32_P150928 | HS2ST1 | chr1: 87153260-87153319 | 0.045678 |
| A_23_P79134 | PLEKHF1 | chr19: 34857629-34857688 | 0.045692 |
| A_32_P486443 | ZNF500 | chr16: 4738427-4738368 | 0.045696 |
| A_24_P398147 | NEBL | chr10: 21110603-21110544 | 0.045710 |
| A_23_P25515 | MTMR6 | chr13: 24721560-24721501 | 0.045734 |
| A_24_P926770 | LRP10 | chr14: 22411797-22412384 | 0.045745 |
| A_32_P158053 | | chr4: 68165870-68165929 | 0.045758 |
| A_24_P376199 | | chr18: 70944603-70944544 | 0.045777 |
| A_24_P372012 | ICA1 | chr7: 8164748-8164689 | 0.045779 |
| A_32_P99347 | | chr9: 114907120-114907061 | 0.045791 |
| A_32_P314783 | | chr20: 45381893-45381952 | 0.045803 |
| A_23_P158485 | TSPYL2 | chrX: 53134385-53134444 | 0.045806 |
| A_23_P153320 | ICAM1 | chr19: 10257298-10257358 | 0.045810 |
| A_23_P119964 | CEBPZ | chr2: 37283505-37283446 | 0.045868 |
| A_23_P105646 | ATF1 | chr12: 49494127-49494186 | 0.045926 |
| A_23_P164918 | LMTK3 | chr19: 53680575-53680516 | 0.045930 |
| A_24_P149902 | SUFU | chr10: 104342443-104343413 | 0.045949 |
| A_23_P42935 | BRAF | chr7: 140080947-140080888 | 0.045966 |
| A_24_P933838 | | chr1: 152510194-152510253 | 0.045980 |
| A_32_P220750 | PLGLB1 | chr2: 87091616-87091557 | 0.046000 |
| A_23_P39131 | GLTSCR2 | chr19: 52946591-52946650 | 0.046042 |
| A_32_P49654 | RPS2 | chr16: 1953237-1954314 | 0.046050 |
| A_32_P113483 | | chr15: 64762585-64762644 | 0.046074 |
| A_23_P71415 | C8orf32 | chr8: 124522781-124522840 | 0.046076 |
| A_23_P390068 | C19orf21 | chr19: 714774-714833 | 0.046104 |
| A_32_P24372 | TBC1D3 | chr17: 33369069-33369127 | 0.046118 |
| A_23_P203743 | GAB2 | chr11: 77604395-77604336 | 0.046153 |
| A_24_P367496 | CRHR1 | chr17: 41258680-41258739 | 0.046162 |
| A_24_P3415 | PPP2R2C | chr4: 6373767-6373708 | 0.046164 |
| A_24_P213643 | TSPAN10 | chr17: 77222951-77223010 | 0.046183 |
| A_23_P108404 | CENTG2 | chr2: 236698003-236698062 | 0.046202 |
| A_23_P30634 | BACH2 | chr6: 90693385-90693326 | 0.046216 |
| A_32_P196411 | | chr19: 057338148-057338089 | 0.046265 |
| A_32_P65530 | | chr10: 75104888-75104947 | 0.046270 |
| A_23_P45864 | TNR | chr1: 173571497-173565963 | 0.046284 |
| A_24_P943218 | | chr1: 143999015-143998956 | 0.046326 |
| A_23_P50000 | FAM57A | chr17: 592388-592447 | 0.046385 |
| A_24_P393582 | | chr13: 57917881-57917822 | 0.046403 |
| A_23_P20660 | DIRAS2 | chr9: 92412800-92412741 | 0.046410 |
| A_23_P80759 | PVRL3 | chr3: 112336350-112336409 | 0.046489 |
| A_24_P922877 | KLC1 | chr14: 103237402-103237461 | 0.046522 |
| A_23_P68547 | MCM8 | chr20: 5923064-5923123 | 0.046531 |
| A_23_P74887 | SDC3 | chr1: 31115887-31115828 | 0.046544 |
| A_24_P75190 | HBD | chr11: 5211893-5211834 | 0.046547 |
| A_32_P783 | RPL37A | chr2: 217072325-217072933 | 0.046550 |
| A_23_P85893 | C1orf85 | chr1: 154529367-154529308 | 0.046554 |
| A_23_P252462 | GALNT9 | chr12: 131247679-131247620 | 0.046603 |
| A_23_P153549 | GRIN2D | chr19: 53638833-53638892 | 0.046610 |
| A_24_P912372 | KIAA0754 | chr1: 39654350-39654409 | 0.046631 |
| A_23_P100141 | C16orf28 | chr16: 1355346-1355287 | 0.046667 |
| A_24_P157606 | | chr5: 34962530-34962589 | 0.046667 |
| A_23_P419411 | | chr3: 116322662-116322721 | 0.046680 |
| A_23_P360387 | C15orf37 | chr15: 78002312-78002371 | 0.046681 |
| A_23_P4387 | KRT24 | chr17: 36108079-36108020 | 0.046683 |
| A_23_P87238 | SAA4 | chr11: 18210619-18210560 | 0.046692 |
| A_23_P137578 | FBXO28 | chr1: 222413339-222413398 | 0.046700 |
| A_24_P206121 | KCNMB1 | chr5: 169744919-169743397 | 0.046735 |
| A_24_P925808 | | chr19: 054185610-054185669 | 0.046754 |
| A_23_P165984 | ZSWIM3 | chr20: 43940742-43940801 | 0.046805 |
| A_32_P797019 | NPEPL1 | chr20: 56727569-56727628 | 0.046843 |
| A_23_P164196 | DLX4 | chr17: 45406865-45406924 | 0.046852 |
| A_23_P19102 | GALNT10 | chr5: 153753992-153754051 | 0.046853 |
| A_23_P102994 | PIK4CA | chr22: 19395051-19394992 | 0.046885 |
| A_24_P299318 | FAM101B | chr17: 290167-290108 | 0.046937 |
| A_24_P90900 | CTRL | chr16: 66521918-66521859 | 0.046941 |
| A_24_P798431 | | chr4: 039228258-039228199 | 0.046985 |
| A_32_P649 | STAU2 | chr8: 74627035-74626976 | 0.047020 |
| A_24_P926993 | | | 0.047030 |
| A_23_P346086 | TPM3 | chr1: 152397957-152397898 | 0.047032 |
| A_24_P608468 | LOC145694 | chr15: 67494290-67494231 | 0.047054 |
| A_24_P179925 | DPM3 | chr1: 153379167-153379108 | 0.047060 |
| A_23_P58072 | | chr3: 110672889-110672830 | 0.047079 |
| A_24_P135276 | USP42 | chr7: 6161178-6161237 | 0.047090 |
| A_23_P407074 | DNM2 | chr19: 10803036-10803095 | 0.047145 |
| A_24_P75456 | | chr7: 28285620-28285561 | 0.047146 |
| A_32_P195346 | | chr5: 34141032-34140973 | 0.047181 |
| A_24_P941450 | | chr14: 054583896-054583837 | 0.047204 |
| A_23_P166823 | TNNC1 | chr3: 52460913-52460854 | 0.047215 |
| A_23_P163467 | FLJ43339 | chr15: 38410992-38410946 | 0.047279 |
| A_32_P226449 | | chr20: 1813401-1813460 | 0.047281 |
| A_24_P231494 | DNPEP | chr2: 219958971-219958651 | 0.047294 |
| A_24_P10674 | SNX26 | chr19: 40970877-40970936 | 0.047380 |
| A_23_P107313 | SDF2 | chr17: 24006603-24006544 | 0.047388 |
| A_23_P127095 | USMG5 | chr10: 105138899-105138840 | 0.047409 |
| A_23_P355455 | TBC1D5 | chr3: 17175042-17174983 | 0.047416 |
| A_23_P154466 | AGBL5 | chr2: 27135644-27135703 | 0.047431 |
| A_23_P86242 | SLC30A10 | chr1: 218155127-218155068 | 0.047466 |
| A_32_P112100 | | chr11: 082121672-082121731 | 0.047473 |
| A_32_P64990 | | chr11: 2922481-2922540 | 0.047500 |
| A_23_P28886 | PCNA | chr20: 5044102-5043957 | 0.047504 |
| A_24_P699526 | | chr1: 100430559-100430618 | 0.047524 |
| A_24_P179339 | | chrM: 2403-2462 | 0.047532 |
| A_23_P350704 | PRKAB2 | chr1: 145097838-145097779 | 0.047537 |
| A_23_P162589 | VDR | chr12: 46521828-46521769 | 0.047624 |
| A_23_P253689 | | chr9: 3470714-3470773 | 0.047630 |
| A_24_P200694 | RING1 | chr6: 33288091-33288150 | 0.047656 |
| A_23_P69863 | PCDHB5 | chr5: 140497639-140497698 | 0.047660 |
| A_23_P43484 | CDKN2A | chr9: 21957839-21957780 | 0.047663 |
| A_23_P124962 | FAM89B | chr11: 65098176-65098235 | 0.047663 |
| A_23_P79259 | SH3BP4 | chr2: 235628045-235628104 | 0.047687 |
| A_23_P58815 | FBXO4 | chr5: 41963019-41963078 | 0.047701 |
| A_32_P205624 | SHC2 | chr19: 367892-367833 | 0.047724 |
| A_24_P932822 | | chr2: 198068790-198068849 | 0.047754 |
| A_24_P858192 | | chr17: 26086437-26086378 | 0.047842 |
| A_23_P162589 | VDR | chr12: 46521828-46521769 | 0.047852 |
| A_23_P155027 | MORC2 | chr22: 29652701-29652642 | 0.047900 |
| A_32_P118013 | | chr6: 159988158-159988099 | 0.047923 |
| A_24_P938553 | | chr1: 211137934-211137875 | 0.047969 |
| A_23_P58102 | EXOSC7 | chr3: 45013719-45018063 | 0.047987 |
| A_23_P46499 | | chr1: 48342551-48342610 | 0.048026 |
| A_23_P259103 | LOC63920 | chr5: 159753488-159753429 | 0.048038 |
| A_24_P100419 | EDA | chrX: 69093644-69093703 | 0.048073 |
| A_23_P35564 | SEC31B | chr10: 102236462-102236403 | 0.048088 |
| A_23_P100730 | SKAP1 | chr17: 43565888-43565829 | 0.048174 |
| A_32_P149246 | | chr5: 138775094-138775035 | 0.048175 |
| A_23_P42626 | BCDIN3 | chr7: 99869199-99869258 | 0.048186 |
| A_24_P41706 | OFCC1 | chr6: 10040999-10016775 | 0.048192 |
| A_23_P7282 | ELMOD2 | chr4: 141694208-141694267 | 0.048202 |
| A_24_P350890 | RANBP2 | chr2: 108750899-108750958 | 0.048272 |
| A_24_P1873 | | | 0.048304 |
| A_23_P322632 | | chr15: 67174284-67174343 | 0.048339 |
| A_23_P259901 | TKTL1 | chrX: 153211818-153211877 | 0.048469 |
| A_23_P136026 | IGHA1 | chr14: 105245260-105245201 | 0.048493 |
| A_23_P116712 | RASSF8 | chr12: 26109211-26109270 | 0.048501 |
| A_23_P103414 | YTHDF2 | chr1: 28968570-28968629 | 0.048539 |
| A_24_P151582 | TEF | chr22: 40124526-40124585 | 0.048570 |
| A_24_P41079 | LOC440295 | chr15_random: 637449-637526 | 0.048607 |
| A_23_P165651 | MRPL35 | chr2: 86287888-86287947 | 0.048733 |
| A_23_P371284 | LCE2D | chr1: 150903308-150903367 | 0.048767 |
| A_24_P927679 | | chr16: 30333028-30333087 | 0.048778 |
| A_32_P82530 | | chr10: 009840819-009840760 | 0.048809 |
| A_23_P212800 | FGF5 | chr4: 81426830-81426889 | 0.048841 |
| A_24_P84738 | LOC392226 | chr8: 63219652-63219593 | 0.048872 |
| A_23_P319270 | AZI1 | chr17: 76778051-76777992 | 0.048898 |
| A_23_P111635 | STYXL1 | chr7: 75489146-75481124 | 0.048935 |
| A_32_P42946 | C1orf210 | chr1: 43521143-43521084 | 0.048951 |
| A_24_P145107 | | chr6: 43611205-43611264 | 0.048961 |
| A_24_P934214 | | chr19: 016583194-016583253 | 0.048968 |
| A_23_P26367 | FLJ12331 | chr16: 67626946-67627005 | 0.049028 |
| A_23_P338113 | | chr6: 29877466-29877525 | 0.049061 |
| A_24_P926693 | | chr1: 54617809-54617750 | 0.049085 |
| A_24_P924452 | | chr2: 064036091-064036150 | 0.049141 |
| A_23_P48246 | PEX5 | chr12: 7254527-7254586 | 0.049150 |
| A_32_P27240 | | chr19: 20974746-20974805 | 0.049152 |
| A_24_P100650 | C9orf11 | chr9: 27286692-27286633 | 0.049154 |
| A_23_P42897 | MGAM | chr7: 141449681-141452114 | 0.049181 |
| A_24_P176493 | ASCIZ | chr16: 79638216-79638275 | 0.049190 |
| A_24_P601654 | | chr2: 37283462-37283521 | 0.049238 |
| A_23_P138899 | COPB1 | chr11: 14439322-14438375 | 0.049252 |
| A_23_P152024 | CSK | chr15: 72881418-72881477 | 0.049357 |
| A_23_P424582 | EGFL8 | chr6: 32243902-32243964 | 0.049371 |
| A_23_P91850 | IL20RB | chr3: 138212488-138212547 | 0.049432 |
| A_23_P153698 | BTBD2 | chr19: 1937040-1936981 | 0.049450 |
| A_24_P752862 | | chr3_random: 14355-14296 | 0.049458 |
| A_23_P416747 | CD3E | chr11: 117691524-117691583 | 0.049476 |
| A_24_P359389 | DKFZP434C153 | chr1: 226555773-226555832 | 0.049519 |
| A_23_P166336 | | chr22: 19388825-19388884 | 0.049531 |
| A_24_P256668 | ZC3H7A | chr16: 11770432-11769844 | 0.049571 |
| A_23_P164691 | ICAM3 | chr19: 10305713-10305654 | 0.049579 |
| A_32_P222751 | | chr6: 3668156-3668215 | 0.049599 |
| A_24_P916547 | | chr7: 57716165-57716106 | 0.049625 |
| A_23_P119143 | ICAM5 | chr19: 10268322-10268381 | 0.049658 |
| A_24_P221445 | OXR1 | chr8: 107823634-107823693 | 0.049694 |
| A_24_P268856 | PLRG1 | chr4: 155676438-155676379 | 0.049719 |
| A_24_P922449 | | chr20: 002493686-002493627 | 0.049763 |
| A_24_P110937 | PCID2 | chr13: 112891230-112891171 | 0.049835 |
| A_23_P20722 | SNAPC4 | chr9: 138389913-138389854 | 0.049920 |
| A_32_P156628 | | chr1: 180620346-180620405 | 0.049935 |
| A_23_P146294 | EFCAB1 | chr8: 49798692-49798633 | 0.049941 |
| A_32_P26034 | | chr11: 14867788-14867847 | 0.049946 |
| A_23_P11862 | C1orf112 | chr1: 168088734-168088793 | 0.049948 |
| A_24_P134372 | KIF3B | chr20: 30384633-30384692 | 0.049965 |
| A_32_P519670 | | chr22: 16416637-16416697 | 0.049988 |
| A_24_P456944 | | chr7: 128082193-128082252 | 0.049994 |

Table 6 lists the genes that were significant (0.05) in both Agilent cohorts.

TABLE 6

| | | | p-value |
|---|---|---|---|
| | | p-value | second |
| Gene Symbol | Genomic Coordinates | first cohort | cohort |
| DDX50 | chr10: 70376457-70376516 | 0.03139 | 0.04723 |
| MREG | chr2: 216516404-216516345 | 0.03381 | 0.03787 |
| RAB6B | chr3: 135030253-135030194 | 0.00752 | 0.04819 |
| C19orf2 | chr19: 35195068-35195127 | 0.03736 | 0.04251 |
| | chr8: 92149966-92149907 | 0.01849 | 0.01357 |
| | chr8: 92149966-92149907 | 0.01849 | 0.02724 |
| C10orf26 | chr10: 104563853-104563912 | 0.02085 | 0.00293 |
| ZNF187 | chr6: 28353058-28353117 | 0.02566 | 0.04062 |
| ENPP1 | chr6: 132253564-132253623 | 0.03416 | 0.03892 |
| FLJ43339 | chr15: 38410992-38410946 | 0.04728 | 0.04246 |
| | chr2: 102826424-102826483 | 0.04206 | 0.03608 |
| MOBKL2C | chr1: 46846412-46846353 | 0.00807 | 0.02846 |
| FNDC4 | chr2: 27568826-27568767 | 0.01516 | 0.00764 |
| DSE | chr6: 116865133-116865192 | 0.03731 | 0.01071 |
| TMEM123 | chr11: 101777616-101777557 | 0.01342 | 0.01201 |
| HDAC4 | chr2: 239635672-239635613 | 0.03391 | 0.01128 |
| WIPI2 | chr7: 5237711-5237770 | 0.03966 | 0.04238 |
| FGF2 | chr4: 124038472-124038531 | 0.01730 | 0.01839 |
| FGF2 | chr4: 124038472-124038531 | 0.01730 | 0.00555 |
| FGF2 | chr4: 124038472-124038531 | 0.01730 | 0.00385 |
| PSAT1 | chr9: 80134598-80134657 | 0.02275 | 0.01464 |
| NF2 | chr22: 28387293-28391002 | 0.04530 | 0.02589 |
| NFKB1 | chr4: 103756902-103756961 | 0.00205 | 0.02018 |
| NFKB1 | chr4: 103756902-103756961 | 0.00205 | 0.01626 |
| DNAJC4 | chr11: 63755978-63757966 | 0.01036 | 0.00018 |
| GATS | chr7: 99636814-99636755 | 0.01291 | 0.00437 |
| | chr9: 97576280-97576339 | 0.03480 | 0.03426 |
| NAP5 | chr2: 133199757-133199698 | 0.01425 | 0.02676 |
| TAPBP | chr6: 33375755-33375696 | 0.00277 | 0.00995 |
| ZNF403 | chr17: 32020040-32020099 | 0.02854 | 0.04551 |
| MTHFR | chr1: 11777062-11777008 | 0.04450 | 0.03464 |
| RAF1 | chr3: 12600670-12600611 | 0.02249 | 0.02502 |
| RAF1 | chr3: 12600670-12600611 | 0.02249 | 0.03280 |
| RAB5B | chr12: 54674293-54674352 | 0.04268 | 0.01332 |
| WDR37 | chr10: 1167673-1167732 | 0.03917 | 0.00195 |
| FAM24B | chr10: 124599980-124598937 | 0.02209 | 0.01946 |
| SLC39A14 | chr8: 22335781-22335840 | 0.02170 | 0.02263 |
| SLC6A6 | chr3: 14488791-14493737 | 0.02260 | 0.02071 |
| CD47 | chr3: 109262312-109261053 | 0.02758 | 0.00505 |
| WBSCR22 | chr7: 72745670-72746260 | 0.00083 | 0.02957 |
| RBP4 | chr10: 95341718-95341659 | 0.00752 | 0.01549 |
| SNX10 | chr7: 26380261-26380320 | 0.03883 | 0.04566 |
| ENTPD7 | chr10: 101455180-101455239 | 0.01975 | 0.02331 |
| KIAA0652 | chr11: 46650404-46650463 | 0.01698 | 0.01994 |
| OAT | chr10: 126076306-126076247 | 0.00526 | 0.02216 |
| ENTPD7 | chr10: 101455019-101455078 | 0.02878 | 0.02768 |
| UBE2D1 | chr10: 59797707-59797766 | 0.03492 | 0.00880 |
| TFAM | chr10: 59825178-59825237 | 0.01236 | 0.03988 |
| SUFU | chr10: 104342443-104343413 | 0.04595 | 0.04484 |
| | chr18: 14468365-14468306 | 0.04568 | 0.04415 |
| PRKD3 | chr2: 37337552-37337493 | 0.00682 | 0.01702 |
| FBXO4 | chr5: 41970094-41970153 | 0.00255 | 0.04054 |
| SERTAD2 | chr2: 64712664-64712605 | 0.03553 | 0.00701 |
| TLX2 | chr2: 74595605-74595664 | 0.04463 | 0.00218 |
| ACVR1B | chr12: 50674874-50674933 | 0.00790 | 0.03608 |
| | chr5: 042589834-042589893 | 0.01461 | 0.04525 |
| SLC11A2 | chr12: 49666625-49666566 | 0.01514 | 0.02153 |
| AASDH | chr4: 56910276-56910217 | 0.00305 | 0.04970 |
| | chr3: 032275758-032275821 | 0.02594 | 0.04518 |
| URM1 | chr9: 130192598-130192653 | 0.02320 | 0.02122 |
| | chr6: 017754043-017754101 | 0.03248 | 0.04629 |
| hCG_2041321 | chr1: 158257140-158257199 | 0.04512 | 0.01519 |
| | chr2: 153055200-153055259 | 0.00698 | 0.01331 |
| SOCS1 | chr16: 11256503-11256444 | 0.00499 | 0.03640 |
| | chr9: 131737515-131737456 | 0.04073 | 0.00450 |
| | chr6: 090581784-090581843 | 0.01033 | 0.04795 |
| | chrX: 014762751-014762686 | 0.00482 | 0.03148 |
| | chr5: 178357888-178357830 | 0.00530 | 0.00740 |
| LOC390427 | chr13: 113982309-113982250 | 0.03269 | 0.00073 |
| NOX4 | chr11: 88699330-88699271 | 0.04545 | 0.01140 |
| | chrX: 053189643-053189584 | 0.03735 | 0.02208 |
| CDKN1B | chr12: 12766207-12766266 | 0.01123 | 0.03329 |
| CDKN1B | chr12: 12766207-12766266 | 0.01185 | 0.03329 |
| CDKN1B | chr12: 12766207-12766266 | 0.01368 | 0.03329 |
| CDKN1B | chr12: 12766207-12766266 | 0.01417 | 0.03329 |
| CDKN1B | chr12: 12766207-12766266 | 0.01938 | 0.03329 |
| CDKN1B | chr12: 12766207-12766266 | 0.01986 | 0.03329 |
| CDKN1B | chr12: 12766207-12766266 | 0.01123 | 0.04906 |
| CDKN1B | chr12: 12766207-12766266 | 0.01185 | 0.04906 |
| CDKN1B | chr12: 12766207-12766266 | 0.01368 | 0.04906 |
| CDKN1B | chr12: 12766207-12766266 | 0.01417 | 0.04906 |
| CDKN1B | chr12: 12766207-12766266 | 0.01938 | 0.04906 |
| CDKN1B | chr12: 12766207-12766266 | 0.01986 | 0.04906 |
| CDKN1B | chr12: 12766207-12766266 | 0.01123 | 0.01228 |
| CDKN1B | chr12: 12766207-12766266 | 0.01185 | 0.01228 |
| CDKN1B | chr12: 12766207-12766266 | 0.01368 | 0.01228 |
| CDKN1B | chr12: 12766207-12766266 | 0.01417 | 0.01228 |
| CDKN1B | chr12: 12766207-12766266 | 0.01938 | 0.01228 |
| CDKN1B | chr12: 12766207-12766266 | 0.01986 | 0.01228 |
| CDKN1B | chr12: 12766207-12766266 | 0.01123 | 0.02271 |
| CDKN1B | chr12: 12766207-12766266 | 0.01185 | 0.02271 |
| CDKN1B | chr12: 12766207-12766266 | 0.01368 | 0.02271 |
| CDKN1B | chr12: 12766207-12766266 | 0.01417 | 0.02271 |
| CDKN1B | chr12: 12766207-12766266 | 0.01938 | 0.02271 |
| CDKN1B | chr12: 12766207-12766266 | 0.01986 | 0.02271 |
| CDKN1B | chr12: 12766207-12766266 | 0.01123 | 0.00345 |
| CDKN1B | chr12: 12766207-12766266 | 0.01185 | 0.00345 |
| CDKN1B | chr12: 12766207-12766266 | 0.01368 | 0.00345 |
| CDKN1B | chr12: 12766207-12766266 | 0.01417 | 0.00345 |
| CDKN1B | chr12: 12766207-12766266 | 0.01938 | 0.00345 |
| CDKN1B | chr12: 12766207-12766266 | 0.01986 | 0.00345 |
| CDKN1B | chr12: 12766207-12766266 | 0.01123 | 0.01616 |
| CDKN1B | chr12: 12766207-12766266 | 0.01185 | 0.01616 |
| CDKN1B | chr12: 12766207-12766266 | 0.01368 | 0.01616 |
| CDKN1B | chr12: 12766207-12766266 | 0.01417 | 0.01616 |
| CDKN1B | chr12: 12766207-12766266 | 0.01938 | 0.01616 |
| CDKN1B | chr12: 12766207-12766266 | 0.01986 | 0.01616 |
| CDKN1B | chr12: 12766207-12766266 | 0.01123 | 0.00594 |
| CDKN1B | chr12: 12766207-12766266 | 0.01185 | 0.00594 |
| CDKN1B | chr12: 12766207-12766266 | 0.01368 | 0.00594 |
| CDKN1B | chr12: 12766207-12766266 | 0.01417 | 0.00594 |
| CDKN1B | chr12: 12766207-12766266 | 0.01938 | 0.00594 |
| CDKN1B | chr12: 12766207-12766266 | 0.01986 | 0.00594 |
| LOC392226 | chr8: 63219652-63219593 | 0.04887 | 0.03945 |
| GPR68 | chr14: 90768741-90768682 | 0.02539 | 0.00995 |
| | chr20: 14476013-14475954 | 0.04280 | 0.00531 |
| ANAPC5 | chr12: 120269135-120269990 | 0.02018 | 0.04114 |
| | chr6: 122806787-122806846 | 0.03979 | 0.00002 |
| | chr10: 76976989-76977048 | 0.02844 | 0.00962 |
| | chr6: 3668156-3668215 | 0.04960 | 0.03354 |
| LOC595101 | chr16: 29444066-29444007 | 0.04382 | 0.01578 |
| SLAIN2 | chr4: 48080467-48080526 | 0.04104 | 0.00295 |
| LOC595101 | chr16: 29444051-29443992 | 0.02696 | 0.01428 |

Table 7 lists genes that were significant (0.05) in one cohort and (0.1) in another cohort.

TABLE 7

| | | | p-value | p-value |
|---|---|---|---|---|
| Probe ID | Gene Symbol | Genomic Coordinates | 1^{st} cohort | 2^{nd} cohort |
| A_23_P102113 | WNT10A | chr2: 219466718-219466777 | 0.056864 | 0.025281 |
| A_23_P104451 | DDX50 | chr10: 70376457-70376516 | 0.031388 | 0.047225 |
| A_23_P108948 | MREG | chr2: 216516404-216516345 | 0.033808 | 0.037866 |
| A_23_P109974 | RAB6B | chr3: 135030253-135030194 | 0.007521 | 0.048191 |
| A_23_P111005 | YIPF3 | chr6: 43587823-43587764 | 0.016072 | 0.069698 |
| A_23_P119143 | ICAM5 | chr19: 10268322-10268381 | 0.049658 | 0.082223 |
| A_23_P119418 | C19orf2 | chr19: 35195068-35195127 | 0.037363 | 0.042512 |
| A_23_P120644 | | chr20: 033929999-033930058 | 0.071034 | 0.006812 |
| A_23_P120784 | HTF9C | chr22: 18479703-18479644 | 0.004787 | 0.068307 |
| A_23_P12241 | MCOLN3 | chr1: 85260616-85260451 | 0.087495 | 0.034509 |
| A_23_P123315 | | chr8: 92149966-92149907 | 0.018489 | 0.013572 |
| A_23_P123315 | | chr8: 92149966-92149907 | 0.018489 | 0.055774 |
| A_23_P123315 | | chr8: 92149966-92149907 | 0.018489 | 0.027237 |
| A_23_P124327 | NSD1 | chr5: 176655180-176655239 | 0.017681 | 0.068069 |
| A_23_P1248 | C10orf26 | chr10: 104563853-104563912 | 0.020846 | 0.002927 |
| A_23_P127367 | POLD4 | chr11: 66875773-66875714 | 0.035440 | 0.082519 |
| A_23_P127565 | LAYN | chr11: 110936670-110936729 | 0.027246 | 0.084374 |
| A_23_P12784 | FRAT2 | chr10: 99082454-99082395 | 0.000835 | 0.098983 |
| A_23_P128503 | IL26 | chr12: 66881570-66881511 | 0.025658 | 0.054453 |
| A_23_P128613 | KDELC1 | chr13: 102234748-102234689 | 0.090453 | 0.029562 |
| A_23_P129031 | ATXN3 | chr14: 91600351-91600292 | 0.090869 | 0.045313 |
| A_23_P13482 | C11orf16 | chr11: 8898277-8898218 | 0.044896 | 0.070682 |
| A_23_P145247 | ZNF187 | chr6: 28353058-28353117 | 0.025657 | 0.040615 |
| A_23_P145978 | VIPR2 | chr7: 158520076-158519741 | 0.068813 | 0.026592 |
| A_23_P15123 | UBFD1 | chr16: 23489532-23489591 | 0.080172 | 0.038426 |
| A_23_P156632 | PGBD1 | chr6: 28377624-28377683 | 0.064922 | 0.012233 |
| A_23_P156880 | ENPP1 | chr6: 132253564-132253623 | 0.034163 | 0.038919 |
| A_23_P159039 | SCRIB | chr8: 144945190-144945131 | 0.052905 | 0.034772 |
| A_23_P159101 | U1SNRNPBP | chr12: 122516498-122516557 | 0.075399 | 0.018916 |
| A_23_P160729 | AP4B1 | chr1: 114239549-114239490 | 0.094614 | 0.040486 |
| A_23_P163117 | C14orf169 | chr14: 73029729-73029788 | 0.099968 | 0.026374 |
| A_23_P163467 | FLJ43339 | chr15: 38410992-38410946 | 0.047279 | 0.042459 |
| A_23_P164650 | APOE | chr19: 50104213-50104272 | 0.095548 | 0.030443 |
| A_23_P164650 | APOE | chr19: 50104213-50104272 | 0.095548 | 0.039469 |
| A_23_P165541 | | chr2: 102826424-102826483 | 0.042061 | 0.036082 |
| A_23_P165984 | ZSWIM3 | chr20: 43940742-43940801 | 0.046805 | 0.074354 |
| A_23_P166 | MOBKL2C | chr1: 46846412-46846353 | 0.008075 | 0.028464 |
| A_23_P166023 | PFDN4 | chr20: 52265292-52265351 | 0.023361 | 0.067965 |
| A_23_P166336 | | chr22: 19388825-19388884 | 0.049531 | 0.072129 |
| A_23_P16834 | FNDC4 | chr2: 27568826-27568767 | 0.015164 | 0.007645 |
| A_23_P168470 | BAIAP2L1 | chr7: 97779483-97779424 | 0.024291 | 0.058937 |
| A_23_P169154 | KIAA1815 | chr9: 5775753-5775694 | 0.015693 | 0.080269 |
| A_23_P169529 | HRB | chr2: 228127807-228127866 | 0.054039 | 0.044260 |
| A_23_P19226 | DSE | chr6: 116865133-116865192 | 0.037312 | 0.010706 |
| A_23_P202496 | NOC3L | chr10: 96083087-96083028 | 0.082447 | 0.042137 |
| A_23_P202964 | TMEM123 | chr11: 101777616-101777557 | 0.013423 | 0.012013 |
| A_23_P204947 | GJB2 | chr13: 19659974-19659915 | 0.037718 | 0.088634 |
| A_23_P204947 | GJB2 | chr13: 19659974-19659915 | 0.037718 | 0.056863 |
| A_23_P204947 | GJB2 | chr13: 19659974-19659915 | 0.037718 | 0.076780 |
| A_23_P204947 | GJB2 | chr13: 19659974-19659915 | 0.037718 | 0.085939 |
| A_23_P207680 | ARL17 | chr17: 41794825-41791505 | 0.092433 | 0.020383 |
| A_23_P210048 | HDAC4 | chr2: 239635672-239635613 | 0.033915 | 0.011283 |
| A_23_P212329 | PTPN23 | chr3: 47429669-47429728 | 0.095160 | 0.014350 |
| A_23_P212800 | FGF5 | chr4: 81426830-81426889 | 0.048841 | 0.056021 |
| A_23_P215421 | WIPI2 | chr7: 5237711-5237770 | 0.039665 | 0.042377 |
| A_23_P216282 | ARHGEF10 | chr8: 1894121-1894180 | 0.008531 | 0.082320 |
| A_23_P218918 | FGF2 | chr4: 124038472-124038531 | 0.017304 | 0.073465 |
| A_23_P218918 | FGF2 | chr4: 124038472-124038531 | 0.063769 | 0.003849 |
| A_23_P218918 | FGF2 | chr4: 124038472-124038531 | 0.017304 | 0.003849 |
| A_23_P218918 | FGF2 | chr4: 124038472-124038531 | 0.063769 | 0.018395 |
| A_23_P218918 | FGF2 | chr4: 124038472-124038531 | 0.017304 | 0.018395 |
| A_23_P218918 | FGF2 | chr4: 124038472-124038531 | 0.063769 | 0.005549 |
| A_23_P218918 | FGF2 | chr4: 124038472-124038531 | 0.017304 | 0.005549 |
| A_23_P23090 | PDC | chr1: 184679988-184679929 | 0.065088 | 0.026958 |
| A_23_P24903 | P2RY2 | chr11: 72624564-72624623 | 0.097786 | 0.028325 |
| A_23_P25019 | PRIM1 | chr12: 55419378-55418551 | 0.009568 | 0.070900 |
| A_23_P250825 | RABGEF1 | chr7: 65911718-65911777 | 0.017283 | 0.081070 |
| A_23_P251342 | GSK3B | chr3: 121065091-121065032 | 0.050691 | 0.049211 |
| A_23_P259292 | C1QTNF5 | chr11: 118715164-118715105 | 0.062905 | 0.036869 |
| A_23_P259632 | TMPIT | chr7: 75454498-75454439 | 0.014409 | 0.073248 |
| A_23_P259692 | PSAT1 | chr9: 80134598-80134657 | 0.022748 | 0.014643 |
| A_23_P27346 | SMAD4 | chr18: 46859058-46859117 | 0.054752 | 0.040374 |
| A_23_P27346 | SMAD4 | chr18: 46859058-46859117 | 0.054752 | 0.018292 |
| A_23_P27346 | SMAD4 | chr18: 46859058-46859117 | 0.054752 | 0.021324 |
| A_23_P27346 | SMAD4 | chr18: 46859058-46859117 | 0.054752 | 0.008429 |
| A_23_P27346 | SMAD4 | chr18: 46859058-46859117 | 0.054752 | 0.029491 |
| A_23_P28318 | PRO1853 | chr2: 37328980-37329039 | 0.011017 | 0.098077 |
| A_23_P29199 | NF2 | chr22: 28387293-28391002 | 0.045300 | 0.025895 |
| A_23_P30024 | NFKB1 | chr4: 103756902-103756961 | 0.002050 | 0.016257 |
| A_23_P30024 | NFKB1 | chr4: 103756902-103756961 | 0.002050 | 0.020177 |
| A_23_P30024 | NFKB1 | chr4: 103756902-103756961 | 0.002050 | 0.061281 |
| A_23_P30217 | SCGB3A1 | chr5: 179950210-179949806 | 0.077201 | 0.025454 |
| A_23_P30411 | | chr5: 001122135-001122076 | 0.006722 | 0.062796 |
| A_23_P304279 | CHRNE | chr17: 4742718-4742659 | 0.063715 | 0.006084 |
| A_23_P311834 | MLLT10 | chr10: 21915257-21924297 | 0.033865 | 0.056507 |
| A_23_P312610 | DNAJC4 | chr11: 63755978-63757966 | 0.010361 | 0.000177 |
| A_23_P319492 | FAM118B | chr11: 125637302-125637361 | 0.087817 | 0.025044 |
| A_23_P32249 | | chr9: 131697777-131697719 | 0.050393 | 0.005969 |
| A_23_P327777 | GATS | chr7: 99636814-99636755 | 0.012915 | 0.004367 |
| A_23_P329271 | MC1R | chr16: 88514777-88514836 | 0.081823 | 0.034220 |
| A_23_P335069 | SLC46A1 | chr17: 23747041-23746982 | 0.008824 | 0.098055 |
| A_23_P336838 | | chr9: 97576280-97576339 | 0.034799 | 0.034258 |
| A_23_P337849 | TNRC4 | chr1: 149941710-149941651 | 0.041823 | 0.079499 |
| A_23_P340308 | TAS2R48 | chr12: 11065647-11065588 | 0.053994 | 0.043753 |
| A_23_P340922 | ZNF414 | chr19: 8483309-8482824 | 0.034430 | 0.076919 |
| A_23_P352619 | TIFA | chr4: 113417400-113417341 | 0.017303 | 0.075262 |
| A_23_P359376 | HCG4P6 | chr6: 30000757-30000698 | 0.058742 | 0.030844 |
| A_23_P360079 | NAP5 | chr2: 133199757-133199698 | 0.014248 | 0.026755 |
| A_23_P361336 | ADARB1 | chr21: 45467377-45467436 | 0.033570 | 0.056321 |
| A_23_P365719 | TAPBP | chr6: 33375755-33375696 | 0.002768 | 0.009954 |
| A_23_P367013 | TAS2R60 | chr7: 142850857-142850916 | 0.097518 | 0.040630 |
| A_23_P378499 | ZNF776 | chr19: 62960370-62960673 | 0.090851 | 0.015958 |
| A_23_P38408 | ZNF403 | chr17: 32020040-32020099 | 0.028544 | 0.045506 |
| A_23_P384329 | DENND4A | chr15: 63740618-63740559 | 0.097621 | 0.040700 |
| A_23_P387630 | STARD8 | chrX: 67862252-67862311 | 0.052859 | 0.013835 |
| A_23_P390032 | TMEM20 | chr10: 95651573-95651632 | 0.074123 | 0.047719 |
| A_23_P39215 | NUDT19 | chr19: 37892091-37894509 | 0.013464 | 0.075912 |
| A_23_P401472 | CHRM3 | chr1: 238138604-238138663 | 0.085744 | 0.021559 |
| A_23_P404045 | MTHFR | chr1: 11777062-11777008 | 0.044501 | 0.034644 |
| A_23_P40952 | RAF1 | chr3: 12600670-12600611 | 0.067400 | 0.025017 |
| A_23_P40952 | RAF1 | chr3: 12600670-12600611 | 0.022491 | 0.025017 |
| A_23_P40952 | RAF1 | chr3: 12600670-12600611 | 0.071013 | 0.025017 |
| A_23_P40952 | RAF1 | chr3: 12600670-12600611 | 0.022491 | 0.068718 |
| A_23_P40952 | RAF1 | chr3: 12600670-12600611 | 0.022491 | 0.073104 |
| A_23_P40952 | RAF1 | chr3: 12600670-12600611 | 0.067400 | 0.032801 |
| A_23_P40952 | RAF1 | chr3: 12600670-12600611 | 0.022491 | 0.032801 |
| A_23_P40952 | RAF1 | chr3: 12600670-12600611 | 0.071013 | 0.032801 |
| A_23_P410998 | RAB5B | chr12: 54674293-54674352 | 0.042677 | 0.013315 |
| A_23_P41159 | PPP4R2 | chr3: 73197352-73197411 | 0.011586 | 0.050394 |
| A_23_P412990 | HRH3 | chr20: 60223873-60223814 | 0.034954 | 0.079484 |
| A_23_P419150 | VENTX | chr10: 134904452-134904511 | 0.078713 | 0.039377 |
| A_23_P434807 | RND2 | chr17: 38431574-38432768 | 0.043028 | 0.098245 |
| A_23_P436145 | | chr11: 130219432-130219373 | 0.089153 | 0.043118 |
| A_23_P45324 | TMEM35 | chrX: 100237886-100237945 | 0.051446 | 0.015485 |
| A_23_P47073 | WDR37 | chr10: 1167673-1167732 | 0.039172 | 0.001954 |
| A_23_P49338 | TNFRSF12A | chr16: 3012181-3012240 | 0.062086 | 0.037804 |
| A_23_P49539 | BAHCC1 | chr17: 77047529-77047588 | 0.063218 | 0.019269 |
| A_23_P502612 | HNRPUL1 | chr19: 46505089-46505148 | 0.062518 | 0.022744 |
| A_23_P50389 | NAT14 | chr19: 60690324-60690382 | 0.028892 | 0.081717 |
| A_23_P51217 | CLCA1 | chr1: 86738403-86738462 | 0.013044 | 0.061919 |
| A_23_P52373 | NDST2 | chr10: 75232068-75232009 | 0.007486 | 0.061204 |
| A_23_P52531 | FAM24B | chr10: 124599980-124598937 | 0.022092 | 0.019465 |
| A_23_P53110 | TUB | chr11: 8080270-8080329 | 0.050468 | 0.008701 |
| A_23_P53137 | HBG1 | chr11: 5226169-5226110 | 0.022463 | 0.077764 |
| A_23_P53137 | HBG1 | chr11: 5226169-5226110 | 0.015474 | 0.077764 |
| A_23_P56949 | SERTAD2 | chr2: 64716407-64716348 | 0.064499 | 0.003602 |
| A_23_P59950 | SLC39A14 | chr8: 22335781-22335840 | 0.021699 | 0.022628 |
| A_23_P62868 | EXOSC10 | chr1: 11050694-11049376 | 0.031948 | 0.054616 |
| A_23_P64083 | SF3B2 | chr11: 65587771-65592046 | 0.069956 | 0.026148 |
| A_23_P69206 | SLC6A6 | chr3: 14488791-14493737 | 0.022599 | 0.020706 |
| A_23_P6935 | CD47 | chr3: 109262312-109261053 | 0.027575 | 0.005049 |
| A_23_P70213 | APC | chr5: 112208721-112208780 | 0.025946 | 0.091613 |
| A_23_P70213 | APC | chr5: 112208721-112208780 | 0.045555 | 0.091613 |
| A_23_P71014 | WBSCR22 | chr7: 72745670-72746260 | 0.000833 | 0.029573 |
| A_23_P71598 | MPDZ | chr9: 13096858-13096799 | 0.091378 | 0.031776 |
| A_23_P73589 | MSN | chrX: 64878326-64878385 | 0.095746 | 0.033231 |
| A_23_P73992 | | chr1: 55307087-55307028 | 0.044281 | 0.083941 |
| A_23_P74928 | MR1 | chr1: 179285969-179286028 | 0.083755 | 0.010199 |
| A_23_P75283 | RBP4 | chr10: 95341718-95341659 | 0.007515 | 0.015490 |
| A_23_P79518 | IL1B | chr2: 113303959-113303900 | 0.053992 | 0.022875 |
| A_23_P79591 | APOB | chr2: 21078063-21078004 | 0.005792 | 0.083147 |
| A_23_P79591 | APOB | chr2: 21078063-21078004 | 0.010441 | 0.083147 |
| A_23_P82913 | TRMT12 | chr8: 125533948-125534007 | 0.035549 | 0.078785 |
| A_23_P8513 | SNX10 | chr7: 26380261-26380320 | 0.038829 | 0.045664 |
| A_23_P865 | FRRS1 | chr1: 99946854-99946847 | 0.061334 | 0.031101 |
| A_23_P86623 | ENTPD7 | chr10: 101455180-101455239 | 0.019755 | 0.023314 |
| A_23_P88163 | C14orf103 | chr14: 95821310-95821251 | 0.045489 | 0.079089 |
| A_23_P95292 | KIAA0652 | chr11: 46650404-46650463 | 0.016976 | 0.019935 |
| A_23_P97123 | RP4-747L4.3 | chr1: 166481733-166481674 | 0.052851 | 0.032825 |
| A_23_P98092 | OAT | chr10: 126076306-126076247 | 0.005255 | 0.022159 |
| A_24_P112447 | ENTPD7 | chr10: 101455019-101455078 | 0.028777 | 0.027676 |
| A_24_P12065 | | chr4: 78309441-78309500 | 0.007222 | 0.098417 |
| A_24_P122403 | TCEB3 | chr1: 23960866-23960925 | 0.034010 | 0.064471 |
| A_24_P123155 | ZNF639 | chr3: 180534583-180534642 | 0.028721 | 0.098038 |
| A_24_P12626 | CAV1 | chr7: 115986237-115986296 | 0.086435 | 0.020574 |
| A_24_P13032 | UBE2D1 | chr10: 59797707-59797766 | 0.034921 | 0.008805 |
| A_24_P132019 | LNPEP | chr5: 96391278-96391337 | 0.069497 | 0.015876 |
| A_24_P134727 | TFAM | chr10: 59825178-59825237 | 0.012358 | 0.039878 |
| A_24_P135748 | GRTP1 | chr13: 113047218-113028388 | 0.004905 | 0.091267 |
| A_24_P149902 | SUFU | chr10: 104342443-104343413 | 0.045949 | 0.044840 |
| A_24_P151727 | NONO | chrX: 70437333-70437392 | 0.068765 | 0.025112 |
| A_24_P161725 | | chr18: 14468365-14468306 | 0.045677 | 0.044154 |
| A_24_P162911 | PRKD3 | chr2: 37337552-37337493 | 0.006815 | 0.017025 |
| A_24_P171873 | FBXO4 | chr5: 41970094-41970153 | 0.002549 | 0.040544 |
| A_24_P195257 | | chr6: 118427073-118427132 | 0.076542 | 0.038023 |
| A_24_P200694 | RING1 | chr6: 33288091-33288150 | 0.047656 | 0.062562 |
| A_24_P201353 | C10orf32 | chr10: 104613777-104613836 | 0.069889 | 0.006169 |
| A_24_P213643 | TSPAN10 | chr17: 77222951-77223010 | 0.046183 | 0.084892 |
| A_24_P217365 | ANKRD28 | chr3: 15683905-15683846 | 0.092105 | 0.030335 |
| A_24_P230576 | | chr18: 26614329-26614387 | 0.076692 | 0.006915 |
| A_24_P247616 | | chr1: 087283336-087283277 | 0.089729 | 0.035713 |
| A_24_P25498 | | chr2: 133404842-133404901 | 0.032110 | 0.091633 |
| A_24_P256060 | LOC649514 | chr10: 8243492-8243551 | 0.076322 | 0.025046 |
| A_24_P274607 | SRA1 | chr5: 139910616-139910557 | 0.053503 | 0.019128 |
| A_24_P282000 | NUP210 | chr3: 13376920-13376861 | 0.096003 | 0.042637 |
| A_24_P294124 | SERTAD2 | chr2: 64712664-64712605 | 0.035526 | 0.007010 |
| A_24_P316363 | GTF3C2 | chr2: 27413898-27413701 | 0.095494 | 0.020802 |
| A_24_P328446 | TLX2 | chr2: 74595605-74595664 | 0.044631 | 0.002179 |
| A_24_P32930 | | chr5: 052745584-052745525 | 0.074144 | 0.020341 |
| A_24_P341897 | ACVR1B | chr12: 50674874-50674933 | 0.007905 | 0.036082 |
| A_24_P348173 | | chr19: 53558400-53555234 | 0.064558 | 0.028852 |
| A_24_P3627 | | chr5: 042589834-042589893 | 0.014610 | 0.045251 |
| A_24_P381494 | SLC11A2 | chr12: 49666625-49666566 | 0.015135 | 0.021535 |
| A_24_P383076 | AASDH | chr4: 56910276-56910217 | 0.003047 | 0.049703 |
| A_24_P393582 | | chr13: 57917881-57917822 | 0.046403 | 0.096563 |
| A_24_P405298 | PPP1CB | chr2: 28877538-28877597 | 0.091203 | 0.013314 |
| A_24_P40795 | LOC283412 | chr5: 43702818-43702759 | 0.070098 | 0.002855 |
| A_24_P409420 | | chr3: 032275758-032275821 | 0.025945 | 0.045180 |
| A_24_P415168 | URM1 | chr9: 130192598-130192653 | 0.023197 | 0.021216 |
| A_24_P456043 | | chr6: 017754043-017754101 | 0.032475 | 0.046295 |
| A_24_P456468 | | chr17: 42755764-42755705 | 0.003777 | 0.082304 |
| A_24_P456884 | | chr6: 100130336-100130395 | 0.071514 | 0.010262 |
| A_24_P464489 | hCG_2041321 | chr1: 158257140-158257199 | 0.045122 | 0.015185 |
| A_24_P466102 | | chr2: 153055200-153055259 | 0.006976 | 0.013310 |
| A_24_P48014 | SOCS1 | chr16: 11256503-11256444 | 0.004992 | 0.036400 |
| A_24_P485105 | | chr9: 131737515-131737456 | 0.040729 | 0.004504 |
| A_24_P487901 | | chr1: 164325697-164325756 | 0.089521 | 0.033557 |
| A_24_P496683 | | chr6: 99827973-99828032 | 0.008709 | 0.071669 |
| A_24_P50328 | | chr6: 090581784-090581843 | 0.010334 | 0.047948 |
| A_24_P50567 | | chr6: 144563421-144563344 | 0.084466 | 0.012831 |
| A_24_P59062 | GGTL4 | chr22: 21318821-21318880 | 0.043167 | 0.099378 |
| A_24_P59239 | | chrX: 014762751-014762686 | 0.004819 | 0.031477 |
| A_24_P632160 | | chr5: 178357888-178357830 | 0.005301 | 0.007396 |
| A_24_P686263 | | | 0.063780 | 0.008188 |
| A_24_P733016 | LOC390427 | chr13: 113982309-113982250 | 0.032692 | 0.000728 |
| A_24_P739344 | NOX4 | chr11: 88699330-88699271 | 0.045454 | 0.011397 |
| A_24_P743869 | LOC92482 | chr10: 112649060-112649001 | 0.045414 | 0.096596 |
| A_24_P74828 | MT1JP | chr16: 55227173-55227232 | 0.089570 | 0.039729 |
| A_24_P76210 | | chrX: 053189643-053189584 | 0.037351 | 0.022081 |
| A_24_P81841 | CDKN1B | chr12: 12766207-12766266 | 0.014175 | 0.022713 |
| A_24_P81841 | CDKN1B | chr12: 12766207-12766266 | 0.013683 | 0.022713 |
| A_24_P81841 | CDKN1B | chr12: 12766207-12766266 | 0.099558 | 0.022713 |
| A_24_P81841 | CDKN1B | chr12: 12766207-12766266 | 0.011851 | 0.022713 |
| A_24_P81841 | CDKN1B | chr12: 12766207-12766266 | 0.011227 | 0.022713 |
| A_24_P81841 | CDKN1B | chr12: 12766207-12766266 | 0.019861 | 0.022713 |
| A_24_P81841 | CDKN1B | chr12: 12766207-12766266 | 0.019384 | 0.022713 |
| A_24_P81841 | CDKN1B | chr12: 12766207-12766266 | 0.014175 | 0.049058 |
| A_24_P81841 | CDKN1B | chr12: 12766207-12766266 | 0.013683 | 0.049058 |
| A_24_P81841 | CDKN1B | chr12: 12766207-12766266 | 0.099558 | 0.049058 |
| A_24_P81841 | CDKN1B | chr12: 12766207-12766266 | 0.011851 | 0.049058 |
| A_24_P81841 | CDKN1B | chr12: 12766207-12766266 | 0.011227 | 0.049058 |
| A_24_P81841 | CDKN1B | chr12: 12766207-12766266 | 0.019861 | 0.049058 |
| A_24_P81841 | CDKN1B | chr12: 12766207-12766266 | 0.019384 | 0.049058 |
| A_24_P81841 | CDKN1B | chr12: 12766207-12766266 | 0.014175 | 0.005945 |
| A_24_P81841 | CDKN1B | chr12: 12766207-12766266 | 0.013683 | 0.005945 |
| A_24_P81841 | CDKN1B | chr12: 12766207-12766266 | 0.099558 | 0.005945 |
| A_24_P81841 | CDKN1B | chr12: 12766207-12766266 | 0.011851 | 0.005945 |
| A_24_P81841 | CDKN1B | chr12: 12766207-12766266 | 0.011227 | 0.005945 |
| A_24_P81841 | CDKN1B | chr12: 12766207-12766266 | 0.019861 | 0.005945 |
| A_24_P81841 | CDKN1B | chr12: 12766207-12766266 | 0.019384 | 0.005945 |
| A_24_P81841 | CDKN1B | chr12: 12766207-12766266 | 0.014175 | 0.012276 |
| A_24_P81841 | CDKN1B | chr12: 12766207-12766266 | 0.013683 | 0.012276 |
| A_24_P81841 | CDKN1B | chr12: 12766207-12766266 | 0.099558 | 0.012276 |
| A_24_P81841 | CDKN1B | chr12: 12766207-12766266 | 0.011851 | 0.012276 |
| A_24_P81841 | CDKN1B | chr12: 12766207-12766266 | 0.011227 | 0.012276 |
| A_24_P81841 | CDKN1B | chr12: 12766207-12766266 | 0.019861 | 0.012276 |
| A_24_P81841 | CDKN1B | chr12: 12766207-12766266 | 0.019384 | 0.012276 |
| A_24_P81841 | CDKN1B | chr12: 12766207-12766266 | 0.014175 | 0.003454 |
| A_24_P81841 | CDKN1B | chr12: 12766207-12766266 | 0.013683 | 0.003454 |
| A_24_P81841 | CDKN1B | chr12: 12766207-12766266 | 0.099558 | 0.003454 |
| A_24_P81841 | CDKN1B | chr12: 12766207-12766266 | 0.011851 | 0.003454 |
| A_24_P81841 | CDKN1B | chr12: 12766207-12766266 | 0.011227 | 0.003454 |
| A_24_P81841 | CDKN1B | chr12: 12766207-12766266 | 0.019861 | 0.003454 |
| A_24_P81841 | CDKN1B | chr12: 12766207-12766266 | 0.019384 | 0.003454 |
| A_24_P81841 | CDKN1B | chr12: 12766207-12766266 | 0.014175 | 0.016158 |
| A_24_P81841 | CDKN1B | chr12: 12766207-12766266 | 0.013683 | 0.016158 |
| A_24_P81841 | CDKN1B | chr12: 12766207-12766266 | 0.099558 | 0.016158 |
| A_24_P81841 | CDKN1B | chr12: 12766207-12766266 | 0.011851 | 0.016158 |
| A_24_P81841 | CDKN1B | chr12: 12766207-12766266 | 0.011227 | 0.016158 |
| A_24_P81841 | CDKN1B | chr12: 12766207-12766266 | 0.019861 | 0.016158 |
| A_24_P81841 | CDKN1B | chr12: 12766207-12766266 | 0.019384 | 0.016158 |
| A_24_P81841 | CDKN1B | chr12: 12766207-12766266 | 0.014175 | 0.033292 |
| A_24_P81841 | CDKN1B | chr12: 12766207-12766266 | 0.013683 | 0.033292 |
| A_24_P81841 | CDKN1B | chr12: 12766207-12766266 | 0.099558 | 0.033292 |
| A_24_P81841 | CDKN1B | chr12: 12766207-12766266 | 0.011851 | 0.033292 |
| A_24_P81841 | CDKN1B | chr12: 12766207-12766266 | 0.011227 | 0.033292 |
| A_24_P81841 | CDKN1B | chr12: 12766207-12766266 | 0.019861 | 0.033292 |
| A_24_P81841 | CDKN1B | chr12: 12766207-12766266 | 0.019384 | 0.033292 |
| A_24_P84738 | LOC392226 | chr8: 63219652-63219593 | 0.048872 | 0.039450 |
| A_24_P917026 | NF1 | chr17: 26728686-26728745 | 0.062786 | 0.019978 |
| A_24_P919304 | FNDC3B | chr3: 173387434-173387493 | 0.069660 | 0.032684 |
| A_24_P930940 | | chr10: 081223930-081223989 | 0.064457 | 0.042124 |
| A_24_P931443 | GPR68 | chr14: 90768741-90768682 | 0.025385 | 0.009953 |
| A_24_P934809 | CSNK2A2 | chr16: 56751432-56751373 | 0.074745 | 0.012561 |
| A_24_P941450 | | chr14: 054583896-054583837 | 0.047204 | 0.074588 |
| A_32_P110042 | | chr19: 39652236-39652177 | 0.073324 | 0.023701 |
| A_32_P125832 | | chr18: 18001927-18001868 | 0.092343 | 0.047489 |
| A_32_P126485 | | chr1: 143052296-143052237 | 0.023414 | 0.081082 |
| A_32_P127812 | | chr10: 70601695-70601636 | 0.051326 | 0.011639 |
| A_32_P133244 | PARN | chr16: 14437452-14437393 | 0.019776 | 0.099752 |
| A_32_P136450 | | chr20: 14476013-14475954 | 0.042805 | 0.005310 |
| A_32_P142881 | SMG1 | chr16: 18778411-18777978 | 0.027441 | 0.055041 |
| A_32_P148199 | | chr2: 63971895-63971836 | 0.075688 | 0.008926 |
| A_32_P149536 | SUMO2 | chr17: 70675569-70675510 | 0.034785 | 0.052702 |
| A_32_P150040 | | chr8: 144756404-144756463 | 0.052039 | 0.037869 |
| A_32_P154121 | | chr13: 48967073-48967132 | 0.058535 | 0.015447 |
| A_32_P155043 | | chr19: 042670572-042670631 | 0.007293 | 0.086686 |
| A_32_P164971 | | chr16: 10944015-10944074 | 0.022964 | 0.094451 |
| A_32_P178842 | | chr1: 036310024-036310083 | 0.004568 | 0.052290 |
| A_32_P195065 | SEMA4F | chr2: 74761974-74762033 | 0.076119 | 0.005389 |
| A_32_P201603 | | chr2: 108669318-108669259 | 0.010935 | 0.089798 |
| A_32_P202042 | ANAPC5 | chr12: 120269135-120269990 | 0.020184 | 0.041140 |
| A_32_P203500 | PCMTD1 | chr8: 52936017-52935958 | 0.053130 | 0.002999 |
| A_32_P203998 | | chr6: 122806787-122806846 | 0.039794 | 0.000022 |
| A_32_P20492 | | chr1: 22291591-22291532 | 0.008252 | 0.050262 |
| A_32_P205624 | SHC2 | chr19: 367892-367833 | 0.047724 | 0.093788 |
| A_32_P20679 | | chr10: 76976989-76977048 | 0.028441 | 0.009624 |
| A_32_P209989 | MRPL46 | chr15: 86811445-86811386 | 0.060293 | 0.025389 |
| A_32_P212024 | LOC653464 | chr1: 120836148-120836207 | 0.093063 | 0.003634 |
| A_32_P219620 | EYA4 | chr6: 133894789-133894848 | 0.006989 | 0.062331 |
| A_32_P220750 | PLGLB1 | chr2: 87091616-87091557 | 0.046000 | 0.098395 |
| A_32_P221723 | | chr12: 40917455-40917514 | 0.061724 | 0.035362 |
| A_32_P222751 | | chr6: 3668156-3668215 | 0.049599 | 0.033540 |
| A_32_P228784 | PPP4R2 | chr3: 73197369-73197428 | 0.044052 | 0.064014 |
| A_32_P34365 | | chr1: 146387818-146387759 | 0.041931 | 0.078398 |
| A_32_P43320 | | chr1: 184916820-184916879 | 0.043221 | 0.052669 |
| A_32_P436743 | NY-REN-7 | chr5: 177070896-177070843 | 0.056661 | 0.002100 |
| A_32_P51524 | LOC595101 | chr16: 29444066-29444007 | 0.043819 | 0.015777 |
| A_32_P64990 | | chr11: 2922481-2922540 | 0.047500 | 0.058682 |
| A_32_P6535 | PLGLB1 | chr2: 87091639-87091580 | 0.017793 | 0.079165 |
| A_32_P78876 | | chr2: 108668681-108668622 | 0.079433 | 0.037458 |
| A_32_P808 | SLAIN2 | chr4: 48080467-48080526 | 0.041042 | 0.002954 |
| A_32_P8351 | LOC595101 | chr16: 29444051-29443992 | 0.026958 | 0.014279 |
| A_32_P92489 | PKD1L2 | chr16: 79692374-79692315 | 0.006239 | 0.099462 |

Table 8 lists first cohort genes that were 0.05 in either the Affymetrix or Agilent screening and 0.1 in the other.

TABLE 8

| Gene | | p-value | p-value |
|---|---|---|---|
| Symbol | Genomic Coordinates | Affymetrix | Agilent |
| 2′-PDE | chr3: 57520095-57520154 | 0.02518 | 0.04332 |
| ABCF2 | chr7: 150541405-150541346 | 0.00954 | 0.03338 |
| ADAMTS7 | chr15: 76850455-76850396 | 0.05513 | 0.03833 |
| ADAMTS7 | chr15: 76850455-76850396 | 0.09241 | 0.03833 |
| ADPRH | chr3: 120789335-120789394 | 0.01178 | 0.05945 |
| AGPAT3 | chr21: 44230639-44230698 | 0.03697 | 0.01704 |
| AGPAT3 | chr21: 44230639-44230698 | 0.01894 | 0.01704 |
| AHCTF1 | chr1: 245079612-245079553 | 0.04049 | 0.09645 |
| ALDH1B1 | chr9: 38387141-38387200 | 0.01177 | 0.01195 |
| ALDH1B1 | chr9: 38387141-38387200 | 0.02861 | 0.01195 |
| ANAPC1 | chr2: 112243327-112243268 | 0.01042 | 0.09405 |
| ANGPTL2 | chr9: 128890833-128890774 | 0.01628 | 0.07374 |
| ANGPTL2 | chr9: 128891100-128891041 | 0.01628 | 0.03202 |
| ANKRD28 | chr3: 15683905-15683846 | 0.07923 | 0.03034 |
| ANKRD28 | chr3: 15693542-15692499 | 0.07923 | 0.01706 |
| ANP32A | chr15: 66866836-66863960 | 0.00454 | 0.02925 |
| ANTXR1 | chr2: 69156228-69156287 | 0.00542 | 0.08079 |
| AP1M1 | chr19: 16206642-16206701 | 0.03578 | 0.04269 |
| AP1S3 | chr2: 224348909-224346301 | 0.04739 | 0.06125 |
| API5 | chr11: 43321943-43322002 | 0.05760 | 0.02620 |
| APOB | chr2: 21078063-21078004 | 0.04657 | 0.08315 |
| ARHGEF11 | chr1: 155174859-155173879 | 0.03634 | 0.04511 |
| ARHGEF2 | chr1: 154184025-154183966 | 0.04753 | 0.06443 |
| ARHGEF7 | chr13: 110755596-110755655 | 0.03218 | 0.01880 |
| ARL2BP | chr16: 55844971-55845030 | 0.03044 | 0.01472 |
| ARL2BP | chr16: 55844971-55845030 | 0.02085 | 0.01472 |
| ARNTL2 | chr12: 27460133-27462332 | 0.04129 | 0.04069 |
| ARNTL2 | chr12: 27460133-27462332 | 0.04714 | 0.04069 |
| ARNTL2 | chr12: 27465419-27465477 | 0.04129 | 0.01383 |
| ARNTL2 | chr12: 27465419-27465477 | 0.04714 | 0.01383 |
| ARPC4 | chr3: 9820571-9820630 | 0.03513 | 0.00199 |
| ARPC4 | chr3: 9818421-9820566 | 0.03513 | 0.09008 |
| ASPHD2 | chr22: 25170444-25170503 | 0.04250 | 0.09017 |
| ATAD1 | chr10: 89503038-89502979 | 0.06596 | 0.00891 |
| ATF2 | chr2: 175647768-175647709 | 0.06872 | 0.02178 |
| ATP1A4 | chr1: 158418431-158422734 | 0.02282 | 0.09396 |
| ATP2C2 | chr16: 83054944-8305503 | 0.00514 | 0.08989 |
| ATXN2L | chr16: 28755883-28755942 | 0.08432 | 0.00776 |
| BCL2L11 | chr2: 111638189-111638248 | 0.09119 | 0.04825 |
| BCL2L11 | chr2: 111638189-111638248 | 0.02776 | 0.04825 |
| BCL2L11 | chr2: 111638203-111638262 | 0.09119 | 0.00272 |
| BCL2L11 | chr2: 111638203-111638262 | 0.02776 | 0.00272 |
| BHLHB2 | chr3: 5001260-5001319 | 0.02939 | 0.05829 |
| BHLHB3 | chr12: 26164958-26164899 | 0.06042 | 0.00707 |
| BHMT | chr5: 78462738-78462797 | 0.03032 | 0.07146 |
| BICC1 | chr10: 60243786-60243845 | 0.02179 | 0.06047 |
| BID | chr22: 16598241-16598182 | 0.06546 | 0.02100 |
| BIN3 | chr8: 22534478-22534419 | 0.08064 | 0.01325 |
| BMP1 | chr8: 22110824-22112484 | 0.06526 | 0.04211 |
| C10orf6 | chr10: 102666704-102666763 | 0.04021 | 0.02490 |
| C10orf6 | chr10: 102714359-102714418 | 0.04021 | 0.05465 |
| C11orf49 | chr11: 47140024-47140083 | 0.07030 | 0.04269 |
| C12orf49 | chr12: 115638268-115638209 | 0.06873 | 0.01591 |
| C13orf15 | chr13: 40940937-40942635 | 0.00488 | 0.00957 |
| C13orf23 | chr13: 38483410-38483351 | 0.03350 | 0.09840 |
| C16orf42 | chr16: 1340067-1339903 | 0.02105 | 0.02082 |
| C16orf5 | chr16: 4501905-4501846 | 0.06046 | 0.02664 |
| C19orf2 | chr19: 35195068-35195127 | 0.08344 | 0.04251 |
| C19orf20 | chr19: 470055-470114 | 0.06742 | 0.04657 |
| C19orf58 | chr19: 17286175-17287778 | 0.04521 | 0.06316 |
| C1orf64 | chr1: 16205540-16205599 | 0.02474 | 0.07982 |
| C1orf83 | chr1: 54350180-54350239 | 0.04611 | 0.07792 |
| C20orf20 | chr20: 60901779-60901838 | 0.03976 | 0.05014 |
| C20orf44 | chr20: 33355017-33354958 | 0.08120 | 0.04845 |
| C21orf66 | chr21: 33028886-33028827 | 0.02847 | 0.06386 |
| C22orf9 | chr22: 43971224-43971165 | 0.00421 | 0.03038 |
| C22orf9 | chr22: 43971282-43971223 | 0.00421 | 0.03310 |
| C3orf63 | chr3: 56629690-56629631 | 0.05580 | 0.02029 |
| C6orf154 | chr6: 43583008-43582949 | 0.02523 | 0.07531 |
| C9orf93 | chr9: 15769112-15774529 | 0.02263 | 0.07497 |
| CADM1 | chr11: 114550765-114550706 | 0.02120 | 0.04661 |
| CADM1 | chr11: 114550765-114550706 | 0.04319 | 0.04661 |
| CALU | chr7: 128197605-128197664 | 0.08105 | 0.00535 |
| CALU | chr7: 128197605-128197664 | 0.01062 | 0.00535 |
| CASP4 | chr11: 104318898-104318839 | 0.00864 | 0.03477 |
| CAV1 | chr7: 115986237-115986296 | 0.01392 | 0.02057 |
| CAV2 | chr7: 115935497-115935556 | 0.03458 | 0.06431 |
| CBX5 | chr12: 52926243-52926184 | 0.06669 | 0.01414 |
| CCDC120 | chrX: 48812226-48812285 | 0.03178 | 0.09704 |
| CCDC50 | chr3: 192592323-192592382 | 0.00355 | 0.00235 |
| CCDC85B | chr11: 65415351-65415410 | 0.02869 | 0.04650 |
| CCDC92 | chr12: 122987222-122987163 | 0.06597 | 0.01619 |
| CCNB1 | chr5: 68507119-68508879 | 0.02753 | 0.07687 |
| CD59 | chr11: 33684213-33684154 | 0.07541 | 0.01796 |
| CD59 | chr11: 33684213-33684154 | 0.07517 | 0.01796 |
| CD59 | chr11: 33684213-33684154 | 0.08271 | 0.01796 |
| CD59 | chr11: 33681381-33681322 | 0.07541 | 0.03374 |
| CD59 | chr11: 33681381-33681322 | 0.07517 | 0.03374 |
| CD59 | chr11: 33681381-33681322 | 0.08271 | 0.03374 |
| CD59 | chr11: 33687179-33687120 | 0.07541 | 0.02571 |
| CD59 | chr11: 33687179-33687120 | 0.07517 | 0.02571 |
| CD59 | chr11: 33687179-33687120 | 0.08271 | 0.02571 |
| CDKN2AIP | chr4: 184605904-184605963 | 0.07877 | 0.04509 |
| CDR2 | chr16: 22265245-22265186 | 0.08754 | 0.00270 |
| CDR2 | chr16: 22265245-22265186 | 0.03867 | 0.00270 |
| CDRT4 | chr17: 15282169-15282110 | 0.07771 | 0.01469 |
| CEP135 | chr4: 56521495-56521554 | 0.03743 | 0.01466 |
| CEP68 | chr2: 65166977-65167036 | 0.02215 | 0.01078 |
| CEP68 | chr2: 65166977-65167036 | 0.01170 | 0.01078 |
| CEP68 | chr2: 65153123-65153182 | 0.02215 | 0.05702 |
| CEP68 | chr2: 65153123-65153182 | 0.01170 | 0.05702 |
| CHRNE | chr17: 4742718-4742659 | 0.00968 | 0.00608 |
| CLIC4 | chr1: 25039105-25039895 | 0.01684 | 0.03933 |
| CLN8 | chr8: 1719039-1719098 | 0.06803 | 0.02813 |
| CLN8 | chr8: 1719039-1719098 | 0.02625 | 0.02813 |
| CLN8 | chr8: 1719039-1719098 | 0.04037 | 0.02813 |
| CLN8 | chr8: 1719293-1719352 | 0.06803 | 0.00545 |
| CLN8 | chr8: 1719293-1719352 | 0.02625 | 0.00545 |
| CLN8 | chr8: 1719293-1719352 | 0.04037 | 0.00545 |
| CLTA | chr9: 36201781-36201840 | 0.05923 | 0.00514 |
| CMYA5 | chr5: 79067786-79067845 | 0.01143 | 0.04880 |
| CNBD1 | chr8: 88367945-88368004 | 0.02168 | 0.03942 |
| COL12A1 | chr6: 75851068-75851009 | 0.00886 | 0.00856 |
| COL12A1 | chr6: 75851068-75851009 | 0.09084 | 0.00856 |
| COL12A1 | chr6: 75851068-75851009 | 0.05257 | 0.00856 |
| COL17A1 | chr10: 105781832-105781773 | 0.04323 | 0.05785 |
| COL8A2 | chr1: 36333509-36333450 | 0.04936 | 0.06710 |
| COTL1 | chr16: 83156898-83156839 | 0.03035 | 0.03176 |
| CPM | chr12: 67535674-67535615 | 0.03023 | 0.09289 |
| CPXM2 | chr10: 125495243-125495184 | 0.02132 | 0.07391 |
| CREB3L1 | chr11: 46299335-46299394 | 0.04596 | 0.07928 |
| CREBL2 | chr12: 12686308-12686367 | 0.03066 | 0.05820 |
| CREBL2 | chr12: 12686308-12686367 | 0.04704 | 0.05820 |
| CREM | chr10: 35540647-35540706 | 0.07578 | 0.04019 |
| CRIPAK | chr4: 1379548-1379607 | 0.00949 | 0.00727 |
| CROP | chr17: 46184287-46184346 | 0.00273 | 0.05665 |
| CROP | chr17: 46184287-46184346 | 0.00518 | 0.05665 |
| CSNK2A2 | chr16: 56751432-56751373 | 0.00466 | 0.01256 |
| CTDSP2 | chr12: 56500379-56500320 | 0.07284 | 0.01966 |
| CTGF | chr6: 132311882-132311823 | 0.04245 | 0.08534 |
| CUGBP2 | chr10: 11416231-11416290 | 0.05674 | 0.03861 |
| CXADR | chr21: 17846048-17846107 | 0.02037 | 0.02092 |
| CXADR | chr21: 17860528-17860587 | 0.02037 | 0.08964 |
| CXorf39 | chrX: 103325502-103325561 | 0.02756 | 0.07263 |
| CYBA | chr16: 87240706-87240090 | 0.03638 | 0.03728 |
| CYP2A13 | chr19: 46293574-46293633 | 0.02304 | 0.07406 |
| CYP2R1 | chr11: 14856267-14856208 | 0.01374 | 0.07210 |
| CYR61 | chr1: 85821032-85821091 | 0.01253 | 0.05840 |
| CYR61 | chr1: 85821759-85821818 | 0.01253 | 0.06608 |
| CYR61 | chr1: 85821541-85821600 | 0.01253 | 0.00119 |
| DAPK1 | chr9: 89512595-89512654 | 0.00886 | 0.02901 |
| DCK | chr4: 72115031-72115090 | 0.07387 | 0.04830 |
| DDX50 | chr10: 70376457-70376516 | 0.01497 | 0.04723 |
| DFFA | chr1: 10443929-10443870 | 0.00785 | 0.07085 |
| DHRS9 | chr2: 169660621-169660680 | 0.00145 | 0.04363 |
| DHX15 | chr4: 24140442-24140383 | 0.09950 | 0.02158 |
| DICER1 | chr14: 94626045-94625986 | 0.08577 | 0.03513 |
| DKK3 | chr11: 11941448-11941389 | 0.09894 | 0.04754 |
| DNAI1 | chr9: 34507408-34510657 | 0.01890 | 0.06626 |
| DNAJA5 | chr5: 34986152-34989794 | 0.00502 | 0.06758 |
| DNAJB5 | chr9: 34988281-34988340 | 0.01217 | 0.02279 |
| DOCK4 | chr7: 111166530-111166471 | 0.00894 | 0.00781 |
| DOK4 | chr16: 56063694-56063635 | 0.02158 | 0.06680 |
| DPYD | chr1: 97958880-97958821 | 0.05928 | 0.04581 |
| DPYD | chr1: 97316547-97316488 | 0.05928 | 0.03372 |
| DRD2 | chr11: 112786811-112786752 | 0.02572 | 0.04892 |
| DUSP3 | chr17: 39202336-39202277 | 0.00170 | 0.09137 |
| EARS2 | chr16: 23441251-23441192 | 0.09544 | 0.04215 |
| ECE2 | chr3: 185493317-185493376 | 0.07180 | 0.01678 |
| EFHB | chr3: 19922239-19922180 | 0.09137 | 0.04728 |
| EFHD2 | chr1: 15629224-15629283 | 0.05932 | 0.01167 |
| EFS | chr14: 22895649-22895590 | 0.01387 | 0.00697 |
| EHD2 | chr19: 52938036-52938095 | 0.03829 | 0.07658 |
| EI24 | chr11: 124958643-124958702 | 0.08733 | 0.04433 |
| EIF4E3 | chr3: 71814918-71814859 | 0.06105 | 0.00158 |
| EIF4EBP2 | chr10: 71849793-71849852 | 0.02088 | 0.03705 |
| ELL2 | chr5: 95248974-95248915 | 0.04664 | 0.03886 |
| ELN | chr7: 73121941-73122000 | 0.02240 | 0.05822 |
| ELN | chr7: 73121535-73121594 | 0.02240 | 0.07408 |
| ENDOD1 | chr11: 94505240-94505299 | 0.02197 | 0.02938 |
| ENTPD7 | chr10: 101455180-101455239 | 0.06841 | 0.02331 |
| ENTPD7 | chr10: 101455019-101455078 | 0.06841 | 0.02768 |
| EPM2A | chr6: 145989099-145989040 | 0.01068 | 0.09999 |
| EPN3 | chr17: 45975055-45975114 | 0.05265 | 0.01760 |
| EPOR | chr19: 11353383-11352946 | 0.00103 | 0.02878 |
| ERCC8 | chr5: 60222532-60222473 | 0.08780 | 0.01657 |
| ERCC8 | chr5: 60222532-60222473 | 0.00834 | 0.01657 |
| EXOC4 | chr7: 133400421-133400480 | 0.00509 | 0.07947 |
| FADS1 | chr11: 61326207-61326148 | 0.05944 | 0.03225 |
| FAM126A | chr7: 22950024-22949965 | 0.01527 | 0.07160 |
| FAM26B | chr10: 105196810-105196751 | 0.03627 | 0.04693 |
| FAM44A | chr4: 13236377-13236318 | 0.03076 | 0.07611 |
| FASLG | chr1: 170901687-170901746 | 0.02956 | 0.08494 |
| FBXO2 | chr1: 11631204-11631145 | 0.01327 | 0.09086 |
| FBXO3 | chr11: 33719902-33719843 | 0.00478 | 0.00529 |
| FBXO4 | chr5: 41970094-41970153 | 0.09181 | 0.04054 |
| FCAR | chr19: 60088612-60088671 | 0.08409 | 0.00387 |
| FCAR | chr19: 60088612-60088671 | 0.06974 | 0.00387 |
| FCGBP | chr19: 45045966-45045907 | 0.01144 | 0.02754 |
| FCHO2 | chr5: 72421695-72421754 | 0.02205 | 0.00559 |
| FGF18 | chr5: 170795863-170808795 | 0.01230 | 0.07207 |
| FGF18 | chr5: 170816574-170816633 | 0.01230 | 0.02708 |
| FKBP7 | chr2: 179038811-179038752 | 0.00702 | 0.08916 |
| FLJ20323 | chr7: 7602521-7602580 | 0.01176 | 0.04103 |
| FLJ20323 | chr7: 7602521-7602580 | 0.08080 | 0.04103 |
| FLJ21062 | chr7: 89776561-89777330 | 0.06487 | 0.00406 |
| FLJ30064 | chr7: 97777448-97777507 | 0.08407 | 0.04499 |
| FLJ31222 | chr17: 38283606-38283547 | 0.03236 | 0.02948 |
| FLJ31818 | chr7: 112246718-112246659 | 0.06304 | 0.00821 |
| FLJ32756 | chr22: 45689975-45689916 | 0.08757 | 0.04683 |
| FLJ38973 | chr2: 200500915-200500974 | 0.05893 | 0.02610 |
| FLJ90680 | chr22: 35573388-35573329 | 0.04470 | 0.08922 |
| FMNL3 | chr12: 48327801-48327408 | 0.02552 | 0.05582 |
| FNDC4 | chr2: 27568826-27568767 | 0.06320 | 0.00764 |
| FOXD1 | chr5: 72778894-72778835 | 0.01654 | 0.07747 |
| FOXQ1 | chr6: 1259629-1259688 | 0.01432 | 0.09756 |
| FSCN1 | chr7: 5611468-5611527 | 0.02504 | 0.03507 |
| FVT1 | chr18: 59149913-59149854 | 0.04890 | 0.03336 |
| FVT1 | chr18: 59149184-59149125 | 0.04890 | 0.00215 |
| FZD1 | chr7: 90735776-90735835 | 0.04592 | 0.01880 |
| GALNT2 | chr1: 228483885-228483944 | 0.04086 | 0.03755 |
| GALNTL2 | chr3: 16244808-16244867 | 0.02058 | 0.09944 |
| GAS7 | chr17: 9760158-9760099 | 0.01827 | 0.04033 |
| GAS7 | chr17: 9760158-9760099 | 0.01085 | 0.04033 |
| GAS7 | chr17: 9755003-9754944 | 0.01827 | 0.01030 |
| GAS7 | chr17: 9755003-9754944 | 0.01085 | 0.01030 |
| GATAD1 | chr7: 91923728-91923787 | 0.02698 | 0.04439 |
| GATS | chr7: 99636814-99636755 | 0.07508 | 0.00437 |
| GCGR | chr17_random: 2329878-2329937 | 0.08128 | 0.04268 |
| GFER | chr16: 1975921-1975980 | 0.01991 | 0.09028 |
| GGA1 | chr22: 36359426-36359485 | 0.03367 | 0.09432 |
| GGA1 | chr22: 36359426-36359485 | 0.01372 | 0.09432 |
| GIT2 | chr12: 108852062-108852003 | 0.03480 | 0.03992 |
| GLE1L | chr9: 130335947-130336006 | 0.01883 | 0.00473 |
| GLIPR1 | chr12: 74179523-74179582 | 0.07449 | 0.04620 |
| GLIPR1 | chr12: 74179523-74179582 | 0.00750 | 0.04620 |
| GLIPR1 | chr12: 74178990-74179049 | 0.07449 | 0.00183 |
| GLIPR1 | chr12: 74178990-74179049 | 0.00750 | 0.00183 |
| GNA11 | chr19: 3072011-3072070 | 0.03523 | 0.01277 |
| GNL3L | chrX: 54601800-54603723 | 0.05892 | 0.04816 |
| GON4L | chr1: 154021140-154021081 | 0.01946 | 0.03083 |
| GOPC | chr6: 117994793-117994734 | 0.00914 | 0.07723 |
| GOPC | chr6: 117988368-117988309 | 0.00914 | 0.01624 |
| GPR114 | chr16: 56168469-56168528 | 0.02297 | 0.03621 |
| GPRC5C | chr17: 69948481-69948540 | 0.01791 | 0.06362 |
| GRID1 | chr10: 87351883-87351824 | 0.04094 | 0.01087 |
| GRRP1 | chr1: 26361581-26361640 | 0.02692 | 0.07471 |
| GSTT1 | chr22: 22706392-22706333 | 0.04626 | 0.05862 |
| GTDC1 | chr2: 144709670-144709611 | 0.00140 | 0.03575 |
| GTF3C2 | chr2: 27413898-27413701 | 0.03624 | 0.02080 |
| GTF3C2 | chr2: 27413898-27413701 | 0.05017 | 0.02080 |
| GTF3C2 | chr2: 27402233-27402210 | 0.03624 | 0.03109 |
| GTF3C2 | chr2: 27402233-27402210 | 0.05017 | 0.03109 |
| H6PD | chr1: 9247209-9247268 | 0.08953 | 0.04642 |
| HDAC4 | chr2: 239635672-239635613 | 0.03118 | 0.01128 |
| HFE | chr6: 26202463-26202522 | 0.00338 | 0.05101 |
| HGSNAT | chr8: 43175304-43175363 | 0.03264 | 0.05429 |
| HIBCH | chr2: 190778046-190777987 | 0.04199 | 0.03300 |
| HIP1 | chr7: 75205469-75205410 | 0.03511 | 0.06775 |
| HIVEP2 | chr6: 143114603-143114544 | 0.09382 | 0.02955 |
| HNRPA3 | chr2: 177796555-177796614 | 0.02822 | 0.08825 |
| HRH1 | chr3: 11278801-11278860 | 0.04948 | 0.08750 |
| HRH1 | chr3: 11277202-11277261 | 0.04948 | 0.08191 |
| HRK | chr12: 115783495-115783436 | 0.00422 | 0.04910 |
| HS2ST1 | chr1: 87347957-87348016 | 0.04984 | 0.02333 |
| HSF2 | chr6: 122795598-122795657 | 0.03427 | 0.00520 |
| HSPBP1 | chr19: 60465851-60465792 | 0.03156 | 0.07409 |
| ICOSLG | chr21: 44479782-44479723 | 0.01961 | 0.05615 |
| IDS | chrX: 148366698-148366639 | 0.02294 | 0.07050 |
| IGF2BP3 | chr7: 23317353-23317294 | 0.01421 | 0.05515 |
| IKIP | chr12: 97543079-97543020 | 0.00995 | 0.01221 |
| IKIP | chr12: 97543079-97543020 | 0.00933 | 0.01221 |
| IKIP | chr12: 97543079-97543020 | 0.09170 | 0.01221 |
| IL17D | chr13: 20194978-20195037 | 0.05185 | 0.03949 |
| IPO8 | chr12: 30678322-30676192 | 0.01994 | 0.04379 |
| IPO8 | chr12: 30678322-30676192 | 0.01994 | 0.05107 |
| IQSEC1 | chr3: 12928194-12928135 | 0.07408 | 0.03970 |
| ITGA2 | chr5: 52425394-52425453 | 0.01449 | 0.05823 |
| ITGA9 | chr3: 37540031-37540090 | 0.00815 | 0.07472 |
| ITGA9 | chr3: 37835561-37835620 | 0.00815 | 0.02932 |
| ITGA9 | chr3: 37835561-37835620 | 0.06022 | 0.02932 |
| ITSN1 | chr21: 34193677-34193736 | 0.05917 | 0.02968 |
| JARID1A | chr12: 264068-264009 | 0.08661 | 0.03401 |
| JTV1 | chr7: 6029564-6029623 | 0.09273 | 0.01194 |
| KCTD10 | chr12: 108371402-108371343 | 0.01774 | 0.02593 |
| KIAA0157 | chr10: 126514856-126514915 | 0.03721 | 0.05746 |
| KIAA0494 | chr1: 46913675-46913616 | 0.00421 | 0.01527 |
| KIAA0513 | chr16: 83666978-83668547 | 0.03470 | 0.05425 |
| KIAA0895 | chr7: 36331187-36331128 | 0.04098 | 0.09361 |
| KIAA1143 | chr3: 44765278-44765242 | 0.02851 | 0.02093 |
| KIAA1641 | chr2: 97490919-97490523 | 0.01947 | 0.07781 |
| KLF2 | chr19: 16297204-16297263 | 0.01858 | 0.08290 |
| KLHL20 | chr1: 171970179-171970238 | 0.08873 | 0.02433 |
| KRR1 | chr12: 74184103-74184044 | 0.05653 | 0.00603 |
| KRTAP4-4 | chr17: 36569734-36569675 | 0.04584 | 0.08585 |
| LAMA4 | chr6: 112537242-112537183 | 0.06946 | 0.00205 |
| LMBR1L | chr12: 47777564-47777505 | 0.03261 | 0.08586 |
| LOC154822 | chr7: 158518881-158518940 | 0.05687 | 0.04774 |
| LOC254559 | chr15: 87739280-87739339 | 0.02400 | 0.07663 |
| LOC339804 | chr2: 61243495-61243554 | 0.02134 | 0.02864 |
| LOC401317 | chr7: 28416632-28416691 | 0.04395 | 0.02488 |
| LOC404266 | chr17: 44036316-44036375 | 0.04313 | 0.04721 |
| LOC441204 | chr7: 26504299-26504358 | 0.01520 | 0.08706 |
| LOC652411 | chr6: 54597720-54597781 | 0.04501 | 0.06215 |
| LPAL2 | chr6: 160841813-160833936 | 0.05284 | 0.03447 |
| LPAL2 | chr6: 160841813-160833936 | 0.08549 | 0.03447 |
| LRP5L | chr22: 24077513-24077454 | 0.09443 | 0.03025 |
| LRRC17 | chr7: 102372062-102372121 | 0.01340 | 0.03775 |
| LRRFIP2 | chr3: 37069859-37069801 | 0.05159 | 0.03569 |
| LSM14B | chr20: 60142770-60142829 | 0.05558 | 0.02582 |
| LST1 | chr6: 31664372-31664431 | 0.02773 | 0.00268 |
| LUM | chr12: 90021836-90021777 | 0.05448 | 0.02139 |
| MAGIX | chrX: 48911032-48911091 | 0.03541 | 0.08162 |
| MAP1LC3A | chr20: 32611316-32611375 | 0.01674 | 0.08425 |
| MAP2K3 | chr17: 21158581-21158640 | 0.07408 | 0.00952 |
| MAP3K11 | chr11: 65122235-65122176 | 0.04442 | 0.03212 |
| MAP3K2 | chr2: 127780999-127780940 | 0.06064 | 0.00109 |
| MAP7D3 | chrX: 135140732-135140673 | 0.00351 | 0.00466 |
| MAPK8IP3 | chr16: 1736275-1736334 | 0.06765 | 0.04176 |
| MAT2B | chr5: 162877891-162877950 | 0.06730 | 0.02133 |
| MBTD1 | chr17: 46624813-46624754 | 0.03843 | 0.04635 |
| MCOLN3 | chr1: 85260616-85260451 | 0.00790 | 0.03451 |
| MDGA1 | chr6: 37708332-37708273 | 0.01263 | 0.03299 |
| MDH1 | chr2: 63687741-63687800 | 0.01267 | 0.03187 |
| MDH1B | chr2: 207328460-207328401 | 0.07206 | 0.03691 |
| MEIS3P1 | chr17: 20435191-20435250 | 0.00343 | 0.05289 |
| MFAP5 | chr12: 8691917-8691858 | 0.03210 | 0.03490 |
| MFI2 | chr3: 198230327-198230268 | 0.01675 | 0.08389 |
| MGC11082 | chr18: 3594125-3594184 | 0.04525 | 0.05504 |
| MGC17403 | chrX: 13592715-13592774 | 0.05936 | 0.00720 |
| MIAT | chr22: 25396497-25396556 | 0.02526 | 0.07612 |
| MIAT | chr22: 25396980-25397039 | 0.09640 | 0.01582 |
| MIAT | chr22: 25396980-25397039 | 0.08268 | 0.01582 |
| MIAT | chr22: 25396980-25397039 | 0.02526 | 0.01582 |
| MLL | chr11: 117867208-117867701 | 0.02265 | 0.04822 |
| MNT | chr17: 2234385-2234326 | 0.04011 | 0.07973 |
| MR1 | chr1: 179291683-179291742 | 0.00404 | 0.02834 |
| MR1 | chr1: 179285969-179286028 | 0.00404 | 0.01020 |
| MRPS12 | chr19: 44115409-44115468 | 0.01328 | 0.09542 |
| MTMR6 | chr13: 24720113-24720054 | 0.04700 | 0.03936 |
| MUC12 | chr7: 100443061-100443120 | 0.07612 | 0.00469 |
| MUC12 | chr7: 100443061-100443120 | 0.02960 | 0.00469 |
| MYADM | chr19: 59071373-59071432 | 0.03122 | 0.01279 |
| MYADM | chr19: 59068804-59068863 | 0.03122 | 0.01377 |
| MYADM | chr19: 59069709-59069768 | 0.03122 | 0.00069 |
| MYLC2PL | chr7: 101043496-101043437 | 0.00410 | 0.03662 |
| MYST4 | chr10: 76461429-76461488 | 0.08016 | 0.00469 |
| NAGA | chr22: 40784989-40784930 | 0.02856 | 0.01295 |
| NBPF11 | chr1: 146045986-146045927 | 0.05828 | 0.04605 |
| NEGR1 | chr1: 71644863-71644804 | 0.04824 | 0.01144 |
| NF2 | chr22: 28387293-28391002 | 0.02791 | 0.02589 |
| NF2 | chr22: 28391004-28394331 | 0.02791 | 0.09607 |
| NFAM1 | chr22: 41137598-41137539 | 0.00234 | 0.08125 |
| NHP2L1 | chr22: 40405506-40405447 | 0.05584 | 0.00777 |
| NKX6-3 | chr8: 41621914-41621855 | 0.00863 | 0.09054 |
| NPFF | chr12: 52187144-52187085 | 0.00497 | 0.06940 |
| NRP2 | chr2: 206370010-206370069 | 0.04290 | 0.02830 |
| NRP2 | chr2: 206370010-206370069 | 0.04653 | 0.02830 |
| NRP2 | chr2: 206370010-206370069 | 0.03416 | 0.02830 |
| NRP2 | chr2: 206370010-206370069 | 0.08971 | 0.02830 |
| NRP2 | chr2: 206349676-206349735 | 0.04290 | 0.00151 |
| NRP2 | chr2: 206349676-206349735 | 0.04653 | 0.00151 |
| NRP2 | chr2: 206349676-206349735 | 0.03416 | 0.00151 |
| NRP2 | chr2: 206349676-206349735 | 0.08971 | 0.00151 |
| NTN2L | chr16: 2462819-2462878 | 0.04333 | 0.08031 |
| ODZ1 | chrX: 123342194-123342135 | 0.08769 | 0.02803 |
| ODZ3 | chr4: 183958377-183958436 | 0.01360 | 0.07981 |
| ODZ3 | chr4: 183958750-183958809 | 0.01360 | 0.05105 |
| OLFML1 | chr11: 7488392-7488451 | 0.03969 | 0.03481 |
| OPCML | chr11: 131790184-131790125 | 0.04645 | 0.09966 |
| OTUD4 | chr4: 146275073-146275014 | 0.07918 | 0.04183 |
| OXT | chr20: 3001096-3001155 | 0.03523 | 0.09960 |
| P18SRP | chr5: 64053252-64053193 | 0.03676 | 0.08267 |
| P4HA2 | chr5: 131571355-131567741 | 0.00072 | 0.09682 |
| PAFAH1B2 | chr11: 116543710-116543769 | 0.00621 | 0.06528 |
| PAFAH1B2 | chr11: 116543710-116543769 | 0.00000 | 0.06528 |
| PAFAH1B2 | chr11: 116546077-116546136 | 0.00621 | 0.00989 |
| PAFAH1B2 | chr11: 116546077-116546136 | 0.00000 | 0.00989 |
| PAK3 | chrX: 110325760-110325819 | 0.03340 | 0.00331 |
| PARVA | chr11: 12505985-12506044 | 0.01087 | 0.07293 |
| PCDH10 | chr4: 134331840-134331899 | 0.00598 | 0.06795 |
| PCGF2 | chr17: 34144575-34144516 | 0.04689 | 0.06040 |
| PCMTD1 | chr8: 52936017-52935958 | 0.08236 | 0.00300 |
| PCMTD1 | chr8: 52893406-52893465 | 0.08236 | 0.00947 |
| PCOLCE | chr7: 100042003-100042062 | 0.06745 | 0.03248 |
| PDCD6IP | chr3: 33885823-33885882 | 0.03742 | 0.02953 |
| PDE4C | chr19: 18181982-18181923 | 0.04721 | 0.00266 |
| PDE4C | chr19: 18181982-18181923 | 0.01709 | 0.00266 |
| PDE4C | chr19: 18181982-18181923 | 0.04721 | 0.00879 |
| PDE4C | chr19: 18181982-18181923 | 0.01709 | 0.00879 |
| PDE4C | chr19: 18181982-18181923 | 0.04721 | 0.00369 |
| PDE4C | chr19: 18181982-18181923 | 0.01709 | 0.00369 |
| PDE4C | chr19: 18181982-18181923 | 0.04721 | 0.00298 |
| PDE4C | chr19: 18181982-18181923 | 0.01709 | 0.00298 |
| PDE4C | chr19: 18181982-18181923 | 0.04721 | 0.00694 |
| PDE4C | chr19: 18181982-18181923 | 0.01709 | 0.00694 |
| PDE4C | chr19: 18181982-18181923 | 0.04721 | 0.00440 |
| PDE4C | chr19: 18181982-18181923 | 0.01709 | 0.00440 |
| PDE4C | chr19: 18181982-18181923 | 0.04721 | 0.00720 |
| PDE4C | chr19: 18181982-18181923 | 0.01709 | 0.00720 |
| PDE4C | chr19: 18181982-18181923 | 0.04721 | 0.00846 |
| PDE4C | chr19: 18181982-18181923 | 0.01709 | 0.00846 |
| PDE4C | chr19: 18181982-18181923 | 0.04721 | 0.00550 |
| PDE4C | chr19: 18181982-18181923 | 0.01709 | 0.00550 |
| PDK4 | chr7: 95054324-95054265 | 0.01742 | 0.09950 |
| PDK4 | chr7: 95054324-95054265 | 0.04752 | 0.09950 |
| PFDN4 | chr20: 52265292-52265351 | 0.02059 | 0.06797 |
| PHF21A | chr11: 45943465-45931717 | 0.01595 | 0.04395 |
| PIGQ | chr16: 567145-567204 | 0.04971 | 0.07506 |
| PLA2G4C | chr19: 53294825-53294766 | 0.04550 | 0.03348 |
| PLA2G4C | chr19: 53294825-53294766 | 0.03136 | 0.03348 |
| PLAGL1 | chr6: 144303811-144303752 | 0.05855 | 0.03657 |
| PLXNC1 | chr12: 93224788-93224847 | 0.06747 | 0.03502 |
| POLS | chr5: 6809843-6809902 | 0.03477 | 0.06154 |
| PPAP2B | chr1: 56733487-56733428 | 0.06862 | 0.04927 |
| PPP3CA | chr4: 102165761-102165702 | 0.07872 | 0.01177 |
| PPP3R1 | chr2: 68259773-68259714 | 0.06235 | 0.03113 |
| PPTC7 | chr12: 109456886-109456827 | 0.05573 | 0.00141 |
| PRELID2 | chr5: 145124756-145124697 | 0.00634 | 0.09236 |
| PRKAR1A | chr17: 64039017-64039076 | 0.08588 | 0.00376 |
| PRKAR1A | chr17: 64039216-64039275 | 0.08588 | 0.00068 |
| PRKD3 | chr2: 37337552-37337493 | 0.00662 | 0.01702 |
| PRKD3 | chr2: 37331584-37331525 | 0.00662 | 0.08442 |
| PRKG1 | chr10: 53724622-53724682 | 0.03934 | 0.08532 |
| PRKG1 | chr10: 53724622-53724682 | 0.01597 | 0.08532 |
| PRKX | chrX: 3532850-3532791 | 0.08034 | 0.00852 |
| PRND | chr20: 4656988-4657047 | 0.09639 | 0.01330 |
| PRODH2 | chr19: 40985007-40984948 | 0.03681 | 0.05035 |
| PRR6 | chr17: 16193980-16192676 | 0.04697 | 0.06136 |
| PRSS23 | chr11: 86199710-86199769 | 0.04116 | 0.05513 |
| PS1TP4 | chr2: 114181365-114181306 | 0.08171 | 0.03316 |
| PSMD11 | chr17: 27831300-27831650 | 0.01403 | 0.09307 |
| PTP4A2 | chr1: 32145132-32145073 | 0.06231 | 0.00496 |
| PTP4A2 | chr1: 32146750-32146692 | 0.06231 | 0.02820 |
| PTPLB | chr3: 124693487-124693428 | 0.03635 | 0.05836 |
| PTPN11 | chr12: 111428416-111428475 | 0.04433 | 0.00005 |
| PTPN11 | chr12: 111431267-111431326 | 0.04433 | 0.01492 |
| PTPN11 | chr12: 111431267-111431326 | 0.04433 | 0.02243 |
| PTPN11 | chr12: 111431267-111431326 | 0.04433 | 0.02727 |
| PTPN11 | chr12: 111431267-111431326 | 0.04433 | 0.03612 |
| PTPN11 | chr12: 111431267-111431326 | 0.04433 | 0.01457 |
| PTPN11 | chr12: 111431267-111431326 | 0.04433 | 0.01266 |
| PTPN11 | chr12: 111431267-111431326 | 0.04433 | 0.05183 |
| PTRF | chr17: 37810546-37810487 | 0.03387 | 0.01738 |
| PTRH1 | chr9: 129516073-129516014 | 0.05812 | 0.03989 |
| PURB | chr7: 44882626-44882567 | 0.04243 | 0.09748 |
| PUS7 | chr7: 104884397-104884338 | 0.01405 | 0.09358 |
| QKI | chr6: 163910518-163910577 | 0.00149 | 0.01523 |
| QKI | chr6: 163910518-163910577 | 0.07139 | 0.01523 |
| QKI | chr6: 163876087-163876146 | 0.00149 | 0.02339 |
| QKI | chr6: 163876087-163876146 | 0.07139 | 0.02339 |
| RAB22A | chr20: 56368866-56368925 | 0.00591 | 0.05525 |
| RAB22A | chr20: 56368866-56368925 | 0.02072 | 0.05525 |
| RAB8B | chr15: 61343176-61343235 | 0.03128 | 0.00057 |
| RAD50 | chr5: 132004378-132005809 | 0.09548 | 0.01804 |
| RAF1 | chr3: 12600670-12600611 | 0.00273 | 0.02502 |
| RAF1 | chr3: 12600670-12600611 | 0.00273 | 0.03280 |
| RAF1 | chr3: 12600670-12600611 | 0.00273 | 0.07310 |
| RAF1 | chr3: 12600670-12600611 | 0.00273 | 0.06872 |
| RALA | chr7: 39702850-39702909 | 0.01729 | 0.04948 |
| RALB | chr2: 120760072-120763628 | 0.00058 | 0.00473 |
| RALB | chr2: 120760072-120763628 | 0.05876 | 0.00473 |
| RAPH1 | chr2: 204007005-204006946 | 0.04065 | 0.00102 |
| RC3H2 | chr9: 124695042-124692620 | 0.02994 | 0.00656 |
| RFX3 | chr9: 3247191-3247132 | 0.01186 | 0.09020 |
| RGS2 | chr1: 191047674-191047733 | 0.04821 | 0.03165 |
| RHBDD1 | chr2: 227569291-227569350 | 0.01294 | 0.02436 |
| RIF1 | chr2: 152039738-152039797 | 0.00185 | 0.06616 |
| RNF138 | chr18: 27964319-27964378 | 0.02308 | 0.09017 |
| ROM1 | chr11: 62139080-62139139 | 0.04386 | 0.06715 |
| RPL10A | chr6: 35546407-35546466 | 0.05150 | 0.04411 |
| RUNX2 | chr6: 45404144-45404403 | 0.03478 | 0.06784 |
| RUNX2 | chr6: 45626194-45626253 | 0.03478 | 0.03285 |
| RUNX2 | chr6: 45626194-45626253 | 0.05325 | 0.03285 |
| RUNX2 | chr6: 45626194-45626253 | 0.05719 | 0.03285 |
| RUNX2 | chr6: 45626194-45626253 | 0.07476 | 0.03285 |
| S100A1 | chr1: 151870900-151870959 | 0.03717 | 0.08040 |
| SAP30L | chr5: 153813177-153813236 | 0.02261 | 0.01625 |
| SAR1A | chr10: 71580544-71580485 | 0.09027 | 0.00141 |
| SDC2 | chr8: 97683904-97689769 | 0.07408 | 0.02852 |
| SEC13 | chr3: 10328684-10322353 | 0.04995 | 0.08329 |
| SEC13 | chr3: 10317717-10317658 | 0.04995 | 0.01827 |
| SEC22C | chr3: 42574118-42572477 | 0.08165 | 0.01097 |
| SEMA6B | chr19: 4493955-4493896 | 0.06027 | 0.04522 |
| SERINC1 | chr6: 122806546-122806487 | 0.02271 | 0.02984 |
| SERPING1 | chr11: 57130467-57130526 | 0.00807 | 0.08447 |
| SERTAD2 | chr2: 64712664-64712605 | 0.07070 | 0.00701 |
| SERTAD2 | chr2: 64716407-64716348 | 0.07070 | 0.00360 |
| SETD2 | chr3: 47100475-47100416 | 0.05223 | 0.04494 |
| SETD2 | chr3: 47033643-47033584 | 0.05223 | 0.00901 |
| SETD7 | chr4: 140652023-140651964 | 0.03216 | 0.03680 |
| SETDB2 | chr13: 48960654-48960713 | 0.08077 | 0.03447 |
| SF3A1 | chr22: 29060385-29060326 | 0.03751 | 0.07728 |
| SHPRH | chr6: 146266241-146266182 | 0.06499 | 0.03471 |
| SLAIN2 | chr4: 48080467-48080526 | 0.02243 | 0.00295 |
| SLAIN2 | chr4: 48080467-48080526 | 0.01921 | 0.00295 |
| SLC16A7 | chr12: 58461094-58461153 | 0.09099 | 0.02403 |
| SLC25A23 | chr19: 6405727-6405668 | 0.03372 | 0.04243 |
| SLC25A28 | chr10: 101360723-101360664 | 0.03452 | 0.02389 |
| SLC25A36 | chr3: 142181169-142181228 | 0.02026 | 0.08567 |
| SLC27A5 | chr19: 63701592-63701533 | 0.01515 | 0.08464 |
| SLC31A2 | chr9: 114966061-114966120 | 0.03693 | 0.02823 |
| SLC35A5 | chr3: 113784531-113784590 | 0.05630 | 0.01713 |
| SLC39A3 | chr19: 2685789-2685730 | 0.00522 | 0.09909 |
| SLC39A3 | chr19: 2685789-2685730 | 0.04060 | 0.09909 |
| SLC7A6OS | chr16: 66892148-66892089 | 0.01603 | 0.03974 |
| SMAD4 | chr18: 46859058-46859117 | 0.02028 | 0.00843 |
| SMAD4 | chr18: 46859058-46859117 | 0.02028 | 0.02132 |
| SMAD4 | chr18: 46859058-46859117 | 0.02028 | 0.04037 |
| SMAD4 | chr18: 46859058-46859117 | 0.02028 | 0.01829 |
| SMAD4 | chr18: 46859058-46859117 | 0.02028 | 0.02949 |
| SMCR7L | chr22: 38241986-38242045 | 0.06008 | 0.01605 |
| SNAG1 | chr5: 53851276-53851335 | 0.01850 | 0.07416 |
| SNX17 | chr2: 27450515-27450744 | 0.04993 | 0.01515 |
| SNX17 | chr2: 27452340-27452533 | 0.04993 | 0.09143 |
| SNX24 | chr5: 122371487-122371546 | 0.03953 | 0.09045 |
| SORBS2 | chr4: 186755142-186755083 | 0.05287 | 0.02369 |
| SPACA4 | chr19: 53802410-53802469 | 0.08012 | 0.04616 |
| SPAG9 | chr17: 46395112-46395053 | 0.08000 | 0.00221 |
| SPSB3 | chr16: 1766844-1766785 | 0.00463 | 0.08375 |
| SRI | chr7: 87673274-87673215 | 0.06266 | 0.01866 |
| ST13 | chr22: 39552435-39552376 | 0.03276 | 0.00421 |
| ST13 | chr22: 39552435-39552376 | 0.03276 | 0.00222 |
| ST13 | chr22: 39551831-39551772 | 0.03276 | 0.03114 |
| STARD13 | chr13: 32576315-32576256 | 0.01295 | 0.02773 |
| STARD5 | chr15: 79403402-79401933 | 0.04022 | 0.07515 |
| STAT3 | chr17: 37719508-37719449 | 0.05734 | 0.01360 |
| STAT3 | chr17: 37719508-37719449 | 0.07855 | 0.01360 |
| STC2 | chr5: 172674652-172674593 | 0.04180 | 0.04943 |
| STC2 | chr5: 172674652-172674593 | 0.08278 | 0.04943 |
| STIM2 | chr4: 26634985-26635044 | 0.06396 | 0.00286 |
| STK24 | chr13: 97902639-97902580 | 0.04663 | 0.05469 |
| STXBP6 | chr14: 24355486-24355427 | 0.02668 | 0.03804 |
| SUSD2 | chr22: 22914978-22915037 | 0.01621 | 0.08859 |
| SYK | chr9: 92697963-92698022 | 0.03396 | 0.08341 |
| SYK | chr9: 92697963-92698022 | 0.02670 | 0.08341 |
| SYK | chr9: 92697963-92698022 | 0.03396 | 0.04523 |
| SYK | chr9: 92697963-92698022 | 0.02670 | 0.04523 |
| SYK | chr9: 92697963-92698022 | 0.03396 | 0.04647 |
| SYK | chr9: 92697963-92698022 | 0.02670 | 0.04647 |
| SYK | chr9: 92697963-92698022 | 0.03396 | 0.07595 |
| SYK | chr9: 92697963-92698022 | 0.02670 | 0.07595 |
| SYK | chr9: 92697963-92698022 | 0.03396 | 0.03355 |
| SYK | chr9: 92697963-92698022 | 0.02670 | 0.03355 |
| SYK | chr9: 92697963-92698022 | 0.03396 | 0.07395 |
| SYK | chr9: 92697963-92698022 | 0.02670 | 0.07395 |
| SYNE2 | chr14: 63527018-63527449 | 0.08754 | 0.01082 |
| SYNGR3 | chr16: 1983587-1983646 | 0.04287 | 0.05531 |
| TAF11 | chr6: 34954124-34954065 | 0.04432 | 0.05922 |
| TBC1D16 | chr17: 75521376-75521317 | 0.04435 | 0.09843 |
| TBC1D5 | chr3: 17177221-17177162 | 0.04652 | 0.04136 |
| TCAP | chr17: 35076266-35076325 | 0.06296 | 0.00870 |
| TCEA3 | chr1: 23586412-23586353 | 0.09078 | 0.04813 |
| TCEAL2 | chrX: 101268876-101268935 | 0.08263 | 0.04290 |
| TCP11L2 | chr12: 105264366-105264425 | 0.04864 | 0.04956 |
| TFAM | chr10: 59825178-59825237 | 0.01205 | 0.03988 |
| TGFB2 | chr1: 216683872-216683931 | 0.02627 | 0.05808 |
| THBS2 | chr6: 169358199-169358136 | 0.07415 | 0.00638 |
| TIMP3 | chr22: 31588540-31588599 | 0.02936 | 0.01149 |
| TIMP3 | chr22: 31588540-31588599 | 0.00446 | 0.01149 |
| TIMP3 | chr22: 31588540-31588599 | 0.03715 | 0.01149 |
| TLE6 | chr19: 2931294-2931353 | 0.03907 | 0.01625 |
| TLK2 | chr17: 57984802-57991121 | 0.09642 | 0.02090 |
| TMCO2 | chr1: 40489860-40489919 | 0.03066 | 0.08193 |
| TMEM106B | chr7: 12238651-12238710 | 0.07798 | 0.01365 |
| TMEM111 | chr3: 9993659-9991132 | 0.02617 | 0.05220 |
| TMEM176B | chr7: 150120141-150120082 | 0.00828 | 0.06560 |
| TMEM19 | chr12: 70379087-70379146 | 0.01240 | 0.09944 |
| TMEM19 | chr12: 70379087-70379146 | 0.03690 | 0.09944 |
| TMEM19 | chr12: 70382251-70382310 | 0.01240 | 0.05794 |
| TMEM19 | chr12: 70382251-70382310 | 0.03690 | 0.05794 |
| TMEM20 | chr10: 95651573-95651632 | 0.09699 | 0.04772 |
| TMEM30B | chr14: 60814500-60814441 | 0.01775 | 0.04307 |
| TMEM35 | chrX: 100237886-100237945 | 0.05676 | 0.01549 |
| TMEM38B | chr9: 107577017-107577076 | 0.04471 | 0.01632 |
| TMF1 | chr3: 69157414-69155976 | 0.00036 | 0.06724 |
| TNFRSF11B | chr8: 120005960-120005901 | 0.07558 | 0.03875 |
| TNRC6A | chr16: 24743111-24743170 | 0.03941 | 0.04341 |
| TOX3 | chr16: 51055571-51055512 | 0.08454 | 0.04433 |
| TOX3 | chr16: 51055571-51055512 | 0.05996 | 0.04433 |
| TPCN2 | chr11: 68685619-68685678 | 0.02443 | 0.08503 |
| TRMT12 | chr8: 125533948-125534007 | 0.02895 | 0.07879 |
| TRPM3 | chr9: 72340151-72340092 | 0.09577 | 0.01598 |
| TRPM3 | chr9: 72340151-72340092 | 0.08009 | 0.01598 |
| TSC22D2 | chr3: 151659872-151659931 | 0.04211 | 0.00311 |
| TTC23 | chr15: 97519518-97514017 | 0.04442 | 0.05369 |
| TTC33 | chr5: 40750413-40750354 | 0.05573 | 0.04611 |
| TTLL7 | chr1: 84128625-84121382 | 0.01248 | 0.08539 |
| TUBGCP3 | chr13: 112188148-112188089 | 0.07546 | 0.01116 |
| UBE1DC1 | chr3: 133877454-133878032 | 0.03663 | 0.09011 |
| UFM1 | chr13: 37834027-37834086 | 0.07333 | 0.04923 |
| USP2 | chr11: 118733738-118733679 | 0.03970 | 0.07182 |
| USP36 | chr17: 74295214-74295155 | 0.03004 | 0.07728 |
| USP47 | chr11: 11937181-11937240 | 0.00461 | 0.08296 |
| UTS2 | chr1: 7830475-7830416 | 0.06382 | 0.02002 |
| VCAM1 | chr1: 100976371-100976430 | 0.09710 | 0.02031 |
| VIPR2 | chr7: 158520076-158519741 | 0.05550 | 0.02659 |
| VPRBP | chr3: 51415621-51411244 | 0.02924 | 0.07696 |
| VPRBP | chr3: 51408997-51408938 | 0.02924 | 0.08823 |
| WBSCR22 | chr7: 72745670-72746260 | 0.03602 | 0.02957 |
| WDR44 | chrX: 117467327-117467386 | 0.00583 | 0.01680 |
| WDR55 | chr5: 140028962-140029105 | 0.09088 | 0.01608 |
| WNK1 | chr12: 888538-888597 | 0.04238 | 0.07840 |
| WNK1 | chr12: 888538-888597 | 0.00728 | 0.07840 |
| WTAP | chr6: 160090013-160090072 | 0.06798 | 0.02869 |
| WWTR1 | chr3: 150719491-150719432 | 0.02614 | 0.07409 |
| XPNPEP3 | chr22: 39588413-39588472 | 0.02071 | 0.00855 |
| XPNPEP3 | chr22: 39658164-39658223 | 0.02071 | 0.02239 |
| XPNPEP3 | chr22: 39652715-39652774 | 0.02071 | 0.09868 |
| XYLT1 | chr16: 17109197-17109138 | 0.05379 | 0.03353 |
| YPEL2 | chr17: 54832347-54832406 | 0.03286 | 0.01866 |
| ZAR1 | chr4: 48190956-48191015 | 0.03901 | 0.06221 |
| ZBTB24 | chr6: 109909180-109909121 | 0.03168 | 0.08707 |
| ZBTB24 | chr6: 109890739-109890680 | 0.03168 | 0.01086 |
| ZC3H11A | chr1: 202089761-202089820 | 0.05822 | 0.00811 |
| ZDHHC21 | chr9: 14648843-14648784 | 0.03009 | 0.09424 |
| ZFAND5 | chr9: 74159645-74159586 | 0.03632 | 0.04143 |
| ZFAND6 | chr15: 78217509-78217568 | 0.02217 | 0.07611 |
| ZNF133 | chr20: 18245522-18245581 | 0.00984 | 0.03917 |
| ZNF179 | chr17: 19261081-19261140 | 0.01330 | 0.02444 |
| ZNF207 | chr17: 27730916-27730975 | 0.03913 | 0.07994 |
| ZNF224 | chr19: 49304003-49304062 | 0.02086 | 0.06151 |
| ZNF230 | chr19: 49206778-49206837 | 0.04026 | 0.08207 |
| ZNF238 | chr1: 242285734-242285793 | 0.07190 | 0.02373 |
| ZNF294 | chr21: 29223348-29223289 | 0.00212 | 0.06934 |
| ZNF294 | chr21: 29223348-29223289 | 0.00092 | 0.06934 |
| ZNF414 | chr19: 8483309-8482824 | 0.02498 | 0.07692 |
| ZNF514 | chr2: 95177753-95177694 | 0.00112 | 0.09860 |
| ZNF655 | chr7: 99000046-99000105 | 0.06445 | 0.02820 |
| ZNF776 | chr19: 62960370-62960673 | 0.08302 | 0.01596 |
| ZNHIT3 | chr17: 31925540-31925599 | 0.03933 | 0.03947 |
| ZNRF1 | chr16: 73699891-73699950 | 0.07144 | 0.02232 |

Table 9 lists first cohort genes that were 0.05 in both Affymetrix and Agilent screening.

TABLE 9

| | | p-value | p-value |
|---|---|---|---|
| Gene Symbol | Genomic Coordinates | Affymetrix | Agilent |
| 2′-PDE | chr3: 57520095-57520154 | 0.025185 | 0.043317 |
| ABCF2 | chr7: 150541405-150541346 | 0.009539 | 0.033377 |
| AGPAT3 | chr21: 44230639-44230698 | 0.018938 | 0.017039 |
| AGPAT3 | chr21: 44230639-44230698 | 0.036971 | 0.017039 |
| ALDH1B1 | chr9: 38387141-38387200 | 0.011769 | 0.011946 |
| ALDH1B1 | chr9: 38387141-38387200 | 0.028607 | 0.011946 |
| ANGPTL2 | chr9: 128891100-128891041 | 0.016283 | 0.032018 |
| ANP32A | chr15: 66866836-66863960 | 0.004541 | 0.029251 |
| AP1M1 | chr19: 16206642-16206701 | 0.035782 | 0.042686 |
| ARHGEF11 | chr1: 155174859-155173879 | 0.036343 | 0.045109 |
| ARHGEF7 | chr13: 110755596-110755655 | 0.032182 | 0.018796 |
| ARL2BP | chr16: 55844971-55845030 | 0.030444 | 0.014720 |
| ARL2BP | chr16: 55844971-55845030 | 0.020849 | 0.014720 |
| ARNTL2 | chr12: 27465419-27465477 | 0.047142 | 0.013831 |
| ARNTL2 | chr12: 27465419-27465477 | 0.041290 | 0.013831 |
| ARNTL2 | chr12: 27460133-27462332 | 0.047142 | 0.040686 |
| ARNTL2 | chr12: 27460133-27462332 | 0.041290 | 0.040686 |
| ARPC4 | chr3: 9820571-9820630 | 0.035132 | 0.001991 |
| BCL2L11 | chr2: 111638189-111638248 | 0.027760 | 0.048254 |
| BCL2L11 | chr2: 111638203-111638262 | 0.027760 | 0.002723 |
| C10orf6 | chr10: 102666704-102666763 | 0.040207 | 0.024905 |
| C13orf15 | chr13: 40940937-40942635 | 0.004878 | 0.009565 |
| C16orf42 | chr16: 1340067-1339903 | 0.021046 | 0.020825 |
| C22orf9 | chr22: 43971224-43971165 | 0.004207 | 0.030384 |
| C22orf9 | chr22: 43971282-43971223 | 0.004207 | 0.033102 |
| CADM1 | chr11: 114550765-114550706 | 0.043186 | 0.046607 |
| CADM1 | chr11: 114550765-114550706 | 0.021198 | 0.046607 |
| CALU | chr7: 128197605-128197664 | 0.010621 | 0.005353 |
| CASP4 | chr11: 104318898-104318839 | 0.008645 | 0.034768 |
| CAV1 | chr7: 115986237-115986296 | 0.013920 | 0.020574 |
| CCDC50 | chr3: 192592323-192592382 | 0.003550 | 0.002351 |
| CCDC85B | chr11: 65415351-65415410 | 0.028694 | 0.046503 |
| CDR2 | chr16: 22265245-22265186 | 0.038671 | 0.002696 |
| CEP135 | chr4: 56521495-56521554 | 0.037435 | 0.014663 |
| CEP68 | chr2: 65166977-65167036 | 0.022149 | 0.010783 |
| CEP68 | chr2: 65166977-65167036 | 0.011698 | 0.010783 |
| CHRNE | chr17: 4742718-4742659 | 0.009675 | 0.006084 |
| CLIC4 | chr1: 25039105-25039895 | 0.016841 | 0.039325 |
| CLN8 | chr8: 1719039-1719098 | 0.026247 | 0.028128 |
| CLN8 | chr8: 1719039-1719098 | 0.040370 | 0.028128 |
| CLN8 | chr8: 1719293-1719352 | 0.026247 | 0.005451 |
| CLN8 | chr8: 1719293-1719352 | 0.040370 | 0.005451 |
| CMYA5 | chr5: 79067786-79067845 | 0.011426 | 0.048796 |
| CNBD1 | chr8: 88367945-88368004 | 0.021677 | 0.039417 |
| COL12A1 | chr6: 75851068-75851009 | 0.008865 | 0.008561 |
| COTL1 | chr16: 83156898-83156839 | 0.030346 | 0.031757 |
| CRIPAK | chr4: 1379548-1379607 | 0.009488 | 0.007270 |
| CSNK2A2 | chr16: 56751432-56751373 | 0.004656 | 0.012561 |
| CXADR | chr21: 17846048-17846107 | 0.020374 | 0.020919 |
| CYBA | chr16: 87240706-87240090 | 0.036383 | 0.037277 |
| CYR61 | chr1: 85821541-85821600 | 0.012530 | 0.001186 |
| DAPK1 | chr9: 89512595-89512654 | 0.008862 | 0.029010 |
| DDX50 | chr10: 70376457-70376516 | 0.014973 | 0.047225 |
| DHRS9 | chr2: 169660621-169660680 | 0.001448 | 0.043633 |
| DNAJB5 | chr9: 34988281-34988340 | 0.012169 | 0.022787 |
| DOCK4 | chr7: 111166530-111166471 | 0.008941 | 0.007808 |
| DRD2 | chr11: 112786811-112786752 | 0.025723 | 0.048918 |
| EFS | chr14: 22895649-22895590 | 0.013872 | 0.006975 |
| EIF4EBP2 | chr10: 71849793-71849852 | 0.020882 | 0.037052 |
| ELL2 | chr5: 95248974-95248915 | 0.046636 | 0.038860 |
| ENDOD1 | chr11: 94505240-94505299 | 0.021970 | 0.029379 |
| EPOR | chr19: 11353383-11352946 | 0.001030 | 0.028776 |
| ERCC8 | chr5: 60222532-60222473 | 0.008340 | 0.016570 |
| FAM26B | chr10: 105196810-105196751 | 0.036266 | 0.046931 |
| FBXO3 | chr11: 33719902-33719843 | 0.004776 | 0.005286 |
| FCGBP | chr19: 45045966-45045907 | 0.011439 | 0.027541 |
| FCHO2 | chr5: 72421695-72421754 | 0.022055 | 0.005593 |
| FGF18 | chr5: 170816574-170816633 | 0.012300 | 0.027075 |
| FLJ20323 | chr7: 7602521-7602580 | 0.011764 | 0.041033 |
| FLJ31222 | chr17: 38283606-38283547 | 0.032361 | 0.029483 |
| FSCN1 | chr7: 5611468-5611527 | 0.025037 | 0.035067 |
| FVT1 | chr18: 59149184-59149125 | 0.048899 | 0.002149 |
| FVT1 | chr18: 59149913-59149854 | 0.048899 | 0.033364 |
| FZD1 | chr7: 90735776-90735835 | 0.045923 | 0.018801 |
| GALNT2 | chr1: 228483885-228483944 | 0.040860 | 0.037553 |
| GAS7 | chr17: 9755003-9754944 | 0.010851 | 0.010298 |
| GAS7 | chr17: 9755003-9754944 | 0.018272 | 0.010298 |
| GAS7 | chr17: 9760158-9760099 | 0.010851 | 0.040328 |
| GAS7 | chr17: 9760158-9760099 | 0.018272 | 0.040328 |
| GATAD1 | chr7: 91923728-91923787 | 0.026980 | 0.044391 |
| GIT2 | chr12: 108852062-108852003 | 0.034797 | 0.039924 |
| GLE1L | chr9: 130335947-130336006 | 0.018829 | 0.004732 |
| GLIPR1 | chr12: 74179523-74179582 | 0.007501 | 0.046200 |
| GLIPR1 | chr12: 74178990-74179049 | 0.007501 | 0.001827 |
| GNA11 | chr19: 3072011-3072070 | 0.035231 | 0.012768 |
| GON4L | chr1: 154021140-154021081 | 0.019464 | 0.030831 |
| GOPC | chr6: 117988368-117988309 | 0.009144 | 0.016240 |
| GPR114 | chr16: 56168469-56168528 | 0.022969 | 0.036213 |
| GRID1 | chr10: 87351883-87351824 | 0.040940 | 0.010866 |
| GTDC1 | chr2: 144709670-144709611 | 0.001402 | 0.035746 |
| GTF3C2 | chr2: 27413898-27413701 | 0.036238 | 0.020802 |
| GTF3C2 | chr2: 27402233-27402210 | 0.036238 | 0.031093 |
| HDAC4 | chr2: 239635672-239635613 | 0.031183 | 0.011283 |
| HIBCH | chr2: 190778046-190777987 | 0.041994 | 0.032997 |
| HRK | chr12: 115783495-115783436 | 0.004221 | 0.049098 |
| HS2ST1 | chr1: 87347957-87348016 | 0.049838 | 0.023333 |
| HSF2 | chr6: 122795598-122795657 | 0.034268 | 0.005200 |
| IKIP | chr12: 97543079-97543020 | 0.009953 | 0.012211 |
| IKIP | chr12: 97543079-97543020 | 0.009330 | 0.012211 |
| IPO8 | chr12: 30678322-30676192 | 0.019940 | 0.043792 |
| ITGA9 | chr3: 37835561-37835620 | 0.008154 | 0.029321 |
| KCTD10 | chr12: 108371402-108371343 | 0.017738 | 0.025929 |
| KIAA0494 | chr1: 46913675-46913616 | 0.004209 | 0.015265 |
| KIAA1143 | chr3: 44765278-44765242 | 0.028511 | 0.020930 |
| LOC339804 | chr2: 61243495-61243554 | 0.021336 | 0.028637 |
| LOC401317 | chr7: 28416632-28416691 | 0.043953 | 0.024877 |
| LOC404266 | chr17: 44036316-44036375 | 0.043129 | 0.047206 |
| LRRC17 | chr7: 102372062-102372121 | 0.013398 | 0.037753 |
| LST1 | chr6: 31664372-31664431 | 0.027729 | 0.002680 |
| MAP3K11 | chr11: 65122235-65122176 | 0.044421 | 0.032122 |
| MAP7D3 | chrX: 135140732-135140673 | 0.003506 | 0.004657 |
| MBTD1 | chr17: 46624813-46624754 | 0.038434 | 0.046353 |
| MCOLN3 | chr1: 85260616-85260451 | 0.007895 | 0.034509 |
| MDGA1 | chr6: 37708332-37708273 | 0.012630 | 0.032986 |
| MDH1 | chr2: 63687741-63687800 | 0.012674 | 0.031874 |
| MFAP5 | chr12: 8691917-8691858 | 0.032104 | 0.034900 |
| MIAT | chr22: 25396980-25397039 | 0.025257 | 0.015825 |
| MLL | chr11: 117867208-117867701 | 0.022655 | 0.048219 |
| MR1 | chr1: 179285969-179286028 | 0.004039 | 0.010199 |
| MR1 | chr1: 179291683-179291742 | 0.004039 | 0.028341 |
| MTMR6 | chr13: 24720113-24720054 | 0.047001 | 0.039362 |
| MUC12 | chr7: 100443061-100443120 | 0.029596 | 0.004688 |
| MYADM | chr19: 59068804-59068863 | 0.031216 | 0.013765 |
| MYADM | chr19: 59071373-59071432 | 0.031216 | 0.012787 |
| MYADM | chr19: 59069709-59069768 | 0.031216 | 0.000689 |
| MYLC2PL | chr7: 101043496-101043437 | 0.004102 | 0.036620 |
| NAGA | chr22: 40784989-40784930 | 0.028561 | 0.012950 |
| NEGR1 | chr1: 71644863-71644804 | 0.048242 | 0.011440 |
| NF2 | chr22: 28387293-28391002 | 0.027914 | 0.025895 |
| NRP2 | chr2: 206370010-206370069 | 0.042897 | 0.028296 |
| NRP2 | chr2: 206370010-206370069 | 0.046533 | 0.028296 |
| NRP2 | chr2: 206370010-206370069 | 0.034161 | 0.028296 |
| NRP2 | chr2: 206349676-206349735 | 0.042897 | 0.001513 |
| NRP2 | chr2: 206349676-206349735 | 0.046533 | 0.001513 |
| NRP2 | chr2: 206349676-206349735 | 0.034161 | 0.001513 |
| OLFML1 | chr11: 7488392-7488451 | 0.039692 | 0.034810 |
| PAFAH1B2 | chr11: 116546077-116546136 | 0.000004 | 0.009894 |
| PAFAH1B2 | chr11: 116546077-116546136 | 0.006207 | 0.009894 |
| PAK3 | chrX: 110325760-110325819 | 0.033399 | 0.003313 |
| PDCD6IP | chr3: 33885823-33885882 | 0.037423 | 0.029530 |
| PDE4C | chr19: 18181982-18181923 | 0.017089 | 0.004403 |
| PDE4C | chr19: 18181982-18181923 | 0.047211 | 0.004403 |
| PDE4C | chr19: 18181982-18181923 | 0.017089 | 0.003687 |
| PDE4C | chr19: 18181982-18181923 | 0.047211 | 0.003687 |
| PDE4C | chr19: 18181982-18181923 | 0.017089 | 0.002660 |
| PDE4C | chr19: 18181982-18181923 | 0.047211 | 0.002660 |
| PDE4C | chr19: 18181982-18181923 | 0.017089 | 0.007197 |
| PDE4C | chr19: 18181982-18181923 | 0.047211 | 0.007197 |
| PDE4C | chr19: 18181982-18181923 | 0.017089 | 0.008790 |
| PDE4C | chr19: 18181982-18181923 | 0.047211 | 0.008790 |
| PDE4C | chr19: 18181982-18181923 | 0.017089 | 0.002984 |
| PDE4C | chr19: 18181982-18181923 | 0.047211 | 0.002984 |
| PDE4C | chr19: 18181982-18181923 | 0.017089 | 0.005498 |
| PDE4C | chr19: 18181982-18181923 | 0.047211 | 0.005498 |
| PDE4C | chr19: 18181982-18181923 | 0.017089 | 0.006944 |
| PDE4C | chr19: 18181982-18181923 | 0.047211 | 0.006944 |
| PDE4C | chr19: 18181982-18181923 | 0.017089 | 0.008464 |
| PDE4C | chr19: 18181982-18181923 | 0.047211 | 0.008464 |
| PHF21A | chr11: 45943465-45931717 | 0.015950 | 0.043950 |
| PLA2G4C | chr19: 53294825-53294766 | 0.045502 | 0.033477 |
| PLA2G4C | chr19: 53294825-53294766 | 0.031358 | 0.033477 |
| PRKD3 | chr2: 37337552-37337493 | 0.006624 | 0.017025 |
| PTPN11 | chr12: 111431267-111431326 | 0.044329 | 0.014917 |
| PTPN11 | chr12: 111431267-111431326 | 0.044329 | 0.027267 |
| PTPN11 | chr12: 111431267-111431326 | 0.044329 | 0.012656 |
| PTPN11 | chr12: 111428416-111428475 | 0.044329 | 0.000049 |
| PTPN11 | chr12: 111431267-111431326 | 0.044329 | 0.036119 |
| PTPN11 | chr12: 111431267-111431326 | 0.044329 | 0.014568 |
| PTPN11 | chr12: 111431267-111431326 | 0.044329 | 0.022429 |
| PTRF | chr17: 37810546-37810487 | 0.033867 | 0.017380 |
| QKI | chr6: 163910518-163910577 | 0.001493 | 0.015227 |
| QKI | chr6: 163876087-163876146 | 0.001493 | 0.023390 |
| RAB8B | chr15: 61343176-61343235 | 0.031283 | 0.000569 |
| RAF1 | chr3: 12600670-12600611 | 0.002731 | 0.025017 |
| RAF1 | chr3: 12600670-12600611 | 0.002731 | 0.032801 |
| RALA | chr7: 39702850-39702909 | 0.017291 | 0.049480 |
| RALB | chr2: 120760072-120763628 | 0.000582 | 0.004733 |
| RAPH1 | chr2: 204007005-204006946 | 0.040646 | 0.001024 |
| RC3H2 | chr9: 124695042-124692620 | 0.029940 | 0.006558 |
| RGS2 | chr1: 191047674-191047733 | 0.048207 | 0.031649 |
| RHBDD1 | chr2: 227569291-227569350 | 0.012940 | 0.024360 |
| RUNX2 | chr6: 45626194-45626253 | 0.034781 | 0.032845 |
| SAP30L | chr5: 153813177-153813236 | 0.022605 | 0.016251 |
| SEC13 | chr3: 10317717-10317658 | 0.049947 | 0.018273 |
| SERINC1 | chr6: 122806546-122806487 | 0.022712 | 0.029838 |
| SETD7 | chr4: 140652023-140651964 | 0.032155 | 0.036804 |
| SLAIN2 | chr4: 48080467-48080526 | 0.022426 | 0.002954 |
| SLAIN2 | chr4: 48080467-48080526 | 0.019207 | 0.002954 |
| SLC25A23 | chr19: 6405727-6405668 | 0.033718 | 0.042435 |
| SLC25A28 | chr10: 101360723-101360664 | 0.034522 | 0.023894 |
| SLC31A2 | chr9: 114966061-114966120 | 0.036926 | 0.028228 |
| SLC7A6OS | chr16: 66892148-66892089 | 0.016034 | 0.039741 |
| SMAD4 | chr18: 46859058-46859117 | 0.020282 | 0.021324 |
| SMAD4 | chr18: 46859058-46859117 | 0.020282 | 0.040374 |
| SMAD4 | chr18: 46859058-46859117 | 0.020282 | 0.029491 |
| SMAD4 | chr18: 46859058-46859117 | 0.020282 | 0.008429 |
| SMAD4 | chr18: 46859058-46859117 | 0.020282 | 0.018292 |
| SNX17 | chr2: 27450515-27450744 | 0.049927 | 0.015151 |
| ST13 | chr22: 39552435-39552376 | 0.032764 | 0.002224 |
| ST13 | chr22: 39551831-39551772 | 0.032764 | 0.031140 |
| ST13 | chr22: 39552435-39552376 | 0.032764 | 0.004208 |
| STARD13 | chr13: 32576315-32576256 | 0.012952 | 0.027734 |
| STC2 | chr5: 172674652-172674593 | 0.041797 | 0.049431 |
| STXBP6 | chr14: 24355486-24355427 | 0.026676 | 0.038038 |
| SYK | chr9: 92697963-92698022 | 0.026699 | 0.046466 |
| SYK | chr9: 92697963-92698022 | 0.033956 | 0.046466 |
| SYK | chr9: 92697963-92698022 | 0.026699 | 0.045226 |
| SYK | chr9: 92697963-92698022 | 0.033956 | 0.045226 |
| SYK | chr9: 92697963-92698022 | 0.026699 | 0.033549 |
| SYK | chr9: 92697963-92698022 | 0.033956 | 0.033549 |
| TBC1D5 | chr3: 17177221-17177162 | 0.046523 | 0.041357 |
| TCP11L2 | chr12: 105264366-105264425 | 0.048643 | 0.049562 |
| TFAM | chr10: 59825178-59825237 | 0.012051 | 0.039878 |
| TIMP3 | chr22: 31588540-31588599 | 0.004461 | 0.011491 |
| TIMP3 | chr22: 31588540-31588599 | 0.037149 | 0.011491 |
| TIMP3 | chr22: 31588540-31588599 | 0.029355 | 0.011491 |
| TLE6 | chr19: 2931294-2931353 | 0.039067 | 0.016252 |
| TMEM30B | chr14: 60814500-60814441 | 0.017751 | 0.043070 |
| TMEM38B | chr9: 107577017-107577076 | 0.044707 | 0.016325 |
| TNRC6A | chr16: 24743111-24743170 | 0.039407 | 0.043411 |
| TSC22D2 | chr3: 151659872-151659931 | 0.042109 | 0.003115 |
| WBSCR22 | chr7: 72745670-72746260 | 0.036022 | 0.029573 |
| WDR44 | chrX: 117467327-117467386 | 0.005828 | 0.016798 |
| XPNPEP3 | chr22: 39658164-39658223 | 0.020707 | 0.022391 |
| XPNPEP3 | chr22: 39588413-39588472 | 0.020707 | 0.008550 |
| YPEL2 | chr17: 54832347-54832406 | 0.032855 | 0.018659 |
| ZBTB24 | chr6: 109890739-109890680 | 0.031677 | 0.010861 |
| ZFAND5 | chr9: 74159645-74159586 | 0.036316 | 0.041428 |
| ZNF133 | chr20: 18245522-18245581 | 0.009835 | 0.039172 |
| ZNF179 | chr17: 19261081-19261140 | 0.013301 | 0.024437 |
| ZNHIT3 | chr17: 31925540-31925599 | 0.039326 | 0.039469 |

Table 10 lists genes that were 0.05 in at least 1 experiment and 0.1 in the other two experiments.

TABLE 10

| | | p-value | p-value |
|---|---|---|---|
| Gene | p-value | Agilent 2^{nd} | Agilent 1^{st} |
| Symbol | Affymetrix | cohort | cohort |
| DDX50 | 0.014973 | 0.031388 | 0.047225 |
| ATP2C2 | 0.005141 | 0.092778 | 0.089886 |
| C19orf2 | 0.083444 | 0.037363 | 0.042512 |
| MCOLN3 | 0.007895 | 0.087495 | 0.034509 |
| VIPR2 | 0.055501 | 0.068813 | 0.026592 |
| ZSWIM3 | 0.087625 | 0.046805 | 0.074354 |
| PFDN4 | 0.020595 | 0.023361 | 0.067965 |
| FNDC4 | 0.063202 | 0.015164 | 0.007645 |
| EPM2A | 0.010675 | 0.095807 | 0.099990 |
| C20orf20 | 0.039762 | 0.082408 | 0.050136 |
| HDAC4 | 0.031183 | 0.033915 | 0.011283 |
| SMAD4 | 0.020282 | 0.054752 | 0.040374 |
| SMAD4 | 0.020282 | 0.054752 | 0.018292 |
| SMAD4 | 0.020282 | 0.054752 | 0.021324 |
| SMAD4 | 0.020282 | 0.054752 | 0.008429 |
| SMAD4 | 0.020282 | 0.054752 | 0.029491 |
| NF2 | 0.027914 | 0.045300 | 0.025895 |
| CHRNE | 0.009675 | 0.063715 | 0.006084 |
| GATS | 0.075084 | 0.012915 | 0.004367 |
| SLC46A1 | 0.090121 | 0.008824 | 0.098055 |
| ZNF414 | 0.024978 | 0.034430 | 0.076919 |
| ADARB1 | 0.070600 | 0.033570 | 0.056321 |
| ZNF776 | 0.083022 | 0.090851 | 0.015958 |
| TMEM20 | 0.096993 | 0.074123 | 0.047719 |
| RAF1 | 0.002731 | 0.022491 | 0.025017 |
| RAF1 | 0.002731 | 0.022491 | 0.068718 |
| RAF1 | 0.002731 | 0.022491 | 0.073104 |
| RAF1 | 0.002731 | 0.022491 | 0.032801 |
| RAF1 | 0.002731 | 0.071013 | 0.025017 |
| RAF1 | 0.002731 | 0.071013 | 0.068718 |
| RAF1 | 0.002731 | 0.071013 | 0.073104 |
| RAF1 | 0.002731 | 0.071013 | 0.032801 |
| RAF1 | 0.002731 | 0.067400 | 0.025017 |
| RAF1 | 0.002731 | 0.067400 | 0.068718 |
| RAF1 | 0.002731 | 0.067400 | 0.073104 |
| RAF1 | 0.002731 | 0.067400 | 0.032801 |
| TMEM35 | 0.056760 | 0.051446 | 0.015485 |
| SERTAD2 | 0.070703 | 0.064499 | 0.003602 |
| WBSCR22 | 0.036022 | 0.000833 | 0.029573 |
| MR1 | 0.004039 | 0.083755 | 0.010199 |
| APOB | 0.046566 | 0.005792 | 0.083147 |
| APOB | 0.046566 | 0.010441 | 0.083147 |
| TRMT12 | 0.028945 | 0.035549 | 0.078785 |
| ANTXR1 | 0.005422 | 0.098682 | 0.080792 |
| ENTPD7 | 0.068410 | 0.019755 | 0.023314 |
| ENTPD7 | 0.068410 | 0.028777 | 0.027676 |
| HGSNAT | 0.032639 | 0.097541 | 0.054292 |
| TCEB3 | 0.091320 | 0.034010 | 0.064471 |
| CAV1 | 0.013920 | 0.086435 | 0.020574 |
| TFAM | 0.012051 | 0.012358 | 0.039878 |
| PRKD3 | 0.006624 | 0.006815 | 0.017025 |
| FBXO4 | 0.091809 | 0.002549 | 0.040544 |
| ANKRD28 | 0.079228 | 0.092105 | 0.030335 |
| SERTAD2 | 0.070703 | 0.035526 | 0.007010 |
| GTF3C2 | 0.050168 | 0.095494 | 0.020802 |
| GTF3C2 | 0.036238 | 0.095494 | 0.020802 |
| CSNK2A2 | 0.004656 | 0.074745 | 0.012561 |
| ITGA2 | 0.014495 | 0.079374 | 0.058232 |
| PCMTD1 | 0.082359 | 0.053130 | 0.002999 |
| EYA4 | 0.097376 | 0.006989 | 0.062331 |
| CXADR | 0.020374 | 0.055973 | 0.089638 |
| IDS | 0.022943 | 0.088290 | 0.070496 |
| SLAIN2 | 0.022426 | 0.041042 | 0.002954 |
| SLAIN2 | 0.019207 | 0.041042 | 0.002954 |
| PKD1L2 | 0.093555 | 0.006239 | 0.099462 |

Table 11 lists genes that were significant (0.05) in all three screening experiments.

TABLE 11

| | p-value | p-value | p-value |
|---|---|---|---|
| Gene | for agilent | for Agilent | for Affymetrix |
| Symbol | first cohort | second cohort | first cohort |
| RAF1 | 0.0225 | 0.032801 | 0.002731 |
| RAF1 | 0.0225 | 0.025017 | 0.002731 |
| DDX50 | 0.0314 | 0.047225 | 0.014973 |
| TFAM | 0.0124 | 0.039878 | 0.012051 |
| HDAC4 | 0.0339 | 0.011283 | 0.031183 |
| NF2 | 0.0453 | 0.025895 | 0.027914 |
| PRKD3 | 0.0068 | 0.017025 | 0.006624 |
| SLAIN2 | 0.0410 | 0.002954 | 0.022426 |
| WBSCR22 | 0.0008 | 0.029573 | 0.036022 |
| SLAIN2 | 0.0410 | 0.002954 | 0.019207 |

None of the suspected candidate genes, including HER2, other members of HER family and their ligands, PTEN, PI3K, and IGF1R, showed a reproducible interaction with trastuzumab in both cohorts. While more than 2000 probes showed interaction in each of the two cohorts, only twelve genes showed interaction p-values less than 0.05 in both the gene discovery and gene refinement steps. Of the twelve genes, 7 were associated with resistance and 5 with sensitivity to trastuzumab. The expression levels of the identified predictive genes did not correlate with those of HER2 gene.

The study was then extended to include 753 cases from NSABP B-31 trial using the Agilent microarray platform, and 380 genes were found with statistically significant interaction with trastuzumab (predictive of degree of benefit from trastuzumab) after adjusting for nodal status and estrogen receptor status (Table 12).

TABLE 12

| | Target ID | | interaction |
|---|---|---|---|
| Probe ID | (GenBank Ref.) | Genomic Coordinates | p-value |
| A_23_P15123 | NM_019116 | chr16: 23489532-23489591 | 3.02E−04 |
| A_23_P210048 | NM_006037 | chr2: 239635672-239635613 | 3.31E−04 |
| A_23_P89310 | NM_014964 | chr17: 19180507-19180566 | 5.62E−04 |
| A_24_P182182 | NM_001152 | chrX: 118488101-118488385 | 7.63E−04 |
| A_23_P56314 | NM_006830 | chr19: 1549153-1549094 | 7.68E−04 |
| A_32_P11372 | NR_003674 | chr9: 46585969-46586028 | 8.83E−04 |
| A_23_P398275 | AF316855 | chr15_random: 664627-664686 | 1.01E−03 |
| A_32_P227525 | AL522622 | chr19: 12773074-12773602 | 1.25E−03 |
| A_23_P5995 | NM_006420 | chr20: 47082037-47082096 | 1.52E−03 |
| A_23_P156880 | NM_006208 | chr6: 132253564-132253623 | 1.93E−03 |
| A_23_P55706 | NM_006509 | chr19: 50233225-50233284 | 2.04E−03 |
| A_23_P28057 | NM_003249 | chr19: 2764491-2764550 | 2.04E−03 |
| A_23_P111194 | NM_012391 | chr6: 34614144-34614085 | 2.08E−03 |
| A_23_P17307 | NM_018270 | chr20: 60901779-60901838 | 2.24E−03 |
| A_24_P333357 | AK090463 | chr16: 596449-596508 | 2.25E−03 |
| A_32_P147189 | BC071732 | chr2: 232286180-232286121 | 2.49E−03 |
| A_23_P33376 | NM_002595 | chr12: 95198729-95198142 | 2.51E−03 |
| A_32_P208713 | | chr19: 023311797-023311856 | 2.66E−03 |
| A_23_P169154 | NM_024896 | chr9: 5775753-5775694 | 2.83E−03 |
| A_24_P310864 | NM_152499 | chr1: 44234715-44234774 | 2.85E−03 |
| A_23_P28869 | NM_002836 | chr20: 2966788-2966847 | 2.88E−03 |
| A_23_P401675 | NM_144724 | chr5: 68775506-68775565 | 3.01E−03 |
| A_24_P711477 | BC001783 | chr1: 26674467-26674408 | 3.13E−03 |
| A_23_P207680 | NM_001039083 | chr17: 41794825-41791505 | 3.23E−03 |
| A_23_P259771 | AF462442 | chr16: 48697667-48697726 | 3.43E−03 |
| A_23_P25935 | NM_007176 | chr14: 75187656-75187597 | 3.44E−03 |
| A_23_P34527 | NM_025207 | chr1: 153227754-153227813 | 3.64E−03 |
| A_23_P22382 | NM_015527 | chr16: 30275997-30275938 | 3.72E−03 |
| A_32_P121755 | THC2672892 | chr17: 17606980-17607039 | 3.81E−03 |
| A_24_P307065 | XM_928637 | chr17: 71734250-71734191 | 4.01E−03 |
| A_23_P392575 | NM_080671 | chr2: 223626768-223626827 | 4.18E−03 |
| A_24_P154006 | NM_005005 | chr8: 125631269-125631328 | 4.47E−03 |
| A_32_P160398 | AA911065 | chr12: 111327461-111327520 | 4.55E−03 |
| A_23_P55319 | NM_004475 | chr17: 24230908-24230849 | 4.70E−03 |
| A_23_P151907 | NM_002570 | chr15: 99662738-99662679 | 5.22E−03 |
| A_24_P362394 | NM_007259 | chr1: 148321535-148330657 | 5.54E−03 |
| A_32_P142881 | NM_015092 | chr16: 18778411-18777978 | 0.005641 |
| A_32_P183356 | THC2608750 | chr4: 99583317-99583302 | 5.76E−03 |
| A_23_P157669 | NM_005005 | chr8: 125631299-125631358 | 5.84E−03 |
| A_23_P65797 | NM_022480 | chr15: 84103636-84103577 | 6.12E−03 |
| A_23_P257924 | NM_005239 | chr21: 39117433-39117492 | 6.26E−03 |
| A_24_P247849 | AK093508 | chr1: 162910490-162910549 | 6.29E−03 |
| A_23_P138271 | NM_138795 | chr1: 200369355-200369296 | 6.65E−03 |
| A_32_P538017 | NM_005995 | chr11: 67155447-67155388 | 6.73E−03 |
| A_24_P393811 | NM_019026 | chr1: 163979091-163979032 | 6.90E−03 |
| A_32_P92355 | NM_001101676 | chr8: 119273718-119273659 | 7.39E−03 |
| A_23_P61042 | ENST00000390539 | chr14: 105124975-105124694 | 8.31E−03 |
| A_24_P125353 | NM_007359 | chr17: 35578184-35579149 | 8.57E−03 |
| A_32_P165823 | AI431972 | chr20: 48242278-48242219 | 9.02E−03 |
| A_32_P76811 | ENST00000383511 | chr6: 29802573-29802514 | 9.39E−03 |
| A_24_P415168 | BC011620 | chr9: 130192598-130192653 | 9.46E−03 |
| A_23_P215454 | NM_000501 | chr7: 73121535-73121594 | 9.81E−03 |
| A_23_P146084 | NM_000637 | chr8: 30656570-30656511 | 1.00E−02 |
| A_32_P9816 | AI857589 | chr16: 1691696-1691637 | 0.01005 |
| A_32_P88626 | NM_001048183 | chr1: 28696227-28696286 | 1.01E−02 |
| A_23_P399112 | NM_001020818 | chr19: 59068804-59068863 | 1.02E−02 |
| A_23_P129103 | AK074778 | chr15_random: 664106-664165 | 1.02E−02 |
| A_24_P636974 | XR_039576 | chr7: 056402763-056402824 | 1.03E−02 |
| A_32_P163169 | NM_003374 | chr5: 133339601-133339542 | 1.05E−02 |
| A_24_P396340 | NM_138428 | chr1: 35093082-35093023 | 1.08E−02 |
| A_23_P19102 | AK021777 | chr5: 153753992-153754051 | 1.10E−02 |
| A_24_P96171 | NM_030578 | chr19: 46552230-46552171 | 1.11E−02 |
| A_32_P162076 | CR749652 | chr1: 20943476-20943535 | 1.12E−02 |
| A_32_P190568 | AK094885 | chr3: 162629838-162629897 | 1.12E−02 |
| A_23_P71300 | NM_018246 | chr8: 27647122-27647063 | 1.15E−02 |
| A_23_P63219 | NM_015100 | chr1: 149643699-149643640 | 0.011556 |
| A_23_P68628 | NM_031232 | chr20: 31709184-31709125 | 1.16E−02 |
| A_23_P385742 | NM_018279 | chr12: 70382251-70382310 | 1.19E−02 |
| A_23_P250982 | NM_016048 | chr5: 128477266-128477325 | 1.21E−02 |
| A_23_P88781 | NM_020313 | chr16: 56020201-56020142 | 1.22E−02 |
| A_23_P349321 | NM_022166 | chr16: 17109197-17109138 | 1.23E−02 |
| A_24_P156113 | NM_014601 | chr19: 52938036-52938095 | 1.26E−02 |
| A_23_P11279 | NM_018466 | chrX: 110814986-110817828 | 1.26E−02 |
| A_23_P374104 | NM_012098 | chr9: 128891100-128891041 | 1.28E−02 |
| A_24_P353794 | NM_004481 | chr1: 228483885-228483944 | 1.28E−02 |
| A_23_P27515 | NM_012268 | chr19: 45575592-45575751 | 0.012825 |
| A_32_P36715 | CR627135 | chr19: 21431227-21431168 | 1.31E−02 |
| A_24_P392436 | | chr16: 002912909-002912850 | 1.31E−02 |
| A_24_P943709 | AW444553 | chr8: 127636198-127636257 | 1.32E−02 |
| A_24_P333106 | CR595167 | chr14: 34025181-34025240 | 0.013296 |
| A_24_P295745 | NM_153201 | chr11: 122435688-122435629 | 1.34E−02 |
| A_23_P127367 | NM_021173 | chr11: 66875773-66875714 | 1.40E−02 |
| A_23_P154855 | NM_000219 | chr21: 34743484-34743425 | 1.41E−02 |
| A_23_P373598 | NM_002360 | chr7: 1548755-1548814 | 0.014104 |
| A_32_P222664 | ENST00000372899 | chr6: 42768660-42768719 | 1.43E−02 |
| A_32_P104861 | AL567699 | chr11: 74358392-74358333 | 1.45E−02 |
| A_24_P183292 | NM_001025105 | chr5: 148855500-148855441 | 1.46E−02 |
| A_23_P31477 | NM_015332 | chr7: 44390273-44390214 | 1.49E−02 |
| A_23_P106056 | NM_001344 | chr14: 22103645-22103633 | 1.51E−02 |
| A_23_P29575 | NM_033281 | chr5: 68560792-68560851 | 1.52E−02 |
| A_23_P140207 | NM_004563 | chr14: 23643036-23643095 | 0.015174 |
| A_24_P114255 | NM_138799 | chr2: 8914684-8914625 | 1.53E−02 |
| A_24_P210829 | NM_005009 | chr16: 389690-390228 | 1.53E−02 |
| A_23_P49842 | NM_005148 | chr17: 23898991-23898932 | 0.015473 |
| A_24_P937931 | | chr17: 034667924-034667983 | 1.59E−02 |
| A_24_P247931 | NM_183420 | chr8: 391406-398380 | 1.60E−02 |
| A_23_P210690 | NM_021158 | chr20: 326107-326166 | 1.63E−02 |
| A_32_P204258 | THC2688486 | chr10: 79324402-79324343 | 1.63E−02 |
| A_24_P200603 | NM_032151 | chr5: 134322630-134322689 | 1.64E−02 |
| A_24_P766204 | NM_005921 | chr5: 056226249-056226308 | 1.64E−02 |
| A_23_P3368 | NM_002569 | chr15: 89227001-89227060 | 1.64E−02 |
| A_24_P264106 | NM_000984 | chr17: 24073905-24073964 | 1.66E−02 |
| A_32_P55144 | BX537816 | chr4: 1139458-1139517 | 1.68E−02 |
| A_24_P134195 | NM_001020818 | chr19: 59069709-59069768 | 1.68E−02 |
| A_32_P155786 | BC020336 | chr16: 1953245-1954323 | 1.68E−02 |
| A_23_P143414 | NM_080748 | chr20: 33752255-33752314 | 0.016891 |
| A_24_P924957 | L32537 | chr12: 51632897-51632837 | 1.70E−02 |
| A_23_P106945 | NM_001114 | chr16: 48907161-48907220 | 1.70E−02 |
| A_23_P351556 | CR594281 | chr14: 21037232-21037173 | 1.70E−02 |
| A_23_P42935 | NM_004333 | chr7: 140080947-140080888 | 1.71E−02 |
| A_32_P139738 | NR_002827 | chr16: 32070719-32070660 | 1.71E−02 |
| A_32_P84289 | NM_152371 | chr1: 2512337-2512396 | 1.74E−02 |
| A_24_P160466 | NM_052899 | chr5: 175955615-175955556 | 1.74E−02 |
| A_23_P9152 | NM_005772 | chr9: 4850885-4850943 | 1.77E−02 |
| A_23_P501887 | NM_013407 | chr19: 12651482-12651344 | 1.78E−02 |
| A_32_P156628 | BQ379494 | chr1: 180620346-180620405 | 0.017961 |
| A_23_P14886 | NM_032520 | chr16: 1352855-1353004 | 1.84E−02 |
| A_23_P127995 | NM_001293 | chr11: 77005043-77004984 | 1.84E−02 |
| A_23_P171223 | NM_022101 | chrX: 118562437-118562378 | 1.85E−02 |
| A_23_P91217 | NM_032221 | chr20: 39502095-39499371 | 1.87E−02 |
| A_24_P169843 | XR_019568 | chr1: 181194873-181194832 | 1.89E−02 |
| A_23_P53976 | NM_206949 | chr14: 93638655-93638714 | 1.89E−02 |
| A_23_P395493 | BC031631 | chr10: 89594388-89594447 | 0.019124 |
| A_23_P218523 | NM_021731 | chr19: 3493415-3493356 | 1.93E−02 |
| A_24_P47870 | NM_001344 | chr14: 22103867-22103808 | 1.95E−02 |
| A_23_P106682 | NM_001424 | chr16: 10534260-10534201 | 1.95E−02 |
| A_24_P126873 | NM_003170 | chr17: 24050992-24051330 | 1.96E−02 |
| A_23_P78958 | NM_004058 | chr19: 5866818-5866877 | 1.96E−02 |
| A_23_P341471 | NM_003348 | chr12: 92327538-92327479 | 2.02E−02 |
| A_24_P354468 | NM_002106 | chr4: 101088651-101088592 | 2.02E−02 |
| A_24_P913178 | THC2515524 | chr22: 36501865-36501806 | 2.02E−02 |
| A_32_P36182 | BE935032 | chrX: 16786767-16786826 | 2.03E−02 |
| A_23_P131954 | NM_014426 | chr20: 17871049-17870990 | 2.10E−02 |
| A_23_P342709 | NM_152676 | chr18: 69891777-69891718 | 2.11E−02 |
| A_23_P379111 | BC020886 | chr4: 185508159-185508100 | 2.13E−02 |
| A_23_P106773 | NM_177528 | chr16: 28510827-28510768 | 2.14E−02 |
| A_23_P16157 | NM_003685 | chr19: 6364914-6364855 | 2.16E−02 |
| A_23_P90470 | XR_037365 | chr19: 015556842-015556901 | 2.17E−02 |
| A_23_P52589 | NM_003475 | chr11: 553924-553983 | 2.18E−02 |
| A_24_P911775 | BU681302 | chr10: 133623241-133623182 | 2.18E−02 |
| A_23_P4754 | NM_006012 | chr19: 6319801-6319860 | 2.19E−02 |
| A_32_P203362 | AL561405 | chr3: 125939505-125939446 | 2.23E−02 |
| A_23_P118246 | NM_016095 | chr16: 84269214-84269155 | 2.23E−02 |
| A_24_P780709 | | chr2: 107811640-107811559 | 2.23E−02 |
| A_23_P73589 | NM_002444 | chrX: 64878326-64878385 | 0.022524 |
| A_23_P169766 | NM_022740 | chr7: 138907963-138907904 | 2.27E−02 |
| A_23_P132405 | NM_014049 | chr3: 130114530-130114589 | 2.27E−02 |
| A_24_P383130 | | chr12: 2761565-2761624 | 2.28E−02 |
| A_23_P163732 | NM_015044 | chr16: 23387798-23387739 | 2.29E−02 |
| A_32_P108387 | CF528315 | chr5: 132231109-132230598 | 2.29E−02 |
| A_23_P77048 | NM_001039355 | chr14: 99827300-99827241 | 2.30E−02 |
| A_32_P103060 | AI150964 | chr3: 130369697-130369756 | 2.30E−02 |
| A_32_P160414 | THC2615996 | chr20: 28228455-28228514 | 2.31E−02 |
| A_23_P12173 | NM_181715 | chr1: 152186871-152186812 | 2.31E−02 |
| A_24_P583350 | NM_001015050 | chr3: 127132120-127131072 | 2.33E−02 |
| A_23_P389907 | NM_002973 | chr12: 110374772-110374713 | 2.33E−02 |
| A_32_P132766 | THC2671036 | chrX: 73356346-73356405 | 2.34E−02 |
| A_23_P79889 | NM_020673 | chr20: 56368866-56368925 | 2.34E−02 |
| A_23_P61987 | NM_025268 | chr14: 105067477-105067536 | 0.02353 |
| A_23_P117683 | NM_016400 | chr15: 41881271-41881330 | 2.36E−02 |
| A_32_P176609 | NM_015042 | chr15: 62753923-62753982 | 2.36E−02 |
| A_23_P210015 | NM_014369 | chr2: 130848938-130848997 | 2.36E−02 |
| A_23_P321320 | NM_052902 | chr2: 220189340-220189399 | 2.37E−02 |
| A_24_P734892 | | chr5: 001386540-001386486 | 2.38E−02 |
| A_24_P50567 | | chr6: 144563421-144563344 | 2.40E−02 |
| A_24_P29260 | NM_054013 | chr5: 179158842-179158702 | 2.41E−02 |
| A_23_P136026 | AK128476 | chr14: 105245260-105245201 | 2.42E−02 |
| A_32_P5480 | NM_203463 | chr2: 169339249-169339308 | 2.43E−02 |
| A_23_P141555 | NM_013351 | chr17: 43178388-43178447 | 2.44E−02 |
| A_23_P257956 | NM_004515 | chr1: 151901351-151901292 | 2.44E−02 |
| A_23_P251387 | NM_004726 | chrX: 17075549-17075608 | 2.44E−02 |
| A_24_P103803 | NM_001497 | chr9: 33100782-33100723 | 2.45E−02 |
| A_23_P23421 | NM_198236 | chr1: 155174859-155173879 | 2.50E−02 |
| A_32_P97489 | NM_145690 | chr8: 102001659-102001600 | 2.53E−02 |
| A_23_P81492 | NM_001025071 | chr5: 149806711-149806652 | 2.54E−02 |
| A_23_P10685 | NM_012267 | chr19: 60465599-60465540 | 2.56E−02 |
| A_32_P93187 | AL517609 | chr18: 028246271-028246330 | 2.56E−02 |
| A_23_P3453 | NM_015540 | chr15: 39596742-39596683 | 2.59E−02 |
| A_24_P148521 | NM_022152 | chr2: 218852052-218851521 | 2.60E−02 |
| A_23_P33791 | NM_012446 | chr5: 80752083-80752024 | 2.63E−02 |
| A_24_P341897 | NM_004302 | chr12: 50674874-50674933 | 2.64E−02 |
| A_24_P924920 | NM_007011 | chr15: 87544400-87544461 | 2.65E−02 |
| A_24_P134834 | NM_032276 | chr2: 227569291-227569350 | 2.66E−02 |
| A_23_P120660 | NM_001024 | chr20: 60396336-60396789 | 2.68E−02 |
| A_23_P51548 | NM_004528 | chr1: 163891312-163891371 | 2.68E−02 |
| A_23_P338134 | ENST00000333235 | chr15: 82744155-82744096 | 2.68E−02 |
| A_24_P710730 | NR_024054 | chr5: 69474003-69473944 | 2.70E−02 |
| A_24_P345451 | NM_024843 | chr2: 172119360-172119419 | 2.70E−02 |
| A_32_P187327 | NM_006088 | chr9: 139257922-139257980 | 2.72E−02 |
| A_23_P115683 | NM_024747 | chr10: 103817665-103817724 | 2.72E−02 |
| A_32_P12504 | BC024745 | chr11: 43921760-43921701 | 2.79E−02 |
| A_23_P17420 | NM_003657 | chr20: 51994940-51994881 | 2.81E−02 |
| A_23_P107612 | NM_004163 | chr18: 50707630-50707689 | 2.81E−02 |
| A_32_P1869 | | chr10: 007367758-007367817 | 2.81E−02 |
| A_23_P33436 | NM_002383 | chr16: 29727512-29728906 | 2.84E−02 |
| A_23_P28707 | NM_007346 | chr20: 60915658-60915717 | 2.84E−02 |
| A_23_P127565 | NM_178834 | chr11: 110936670-110936729 | 2.85E−02 |
| A_32_P22750 | AL568358 | chr21: 46376586-46376527 | 2.86E−02 |
| A_23_P38712 | NM_001099733 | chr18: 900632-900691 | 0.028852 |
| A_23_P64010 | NM_014342 | chr11: 47597047-47596988 | 2.92E−02 |
| A_24_P26170 | NM_014703 | chr3: 51415621-51411244 | 2.94E−02 |
| A_24_P830667 | NM_000982 | chr13: 26726361-26727379 | 2.95E−02 |
| A_23_P52127 | NM_032360 | chr1: 178524149-178524090 | 2.98E−02 |
| A_32_P437227 | NM_174911 | chr8: 127638820-127638761 | 3.01E−02 |
| A_32_P116219 | | chr11: 000497468-000497409 | 3.03E−02 |
| A_23_P110445 | NM_006051 | chr5: 139918331-139918272 | 3.04E−02 |
| A_24_P358138 | NM_173632 | chr19: 62956599-62956658 | 3.05E−02 |
| A_24_P319369 | NM_016946 | chr1: 159233364-159233305 | 3.07E−02 |
| A_24_P316363 | NM_001521 | chr2: 27413898-27413701 | 3.07E−02 |
| A_23_P133279 | NM_199133 | chr5: 10279717-10279658 | 3.11E−02 |
| A_32_P68459 | NM_021130 | chr7: 44807530-44807589 | 3.13E−02 |
| A_32_P186450 | NR_024271 | chr7: 45742970-45742911 | 3.14E−02 |
| A_23_P156087 | NM_000163 | chr5: 42755379-42755438 | 3.14E−02 |
| A_23_P25187 | NM_002281 | chr12: 50966199-50966140 | 3.16E−02 |
| A_24_P911676 | NM_003107 | chr6: 21705980-21706039 | 3.18E−02 |
| A_32_P32496 | AK126241 | chr4_random: 742430-742371 | 3.21E−02 |
| A_32_P26017 | DB380193 | chr14: 22565054-22565113 | 3.22E−02 |
| A_23_P216984 | NM_016322 | chr9: 122983349-122983290 | 0.032345 |
| A_24_P914376 | CR610088 | chr19: 11353383-11352946 | 3.24E−02 |
| A_23_P81087 | NM_020395 | chr4: 106827299-106823866 | 3.26E−02 |
| A_23_P75220 | NM_031212 | chr10: 101360723-101360664 | 3.28E−02 |
| A_23_P42811 | NM_176813 | chr7: 16879992-16879933 | 3.30E−02 |
| A_24_P679984 | | chr1: 159455581-159455522 | 3.30E−02 |
| A_23_P95292 | NM_014741 | chr11: 46650404-46650463 | 3.32E−02 |
| A_23_P109733 | NM_144718 | chr3: 114646435-114646376 | 3.34E−02 |
| A_23_P390044 | NM_002359 | chr17: 77473134-77473075 | 0.033463 |
| A_23_P99906 | NM_199330 | chr15: 81315256-81315197 | 3.36E−02 |
| A_23_P137830 | XM_060887 | chr1: 45226866-45226807 | 3.39E−02 |
| A_32_P8251 | NM_020901 | chr11: 601931-601990 | 3.42E−02 |
| A_23_P147404 | | | 3.43E−02 |
| A_23_P218654 | NM_032527 | chr20: 61837852-61837911 | 3.45E−02 |
| A_23_P165783 | NM_024101 | chr2: 238122637-238125754 | 3.46E−02 |
| A_23_P369574 | NM_015097 | chr3: 33514524-33514465 | 3.47E−02 |
| A_32_P47200 | THC2519111 | chr16: 21330101-21330160 | 3.47E−02 |
| A_24_P142442 | NM_015584 | chr17: 23698515-23698456 | 3.48E−02 |
| A_24_P99152 | NM_004926 | chr14: 68326087-68326028 | 3.48E−02 |
| A_23_P166677 | NM_022736 | chr3: 160030005-160030064 | 3.50E−02 |
| A_23_P27247 | NM_005121 | chr17: 57383086-57383027 | 3.52E−02 |
| A_24_P109941 | | chr17: 32005102-32005043 | 3.52E−02 |
| A_23_P215461 | NM_002314 | chr7: 73174599-73174658 | 0.035269 |
| A_23_P139722 | NM_001065 | chr12: 6308384-6308325 | 0.035361 |
| A_32_P83221 | BE064950 | chr17: 1273194-1273253 | 3.54E−02 |
| A_23_P342654 | NM_147150 | chr9: 111971916-111971975 | 3.54E−02 |
| A_32_P196483 | NM_001006 | chr4: 152240300-152241129 | 3.55E−02 |
| A_23_P30913 | NM_033554 | chr6: 33140885-33140826 | 3.57E−02 |
| A_23_P33022 | NM_021128 | chr11: 830281-830222 | 3.59E−02 |
| A_24_P332532 | | chr11: 66285652-66285622 | 3.60E−02 |
| A_23_P32500 | NM_015136 | chr3: 52532723-52532782 | 3.61E−02 |
| A_23_P30175 | NM_018695 | chr5: 65411596-65411655 | 3.61E−02 |
| A_24_P383356 | NM_015359 | chr8: 22328312-22329237 | 3.63E−02 |
| A_24_P102406 | BC014871 | chr4_random: 768033-767974 | 3.64E−02 |
| A_24_P367138 | NM_031309 | chr8: 145525625-145525566 | 3.66E−02 |
| A_24_P392632 | NM_206948 | chr9: 72667743-72667684 | 3.69E−02 |
| A_23_P165295 | NM_002501 | chr19: 12997196-12997255 | 3.70E−02 |
| A_24_P255253 | NM_203477 | chr22: 49584135-49584194 | 3.72E−02 |
| A_24_P385119 | NM_003184 | chr8: 120812707-120812648 | 3.73E−02 |
| A_32_P222450 | NM_015444 | chr3: 45242210-45242151 | 3.74E−02 |
| A_23_P104892 | NM_001418 | chr11: 10775203-10775169 | 3.74E−02 |
| A_24_P417984 | NM_138689 | chr11: 63769321-63769262 | 0.037376 |
| A_23_P30377 | NM_014829 | chr5: 134192411-134192470 | 0.037435 |
| A_23_P207736 | AK023077 | chr17: 74303782-74303723 | 3.76E−02 |
| A_32_P46171 | CA314185 | chr4_random: 766703-766644 | 3.76E−02 |
| A_24_P392195 | | chr1: 209413550-209413609 | 3.76E−02 |
| A_23_P75647 | NM_006389 | chr11: 118421066-118421007 | 3.76E−02 |
| A_24_P58477 | | chr15: 33201170-33201111 | 3.77E−02 |
| A_23_P86434 | BC007866 | chr10: 64896047-64896106 | 3.77E−02 |
| A_23_P57733 | NM_033663 | chr3: 115373284-115361370 | 3.77E−02 |
| A_23_P254226 | NM_003611 | chrX: 13688354-13688413 | 3.77E−02 |
| A_23_P40952 | NM_002880 | chr3: 12600670-12600611 | 3.78E−02 |
| A_23_P386241 | NM_001042353 | chr20: 774839-774898 | 3.79E−02 |
| A_32_P45894 | NM_018991 | chr7: 74834197-74834256 | 3.79E−02 |
| A_24_P912366 | NM_001109977 | chr4: 152718267-152718326 | 3.81E−02 |
| A_24_P269895 | NM_194247 | chr2: 177788575-177788634 | 3.82E−02 |
| A_23_P26745 | AK026675 | chr17: 17388803-17388744 | 3.82E−02 |
| A_23_P17204 | NM_022662 | chr2: 112243327-112243268 | 3.83E−02 |
| A_23_P11461 | NM_001032288 | chr20: 48134098-48132825 | 3.85E−02 |
| A_32_P120977 | NM_013237 | chr5: 176665511-176665570 | 3.86E−02 |
| A_24_P134319 | ENST00000349014 | chr20: 48939419-48939360 | 3.87E−02 |
| A_23_P255884 | NM_198252 | chr9: 123134592-123134651 | 3.88E−02 |
| A_23_P207896 | NM_001893 | chr17: 77795992-77795933 | 3.90E−02 |
| A_32_P234996 | THC2682560 | chr20: 30910079-30910138 | 3.90E−02 |
| A_23_P99985 | NM_002128 | chr13: 29932420-29932361 | 0.039271 |
| A_23_P27873 | NM_018049 | chr19: 2184225-2184166 | 3.93E−02 |
| A_23_P28434 | NM_003761 | chr2: 85662543-85662602 | 3.93E−02 |
| A_23_P134935 | NM_001394 | chr8: 29250360-29250301 | 3.94E−02 |
| A_23_P159325 | NM_139314 | chr19: 8345169-8345228 | 3.96E−02 |
| A_23_P354193 | NM_144628 | chr20: 367885-367826 | 3.97E−02 |
| A_24_P478153 | DA572835 | chr17: 19790635-19790694 | 3.98E−02 |
| A_23_P203737 | NM_033547 | chr11: 77267702-77267643 | 3.99E−02 |
| A_23_P359897 | NM_030647 | chr7: 139431574-139431515 | 4.00E−02 |
| A_23_P304287 | NM_002803 | chr7: 102795233-102795292 | 4.00E−02 |
| A_24_P823219 | BC047380 | chr22: 24181770-24183247 | 4.01E−02 |
| A_32_P113436 | NM_001011724 | chr13: 52114922-52114981 | 4.02E−02 |
| A_24_P316532 | BC040881 | chr6: 44385206-44385265 | 4.02E−02 |
| A_32_P77416 | BF368414 | chr13: 98801902-98801843 | 4.02E−02 |
| A_24_P303118 | NM_033625 | chr4: 109765754-109765813 | 4.04E−02 |
| A_23_P33433 | NM_001042539 | chr16: 29729134-29729193 | 4.06E−02 |
| A_24_P940086 | NM_007225 | chr17: 45015492-45015551 | 4.06E−02 |
| A_24_P184555 | NM_002859 | chr12: 119132707-119132648 | 0.040733 |
| A_23_P130194 | NM_006907 | chr17: 77483681-77483622 | 4.09E−02 |
| A_23_P139476 | NM_001780 | chr12: 54405598-54405539 | 4.10E−02 |
| A_24_P924202 | NM_032223 | chr11: 65161147-65161206 | 4.12E−02 |
| A_23_P164089 | NM_057178 | chr17: 30360383-30360324 | 4.12E−02 |
| A_24_P182183 | NM_001152 | chrX: 118488366-118488425 | 4.14E−02 |
| A_24_P89284 | NM_002109 | chr5: 140034842-140034592 | 4.15E−02 |
| A_24_P818010 | AK097080 | chr1: 124783-124724 | 0.041698 |
| A_23_P51951 | NM_006850 | chr1: 205143594-205143653 | 0.042038 |
| A_23_P391764 | NM_198679 | chr9: 133444470-133444411 | 4.21E−02 |
| A_24_P59485 | AK095300 | chr2: 242032343-242032402 | 4.21E−02 |
| A_23_P337033 | NM_012384 | chr20: 61689990-61689931 | 4.22E−02 |
| A_23_P155079 | NM_013356 | chr22: 36804339-36804280 | 4.22E−02 |
| A_23_P121499 | NM_006005 | chr4: 6355336-6355395 | 4.23E−02 |
| A_24_P919330 | NM_002032 | chr11: 61491601-61491542 | 0.043246 |
| A_24_P485742 | THC2689427 | chr2: 219643790-219643731 | 4.36E−02 |
| A_32_P212802 | XM_001720024 | chr7: 101781825-101781766 | 4.36E−02 |
| A_23_P100203 | NM_001537 | chr16: 82403745-82403804 | 4.39E−02 |
| A_23_P109254 | NM_184234 | chr20: 33755844-33755785 | 4.39E−02 |
| A_24_P374962 | NM_018991 | chr7: 74834647-74834706 | 4.39E−02 |
| A_23_P38190 | NM_139280 | chr17: 35332923-35332438 | 4.43E−02 |
| A_23_P354547 | ENST00000324076 | chr14: 23706007-23706066 | 4.44E−02 |
| A_23_P79161 | NM_013237 | chr5: 176665528-176665587 | 4.46E−02 |
| A_23_P377819 | NM_001039465 | chr14: 69307886-69307945 | 4.49E−02 |
| A_32_P184367 | NM_006013 | chrX: 153282383-153282443 | 4.50E−02 |
| A_32_P131090 | | chr7: 129543644-129543585 | 4.53E−02 |
| A_32_P38500 | | chr19: 052856683-052856742 | 4.55E−02 |
| A_23_P257296 | NM_003226 | chr21: 42605365-42605306 | 0.045488 |
| A_32_P158302 | NR_024412 | chr7: 112544823-112544764 | 4.56E−02 |
| A_23_P354175 | NM_138385 | chr4: 1687570-1687511 | 4.57E−02 |
| A_23_P15751 | NM_000160 | chr17_random: 2329878-2329937 | 4.57E−02 |
| A_23_P102994 | NM_002650 | chr22: 19395051-19394992 | 4.58E−02 |
| A_23_P36448 | NM_006815 | chr12: 122647111-122647170 | 4.58E−02 |
| A_23_P130995 | NM_144779 | chr19: 40349051-40352350 | 4.59E−02 |
| A_23_P80278 | NM_001007467 | chr22: 30344430-30344489 | 4.59E−02 |
| A_24_P173790 | AK055963 | chr1: 226850835-226850894 | 4.61E−02 |
| A_23_P399001 | NM_016463 | chr5: 139042720-139042779 | 4.62E−02 |
| A_23_P86021 | NM_003944 | chr1: 149603530-149603471 | 4.63E−02 |
| A_23_P47357 | NM_001028 | chr11: 118391826-118391767 | 0.046387 |
| A_24_P301240 | NM_006156 | chr14: 23756159-23756100 | 4.66E−02 |
| A_24_P33552 | XM_001133763 | chr6: 133360475-133360416 | 4.66E−02 |
| A_23_P134925 | NM_004331 | chr8: 26326203-26326262 | 4.66E−02 |
| A_23_P370005 | NM_003804 | chr6: 3058940-3058999 | 4.67E−02 |
| A_23_P43273 | NM_000127 | chr8: 118888679-118886297 | 4.69E−02 |
| A_23_P132294 | NM_001001560 | chr22: 36359426-36359485 | 4.69E−02 |
| A_23_P4060 | AK026312 | chr17: 19063230-19063289 | 4.70E−02 |
| A_24_P104538 | BC000924 | chr17: 41720014-41719955 | 4.70E−02 |
| A_23_P23960 | NM_001001342 | chr10: 102025068-102025009 | 4.73E−02 |
| A_23_P150255 | NM_006328 | chr11: 66151269-66151328 | 4.74E−02 |
| A_24_P342572 | NM_138346 | chr1: 11905540-11905481 | 4.75E−02 |
| A_23_P69362 | NM_024661 | chr3: 48448903-48448844 | 4.75E−02 |
| A_24_P50908 | ENST00000366699 | chr1: 226650225-226650166 | 4.75E−02 |
| A_24_P340679 | NM_021130 | chr7: 44805546-44805826 | 0.047544 |
| A_24_P400473 | NM_014655 | chr1: 154448977-154449036 | 4.77E−02 |
| A_23_P85922 | NM_181809 | chr1: 39767531-39767590 | 4.77E−02 |
| A_24_P829789 | THC2770735 | chr16: 24648579-24648638 | 4.77E−02 |
| A_32_P116323 | NM_015092 | chr16: 18777984-18777925 | 4.78E−02 |
| A_24_P305764 | NM_004595 | chrX: 21920730-21922388 | 4.80E−02 |
| A_24_P331128 | NM_002068 | chr19: 3108749-3108808 | 4.81E−02 |
| A_24_P651736 | AI859463 | chr14: 21040395-21040454 | 4.83E−02 |
| A_23_P132611 | AF088066 | chr9: 035433128-035433495 | 0.048271 |
| A_24_P347378 | NM_001629 | chr13: 30236155-30236214 | 4.83E−02 |
| A_23_P58031 | NM_004721 | chr3: 186682862-186682921 | 4.83E−02 |
| A_32_P77178 | AA085955 | chr1: 152822085-152822144 | 4.84E−02 |
| A_23_P366455 | NM_133264 | chr17: 35689355-35689414 | 4.84E−02 |
| A_23_P71644 | NM_004629 | chr9: 35063994-35063935 | 4.86E−02 |
| A_23_P213718 | NM_014402 | chr5: 132231194-132231253 | 4.86E−02 |
| A_24_P336596 | NM_030663 | chr1: 151123583-151123642 | 4.87E−02 |
| A_23_P310350 | NM_013276 | chr17: 3458806-3458747 | 4.87E−02 |
| A_23_P93818 | AL137492 | chr7: 74857450-74857509 | 4.88E−02 |
| A_23_P344594 | NM_153217 | chr5: 72505922-72505981 | 4.95E−02 |
| A_24_P323805 | | chr4: 043106956-043106893 | 4.95E−02 |
| A_24_P692566 | AF283771 | chr13: 29933154-29933213 | 4.95E−02 |
| A_23_P166548 | NM_025204 | chr22: 48979115-48979174 | 4.95E−02 |
| A_23_P82503 | NM_001040152 | chr7: 94136500-94136559 | 4.96E−02 |
| A_23_P365817 | NM_138689 | chr11: 63768626-63768567 | 4.97E−02 |
| A_23_P31532 | NM_080660 | chr7: 138362032-138361826 | 4.97E−02 |
| A_24_P162911 | NM_005813 | chr2: 37337552-37337493 | 4.97E−02 |
| A_23_P156708 | NM_032470 | chr6: 32117001-32116943 | 4.98E−02 |
| A_24_P761130 | AK097080 | chr1: 124796-124737 | 4.98E−02 |
| A_23_P358555 | NM_032356 | chr17: 7700841-7700782 | 4.99E−02 |
| A_32_P56434 | BG184978 | chr18: 9944415-9944356 | 4.99E−02 |

An alternative way to estimate the degree of benefit is by creating two independent prognostic models for chemotherapy treated patients and trastuzumab plus chemotherapy, and get a differential between the two estimates. Therefore prognostic genes were examined in these two treatment arms. Prognostic genes for chemotherapy patients based on the data from the extended study of 753 patients using Agilent microarrays are listed in Table 13, and prognostic genes for herceptin patients based on the data from the extended study of 753 patients using Agilent microarrays are listed in Table 14.

TABLE 13

| | TargetID | | hazard | |
|---|---|---|---|---|
| ProbeID | (GenBank Ref.) | Genomic Coordinates | ratio | p-value |
| A_24_P510357 | ENST00000390312 | chr22: 21431631-21431690 | 0.768346 | 0.000571 |
| A_24_P101642 | XM_001714632 | chr16: 33513077-33513136 | 0.814761 | 0.000843 |
| A_23_P49517 | NM_001078166 | chr17: 53436290-53436231 | 2.049707 | 0.00086 |
| A_24_P711477 | BC001783 | chr1: 26674467-26674408 | 2.728465 | 0.000887 |
| A_24_P639701 | AY062331 | chr2: 89107832-89107773 | 0.806419 | 0.001164 |
| A_24_P604784 | AF103312 | chr14: 106282025-106281966 | 0.788137 | 0.00119 |
| A_24_P298805 | ENST00000360102 | chr16: 33554992-33554933 | 0.781076 | 0.001221 |
| A_23_P38190 | NM_139280 | chr17: 35332923-35332438 | 1.303307 | 0.001282 |
| A_24_P517918 | ENST00000390596 | chr14: 105549442-105549383 | 0.751419 | 0.001287 |
| A_24_P341126 | | chr16: 033569122-033569061 | 0.784705 | 0.001409 |
| A_24_P289726 | NM_002809 | chr17: 35406100-35407150 | 1.25279 | 0.001417 |
| A_23_P163992 | NM_005310 | chr17: 35155907-35155966 | 1.405369 | 0.001559 |
| A_24_P813550 | ENST00000390630 | chr14: 106166275-106166216 | 0.839293 | 0.001771 |
| A_24_P315862 | ENST00000390630 | chr14: 106166308-106166249 | 0.833301 | 0.001805 |
| A_24_P66578 | ENST00000405516 | chr9: 69067373-69067315 | 0.79922 | 0.001906 |
| A_23_P136026 | AK128476 | chr14: 105245260-105245201 | 0.817496 | 0.002037 |
| A_23_P7313 | NM_001040058 | chr4: 89122944-89123003 | 1.247364 | 0.002054 |
| A_24_P505790 | NM_001033723 | chr8: 81896402-81896343 | 1.520337 | 0.002121 |
| A_24_P860662 | ENST00000390610 | chr14: 105804253-105804194 | 0.80808 | 0.002189 |
| A_32_P39440 | BC030813 | chr2: 89165879-89165820 | 0.798639 | 0.002424 |
| A_24_P384604 | | chr2: 097362969-097362908 | 0.767448 | 0.002443 |
| A_24_P677559 | BC032451 | chr2: 89223369-89223310 | 0.799262 | 0.002587 |
| A_23_P213126 | AB015331 | chr4: 80052403-80052462 | 1.68244 | 0.002607 |
| A_23_P147224 | ENST00000390594 | chr14: 105523963-105523904 | 0.839452 | 0.002611 |
| A_24_P408801 | ENST00000376209 | chr19: 63045236-63045295 | 1.639943 | 0.002802 |
| A_32_P437227 | NM_174911 | chr8: 127638820-127638761 | 1.429721 | 0.002846 |
| A_24_P142531 | NM_007372 | chr17: 59243210-59243269 | 1.49612 | 0.003025 |
| A_24_P169873 | ENST00000390539 | chr14: 105124380-105124321 | 0.763463 | 0.003183 |
| A_32_P147189 | BC071732 | chr2: 232286180-232286121 | 2.203027 | 0.003226 |
| A_23_P117558 | NM_002013 | chr14: 44657078-44657019 | 1.705079 | 0.003353 |
| A_23_P73328 | AY003763 | | 0.837924 | 0.003416 |
| A_24_P101226 | ENST00000390603 | chr14: 105681464-105681405 | 0.864132 | 0.003604 |
| A_24_P39843 | NM_001017916 | chr17: 58863660-58863601 | 1.292425 | 0.003707 |
| A_23_P32793 | DQ926472 | chr14: 105712855-105712796 | 0.85372 | 0.003741 |
| A_24_P247014 | | chr2: 68206320-68206379 | 2.340663 | 0.003785 |
| A_24_P315854 | ENST00000390630 | chr14: 106166255-106166196 | 0.844855 | 0.003836 |
| A_23_P50807 | NM_021107 | chr19: 44115409-44115468 | 1.608896 | 0.003894 |
| A_23_P66487 | NM_003077 | chr17: 59263905-59263846 | 1.372477 | 0.003946 |
| A_24_P272146 | BC067092 | chr2: 89120903-89120844 | 0.823927 | 0.004003 |
| A_23_P164047 | NM_012329 | chr17: 50825222-50825163 | 1.557044 | 0.004031 |
| A_24_P100684 | ENST00000390615 | chr14: 105886908-105886849 | 0.857314 | 0.004085 |
| A_32_P68459 | NM_021130 | chr7: 44807530-44807589 | 2.179572 | 0.004143 |
| A_23_P164035 | NM_002805 | chr17: 59261020-59261262 | 1.450399 | 0.004279 |
| A_23_P139476 | NM_001780 | chr12: 54405598-54405539 | 2.167302 | 0.004322 |
| A_24_P357847 | BC030813 | chr2: 89165852-89165793 | 0.830101 | 0.004336 |
| A_23_P21800 | BC032451 | chr2: 89223399-89223340 | 0.806128 | 0.004337 |
| A_24_P490109 | | chr9: 069434014-069433963 | 0.817168 | 0.00435 |
| A_24_P173566 | X57818 | chr22: 021553308-021553367 | 0.786387 | 0.004491 |
| A_32_P24080 | | chr20: 005518551-005518610 | 1.388626 | 0.004562 |
| A_23_P26783 | NM_002809 | chr17: 35407673-35407732 | 1.253778 | 0.004568 |
| A_32_P190951 | ENST00000390622 | chr14: 106038205-106038146 | 0.810681 | 0.004569 |
| A_24_P203886 | | chr2: 097367246-097367188 | 0.851234 | 0.00464 |
| A_24_P110242 | ENST00000360102 | chr16: 33554939-33554880 | 0.807989 | 0.004646 |
| A_32_P8067 | THC2640786 | cbr14: 73339402-73339461 | 0.71749 | 0.00467 |
| A_32_P1036 | THC2532114 | chr11: 64949772-64949713 | 1.694317 | 0.004673 |
| A_23_P383532 | NM_017813 | chr8: 58035771-58035712 | 1.60229 | 0.004689 |
| A_23_P390206 | | chr14: 105401907-105401855 | 0.774717 | 0.004749 |
| A_23_P343977 | AK125510 | chr1: 153553774-153553715 | 1.596259 | 0.004873 |
| A_32_P1360 | THC2670603 | chr21: 43204883-43204942 | 0.809071 | 0.004983 |
| A_23_P214046 | NM_012300 | chr5: 171221669-171221610 | 1.740354 | 0.004988 |
| A_24_P169713 | THC2497143 | chr22: 021431506-021431447 | 0.842178 | 0.004989 |
| A_24_P391604 | NM_181605 | chr21: 30886864-30886923 | 0.812442 | 0.005047 |
| A_24_P358321 | ENST00000390267 | chr2: 89624196-89624255 | 0.800264 | 0.005047 |
| A_24_P605563 | AY172962 | chr22: 021578642-021578701 | 0.825511 | 0.005073 |
| A_24_P750327 | AJ245002 | chr15: 19974257-19974198 | 0.830155 | 0.005113 |
| A_23_P344451 | NM_016073 | chr15: 81598311-81598252 | 1.43715 | 0.005166 |
| A_32_P148118 | XM_001717196 | chr2: 91369939-91369998 | 0.824482 | 0.005257 |
| A_24_P921933 | NM_001078166 | chr17: 53433448-53433389 | 1.328395 | 0.005346 |
| A_24_P144346 | Y11328 | chr16: 033568993-033568934 | 0.839502 | 0.005365 |
| A_23_P27247 | NM_005121 | chr17: 57383086-57383027 | 1.459311 | 0.005405 |
| A_23_P135769 | NM_001101 | chr7: 5533766-5533707 | 2.215684 | 0.005501 |
| A_23_P259763 | AF471475 | chr14: 105582967-105582910 | 0.851756 | 0.00557 |
| A_23_P52176 | NM_003431 | chr1: 245386141-245386082 | 1.556702 | 0.005591 |
| A_23_P20894 | NM_024757 | chr9: 139832347-139832406 | 1.477416 | 0.005664 |
| A_24_P405850 | NM_006320 | chr4: 129410846-129410787 | 1.987947 | 0.005667 |
| A_23_P11279 | NM_018466 | chrX: 110814986-110817828 | 1.781433 | 0.005672 |
| A_23_P342067 | NM_175866 | chr1: 160759679-160759738 | 1.663864 | 0.005714 |
| A_23_P100632 | NM_001002033 | chr17: 70643757-70643698 | 1.324269 | 0.005747 |
| A_23_P152651 | NM_007372 | chr17: 59249724-59249783 | 1.469079 | 0.005821 |
| A_32_P56434 | BG184978 | chr18: 9944415-9944356 | 1.684178 | 0.00583 |
| A_23_P4036 | NM_018469 | chr17: 59579211-59579152 | 1.419632 | 0.005843 |
| A_23_P104522 | NM_006393 | chr10: 21114445-21114386 | 1.341532 | 0.005877 |
| A_24_P51360 | NM_020117 | chr5: 145528004-145527945 | 1.861798 | 0.005881 |
| A_24_P683861 | ENST00000390594 | chr14: 105523996-105523937 | 0.808201 | 0.005892 |
| A_32_P157927 | ENST00000390243 | chr2: 88966535-88966594 | 0.855098 | 0.005916 |
| A_23_P158868 | | | 0.844719 | 0.005916 |
| A_24_P305764 | NM_004595 | chrX: 21920730-21922388 | 1.517796 | 0.005938 |
| A_23_P255884 | NM_198252 | chr9: 123134592-123134651 | 0.638471 | 0.005987 |
| A_24_P354468 | NM_002106 | chr4: 101088651-101088592 | 1.574216 | 0.006088 |
| A_23_P26799 | NM_004870 | chr17: 7431806-7431865 | 1.71062 | 0.006095 |
| A_24_P849319 | AF038194 | chr1: 191412006-191412065 | 1.661173 | 0.006171 |
| A_24_P626951 | ENST00000390253 | chr2: 89180703-89180644 | 0.776081 | 0.006177 |
| A_32_P65022 | BC144530 | chr22: 15775318-15775259 | 0.798617 | 0.006227 |
| A_32_P5480 | NM_203463 | chr2: 169339249-169339308 | 1.489906 | 0.006232 |
| A_32_P173298 | NM_012426 | chr16: 69120424-69120483 | 1.783893 | 0.006279 |
| A_24_P943709 | AW444553 | chr8: 127636198-127636257 | 1.291037 | 0.006409 |
| A_24_P240732 | NM_017813 | chr8: 58053183-58041427 | 1.534725 | 0.006461 |
| A_24_P229447 | ENST00000360102 | chr16: 33554938-33554879 | 0.828577 | 0.006626 |
| A_23_P7783 | NM_016604 | chr5: 137800264-137800323 | 1.742923 | 0.006639 |
| A_32_P95672 | AW875618 | chr17: 053437944-053438203 | 1.636333 | 0.006698 |
| A_24_P385119 | NM_003184 | chr8: 120812707-120812648 | 1.433703 | 0.006843 |
| A_23_P72330 | | | 0.831264 | 0.006927 |
| A_32_P20535 | AA279208 | chr9: 109132642-109132583 | 1.909255 | 0.006952 |
| A_24_P367432 | BC070333 | chr14: 106241155-106241096 | 0.853332 | 0.006992 |
| A_32_P102111 | BM091498 | chr6: 2894259-2898292 | 1.449831 | 0.007022 |
| A_32_P722809 | BC034142 | chr2: 89302371-89302312 | 0.811973 | 0.007118 |
| A_24_P384119 | ENST00000354689 | chr16: 033537503-033537562 | 0.853739 | 0.007142 |
| A_32_P10231 | AA973155 | chr4: 52472071-52472012 | 1.882251 | 0.007227 |
| A_24_P829261 | NR_002819 | chr11: 65026718-65026777 | 1.669172 | 0.007245 |
| A_32_P200144 | AK130614 | chr14: 105278810-105278751 | 0.850013 | 0.007247 |
| A_24_P323610 | NM_012335 | chr19: 8524273-8524093 | 0.664131 | 0.007254 |
| A_32_P65589 | AJ252276 | chr2: 094982763-094982710 | 0.844651 | 0.007263 |
| A_32_P233860 | NM_145690 | chr8: 102030031-102006416 | 1.442582 | 0.007293 |
| A_32_P159192 | ENST00000390281 | chr2: 89911481-89911540 | 0.828374 | 0.007332 |
| A_23_P61042 | ENST00000390539 | chr14: 105124975-105124694 | 0.82856 | 0.007464 |
| A_23_P124632 | AF067420 | chr14: 105724408-105401473 | 0.861108 | 0.007593 |
| A_24_P161853 | XM_001718037 | chr16: 032913565-032914075 | 0.794039 | 0.007721 |
| A_23_P133146 | NM_002106 | chr4: 101088527-101088468 | 1.674927 | 0.007739 |
| A_23_P167168 | NM_144646 | chr4: 71740797-71740738 | 0.828583 | 0.007777 |
| A_24_P702749 | AF471454 | chr14: 105886882-105886823 | 0.839407 | 0.007969 |
| A_23_P157669 | NM_005005 | chr8: 125631299-125631358 | 1.41493 | 0.00797 |
| A_24_P465799 | AF063695 | | 0.842099 | 0.008007 |
| A_23_P81805 | NM_001025366 | chr6: 43860503-43860562 | 1.501272 | 0.008066 |
| A_32_P43664 | AW136683 | chr4: 71741006-71741065 | 0.807138 | 0.008091 |
| A_32_P142407 | | chr14: 101617849-101617908 | 1.586477 | 0.008136 |
| A_23_P137391 | NM_001428 | chr1: 8846700-8846641 | 1.64736 | 0.008169 |
| A_23_P68106 | NM_021103 | chr2: 84986309-84986652 | 1.84715 | 0.008189 |
| A_32_P226149 | NM_145690 | chr8: 102000343-102000284 | 1.536693 | 0.008269 |
| A_24_P104980 | AJ319669 | chr22: 21573559-21573617 | 0.819997 | 0.008332 |
| A_23_P318396 | NM_198700 | chr11: 47446813-47446754 | 1.901308 | 0.008384 |
| A_24_P255543 | AL390214 | chr5: 14483194-14483253 | 1.528683 | 0.008395 |
| A_24_P519504 | X57802 | chr22: 21553467-21553526 | 0.815508 | 0.008526 |
| A_24_P910724 | DB352211 | chr13: 113188018-113188077 | 1.430715 | 0.008577 |
| A_23_P350782 | ENST00000390261 | chr2: 89590337-89590396 | 0.843814 | 0.008603 |
| A_23_P118815 | NM_001012271 | chr17: 73732315-73732374 | 1.257662 | 0.008636 |
| A_23_P20275 | NM_024613 | chr8: 96237936-96237995 | 1.266364 | 0.008651 |
| A_24_P184555 | NM_002859 | chr12: 119132707-119132648 | 1.767072 | 0.008787 |
| A_24_P315941 | ENST00000390625 | chr14: 106084108-106084049 | 0.873156 | 0.008821 |
| A_24_P323298 | | chr14: 106270026-106269967 | 0.855046 | 0.008825 |
| A_24_P114255 | NM_138799 | chr2: 8914684-8914625 | 1.353499 | 0.008857 |
| A_23_P30175 | NM_018695 | chr5: 65411596-65411655 | 1.464357 | 0.008977 |
| A_23_P21260 | ENST00000390263 | chr2: 89400739-89400680 | 0.838413 | 0.008979 |
| A_24_P311845 | CR612518 | chr5: 167909603-167909544 | 1.561895 | 0.009022 |
| A_24_P933108 | S57296 | chr17: 35127366-35127424 | 1.167764 | 0.00906 |
| A_24_P148321 | NM_003528 | chr1: 148123097-148123038 | 1.291811 | 0.009089 |
| A_23_P390209 | ENST00000390593 | chr14: 105476789-105476730 | 0.87636 | 0.009125 |
| A_24_P306905 | BC063599 | chr2: 89257701-89257199 | 0.843565 | 0.009156 |
| A_24_P95038 | NM_021130 | chr7: 44807530-44807589 | 2.040498 | 0.009157 |
| A_23_P3982 | NM_032339 | chr17: 35139053-35138994 | 1.268599 | 0.009167 |
| A_24_P278192 | NM_000476 | chr9: 129669056-129668997 | 0.715408 | 0.009248 |
| A_32_P196047 | NM_181787 | chr8: 95872869-95872928 | 1.610477 | 0.009364 |
| A_23_P19084 | NM_004499 | chr5: 177566728-177566787 | 1.528887 | 0.00938 |
| A_24_P204574 | | chr22: 021042558-021042617 | 0.817264 | 0.009487 |
| A_24_P18802 | NM_020857 | chr15: 38983178-38983237 | 0.838441 | 0.009565 |
| A_23_P76364 | NM_001769 | chr12: 6212886-6214687 | 1.415286 | 0.00958 |
| A_23_P251303 | NM_014866 | chr9: 138455189-138455130 | 1.585524 | 0.009686 |
| A_23_P62070 | NM_001003841 | chr5: 1275535-1275594 | 0.836587 | 0.009786 |
| A_23_P52589 | NM_003475 | chr11: 553924-553983 | 1.495588 | 0.009828 |
| A_23_P71430 | NM_001001481 | chr8: 74867582-74867523 | 1.469854 | 0.009864 |
| A_24_P257539 | NM_012300 | chr5: 171228351-171228292 | 1.76611 | 0.010004 |
| A_23_P101281 | NM_032828 | chr19: 63067990-63068049 | 1.461144 | 0.010041 |
| A_23_P45389 | NM_004251 | chrX: 13637278-13637337 | 1.60585 | 0.010069 |
| A_24_P33341 | ENST00000360102 | chr16: 33554809-33554750 | 0.850297 | 0.010106 |
| A_24_P183375 | NM_002715 | chr5: 133562732-133561432 | 1.707807 | 0.010161 |
| A_24_P276853 | NM_001037533 | chr1: 153987058-153986999 | 0.502129 | 0.010293 |
| A_32_P204019 | NM_000992 | chr3: 52003043-52002984 | 0.630111 | 0.010372 |
| A_23_P336034 | NM_148962 | chr2: 42844482-42844423 | 0.771989 | 0.010394 |
| A_24_P80633 | NM_001903 | chr5: 138296287-138297435 | 1.760294 | 0.01048 |
| A_32_P30926 | NM_001378 | chr2: 172310631-172312538 | 1.688276 | 0.010523 |
| A_24_P15550 | ENST00000390260 | chr2: 89325437-89325378 | 0.823838 | 0.010538 |
| A_32_P11372 | NR_003674 | chr9: 46585969-46586028 | 1.309756 | 0.010539 |
| A_32_P229365 | BC005884 | chr22: 16638397-16638338 | 1.469583 | 0.010665 |
| A_23_P43979 | M87790 | chr22: 21573548-21573605 | 0.83236 | 0.010667 |
| A_24_P209571 | NM_145690 | chr8: 102000474-102000415 | 1.415456 | 0.010687 |
| A_23_P210048 | NM_006037 | chr2: 239635672-239635613 | 0.634764 | 0.010698 |
| A_32_P56415 | THC2557134 | chr14: 89942431-89942372 | 1.655545 | 0.010726 |
| A_32_P118686 | BC021035 | chr17: 69770671-69770730 | 2.022748 | 0.010794 |
| A_23_P416314 | BC034222 | chr11: 62985670-62985611 | 0.821145 | 0.010873 |
| A_23_P502343 | NM_025220 | chr20: 3597151-3597092 | 0.743246 | 0.010888 |
| A_23_P134637 | NM_014855 | chr7: 4797370-4797429 | 0.833389 | 0.011055 |
| A_24_P203710 | NM_003934 | chr9: 132502862-132502921 | 1.777204 | 0.011102 |
| A_23_P401568 | NM_005911 | chr2: 85625440-85625499 | 1.697991 | 0.011172 |
| A_23_P501515 | NM_031844 | chr1: 243084006-243083947 | 1.714914 | 0.011242 |
| A_24_P6381 | NM_178276 | chr5: 79477776-79477717 | 1.575919 | 0.011255 |
| A_32_P39324 | CB993898 | chr12: 119204769-119204710 | 1.320852 | 0.011271 |
| A_23_P162866 | NM_005348 | chr14: 101618204-101617856 | 1.533155 | 0.011323 |
| A_32_P97489 | NM_145690 | chr8: 102001659-102001600 | 1.589961 | 0.011423 |
| A_32_P131377 | NM_005669 | chr5: 112250624-112242386 | 1.49564 | 0.011497 |
| A_32_P132766 | THC2671036 | chrX: 73356346-73356405 | 1.561781 | 0.011589 |
| A_23_P435390 | | | 0.835814 | 0.011727 |
| A_23_P42969 | NM_006682 | chr7: 76663724-76663665 | 0.666845 | 0.01175 |
| A_24_P47870 | NM_001344 | chr14: 22103867-22103808 | 1.583445 | 0.011811 |
| A_23_P85800 | NM_001803 | chr1: 26519472-26519531 | 0.726579 | 0.011859 |
| A_23_P113056 | | chr14: 106354036-106353986 | 0.861538 | 0.012043 |
| A_24_P221914 | AF144054 | chr13: 106392622-106392563 | 0.830869 | 0.01205 |
| A_24_P102080 | S48723 | chr8: 61696171-61696230 | 1.386468 | 0.012178 |
| A_24_P202555 | NM_015073 | chr19: 43390645-43390704 | 1.359326 | 0.01224 |
| A_23_P166848 | NM_002343 | chr3: 46454493-46452724 | 0.881928 | 0.012318 |
| A_24_P101960 | | chr12: 25011453-25011394 | 0.675039 | 0.01249 |
| A_32_P52590 | AA887631 | chr17: 38248658-38247763 | 0.774447 | 0.012508 |
| A_24_P35228 | NM_024915 | chr8: 102750748-102750807 | 1.406331 | 0.012571 |
| A_24_P341626 | | chr8: 142398210-142398151 | 0.750775 | 0.012697 |
| A_32_P187827 | NR_003610 | chr16: 68577805-68574123 | 1.447129 | 0.012698 |
| A_24_P325533 | NM_000298 | chr1: 153526236-153526177 | 1.43955 | 0.012761 |
| A_23_P158817 | AK130614 | chr14: 105180647-105180588 | 0.872964 | 0.013014 |
| A_23_P7221 | NM_033625 | chr4: 109765754-109765813 | 1.97327 | 0.013197 |
| A_23_P70398 | NM_001025370 | chr6: 43854201-43854612 | 1.342304 | 0.013332 |
| A_24_P472081 | ENST00000390294 | chr22: 21042416-21042475 | 0.822477 | 0.013357 |
| A_32_P148122 | BC095489 | chr2: 89198231-89198172 | 0.797452 | 0.01341 |
| A_23_P369960 | NM_014551 | chr22: 49303554-49303983 | 0.875096 | 0.013569 |
| A_23_P361654 | BC073764 | chr2: 89121043-89120986 | 0.867704 | 0.013577 |
| A_23_P381945 | NM_005556 | chr12: 50917564-50918733 | 1.307403 | 0.013679 |
| A_32_P199923 | AX229882 | chr14: 094625950-094626009 | 1.643176 | 0.013738 |
| A_23_P319520 | NM_145003 | chr8: 143308796-143291578 | 0.781604 | 0.01379 |
| A_23_P44053 | | chr2: 089349024-089348968 | 0.850547 | 0.013894 |
| A_24_P161764 | ENST00000390595 | chr14: 105542527-105542468 | 0.870867 | 0.013969 |
| A_24_P198844 | NM_004870 | chr17: 7431735-7431794 | 1.626608 | 0.014019 |
| A_24_P245330 | AK098497 | chr18: 203852-203911 | 1.469951 | 0.014098 |
| A_23_P254179 | NM_015339 | chr20: 48940879-48940820 | 1.513824 | 0.014108 |
| A_32_P181131 | AW467174 | chr1: 65465226-65465167 | 1.312212 | 0.014154 |
| A_32_P222383 | NM_005517 | chr1: 26674779-26674838 | 2.193387 | 0.014228 |
| A_23_P435002 | NM_152546 | chr5: 121391152-121391211 | 1.533965 | 0.014246 |
| A_23_P3956 | NM_198594 | chr17: 74556765-74556824 | 0.785106 | 0.014274 |
| A_23_P85936 | AL137472 | chr1: 226329115-226329056 | 0.759835 | 0.014354 |
| A_32_P92355 | NM_001101676 | chr8: 119273718-119273659 | 1.333377 | 0.014374 |
| A_32_P96752 | AW946823 | chr6: 21705811-21705752 | 1.348878 | 0.014411 |
| A_23_P162874 | NM_005348 | chr14: 101619265-101619206 | 1.515579 | 0.014438 |
| A_23_P312920 | NM_006235 | chr11: 110728273-110728214 | 0.873653 | 0.014459 |
| A_24_P92683 | AK128476 | chr14: 106202515-105937729 | 0.810861 | 0.014482 |
| A_24_P230100 | ENST00000397494 | chr17: 077870685-077870626 | 0.810315 | 0.014496 |
| A_23_P167066 | NM_003359 | chr4: 39178216-39178157 | 1.33907 | 0.014608 |
| A_24_P183292 | NM_001025105 | chr5: 148855500-148855441 | 1.690278 | 0.014675 |
| A_23_P108394 | THC2783023 | chr2: 223515847-223515902 | 1.352847 | 0.014749 |
| A_23_P344037 | NM_018223 | chr12: 131927844-131927785 | 1.565774 | 0.014789 |
| A_24_P921260 | AK022030 | chr1: 191327214-191327273 | 1.423739 | 0.014884 |
| A_24_P153680 | NM_018660 | chr8: 28274572-28274513 | 0.839501 | 0.01499 |
| A_23_P43504 | NM_001606 | chr9: 139022180-139022121 | 0.840892 | 0.015001 |
| A_32_P51988 | EF178107 | chr14: 106119770-106119711 | 0.848247 | 0.015045 |
| A_24_P369898 | ENST00000293201 | chr17: 71128275-71128334 | 0.800956 | 0.015051 |
| A_24_P271285 | NM_052876 | chr19: 13110965-13111024 | 0.766772 | 0.015078 |
| A_24_P247849 | AK093508 | chr1: 162910490-162910549 | 1.403004 | 0.015152 |
| A_24_P132039 | NM_004290 | chr5: 141348281-141348340 | 1.488416 | 0.015207 |
| A_23_P206981 | NM_007146 | chr17: 53405525-53405466 | 1.399775 | 0.015262 |
| A_23_P118266 | NM_006885 | chr16: 71378607-71378548 | 1.595004 | 0.015289 |
| A_32_P234405 | CK570316 | chr1: 205168681-205168740 | 0.855901 | 0.015539 |
| A_23_P157452 | NM_005034 | chr8: 101234737-101234796 | 1.346558 | 0.015823 |
| A_23_P216984 | NM_016322 | chr9: 122983349-122983290 | 1.665089 | 0.015828 |
| A_24_P182586 | CR591805 | chr1: 201260360-201260419 | 1.604141 | 0.015843 |
| A_32_P157385 | AK124175 | chr20: 47338014-47338073 | 1.425671 | 0.015866 |
| A_23_P119526 | NM_003938 | chr19: 2060157-2060098 | 1.691216 | 0.015952 |
| A_23_P341325 | NM_080746 | chr14: 46190323-46190264 | 0.628176 | 0.016014 |
| A_24_P620621 | NM_001127322 | chr12: 52917758-52917699 | 1.558601 | 0.016134 |
| A_23_P146855 | NM_001044370 | chr22: 42233981-42234040 | 0.856705 | 0.016259 |
| A_24_P346644 | AL050206 | chr8: 26285395-26285454 | 1.483193 | 0.016264 |
| A_23_P254816 | NM_004609 | chr20: 533004-532945 | 0.817362 | 0.016266 |
| A_24_P370887 | NM_004781 | chr1: 7763543-7763602 | 1.730925 | 0.016335 |
| A_23_P167692 | NM_002752 | chr5: 179602332-179602273 | 1.455487 | 0.016337 |
| A_23_P143242 | NM_005194 | chr20: 48241320-48241379 | 0.813411 | 0.01634 |
| A_23_P84791 | | | 0.870834 | 0.016509 |
| A_23_P30377 | NM_014829 | chr5: 134192411-134192470 | 1.726329 | 0.016521 |
| A_23_P104054 | NM_016227 | chr1: 170847379-170847438 | 1.391787 | 0.016542 |
| A_23_P69988 | NM_002887 | chr5: 167878715-167878774 | 1.605237 | 0.016577 |
| A_24_P775659 | CR593500 | chr19: 3803247-3803188 | 0.838266 | 0.016613 |
| A_23_P21249 | DQ100840 | chr14: 106065079-106065020 | 0.858479 | 0.016811 |
| A_23_P401 | NM_016343 | chr1: 212903931-212903990 | 1.314742 | 0.016814 |
| A_24_P649747 | NM_014753 | chr10: 42637528-42637587 | 1.735099 | 0.016914 |
| A_32_P41405 | NM_032361 | chr5: 175319439-175319380 | 1.419415 | 0.016982 |
| A_24_P154006 | NM_005005 | chr8: 125631269-125631328 | 1.396871 | 0.016982 |
| A_24_P604649 | NM_000291 | chrX: 77265063-77265367 | 1.409986 | 0.017002 |
| A_24_P151959 | AK021860 | chr19: 44127908-44127849 | 0.821435 | 0.017151 |
| A_32_P76122 | THC2527474 | chr1: 149685167-149685226 | 1.550966 | 0.017184 |
| A_32_P224345 | CB852269 | chr8: 102750799-102750740 | 1.366915 | 0.017203 |
| A_23_P4503 | NM_017512 | chr18: 678576-675952 | 1.443281 | 0.017349 |
| A_24_P608268 | ENST00000390636 | chr14: 106282059-106282000 | 0.889519 | 0.017357 |
| A_23_P89249 | NM_001005862 | chr17: 35138355-35138414 | 1.246911 | 0.017437 |
| A_24_P7211 | NM_033551 | chr5: 154162139-154163088 | 1.622964 | 0.017458 |
| A_23_P104188 | NM_004433 | chr1: 200251052-200251111 | 1.317724 | 0.017634 |
| A_23_P430442 | NM_020772 | chr17: 24613046-24612987 | 1.283737 | 0.017658 |
| A_32_P189974 | BE328474 | chr16: 18329519-18329578 | 1.876039 | 0.017862 |
| A_23_P436179 | NM_001152 | chrX: 118489013-118489072 | 1.474912 | 0.018117 |
| A_24_P238744 | NR_004845 | chr5: 79631567-79631508 | 1.921135 | 0.018287 |
| A_23_P208373 | NM_000767 | chr19: 46215365-46215424 | 0.812687 | 0.018288 |
| A_23_P165028 | NM_001348 | chr19: 3909981-3909922 | 0.847065 | 0.018384 |
| A_23_P163942 | NM_003168 | chr17: 53778348-53778289 | 1.384081 | 0.018463 |
| A_32_P124978 | THC2692453 | chr17: 72862112-72862171 | 1.324345 | 0.018592 |
| A_23_P211363 | NM_002073 | chr22: 21795791-21795850 | 0.795758 | 0.018922 |
| A_24_P75190 | NM_000519 | chr11: 5211893-5211834 | 0.823001 | 0.019106 |
| A_23_P75741 | NM_198183 | chr11: 57076532-57076473 | 0.701985 | 0.019109 |
| A_23_P430140 | AF090930 | chr17: 78073503-78073444 | 0.643006 | 0.019134 |
| A_23_P88963 | NM_000034 | chr16: 29988825-29988971 | 1.445762 | 0.019171 |
| A_32_P223786 | W80819 | chr1: 167367552-167367493 | 1.285298 | 0.019182 |
| A_24_P204727 | ENST00000390624 | chr14: 106065034-106064975 | 0.845539 | 0.019308 |
| A_24_P47547 | NM_006325 | chr12: 129923380-129923521 | 1.730721 | 0.019339 |
| A_32_P108387 | CF528315 | chr5: 132231109-132230598 | 1.475502 | 0.019379 |
| A_23_P123853 | NM_006274 | chr9: 34679665-34679606 | 0.789981 | 0.01939 |
| A_24_P834210 | | chr17: 044065804-044065863 | 0.76945 | 0.019399 |
| A_32_P226735 | BX090520 | chr16: 53506531-53506472 | 1.262843 | 0.019415 |
| A_32_P104063 | AF275804 | chr16: 53510327-53510282 | 1.344375 | 0.019458 |
| A_23_P355385 | NM_002721 | chr9: 126951375-126951316 | 1.662122 | 0.019472 |
| A_23_P145863 | NM_005620 | chr1: 150271843-150271784 | 1.443972 | 0.019548 |
| A_23_P57950 | NM_000539 | chr3: 130735807-130735866 | 0.831798 | 0.019715 |
| A_24_P367473 | NM_001837 | chr3: 46281799-46281858 | 0.824013 | 0.019874 |
| A_24_P395415 | AF343666 | chr1: 155833781-155833722 | 0.843684 | 0.019887 |
| A_23_P81392 | NM_015238 | chr5: 167828705-167828764 | 1.402943 | 0.020108 |
| A_23_P41021 | NM_007184 | chr3: 52501641-52501700 | 0.783416 | 0.020151 |
| A_24_P291016 | NM_005157 | chr9: 132700653-132700712 | 0.758324 | 0.020155 |
| A_23_P29504 | NM_152699 | chr3: 198142823-198142882 | 1.890846 | 0.020183 |
| A_24_P15388 | BC024289 | chr14: 105762789-105762730 | 0.87075 | 0.02035 |
| A_32_P29507 | NM_005517 | chr1: 26674722-26674781 | 1.470016 | 0.020376 |
| A_24_P202558 | NM_015073 | chr19: 43390609-43390668 | 1.292075 | 0.020408 |
| A_32_P106646 | NM_198076 | chr1: 243072144-243072985 | 0.606603 | 0.020497 |
| A_23_P324885 | NM_000648 | chr3: 46375710-46375769 | 0.836876 | 0.020512 |
| A_23_P24384 | NM_032251 | chr11: 63881511-63881570 | 0.734308 | 0.020704 |
| A_32_P198923 | NM_145690 | chr8: 102030071-102030012 | 1.335605 | 0.020713 |
| A_32_P28264 | THC2663978 | chr16: 30176484-30176543 | 1.84305 | 0.020724 |
| A_23_P35230 | NM_172350 | chr1: 206007060-206007119 | 1.43223 | 0.020759 |
| A_32_P68443 | BE714307 | chr5: 127553008-127552949 | 1.181836 | 0.020806 |
| A_32_P49116 | CR617865 | chr13: 110344477-110344536 | 1.453201 | 0.021164 |
| A_24_P480722 | | chr6: 168992288-168992229 | 0.837048 | 0.02122 |
| A_23_P22273 | D00022 | chr12: 55318360-55318302 | 1.590452 | 0.021287 |
| A_32_P200947 | BC035518 | chr3: 115516771-115516712 | 0.640108 | 0.021346 |
| A_24_P766204 | NM_005921 | chr5: 056226249-056226308 | 1.38409 | 0.021356 |
| A_23_P49521 | NM_001078166 | chr17: 53437448-53437389 | 1.426825 | 0.021462 |
| A_23_P333227 | NM_001031711 | chr5: 172310592-172310651 | 1.377781 | 0.021465 |
| A_23_P65609 | NM_015859 | chr14: 80728621-80721664 | 1.595162 | 0.021481 |
| A_32_P162004 | NM_000992 | chr3: 52003051-52002992 | 0.663331 | 0.021507 |
| A_23_P102816 | BX647159 | chr20: 30238694-30238635 | 0.719428 | 0.021515 |
| A_23_P402751 | BC000228 | chr9: 83388455-83388396 | 1.457956 | 0.021518 |
| A_23_P141405 | NM_002512 | chr17: 46603918-46603977 | 1.262939 | 0.021605 |
| A_24_P829882 | | chr16: 029832202-029832261 | 1.499724 | 0.021607 |
| A_32_P119233 | AI457687 | chr4: 24138684-24138743 | 1.671997 | 0.021623 |
| A_23_P85392 | NM_007033 | chr1: 2323576-2323635 | 1.53889 | 0.021791 |
| A_23_P118643 | NM_001968 | chr4: 100020603-100020544 | 1.415685 | 0.021793 |
| A_24_P366445 | | chr19: 035104314-035104382 | 1.897129 | 0.021851 |
| A_32_P152400 | THC2568538 | chr1: 2322927-2322889 | 1.410453 | 0.022055 |
| A_24_P928658 | NM_005411 | chr10: 81040721-81041115 | 0.865874 | 0.022223 |
| A_32_P208621 | THC2687042 | chr8: 106469601-106469660 | 0.831868 | 0.022233 |
| A_24_P145377 | NM_001017963 | chr14: 101617198-101617145 | 1.305039 | 0.022406 |
| A_23_P167067 | NM_003359 | chr4: 39178256-39178197 | 1.321746 | 0.022418 |
| A_23_P99540 | NM_004926 | chr14: 68324288-68324229 | 1.578746 | 0.022464 |
| A_23_P123974 | NM_012145 | chr2_random: 93045-92986 | 1.313142 | 0.022491 |
| A_24_P914479 | BC002724 | chr20: 17896427-17896368 | 1.381614 | 0.022553 |
| A_24_P313504 | NM_005030 | chr16: 23609129-23609188 | 1.258856 | 0.022652 |
| A_23_P65757 | NM_004701 | chr15: 57204431-57204490 | 1.295399 | 0.022741 |
| A_23_P346093 | NM_152468 | chr17: 73650304-73650363 | 0.766715 | 0.022771 |
| A_32_P206549 | BC035156 | chr8: 102262354-102262295 | 1.337764 | 0.022812 |
| A_32_P104407 | CB243784 | chr1: 206034365-206034306 | 1.484468 | 0.022822 |
| A_23_P133474 | NM_002084 | chr5: 150388052-150388111 | 0.739845 | 0.022876 |
| A_23_P30223 | NM_001047 | chr5: 6721693-6721752 | 1.358663 | 0.022962 |
| A_23_P343900 | ENST00000390638 | chr14: 106330534-106330475 | 0.848309 | 0.023013 |
| A_24_P267664 | NR_026543 | chr21: 39903445-39903386 | 0.846964 | 0.023024 |
| A_24_P135427 | AK094748 | chr19: 63209320-63209379 | 1.371607 | 0.023091 |
| A_32_P137939 | NM_001101 | chr7: 5533372-5533313 | 1.898672 | 0.023097 |
| A_24_P941441 | NM_006572 | chr17: 60437769-60437710 | 1.443877 | 0.023104 |
| A_23_P15511 | NM_004396 | chr17: 59926806-59926747 | 1.342196 | 0.023162 |
| A_23_P41645 | NM_012081 | chr5: 95248974-95248915 | 1.316339 | 0.023168 |
| A_32_P12214 | NM_001013699 | chr12: 31835751-31835692 | 1.384085 | 0.02317 |
| A_24_P380330 | NM_024594 | chr5: 167915509-167915450 | 1.363799 | 0.023228 |
| A_24_P127701 | | chr11: 67490022-67489963 | 0.635142 | 0.02324 |
| A_24_P111096 | NM_004566 | chr10: 6314902-6314961 | 0.806724 | 0.023284 |
| A_23_P70417 | NM_018141 | chr6: 42283881-42283822 | 1.519679 | 0.023309 |
| A_23_P429184 | NM_198066 | chr14: 52311844-52311785 | 1.461639 | 0.023311 |
| A_23_P251002 | | chr6: 026389784-026389729 | 1.195059 | 0.02333 |
| A_24_P392195 | | chr1: 209413550-209413609 | 0.654482 | 0.023429 |
| A_24_P937911 | BF813638 | chrX: 73375785-73375844 | 1.453816 | 0.023442 |
| A_23_P129989 | NM_001524 | chr17: 37589696-37589637 | 0.806128 | 0.02352 |
| A_23_P117683 | NM_016400 | chr15: 41881271-41881330 | 1.371049 | 0.02358 |
| A_23_P55706 | NM_006509 | chr19: 50233225-50233284 | 0.691035 | 0.023597 |
| A_23_P6041 | NM_030931 | chr20: 74236-74295 | 0.806468 | 0.0238 |
| A_24_P362646 | NM_005783 | chr2: 99310441-99305057 | 1.510748 | 0.023911 |
| A_23_P86493 | NM_006562 | chr10: 102976808-102976749 | 0.814171 | 0.023921 |
| A_23_P164057 | NM_002404 | chr17: 19227465-19227406 | 0.770818 | 0.023923 |
| A_32_P153604 | THC2519072 | chr2: 242611537-242611478 | 0.738423 | 0.024109 |
| A_23_P60130 | NM_052886 | chr8: 120326738-120326797 | 1.257877 | 0.02418 |
| A_24_P118422 | | chr6: 137263768-137263705 | 0.6525 | 0.024224 |
| A_23_P93258 | NM_003537 | chr6: 26139960-26139901 | 1.336014 | 0.024257 |
| A_24_P503492 | AA805504 | chr1: 172100110-172100169 | 1.386242 | 0.024418 |
| A_24_P180680 | NM_018407 | chr8: 98933757-98933816 | 1.250139 | 0.024458 |
| A_24_P236445 | NM_006141 | chr16: 65313360-65313301 | 1.592733 | 0.024484 |
| A_32_P163169 | NM_003374 | chr5: 133339601-133339542 | 1.598442 | 0.024634 |
| A_23_P37856 | NM_000558 | chr16: 167382-167441 | 0.795316 | 0.024753 |
| A_23_P126803 | NM_005717 | chr1: 181863213-181863154 | 1.596886 | 0.025074 |
| A_32_P24950 | AK056882 | chr9: 84785253-84785194 | 1.278574 | 0.025078 |
| A_24_P288722 | NM_003688 | chrX: 41264528-41264469 | 1.364461 | 0.025088 |
| A_24_P933011 | NM_001080209 | chr18: 5881958-5881899 | 0.847391 | 0.025186 |
| A_23_P204702 | NM_003217 | chr12: 48438529-48438790 | 1.621802 | 0.025223 |
| A_23_P81087 | NM_020395 | chr4: 106827299-106823866 | 1.189324 | 0.025316 |
| A_23_P255273 | NM_002874 | chr9: 109132643-109132702 | 1.543985 | 0.025361 |
| A_23_P37111 | NM_177438 | chr14: 94626045-94625986 | 1.494734 | 0.025382 |
| A_32_P16662 | THC2567500 | chr9: 138454656-138454715 | 1.447718 | 0.025434 |
| A_24_P325281 | AF130093 | chr6: 161418933-161418874 | 1.288764 | 0.025477 |
| A_32_P58937 | NM_017643 | chr17: 46610398-46610339 | 1.318845 | 0.025494 |
| A_32_P175198 | NM_001614 | chr17: 77091659-77091609 | 1.921996 | 0.025581 |
| A_24_P940125 | NM_015455 | chr5: 179937504-179937563 | 1.432495 | 0.02559 |
| A_23_P415827 | NM_015146 | chr5: 132120209-132120150 | 1.478562 | 0.025607 |
| A_23_P141032 | NM_001861 | chr16: 84392353-84396070 | 1.581894 | 0.025754 |
| A_24_P248251 | NM_139048 | chr3: 150276400-150274809 | 1.360541 | 0.025766 |
| A_23_P15146 | NM_001012631 | chr16: 3059309-3059368 | 0.73843 | 0.02578 |
| A_23_P136232 | NM_017813 | chr8: 58038252-58038193 | 1.352812 | 0.02578 |
| A_23_P156807 | AK024077 | chr5: 1640079-1640020 | 2.35 | 0.025819 |
| A_24_P172768 | NM_004124 | chr14: 54011419-54011360 | 1.496706 | 0.025831 |
| A_23_P42746 | NM_000265 | chr7: 73837548-73840289 | 0.78997 | 0.02585 |
| A_32_P179910 | THC2667666 | chr1: 20558503-20558562 | 0.849059 | 0.025978 |
| A_24_P120970 | NM_032937 | chr9: 139630359-139630300 | 1.27801 | 0.025999 |
| A_23_P161501 | NM_024954 | chr10: 99320431-99320490 | 0.835531 | 0.026007 |
| A_23_P69826 | NM_001358 | chr4: 24140442-24140383 | 1.736957 | 0.02605 |
| A_32_P144815 | AA372247 | chr2: 9641907-9641966 | 1.722561 | 0.026092 |
| A_23_P413456 | NR_023312 | chr19: 1225013-1225072 | 1.384867 | 0.026121 |
| A_24_P177964 | NM_001037806 | chr12: 48471273-48471214 | 0.668958 | 0.026147 |
| A_23_P168629 | NM_018077 | chr7: 127738104-127738045 | 0.787404 | 0.026261 |
| A_24_P319369 | NM_016946 | chr1: 159233364-159233305 | 1.357748 | 0.026317 |
| A_24_P911676 | NM_003107 | chr6: 21705980-21706039 | 1.455463 | 0.026345 |
| A_24_P239076 | NM_001013618 | chr22: 24045796-24045855 | 0.858847 | 0.026367 |
| A_32_P199824 | THC2541992 | chr1: 200166856-200166915 | 0.861013 | 0.026377 |
| A_23_P131020 | NM_017660 | chr19: 19478471-19478530 | 1.475814 | 0.026483 |
| A_23_P113634 | NM_001755 | chr16: 65692131-65692190 | 1.402948 | 0.026541 |
| A_24_P126325 | AK027318 | chr1: 25443570-25443511 | 1.441961 | 0.02661 |
| A_23_P144578 | NM_138335 | chr4: 44399194-44399135 | 1.425093 | 0.026614 |
| A_23_P131255 | NM_015535 | chr2: 201051273-201051332 | 1.474952 | 0.026649 |
| A_23_P165086 | NM_024407 | chr19: 1346507-1346566 | 0.841375 | 0.026652 |
| A_23_P2026 | NM_005706 | chr11: 2381355-2381414 | 0.808217 | 0.026682 |
| A_23_P377616 | NR_023312 | chr19: 1225007-1225066 | 1.394385 | 0.026726 |
| A_24_P417546 | BC004936 | chr4: 83845462-83845403 | 0.856797 | 0.026807 |
| A_23_P108294 | NM_177543 | chr19: 233253-233194 | 1.282695 | 0.026864 |
| A_24_P101261 | | chr5: 55170354-55170334 | 1.515543 | 0.026986 |
| A_24_P227326 | NM_173587 | chr11: 63436045-63435986 | 0.834416 | 0.027052 |
| A_24_P221007 | ENST00000358984 | chr18: 14821396-14827210 | 1.10844 | 0.027125 |
| A_23_P60089 | NM_024940 | chr8: 25325088-25325147 | 1.425493 | 0.027154 |
| A_23_P254091 | NM_004520 | chr5: 61717769-61717828 | 1.573382 | 0.027158 |
| A_24_P488083 | AJ519285 | chr14: 105796321-105796262 | 0.881327 | 0.027159 |
| A_23_P17998 | NM_005524 | chr3: 195338870-195338929 | 1.338146 | 0.02719 |
| A_24_P145653 | NM_004672 | chr1: 27554326-27554267 | 1.519252 | 0.027307 |
| A_23_P256342 | NM_015132 | chr7: 17799719-17799660 | 1.647324 | 0.027323 |
| A_24_P289447 | NM_014827 | chr1: 202088118-202088177 | 1.530216 | 0.027381 |
| A_24_P284420 | NM_001005862 | chr17: 35120176-35120235 | 1.219462 | 0.027389 |
| A_23_P67339 | NM_020650 | chr19: 54737809-54738221 | 0.787484 | 0.02743 |
| A_23_P3922 | AK021772 | chr17: 52712285-52712344 | 0.75561 | 0.027509 |
| A_23_P428373 | NM_172239 | chr8: 86756320-86756261 | 0.868349 | 0.027528 |
| A_24_P83944 | NM_017652 | chr19: 62979749-62979807 | 1.461613 | 0.027613 |
| A_23_P112113 | NM_014846 | chr8: 126106049-126105990 | 1.364022 | 0.027632 |
| A_23_P141194 | NM_001545 | chr17: 70528873-70528932 | 1.347583 | 0.027774 |
| A_24_P778649 | NM_002393 | chr1: 202789445-202789504 | 1.47771 | 0.027803 |
| A_23_P117527 | NM_017943 | chr14: 54889561-54889620 | 1.672576 | 0.028118 |
| A_24_P50738 | AF035020 | chr14: 105523839-105523787 | 0.873991 | 0.028221 |
| A_24_P494425 | ENST00000390275 | chr2: 89830567-89830626 | 0.86517 | 0.028224 |
| A_23_P2960 | NM_005163 | chr14: 104310384-104310325 | 1.381726 | 0.028255 |
| A_23_P117298 | NM_000131 | chr13: 112821879-112821938 | 0.793556 | 0.028264 |
| A_23_P152181 | NM_018119 | chr16: 22250992-22252509 | 1.719977 | 0.028304 |
| A_23_P170110 | AF227517 | chr5: 141667671-141667612 | 1.315713 | 0.028483 |
| A_32_P154256 | NM_006826 | chr2: 9642004-9641945 | 1.470885 | 0.028488 |
| A_24_P92174 | ENST00000390597 | chr14: 105565315-105565256 | 0.882992 | 0.028491 |
| A_23_P428999 | NM_006540 | chr8: 71186978-71186919 | 1.332432 | 0.028626 |
| A_23_P110846 | NM_004779 | chr5: 154236006-154236065 | 1.418569 | 0.02874 |
| A_23_P211488 | NM_145637 | chr22: 34953586-34953527 | 0.731131 | 0.028787 |
| A_23_P213038 | NM_024751 | chr4: 106986481-106986540 | 1.34839 | 0.028811 |
| A_24_P566916 | NR_002802 | chr11: 64949850-64949909 | 1.468306 | 0.028822 |
| A_23_P257755 | NM_022780 | chr2: 86854542-86854601 | 1.426526 | 0.028879 |
| A_24_P664995 | NM_001127322 | chr12: 52911121-52911062 | 1.413988 | 0.028938 |
| A_23_P251893 | NM_152743 | chr7: 2544098-2544039 | 1.381686 | 0.029025 |
| A_23_P119502 | NM_003775 | chr19: 3131249-3131308 | 0.809373 | 0.029048 |
| A_23_P4069 | NM_001004313 | chr17: 10569088-10567349 | 0.834926 | 0.029158 |
| A_23_P206998 | NM_004687 | chr17: 53924907-53924178 | 1.389275 | 0.029186 |
| A_23_P342654 | NM_147150 | chr9: 111971916-111971975 | 0.815973 | 0.029266 |
| A_32_P1291 | | chr20: 062165036-062164977 | 0.79162 | 0.029301 |
| A_32_P121483 | | chr18: 011624446-011624505 | 1.578417 | 0.029323 |
| A_23_P7873 | NM_002388 | chr6: 52236911-52236852 | 1.586556 | 0.029375 |
| A_23_P43283 | NM_032777 | chr8: 37819862-37819921 | 0.63161 | 0.029464 |
| A_23_P165280 | NM_002695 | chr19: 1041116-1040933 | 0.8521 | 0.029548 |
| A_24_P692566 | AF283771 | chr13: 29933154-29933213 | 1.716214 | 0.02961 |
| A_24_P173790 | AK055963 | chr1: 226850835-226850894 | 0.725936 | 0.029754 |
| A_24_P362394 | NM_007259 | chr1: 148321535-148330657 | 1.526409 | 0.029781 |
| A_23_P169154 | NM_024896 | chr9: 5775753-5775694 | 1.364125 | 0.02983 |
| A_24_P398898 | NM_206839 | chr15: 76976728-76976787 | 1.817059 | 0.029831 |
| A_24_P50157 | XR_017483 | chr6: 151588395-151588356 | 0.706265 | 0.030006 |
| A_24_P2995 | NM_015317 | chr2: 20313285-20313226 | 1.497309 | 0.030014 |
| A_24_P25470 | BC027922 | chr19: 63003346-63003405 | 1.323173 | 0.030059 |
| A_32_P27636 | BX099985 | chr1: 031184633-031184692 | 1.541096 | 0.030093 |
| A_24_P522631 | NM_001130924 | chr1: 9597245-9597303 | 0.830335 | 0.030183 |
| A_32_P119154 | BE049152 | chr1: 154762068-154762127 | 1.252795 | 0.030292 |
| A_24_P316381 | BC006271 | chr17: 41526911-41526852 | 1.423726 | 0.030323 |
| A_32_P96838 | AF119911 | chr5: 148856398-148856457 | 1.410752 | 0.03033 |
| A_23_P136753 | THC2559929 | chr14: 105241401-105241343 | 0.844114 | 0.030489 |
| A_23_P251945 | NM_016221 | chr5: 150069117-150069058 | 1.578337 | 0.030549 |
| A_23_P218079 | NM_018976 | chr12: 45040951-45040892 | 1.378673 | 0.030585 |
| A_23_P201619 | NM_133494 | chr1: 196556743-196556802 | 1.472258 | 0.030608 |
| A_24_P267814 | BI521983 | chr22: 021573253-021573194 | 0.880872 | 0.030692 |
| A_24_P178602 | NM_198457 | chr19: 57961442-57961383 | 1.453154 | 0.030748 |
| A_23_P143303 | NM_080414 | chr20: 2794106-2794881 | 1.639964 | 0.030922 |
| A_24_P524262 | THC2522342 | chr15: 30602568-30593602 | 0.728651 | 0.031006 |
| A_23_P151159 | NM_032300 | chr12: 108839020-108839079 | 1.68086 | 0.03105 |
| A_23_P502224 | NM_007326 | chr22: 41344873-41344814 | 1.523171 | 0.031101 |
| A_23_P159893 | NM_145234 | chrX: 109805902-109805843 | 0.795284 | 0.031225 |
| A_23_P306507 | NM_033360 | chr12: 25250478-25250419 | 1.337237 | 0.031287 |
| A_24_P1910 | NM_003168 | chr17: 53777730-53777671 | 1.353359 | 0.03137 |
| A_23_P141863 | NM_014480 | chr19: 63466011-63466071 | 1.595351 | 0.031405 |
| A_24_P199905 | NM_006826 | chr2: 9642024-9641965 | 1.544834 | 0.03163 |
| A_24_P573978 | ENST00000409764 | chr7: 025131375-025130961 | 1.480536 | 0.031688 |
| A_23_P77048 | NM_001039355 | chr14: 99827300-99827241 | 1.273083 | 0.031743 |
| A_23_P49674 | NM_173728 | chr17: 8165669-8165728 | 0.767894 | 0.031747 |
| A_32_P92642 | BE710245 | chr2: 215938588-215940879 | 1.271372 | 0.031783 |
| A_32_P202719 | THC2644672 | chr16: 1138709-1138650 | 0.854257 | 0.031834 |
| A_23_P399001 | NM_016463 | chr5: 139042720-139042779 | 1.444208 | 0.031888 |
| A_23_P130780 | AF182077 | chr19: 52898036-52898097 | 0.806822 | 0.031973 |
| A_23_P165691 | NM_005805 | chr2: 161973673-161973732 | 1.459458 | 0.032166 |
| A_23_P71148 | NM_000712 | chr7: 43813367-43813426 | 1.382051 | 0.032183 |
| A_23_P92954 | NM_000414 | chr5: 118894966-118900043 | 1.456996 | 0.03229 |
| A_24_P369154 | NM_002721 | chr9: 126951892-126951833 | 1.591958 | 0.032378 |
| A_24_P110487 | | chr16: 033554888-033554839 | 0.880437 | 0.032521 |
| A_32_P351277 | BC004968 | chr16: 668578-668519 | 0.869132 | 0.032546 |
| A_23_P108761 | NM_017546 | chr2: 101252237-101252296 | 1.511241 | 0.032551 |
| A_23_P130466 | NM_021089 | chr19: 63498854-63498913 | 1.756433 | 0.032561 |
| A_24_P942760 | | chr15: 056938987-056938928 | 1.420764 | 0.032621 |
| A_24_P237328 | NM_014507 | chr22: 41867185-41867126 | 0.794457 | 0.032649 |
| A_23_P157435 | NM_015878 | chr8: 103909610-103909551 | 1.330274 | 0.032715 |
| A_32_P79115 | XR_039086 | chr1: 147940002-147939943 | 0.851052 | 0.032726 |
| A_24_P63608 | NM_017838 | chr5: 177510569-177510510 | 1.615523 | 0.03273 |
| A_23_P51548 | NM_004528 | chr1: 163891312-163891371 | 1.432034 | 0.032733 |
| A_32_P168247 | NM_004373 | chr12: 119362800-119362859 | 1.314326 | 0.032742 |
| A_23_P414913 | NM_022343 | chr9: 36153763-36153822 | 0.804745 | 0.032863 |
| A_23_P103743 | AF118066 | chr1: 110688818-110688759 | 1.325611 | 0.032867 |
| A_23_P64669 | NM_017612 | chr12: 121523731-121523672 | 1.343247 | 0.032938 |
| A_24_P523325 | THC2547195 | chr4: 039225662-039225721 | 1.356715 | 0.033144 |
| A_23_P155197 | ENST00000383657 | chr3: 124695221-124695162 | 1.321899 | 0.03321 |
| A_23_P41280 | NM_001079525 | chr4: 57020295-57020354 | 1.392134 | 0.033212 |
| A_24_P927614 | AY358247 | chr3: 131245191-131245250 | 0.740619 | 0.033322 |
| A_24_P39654 | NM_002086 | chr17: 70840417-70833679 | 1.359786 | 0.03334 |
| A_23_P257672 | NM_177458 | chr8: 143842833-143842774 | 0.868668 | 0.033417 |
| A_23_P356484 | NM_001014 | chr6: 34493274-34493215 | 1.683713 | 0.033453 |
| A_23_P68628 | NM_031232 | chr20: 31709184-31709125 | 1.422222 | 0.033502 |
| A_23_P327307 | NM_002577 | chr3: 198039598-198039657 | 1.706781 | 0.03352 |
| A_23_P93046 | NM_007277 | chr5: 517408-517467 | 1.349113 | 0.033576 |
| A_23_P19936 | NM_006854 | chr7: 6468890-6468831 | 1.305042 | 0.033581 |
| A_24_P8130 | AK092421 | chr11: 000871417-000871358 | 0.782442 | 0.03368 |
| A_23_P9688 | NM_006141 | chr16: 65315211-65315152 | 1.611943 | 0.033711 |
| A_23_P424080 | NM_032312 | chr2: 32384445-32384504 | 1.523212 | 0.033742 |
| A_23_P100660 | NM_002615 | chr17: 1627418-1627477 | 0.731354 | 0.03376 |
| A_23_P118392 | NM_016084 | chr17: 17338571-17338512 | 0.830591 | 0.033978 |
| A_24_P384369 | | chr5: 36922186-36922245 | 0.758762 | 0.034007 |
| A_23_P404821 | NM_001080392 | chr7: 141003503-141003444 | 1.357862 | 0.03404 |
| A_23_P206568 | | chr16: 030004453-030004394 | 0.811422 | 0.034111 |
| A_23_P106056 | NM_001344 | chr14: 22103645-22103633 | 1.451023 | 0.034255 |
| A_32_P71571 | NM_182522 | chr3: 68863714-68863660 | 0.901523 | 0.034322 |
| A_23_P13969 | BC052611 | chr12: 119140440-119140381 | 0.777679 | 0.034366 |
| A_23_P80643 | NM_006515 | chr3: 4331190-4331249 | 0.704318 | 0.03443 |
| A_23_P200507 | NM_014184 | chr1: 222630162-222630221 | 1.462656 | 0.034455 |
| A_32_P85500 | NM_006674 | chr6: 31540998-31541057 | 0.76935 | 0.034558 |
| A_23_P214108 | NM_000367 | chr6: 18238959-18238900 | 1.384351 | 0.034709 |
| A_32_P146450 | AW948903 | chr12: 95187353-95187334 | 0.804985 | 0.034805 |
| A_23_P502575 | NM_177559 | chr20: 412048-411989 | 1.595477 | 0.034956 |
| A_24_P12181 | NM_152277 | chr5: 171571530-171571471 | 1.525686 | 0.035026 |
| A_23_P55076 | NM_130766 | chr17: 1344681-1344622 | 0.784175 | 0.035051 |
| A_23_P108604 | NM_145686 | chr2: 101871744-101871803 | 1.512454 | 0.03511 |
| A_23_P252250 | AK022183 | chr5: 179137866-179137925 | 0.863498 | 0.035171 |
| A_24_P188071 | NM_032704 | chr12: 47953297-47953356 | 1.285629 | 0.035208 |
| A_23_P6355 | NR_024591 | chr22: 17226105-17226164 | 0.807529 | 0.035247 |
| A_23_P79043 | NM_032635 | chr19: 40729743-40729891 | 1.448511 | 0.035251 |
| A_23_P56314 | NM_006830 | chr19: 1549153-1549094 | 1.333115 | 0.035258 |
| A_24_P212024 | ENST00000390258 | chr2: 89294168-89294109 | 0.84118 | 0.03526 |
| A_23_P70355 | NM_004568 | chr6: 2900770-2899897 | 1.461822 | 0.035336 |
| A_32_P157965 | NM_003908 | chr20: 32145142-32142011 | 1.548486 | 0.035348 |
| A_23_P150446 | NM_006360 | chr11: 32579942-32580457 | 1.398667 | 0.035456 |
| A_23_P2366 | NM_199040 | chr12: 92317594-92317653 | 1.415584 | 0.03551 |
| A_23_P8906 | NM_013437 | chr8: 105571698-105571639 | 1.237529 | 0.035952 |
| A_24_P260582 | CR603450 | chr5: 180615030-180614971 | 1.341204 | 0.035993 |
| A_23_P218523 | NM_021731 | chr19: 3493415-3493356 | 1.739159 | 0.03613 |
| A_24_P3973 | NM_002137 | chr7: 26199500-26199441 | 1.504426 | 0.036334 |
| A_23_P126398 | NM_012432 | chr1: 149202643-149202702 | 1.48898 | 0.036366 |
| A_24_P23245 | NM_002490 | chr22: 40813023-40812219 | 1.409136 | 0.03645 |
| A_23_P25735 | NM_002791 | chr14: 34856247-34856306 | 1.578929 | 0.036713 |
| A_23_P251387 | NM_004726 | chrX: 17075549-17075608 | 1.275275 | 0.036907 |
| A_23_P165535 | NM_001959 | chr2: 206733654-206734348 | 0.7652 | 0.036942 |
| A_24_P524588 | | chr4: 548835-548776 | 0.87648 | 0.037163 |
| A_24_P38387 | NM_006096 | chr8: 134335984-134331930 | 1.26501 | 0.037282 |
| A_32_P36778 | THC2663978 | chr16: 30176473-30176532 | 1.714688 | 0.037319 |
| A_23_P148297 | NM_003022 | chrX: 80440198-80440257 | 1.255688 | 0.037352 |
| A_23_P97157 | NM_018085 | chr1: 200110680-200110954 | 1.496679 | 0.037431 |
| A_23_P108708 | NM_016494 | chr2: 85677497-85677556 | 1.57306 | 0.037506 |
| A_24_P24371 | ENST00000390543 | chr14: 105163190-105162739 | 0.889766 | 0.037598 |
| A_23_P503182 | NM_021962 | chr17: 853700-853641 | 1.530976 | 0.037645 |
| A_24_P89284 | NM_002109 | chr5: 140034842-140034592 | 1.592148 | 0.03771 |
| A_24_P326957 | NM_025230 | chr14: 23662877-23662936 | 0.771131 | 0.037749 |
| A_23_P96623 | NM_000513 | chrX: 153108745-153108804 | 0.844817 | 0.037786 |
| A_23_P207020 | NM_019613 | chr17: 78165910-78165851 | 1.264069 | 0.037866 |
| A_23_P33256 | NM_000174 | chr3: 130263741-130263800 | 0.837393 | 0.037897 |
| A_23_P259272 | NM_018639 | chr12: 116955220-116955161 | 1.494159 | 0.038013 |
| A_24_P124097 | NM_022833 | chr9: 129309127-129309068 | 0.820753 | 0.038145 |
| A_23_P417404 | AF151783 | chr9: 129307548-129307489 | 1.446852 | 0.038191 |
| A_23_P144224 | NM_003262 | chr3: 171193472-171193531 | 1.506162 | 0.038213 |
| A_24_P929650 | | chr9: 139256976-139257034 | 1.348773 | 0.038252 |
| A_24_P347854 | NM_004710 | chr17: 73678502-73678561 | 1.318786 | 0.038259 |
| A_23_P33433 | NM_001042539 | chr16: 29729134-29729193 | 1.30347 | 0.038304 |
| A_24_P910733 | NM_178335 | chr3: 192583279-192590008 | 0.700609 | 0.038532 |
| A_23_P306956 | NM_032838 | chr19: 41631076-41631017 | 1.417062 | 0.038533 |
| A_23_P120237 | NM_020151 | chr2: 96214787-96214728 | 1.562521 | 0.038616 |
| A_24_P105794 | NM_001099693 | chr2: 100989162-100989221 | 1.246768 | 0.038627 |
| A_23_P76529 | NM_000889 | chr12: 51871483-51871424 | 0.733734 | 0.038643 |
| A_23_P34983 | NM_006694 | chr1: 152215845-152214999 | 1.56636 | 0.038648 |
| A_23_P373126 | BC035647 | chr6: 30004277-30004779 | 0.773204 | 0.038657 |
| A_24_P313804 | NM_032828 | chr19: 63065975-63066034 | 1.425655 | 0.038736 |
| A_24_P182858 | NM_030915 | chr2: 30333823-30333882 | 0.875649 | 0.038764 |
| A_24_P358138 | NM_173632 | chr19: 62956599-62956658 | 1.44028 | 0.038779 |
| A_23_P4821 | NM_002229 | chr19: 12765047-12765106 | 0.801317 | 0.038826 |
| A_23_P107369 | U91329 | chr17: 4868551-4868605 | 0.816808 | 0.038865 |
| A_23_P127175 | NM_020150 | chr10: 71582273-71582214 | 1.517216 | 0.038902 |
| A_24_P350252 | | chr11: 000970519-000970460 | 0.861205 | 0.038914 |
| A_24_P374652 | NM_022731 | chr1: 203953835-203953776 | 1.394759 | 0.038922 |
| A_23_P66798 | NM_002276 | chr17: 36933503-36933444 | 1.294414 | 0.039125 |
| A_24_P194000 | NM_005500 | chr19: 52405205-52405264 | 1.33191 | 0.039138 |
| A_23_P218126 | BC024289 | chr14: 105279355-105279296 | 0.879021 | 0.039138 |
| A_24_P105501 | NM_001428 | chr1: 8846607-8846548 | 1.430823 | 0.039422 |
| A_32_P98423 | NM_004772 | chr5: 111092923-111092892 | 1.396499 | 0.039617 |
| A_23_P205098 | NM_015032 | chr13: 32249525-32249584 | 1.325589 | 0.039759 |
| A_24_P924591 | NM_007146 | chr17: 53404011-53403952 | 1.393048 | 0.039808 |
| A_23_P90845 | NM_022173 | chr2: 70293319-70293260 | 1.46995 | 0.039809 |
| A_23_P132358 | NM_173467 | chr22: 41859137-41859078 | 0.870977 | 0.03981 |
| A_24_P392333 | NM_001040453 | chr15: 56926846-56926905 | 1.465075 | 0.039999 |
| A_23_P401675 | NM_144724 | chr5: 68775506-68775565 | 1.337899 | 0.040002 |
| A_32_P86517 | AI669361 | chr12: 45040852-45040911 | 1.310954 | 0.040094 |
| A_24_P22163 | NM_177438 | chr14: 94627337-94627185 | 1.777241 | 0.040101 |
| A_23_P148541 | NM_139250 | chrX: 153468202-153468261 | 0.892633 | 0.040154 |
| A_32_P387905 | AK024224 | chr14: 52177444-52177385 | 1.28101 | 0.040353 |
| A_23_P107051 | NM_003673 | chr17: 35076266-35076325 | 1.163285 | 0.04039 |
| A_24_P159135 | NM_006156 | chr14: 23771296-23757251 | 1.419289 | 0.040562 |
| A_24_P195749 | | chr4: 039651569-039651508 | 1.325696 | 0.040671 |
| A_32_P162726 | ENST00000288063 | chr16: 68838310-68838251 | 1.469384 | 0.040814 |
| A_24_P289366 | NM_203463 | chr2: 169337799-169337858 | 1.257885 | 0.040822 |
| A_23_P129786 | NM_001005291 | chr17: 17656203-17656144 | 1.234005 | 0.040983 |
| A_23_P153022 | NM_033184 | chr17: 36474895-36474846 | 0.868289 | 0.041062 |
| A_32_P102745 | R78313 | chr11: 71610548-71610489 | 0.768422 | 0.04114 |
| A_24_P942092 | AK024362 | chr19: 56900148-56900207 | 1.349406 | 0.041182 |
| A_24_P12149 | NM_014666 | chr5: 157146444-157146385 | 1.259475 | 0.041239 |
| A_23_P28057 | NM_003249 | chr19: 2764491-2764550 | 1.51488 | 0.041281 |
| A_23_P51572 | NM_005999 | chr1: 229767022-229767081 | 1.467748 | 0.041289 |
| A_32_P234780 | THC2519484 | chr4: 120598643-120598702 | 1.375414 | 0.041353 |
| A_23_P51951 | NM_006850 | chr1: 205143594-205143653 | 0.832532 | 0.041413 |
| A_23_P72550 | NM_003565 | chr12: 130973529-130973588 | 1.405094 | 0.041506 |
| A_23_P150057 | NM_024756 | chr10: 88685874-88685815 | 0.709037 | 0.041664 |
| A_32_P162076 | CR749652 | chr1: 20943476-20943535 | 1.467955 | 0.041698 |
| A_32_P55144 | BX537816 | chr4: 1139458-1139517 | 0.855107 | 0.041809 |
| A_24_P627678 | | chr1: 026674404-026674455 | 1.416217 | 0.041932 |
| A_24_P826646 | NM_001037738 | chr5: 170747510-170747567 | 1.585383 | 0.04205 |
| A_23_P90444 | NM_024321 | chr19: 40819992-40820051 | 1.540323 | 0.042161 |
| A_23_P31323 | NM_001101 | chr7: 5534460-5534306 | 1.662553 | 0.042226 |
| A_32_P36182 | BE935032 | chrX: 16786767-16786826 | 1.37089 | 0.042236 |
| A_32_P805871 | NM_033310 | chr11: 63820981-63821040 | 0.877374 | 0.042352 |
| A_23_P88439 | NM_152332 | chr14: 91317428-91317369 | 1.247652 | 0.042368 |
| A_23_P73559 | NM_004463 | chrX: 54488965-54488906 | 0.796551 | 0.042387 |
| A_23_P353744 | NM_032239 | chr4: 129249416-129249475 | 1.414361 | 0.042427 |
| A_24_P42122 | NM_001533 | chr19: 44020125-44019953 | 1.597803 | 0.042431 |
| A_23_P33216 | NM_001686 | chr12: 55322579-55322520 | 1.480477 | 0.042531 |
| A_24_P315564 | | chr8: 11920231-11920172 | 0.657183 | 0.042553 |
| A_23_P142310 | NM_017572 | chr19: 1988572-1988513 | 1.440809 | 0.042557 |
| A_32_P83221 | BE064950 | chr17: 1273194-1273253 | 1.325968 | 0.042892 |
| A_23_P148858 | AF010144 | chr1: 23905250-23905191 | 0.768747 | 0.042988 |
| A_32_P114918 | THC2771832 | chrX: 48655687-48655746 | 0.818087 | 0.043217 |
| A_23_P128991 | NM_031210 | chr14: 77251917-77253592 | 1.433939 | 0.043267 |
| A_23_P207493 | NM_016424 | chr17: 46184287-46184346 | 1.229976 | 0.043295 |
| A_24_P912366 | NM_001109977 | chr4: 152718267-152718326 | 1.33005 | 0.043373 |
| A_32_P177595 | | chr5: 127552225-127552166 | 1.187251 | 0.043381 |
| A_23_P259413 | NM_017548 | chr3: 134788150-134788209 | 0.658902 | 0.043418 |
| A_23_P69877 | NM_152283 | chr5_random: 12747-12688 | 1.31876 | 0.043648 |
| A_32_P46171 | CA314185 | chr4_random: 766703-766644 | 0.792042 | 0.043816 |
| A_24_P393811 | NM_019026 | chr1: 163979091-163979032 | 1.435705 | 0.043925 |
| A_24_P809567 | AA807748 | chr1: 23484439-23484498 | 1.331251 | 0.044038 |
| A_23_P124760 | NM_014815 | chr17: 35431822-35431763 | 1.185512 | 0.044074 |
| A_32_P198303 | NM_181706 | chr11: 31410597-31410656 | 1.481357 | 0.044088 |
| A_23_P132405 | NM_014049 | chr3: 130114530-130114589 | 1.495325 | 0.044111 |
| A_24_P848662 | CR594528 | chr16: 645331-645272 | 0.871024 | 0.044245 |
| A_23_P200741 | NM_001937 | chr1: 166931431-166931372 | 0.822171 | 0.044352 |
| A_23_P356624 | NM_152905 | chr12: 95870046-95870105 | 1.39397 | 0.044552 |
| A_32_P97513 | THC2679050 | chr20: 015711101-015711160 | 0.864301 | 0.044716 |
| A_23_P386241 | NM_001042353 | chr20: 774839-774898 | 1.349733 | 0.044834 |
| A_23_P46070 | | chr1: 165661446-165661387 | 0.846161 | 0.044854 |
| A_23_P384072 | NM_013333 | chr19: 60896212-60897959 | 0.860847 | 0.044945 |
| A_23_P156732 | NM_024165 | chr6: 33491907-33491966 | 0.818608 | 0.045006 |
| A_32_P191895 | BC045716 | chr8: 71017698-71017757 | 1.470571 | 0.045008 |
| A_23_P50504 | NM_000146 | chr19: 54160543-54160602 | 1.449956 | 0.045019 |
| A_32_P41035 | AK092942 | chr7: 051421249-051421190 | 1.340406 | 0.045088 |
| A_24_P642240 | THC2659814 | chr19: 13852316-13852257 | 0.876398 | 0.04526 |
| A_24_P276583 | NM_019026 | chr1: 163979071-163963967 | 1.275242 | 0.045264 |
| A_32_P170368 | THC2538856 | chr5: 176913753-176913812 | 0.834095 | 0.045317 |
| A_23_P323166 | NM_016333 | chr16: 2761189-2761248 | 0.713253 | 0.045324 |
| A_24_P341958 | NM_178862 | chr3: 31636191-31636250 | 1.440135 | 0.045374 |
| A_32_P145443 | AI161396 | chr14: 76560740-76560799 | 1.283095 | 0.045423 |
| A_23_P250042 | NM_016275 | chr3: 151828263-151828322 | 1.459493 | 0.045441 |
| A_32_P221799 | NM_003514 | chr6: 27968545-27968486 | 1.294028 | 0.04551 |
| A_23_P25935 | NM_007176 | chr14: 75187656-75187597 | 1.471544 | 0.045666 |
| A_24_P786912 | | chr8: 000735716-000735657 | 0.885348 | 0.045743 |
| A_23_P383819 | NM_016569 | chr12: 113593282-113593223 | 1.279813 | 0.04579 |
| A_32_P5417 | CA946373 | chr3: 46454514-46454573 | 0.895303 | 0.045794 |
| A_24_P410017 | NM_001083538 | chr2: 131131616-131131675 | 1.722324 | 0.046074 |
| A_24_P411899 | NM_183419 | chr8: 101339026-101338967 | 1.273718 | 0.046098 |
| A_23_P132973 | NM_001042616 | chr4: 89661354-89661295 | 1.671806 | 0.046155 |
| A_24_P333364 | NM_001042535 | chr7: 150451459-150451518 | 0.843529 | 0.046248 |
| A_32_P67414 | BC033990 | chr2: 241146839-241146898 | 0.818406 | 0.046308 |
| A_23_P502915 | NM_017491 | chr4: 9685449-9685390 | 1.575468 | 0.046313 |
| A_23_P258641 | NM_002709 | chr2: 28865114-28865172 | 1.384135 | 0.046339 |
| A_23_P100203 | NM_001537 | chr16: 82403745-82403804 | 1.210966 | 0.046399 |
| A_32_P62469 | BQ307884 | chr1: 149643199-149643258 | 1.445595 | 0.046469 |
| A_23_P325891 | NM_015635 | chr9: 127165025-127165084 | 1.372887 | 0.046645 |
| A_23_P200866 | NM_203401 | chr1: 26099739-26099680 | 1.368866 | 0.046728 |
| A_23_P214587 | NM_003449 | chr6: 30260900-30260841 | 1.659112 | 0.046772 |
| A_23_P100196 | NM_005153 | chr16: 83370332-83370391 | 1.540506 | 0.046796 |
| A_23_P395534 | NM_006511 | chr1: 15860726-15860785 | 1.595613 | 0.046886 |
| A_23_P203095 | NM_004716 | chr11: 116583043-116582716 | 0.636352 | 0.046922 |
| A_24_P565496 | | chr14: 103745190-103745249 | 0.870809 | 0.046959 |
| A_23_P257924 | NM_005239 | chr21: 39117433-39117492 | 0.658329 | 0.046977 |
| A_32_P108655 | NM_001005353 | chr1: 65465220-65465279 | 1.266594 | 0.046979 |
| A_32_P25437 | NM_001046 | chr5: 127552627-127552686 | 1.192342 | 0.047007 |
| A_24_P330691 | NM_006937 | chr17: 70675787-70675728 | 1.493449 | 0.047023 |
| A_32_P167791 | NM_003932 | chr22: 39550554-39550527 | 0.686139 | 0.047065 |
| A_32_P175313 | THC2521437 | chr1: 28696846-28696787 | 0.84746 | 0.047082 |
| A_23_P2143 | NM_014752 | chr11: 74366137-74366196 | 1.314624 | 0.047209 |
| A_23_P106299 | NM_001018108 | chr15: 41873224-41873283 | 1.494451 | 0.047227 |
| A_23_P53397 | NM_138473 | chr12: 52091499-52091558 | 1.636345 | 0.047417 |
| A_23_P138271 | NM_138795 | chr1: 200369355-200369296 | 0.680679 | 0.047425 |
| A_23_P151653 | NM_080649 | chr14: 19994086-19994711 | 1.427042 | 0.047471 |
| A_23_P58647 | NM_001903 | chr5: 138298286-138298345 | 1.277982 | 0.047474 |
| A_23_P97283 | NM_024897 | chr1: 154480041-154479982 | 0.843583 | 0.047563 |
| A_23_P314070 | NM_004308 | chr11: 46655301-46655242 | 1.415857 | 0.047584 |
| A_24_P13230 | NM_002869 | chr11: 73065020-73064961 | 1.316306 | 0.047638 |
| A_24_P861009 | NM_001007246 | chr21: 39590863-39590804 | 1.297749 | 0.047671 |
| A_32_P144629 | | chr8: 100094336-100094277 | 1.425409 | 0.047788 |
| A_23_P79628 | NM_014614 | chr2: 53946122-53946063 | 1.529893 | 0.047789 |
| A_24_P925222 | | chr22: 016955961-016956021 | 1.33225 | 0.047973 |
| A_23_P54956 | NM_032478 | chr17: 71406385-71406326 | 1.391902 | 0.048028 |
| A_23_P85969 | NM_182976 | chr1: 90266294-90266353 | 1.421634 | 0.048047 |
| A_23_P118544 | NM_004859 | chr17: 55126681-55126740 | 1.276686 | 0.048145 |
| A_24_P141995 | NM_032632 | chr14: 96102917-96102976 | 1.477176 | 0.048395 |
| A_24_P381962 | NM_001030007 | chr16: 70320728-70320669 | 1.369548 | 0.048446 |
| A_24_P97770 | NM_001031711 | chr5: 172269310-172269369 | 1.410889 | 0.048461 |
| A_32_P56382 | THC2699247 | chr8: 142072250-142072309 | 1.297438 | 0.048515 |
| A_23_P46627 | NM_015999 | chr1: 201176948-201176889 | 1.383045 | 0.048726 |
| A_24_P44916 | NM_145057 | chr19: 59668157-59668098 | 0.850559 | 0.048763 |
| A_24_P849801 | NM_000983 | chr1: 6168685-6168626 | 1.418497 | 0.048809 |
| A_32_P132422 | | chr19: 061291424-061291483 | 0.841766 | 0.048839 |
| A_23_P94857 | NM_016219 | chr9: 139122689-139122748 | 1.349899 | 0.048848 |
| A_23_P79545 | NM_003849 | chr2: 84512281-84512222 | 1.456603 | 0.048919 |
| A_23_P71680 | BC034271 | chr9: 96925551-96925611 | 0.825243 | 0.048945 |
| A_23_P119812 | NM_000408 | chr2: 157147901-157147960 | 1.275792 | 0.049008 |
| A_23_P312863 | NM_006088 | chr9: 139257467-139257526 | 1.379121 | 0.049199 |
| A_23_P28797 | THC2525955 | chr20: 47184716-47184775 | 1.480611 | 0.049251 |
| A_23_P303851 | NM_176886 | | 1.247055 | 0.049385 |
| A_24_P82957 | NM_018683 | chr20: 48003030-48003089 | 1.427302 | 0.04954 |
| A_32_P212817 | AI335947 | chr5: 132298391-132298450 | 1.183238 | 0.049577 |
| A_23_P55091 | NM_017647 | chr17: 59250991-59250932 | 1.292043 | 0.049609 |
| A_32_P175739 | NM_000189 | chr2: 74973839-74973898 | 1.281761 | 0.049621 |
| A_23_P138899 | NM_016451 | chr11: 14439322-14438375 | 1.440983 | 0.049723 |
| A_24_P75308 | | chr14: 21289423-21289482 | 0.794978 | 0.049898 |
| A_23_P27724 | NM_003009 | chr19: 52979639-52979698 | 1.494376 | 0.0499 |
| A_23_P201605 | NM_144977 | chr1: 195831003-195818990 | 1.309349 | 0.049941 |
| A_32_P56618 | XM_001726460 | chr7: 62861124-62861117 | 0.866754 | 0.049944 |

TABLE 14

| | TargetID | | Hazard | |
|---|---|---|---|---|
| ProbeID | (GenBank Ref.) | Genomic Coordinates | ratio | p-value |
| A_23_P154065 | NM_006000 | chr2: 219823373-219823314 | 0.70555 | 0.015179 |
| A_24_P267997 | NM_015602 | chr1: 178153793-178153852 | 1.792605 | 0.036652 |
| A_23_P60977 | NM_014387 | chr16: 28905470-28905529 | 0.658161 | 0.033553 |
| A_23_P502274 | NM_002751 | chr22: 49044413-49044354 | 0.744231 | 0.015208 |
| A_24_P940086 | NM_007225 | chr17: 45015492-45015551 | 0.768989 | 0.002654 |
| A_23_P88228 | NM_138790 | chr14: 104467387-104467446 | 0.758539 | 0.008129 |
| A_32_P93328 | NR_024454 | chr7: 140042807-140042748 | 0.812596 | 0.008124 |
| A_23_P87329 | NM_024662 | chr11: 34124566-34124625 | 0.679178 | 0.007668 |
| A_32_P20492 | THC2560850 | chr1: 22291591-22291532 | 1.478865 | 0.045542 |
| A_24_P210829 | NM_005009 | chr16: 389690-390228 | 0.613786 | 0.04949 |
| A_23_P322043 | NM_015327 | chr1: 154485978-154485919 | 1.600805 | 0.047654 |
| A_23_P47867 | NM_003622 | chr12: 27737226-27737285 | 1.591339 | 0.0168 |
| A_23_P98898 | NM_001798 | chr12: 54652056-54652115 | 2.161649 | 0.015927 |
| A_23_P256663 | NM_003614 | chr22: 36551272-36551331 | 0.790395 | 0.026329 |
| A_23_P12329 | NM_016022 | chr1: 148504949-148504890 | 1.667644 | 0.024454 |
| A_24_P384990 | ENST00000400362 | chr21: 45534838-45534897 | 0.606583 | 0.001683 |
| A_23_P104199 | NM_133376 | chr10: 33240572-33240513 | 1.722096 | 0.027718 |
| A_23_P16762 | NM_017892 | chr2: 153221128-153221069 | 2.10584 | 0.004079 |
| A_32_P208713 | | chr19: 023311797-023311856 | 0.565266 | 0.009235 |
| A_23_P5075 | NM_031213 | chr19: 1828241-1828182 | 0.767993 | 0.010886 |
| A_23_P98431 | NM_000190 | chr11: 118469339-118469398 | 0.536304 | 0.019554 |
| A_23_P42935 | NM_004333 | chr7: 140080947-140080888 | 1.676591 | 0.036316 |
| A_24_P624792 | | chr11: 071292429-071292488 | 0.775874 | 0.043964 |
| A_23_P40952 | NM_002880 | chr3: 12600670-12600611 | 0.451008 | 0.002215 |
| A_23_P86434 | BC007866 | chr10: 64896047-64896106 | 1.949552 | 0.029819 |
| A_23_P107394 | NM_032580 | chr17: 7965871-7965812 | 0.799889 | 0.032174 |
| A_23_P57199 | NM_178311 | chr20: 23913799-23913740 | 0.69932 | 0.012074 |
| A_23_P35970 | NM_001467 | chr11: 118400689-118400630 | 0.608108 | 0.031657 |
| A_24_P383356 | NM_015359 | chr8: 22328312-22329237 | 1.934128 | 0.004556 |
| A_32_P143496 | H81180 | chr1: 148124240-148124299 | 1.311222 | 0.0224 |
| A_23_P1962 | NM_004585 | chr11: 63070403-63070462 | 0.747038 | 0.015152 |
| A_24_P401036 | | chr9: 78354315-78354256 | 0.680697 | 0.049126 |
| A_24_P134834 | NM_032276 | chr2: 227569291-227569350 | 2.036474 | 0.020839 |
| A_24_P246397 | NM_021147 | chr5: 54564808-54564749 | 0.845471 | 0.023664 |
| A_23_P30745 | NM_014623 | chr6: 43088148-43088089 | 0.641773 | 0.041844 |
| A_23_P75978 | NM_030813 | chr11: 71682255-71682196 | 0.55726 | 0.009403 |
| A_23_P113101 | | | 0.813013 | 0.016973 |
| A_23_P359540 | NM_003540 | chr6: 26348797-26348856 | 1.616688 | 0.034961 |
| A_24_P898583 | NM_003449 | chr6: 30260406-30260347 | 0.820262 | 0.033902 |
| A_24_P916624 | N40973 | chr19: 41630420-41630479 | 1.487999 | 0.040691 |
| A_23_P385206 | NM_177424 | chr1: 28023223-28023282 | 0.686226 | 0.022613 |
| A_23_P142849 | NM_005168 | chr2: 151033412-151033353 | 1.729619 | 0.004473 |
| A_32_P88626 | NM_001048183 | chr1: 28696227-28696286 | 0.762944 | 0.000318 |
| A_24_P13376 | NM_024551 | chr12: 1760340-1760399 | 1.496091 | 0.037038 |
| A_23_P59179 | NM_021976 | chr6: 33270223-33270164 | 1.663097 | 0.03496 |
| A_23_P134637 | NM_014855 | chr7: 4797370-4797429 | 0.789966 | 0.025002 |
| A_23_P167093 | NM_000203 | chr4: 987810-987869 | 2.15141 | 0.001957 |
| A_23_P95165 | NM_020210 | chr15: 88572536-88572595 | 0.673603 | 0.014678 |
| A_24_P583350 | NM_001015050 | chr3: 127132120-127131072 | 0.69023 | 0.000947 |
| A_32_P67414 | BC033990 | chr2: 241146839-241146898 | 0.645689 | 0.006872 |
| A_23_P70588 | NM_000841 | chr6: 34098587-34098528 | 0.763658 | 0.018205 |
| A_32_P115077 | BF718543 | chr9: 130090921-130090862 | 0.639782 | 0.02379 |
| A_23_P326319 | NM_033201 | chr16: 15589109-15589168 | 1.466762 | 0.02433 |
| A_23_P15478 | NM_004822 | chr17: 9026980-9065237 | 0.809977 | 0.010899 |
| A_24_P714750 | | chr11: 114337042-114336983 | 0.750055 | 0.025709 |
| A_23_P104413 | NM_033178 | chr10: 135331624-135331676 | 0.71998 | 0.016035 |
| A_23_P125772 | NM_014370 | chrX: 152704060-152704119 | 0.785311 | 0.013791 |
| A_23_P101272 | NM_017908 | chr19: 63684277-63684336 | 0.741233 | 0.04283 |
| A_23_P137423 | NM_052868 | chr1: 158328279-158328021 | 1.605533 | 0.035418 |
| A_23_P168567 | NM_032999 | chr7: 73812836-73812895 | 1.793219 | 0.032729 |
| A_32_P69987 | THC2548567 | chr10: 33659750-33659809 | 1.628715 | 0.028329 |
| A_23_P168531 | NM_003088 | chr7: 5611468-5611527 | 0.808278 | 0.029286 |
| A_23_P258570 | NM_002814 | chrX: 107214865-107214806 | 1.757435 | 0.024848 |
| A_23_P379111 | BC020886 | chr4: 185508159-185508100 | 1.669936 | 0.029467 |
| A_23_P158725 | NM_001042422 | chr17: 77790577-77790636 | 1.405123 | 0.048396 |
| A_24_P929621 | NP111770 | chr4: 191173470-191173421 | 0.732975 | 0.012063 |
| A_24_P109941 | | chr17: 32005102-32005043 | 0.36779 | 0.001079 |
| A_24_P696507 | AK092810 | chr2: 206688686-206688627 | 1.794537 | 0.013854 |
| A_23_P150249 | NM_006848 | chr11: 65415351-65415410 | 0.843667 | 0.046984 |
| A_24_P889527 | CF142843 | chr11: 3833083-3833142 | 0.727519 | 0.012166 |
| A_24_P54879 | NM_005506 | chr4: 77306445-77303520 | 1.640225 | 0.046647 |
| A_32_P107029 | NM_004851 | chr19: 55556079-55556020 | 0.686391 | 0.028879 |
| A_23_P3496 | NM_080861 | chr16: 1766844-1766785 | 0.634326 | 0.019097 |
| A_23_P6362 | NM_198440 | chr22: 22509040-22508981 | 0.806437 | 0.020487 |
| A_32_P113646 | | chr2: 227368890-227369000 | 0.784624 | 0.018266 |
| A_23_P321320 | NM_052902 | chr2: 220189340-220189399 | 0.526824 | 0.007052 |
| A_23_P95292 | NM_014741 | chr11: 46650404-46650463 | 2.319115 | 0.001433 |
| A_24_P349676 | NM_017908 | chr19: 63680418-63680477 | 0.79529 | 0.010553 |
| A_24_P155058 | NM_001556 | chr8: 42270126-42270185 | 0.667032 | 0.042173 |
| A_23_P217079 | NM_003863 | chr9: 129737476-129737417 | 1.466866 | 0.031603 |
| A_24_P142442 | NM_015584 | chr17: 23698515-23698456 | 1.641633 | 0.001432 |
| A_24_P916006 | AA435826 | chr16: 002103265-002103206 | 1.886369 | 0.01347 |
| A_24_P418086 | NM_015014 | chr1: 233384822-233382648 | 1.970352 | 0.021059 |
| A_32_P216241 | NM_182832 | chr21: 41473295-41473236 | 0.765043 | 0.040801 |
| A_32_P104861 | AL567699 | chr11: 74358392-74358333 | 0.573702 | 0.005323 |
| A_24_P415168 | BC011620 | chr9: 130192598-130192653 | 2.255149 | 0.010685 |
| A_24_P126325 | AK027318 | chr1: 25443570-25443511 | 1.55784 | 0.040398 |
| A_32_P40493 | AK026691 | chr6: 58345073-58345014 | 1.743165 | 0.008387 |
| A_24_P307065 | XM_928637 | chr17: 71734250-71734191 | 2.370558 | 0.002067 |
| A_24_P331711 | ENST00000368817 | chr1: 150085988-150085929 | 0.764721 | 0.048917 |
| A_23_P369758 | | chr1_random: | 1.474488 | 0.049378 |
| | | 000367520-000367574 | | |
| A_24_P611965 | THC2560004 | chr11: 005649052-005649111 | 1.618644 | 0.025468 |
| A_24_P339201 | NM_024042 | chr16: 707091-707150 | 0.751791 | 0.023004 |
| A_32_P92171 | BC052596 | chr19: 60586905-60582492 | 0.689155 | 0.018859 |
| A_23_P120316 | NM_001040409 | chr2: 74295493-74295552 | 1.444838 | 0.039571 |
| A_23_P151907 | NM_002570 | chr15: 99662738-99662679 | 0.676202 | 0.00551 |
| A_32_P121755 | THC2672892 | chr17: 17606980-17607039 | 0.431674 | 0.00136 |
| A_24_P8454 | NM_001130528 | chr17: 46422858-46422222 | 1.595292 | 0.022707 |
| A_23_P60627 | NM_001141 | chr17: 7892907-7892966 | 1.219793 | 0.016756 |
| A_23_P207614 | NM_014680 | chr17: 23966120-23966061 | 1.433469 | 0.047306 |
| A_23_P18276 | NM_005778 | chr3: 50131141-50131200 | 1.989116 | 0.025967 |
| A_32_P46954 | THC2562523 | chr9: 130107084-130107025 | 0.815733 | 0.020717 |
| A_23_P141484 | NM_018182 | chr17: 24107968-24107909 | 1.461425 | 0.012598 |
| A_32_P76441 | AA581571 | chr10: 122104284-122104343 | 0.351543 | 0.006929 |
| A_24_P96171 | NM_030578 | chr19: 46552230-46552171 | 0.563928 | 0.003623 |
| A_23_P120194 | NM_016467 | chr2: 190344512-190344453 | 0.574355 | 0.03716 |
| A_23_P88781 | NM_020313 | chr16: 56020201-56020142 | 0.694563 | 0.000517 |
| A_24_P30257 | NM_002478 | chr11: 17699518-17699577 | 0.792822 | 0.039826 |
| A_23_P49638 | NM_006613 | chr17: 18983778-18983837 | 0.795461 | 0.016667 |
| A_24_P315346 | NR_024583 | chr22: 22978645-22978704 | 0.721257 | 0.001313 |
| A_23_P207736 | AK023077 | chr17: 74303782-74303723 | 0.633313 | 0.042579 |
| A_24_P7785 | | chr2: 001551245-001551181 | 0.766894 | 0.047046 |
| A_24_P254949 | NM_021965 | chr9: 70183028-70183087 | 0.662866 | 0.001786 |
| A_23_P330262 | NM_012398 | chr19: 3581729-3581670 | 1.934406 | 0.027833 |
| A_23_P2216 | NM_007043 | chr12: 74184103-74184044 | 2.069774 | 0.022319 |
| A_32_P144708 | NM_022802 | chr17_random: 117906-117965 | 0.660523 | 0.004217 |
| A_23_P359897 | NM_030647 | chr7: 139431574-139431515 | 1.605824 | 0.016539 |
| A_23_P377434 | NM_001080495 | chr7: 5313040-5312981 | 1.68718 | 0.012384 |
| A_23_P106472 | AF357221 | chr15: 73818030-73818089 | 0.767307 | 0.014381 |
| A_23_P140405 | NM_005197 | chr14: 88697624-88697565 | 1.685384 | 0.03507 |
| A_32_P200600 | THC2591088 | chr19: 57417294-57417254 | 0.598362 | 0.031047 |
| A_23_P141549 | NM_001011 | chr2: 3605654-3605713 | 0.734999 | 0.034374 |
| A_32_P733356 | AK026367 | chrY: 21954784-21954725 | 0.747736 | 0.033217 |
| A_23_P214464 | NM_001760 | chr6: 42017191-42016266 | 0.760841 | 0.048731 |
| A_23_P161501 | NM_024954 | chr10: 99320431-99320490 | 0.714004 | 0.006913 |
| A_23_P65797 | NM_022480 | chr15: 84103636-84103577 | 0.574528 | 0.006582 |
| A_24_P283834 | NM_000795 | chr11: 112786811-112786752 | 0.774617 | 0.002338 |
| A_23_P16648 | NM_017573 | chr19: 1432543-1432484 | 0.699349 | 0.029691 |
| A_24_P10674 | NM_052948 | chr19: 40970877-40970936 | 0.817097 | 0.025521 |
| A_23_P39386 | NM_014266 | chr19: 41086900-41086959 | 0.732964 | 0.034884 |
| A_24_P374962 | NM_018991 | chr7: 74834647-74834706 | 1.771267 | 0.020745 |
| A_32_P171124 | THC2717023 | chr16: 27149885-27149944 | 0.740675 | 0.037632 |
| A_24_P101879 | XM_001720960 | chrX_random: | 0.727926 | 0.006605 |
| | | 000288572-000288631 | | |
| A_23_P36757 | NM_000545 | chr12: 119921743-119921802 | 0.788618 | 0.01563 |
| A_23_P119502 | NM_003775 | chr19: 3131249-3131308 | 0.726843 | 0.020371 |
| A_23_P210015 | NM_014369 | chr2: 130848938-130848997 | 0.496582 | 0.003404 |
| A_23_P340717 | NM_004498 | chr15: 50837102-50837043 | 0.858319 | 0.013454 |
| A_24_P399454 | NM_138568 | chr19: 50408315-50408256 | 0.758134 | 0.017594 |
| A_23_P215175 | NM_005692 | chr7: 150542945-150542199 | 0.632111 | 0.003639 |
| A_23_P13048 | NM_005553 | chr11: 70937694-70937753 | 0.661842 | 0.003807 |
| A_32_P22263 | NM_004965 | chr21: 39636946-39636887 | 1.705518 | 0.045574 |
| A_23_P97573 | NM_004698 | chr1: 148585571-148588295 | 1.921231 | 0.014608 |
| A_24_P172685 | NM_000486 | chr12: 48634694-48634753 | 0.820829 | 0.034014 |
| A_23_P133904 | NM_001011700 | chr6: 31605882-31605939 | 0.705318 | 0.001906 |
| A_24_P357100 | NM_014310 | chr22: 34279556-34279615 | 0.637293 | 0.000867 |
| A_23_P34527 | NM_025207 | chr1: 153227754-153227813 | 0.669117 | 0.003767 |
| A_32_P13823 | THC2719076 | chr3: 50279161-50279102 | 0.695674 | 0.046981 |
| A_32_P202719 | THC2644672 | chr16: 1138709-1138650 | 0.730823 | 0.006691 |
| A_32_P147790 | BC071732 | chr2: 232285901-232285847 | 1.932154 | 0.038236 |
| A_24_P350644 | NM_013318 | chr9: 133365149-133365208 | 0.572439 | 0.036119 |
| A_23_P33376 | NM_002595 | chr12: 95198729-95198142 | 0.625364 | 0.026768 |
| A_24_P279718 | NM_015526 | chr19: 41200159-41199694 | 1.842015 | 0.006059 |
| A_23_P69326 | NM_183393 | chr3: 62359875-62359816 | 0.85719 | 0.022881 |
| A_24_P308096 | NM_000215 | chr19: 17798585-17798526 | 0.699342 | 0.034597 |
| A_32_P18251 | NM_023934 | chr2: 84372288-84372347 | 1.720847 | 0.017029 |
| A_23_P314086 | NM_194460 | chr19: 598645-598586 | 0.659039 | 0.049915 |
| A_24_P278602 | NM_173042 | chr11: 71389077-71389136 | 0.732861 | 0.015184 |
| A_32_P9861 | | chr2: 061618328-061618387 | 2.188319 | 0.007247 |
| A_32_P93209 | AY203961 | chr19: 60901004-60901063 | 0.814705 | 0.005545 |
| A_23_P7313 | NM_001040058 | chr4: 89122944-89123003 | 1.233344 | 0.03645 |
| A_24_P477610 | | chr6: 168982226-168982167 | 0.81945 | 0.028081 |
| A_24_P826858 | THC2742812 | chr16: 27638128-27638187 | 0.826176 | 0.018841 |
| A_23_P15751 | NM_000160 | chr17_random: | 0.694999 | 0.002934 |
| | | 2329878-2329937 | | |
| A_23_P19510 | NR_003937 | chr6: 32834772-32834713 | 0.685468 | 0.029169 |
| A_24_P565496 | | chr14: 103745190-103745249 | 0.780351 | 0.01849 |
| A_24_P107142 | NM_145869 | chr10: 81904945-81904886 | 1.41864 | 0.043781 |
| A_32_P92274 | | chr1_random: | 0.772303 | 0.002521 |
| | | 000714392-000714451 | | |
| A_24_P125434 | NM_032243 | chr18: 9877992-9878051 | 0.80676 | 0.035285 |
| A_32_P108722 | AK127572 | chr21: 46699375-46699316 | 0.5843 | 0.004158 |
| A_23_P819 | NM_005101 | chr1: 939430-939489 | 0.739524 | 0.040862 |
| A_23_P14369 | AB028979 | chr14: 23067826-23067767 | 0.762373 | 0.031955 |
| A_23_P368558 | NM_015348 | chr2: 97739765-97739706 | 1.907078 | 0.0216 |
| A_24_P391748 | | | 0.543669 | 0.013724 |
| A_23_P139722 | NM_001065 | chr12: 6308384-6308325 | 1.567084 | 0.039193 |
| A_24_P347378 | NM_001629 | chr13: 30236155-30236214 | 1.646645 | 0.033603 |
| A_23_P127565 | NM_178834 | chr11: 110936670-110936729 | 0.712029 | 0.016119 |
| A_23_P92065 | AF116626 | chr3: 173844569-173844628 | 0.782579 | 0.039486 |
| A_23_P105592 | NM_000431 | chr12: 108519324-108519383 | 0.677254 | 0.010751 |
| A_23_P31372 | NM_005918 | chr7: 75532132-75532191 | 1.90736 | 0.015258 |
| A_24_P92174 | ENST00000390597 | chr14: 105565315-105565256 | 0.82878 | 0.02536 |
| A_23_P90855 | NM_014808 | chr2: 242081044-242081103 | 0.692294 | 0.003817 |
| A_23_P412491 | NM_005599 | chr1: 116182455-116182396 | 0.836056 | 0.046795 |
| A_24_P41042 | NM_032848 | chr12: 112109227-112113547 | 0.759385 | 0.012877 |
| A_24_P215445 | NM_005169 | chr11: 71628703-71628644 | 0.826224 | 0.045501 |
| A_23_P369641 | NM_001100878 | chr8: 144720108-144720049 | 0.80348 | 0.009414 |
| A_23_P218456 | NM_012218 | chr19: 10661232-10661291 | 1.712791 | 0.044937 |
| A_23_P55706 | NM_006509 | chr19: 50233225-50233284 | 1.513606 | 0.041475 |
| A_24_P121535 | NM_005159 | chr15: 32872843-32872784 | 1.799335 | 0.042997 |
| A_24_P84508 | XR_018553 | chr9: 6629362-6629423 | 1.402728 | 0.03372 |
| A_23_P79066 | NM_001077238 | chr19: 2304022-2304081 | 0.735614 | 0.012322 |
| A_23_P14863 | NM_024111 | chr15: 39035814-39035873 | 0.783054 | 0.019765 |
| A_24_P107303 | NM_001558 | chr11: 117376908-117376967 | 0.687551 | 0.010039 |
| A_23_P94736 | NM_175039 | chr9: 129710680-129710621 | 0.667551 | 0.001429 |
| A_23_P148541 | NM_139250 | chrX: 153468202-153468261 | 0.823448 | 0.016696 |
| A_24_P341603 | | chr5: 137928-137869 | 0.779049 | 0.013793 |
| A_24_P22939 | NM_213604 | chr19: 1457006-1456947 | 0.744761 | 0.005099 |
| A_23_P50928 | NM_182528 | chr2: 119630951-119630892 | 0.814471 | 0.007692 |
| A_32_P151823 | BC040619 | chr8: 107354042-107354101 | 0.671641 | 0.001035 |
| A_32_P158302 | NR_024412 | chr7: 112544823-112544764 | 0.517554 | 0.002596 |
| A_24_P761130 | AK097080 | chr1: 124796-124737 | 2.19372 | 0.001573 |
| A_23_P256021 | NM_031206 | chrX: 64649302-64649243 | 0.628343 | 0.011104 |
| A_23_P209135 | NM_002287 | chr19: 59558517-59558458 | 0.69837 | 0.014196 |
| A_23_P259771 | AF462442 | chr16: 48697667-48697726 | 0.707261 | 0.026978 |
| A_23_P88865 | NM_144601 | chr16: 65205057-65205116 | 1.747049 | 0.025513 |
| A_23_P7535 | NM_022304 | chr5: 175043397-175043456 | 0.700973 | 0.002708 |
| A_24_P532653 | AA242987 | chr12: 8691188-8691247 | 1.361687 | 0.040643 |
| A_23_P159172 | NM_001080395 | chr17: 76707782-76706786 | 0.781078 | 0.049082 |
| A_24_P204454 | | chr17: 076130791-076130851 | 0.557824 | 0.003344 |
| A_23_P79069 | NM_022904 | chr19: 15423512-15423453 | 0.664693 | 0.023582 |
| A_24_P927614 | AY358247 | chr3: 131245191-131245250 | 0.620661 | 0.016018 |
| A_24_P273214 | | chr10: 001272027-001272087 | 0.773627 | 0.007958 |
| A_24_P166431 | NM_006778 | chr6: 30229952-30229893 | 0.807168 | 0.018379 |
| A_23_P18641 | NM_031953 | chr4: 186520100-186520159 | 1.794279 | 0.024786 |
| A_23_P361679 | BC008001 | chr9: 46955881-46955940 | 0.826107 | 0.031555 |
| A_24_P381604 | NM_021999 | chr13: 47733914-47733973 | 1.615022 | 0.037735 |
| A_23_P156471 | NM_001253 | chr6: 44521465-44521524 | 2.169659 | 0.024647 |
| A_23_P148852 | AF010144 | chr1: 23904722-23904663 | 0.678122 | 0.035465 |
| A_24_P121891 | NM_025129 | chr19: 55003494-55002396 | 0.804824 | 0.043894 |
| A_23_P341532 | NM_031915 | chr13: 48963912-48963971 | 0.691856 | 0.023905 |
| A_24_P101128 | NM_005509 | chr5: 118496745-118496804 | 1.822711 | 0.016292 |
| A_23_P320887 | NM_001038707 | chr1: 149290076-149290061 | 2.067523 | 0.023788 |
| A_23_P39925 | NM_003494 | chr2: 71767254-71767313 | 0.751643 | 0.037243 |
| A_23_P218685 | NM_001032288 | chr20: 48132759-48132700 | 0.636677 | 0.03449 |
| A_23_P252903 | NM_001037633 | chr5: 138310787-138310728 | 0.772333 | 0.021902 |
| A_24_P163113 | NM_017548 | chr3: 134775798-134776611 | 1.570914 | 0.034575 |
| A_23_P111843 | NM_006349 | chr7: 100654059-100654118 | 0.599028 | 0.01709 |
| A_24_P40010 | NM_001031 | chr19: 8292404-8292549 | 0.662706 | 0.005265 |
| A_24_P636974 | XR_039576 | chr7: 056402763-056402824 | 1.441637 | 0.034861 |
| A_24_P50908 | ENST00000366699 | chr1: 226650225-226650166 | 0.572629 | 0.008844 |
| A_23_P126089 | NR_003062 | chr1: 151379324-151379265 | 0.836739 | 0.046828 |
| A_23_P91640 | NM_020437 | chr22: 25168475-25168534 | 0.782028 | 0.003631 |
| A_24_P125353 | NM_007359 | chr17: 35578184-35579149 | 0.659907 | 0.014431 |
| A_23_P92490 | NM_020139 | chr4: 104220352-104220293 | 0.640007 | 0.007681 |
| A_23_P434442 | NM_001006933 | chrX: 102751321-102751380 | 0.618154 | 0.014045 |
| A_23_P123672 | NM_014290 | chr9: 99298057-99298116 | 0.746555 | 0.022608 |
| A_24_P401432 | AF289611 | chr14: 104963490-104963549 | 0.758113 | 0.020506 |
| A_24_P921584 | NM_184041 | chr16: 29984584-29984643 | 0.799296 | 0.034003 |
| A_23_P357995 | NM_178547 | chr1: 32870023-32860123 | 2.139635 | 0.033816 |
| A_23_P168105 | NM_030652 | chr6: 32241927-32241986 | 0.774056 | 0.031666 |
| A_23_P32223 | NM_012127 | chr9: 129968910-129968499 | 0.653069 | 0.001019 |
| A_32_P10894 | NR_015419 | chr15: 54996656-54996597 | 0.757344 | 0.031251 |
| A_23_P143748 | NM_015140 | chr22: 41892845-41892786 | 0.74881 | 0.024921 |
| A_23_P86682 | NM_013451 | chr10: 95056569-95056510 | 1.832442 | 0.027037 |
| A_32_P208370 | | chr10: 035898567-035898508 | 1.742344 | 0.012555 |
| A_23_P80643 | NM_006515 | chr3: 4331190-4331249 | 0.689132 | 0.021373 |
| A_23_P414913 | NM_022343 | chr9: 36153763-36153822 | 0.711305 | 0.042599 |
| A_24_P786912 | | chr8: 000735716-000735657 | 0.753372 | 0.001554 |
| A_23_P12173 | NM_181715 | chr1: 152186871-152186812 | 0.68268 | 0.033163 |
| A_24_P26170 | NM_014703 | chr3: 51415621-51411244 | 0.774007 | 0.001074 |
| A_24_P104926 | NM_080825 | chr20: 31715080-31715139 | 0.769887 | 0.007539 |
| A_23_P369634 | NM_001100878 | chr8: 144719583-144719524 | 0.702569 | 0.032196 |
| A_23_P84651 | NM_003418 | chr3: 130371160-130371122 | 1.893828 | 0.037821 |
| A_23_P83953 | S80864 | chr4: 064904680-064904741 | 0.527658 | 0.021984 |
| A_24_P40229 | NM_016652 | chr20: 19966171-19966112 | 1.608347 | 0.046578 |
| A_32_P160414 | THC2615996 | chr20: 28228455-28228514 | 1.585314 | 0.016963 |
| A_24_P264106 | NM_000984 | chr17: 24073905-24073964 | 1.544757 | 0.008886 |
| A_32_P218030 | BU633383 | chr11: 63434775-63434716 | 0.588696 | 0.017097 |
| A_24_P19228 | NM_138924 | chr19: 1350172-1350012 | 0.662201 | 0.006993 |
| A_23_P112470 | NM_002989 | chr9: 34699812-34699659 | 0.737367 | 0.000972 |
| A_23_P136232 | NM_017813 | chr8: 58038252-58038193 | 1.478228 | 0.033157 |
| A_23_P89343 | NM_152244 | chr17: 43554800-43554859 | 0.563236 | 0.008443 |
| A_23_P49842 | NM_005148 | chr17: 23898991-23898932 | 1.726178 | 0.004488 |
| A_32_P120534 | BC019044 | chr19: 18546757-18546698 | 0.491101 | 0.015253 |
| A_23_P61987 | NM_025268 | chr14: 105067477-105067536 | 0.710052 | 0.002656 |
| A_23_P162199 | NM_024779 | chr12: 56282893-56282952 | 1.730977 | 0.024009 |
| A_23_P130194 | NM_006907 | chr17: 77483681-77483622 | 0.612324 | 0.006859 |
| A_32_P71551 | NM_001129979 | chr16: 75804333-75804392 | 0.830777 | 0.036603 |
| A_23_P41553 | AF241539 | chr4: 069660175-069660116 | 0.80118 | 0.031846 |
| A_24_P413470 | NM_005427 | chr1: 3639401-3639460 | 0.845237 | 0.043774 |
| A_23_P384405 | NM_001440 | chr8: 28666942-28667001 | 0.744901 | 0.02571 |
| A_24_P367242 | NM_181607 | chr21: 30774343-30774284 | 0.839153 | 0.037946 |
| A_24_P681011 | NM_022740 | chr7: 138897510-138897451 | 1.529826 | 0.017203 |
| A_24_P849628 | NM_002904 | chr6: 32028030-32027971 | 1.710604 | 0.038813 |
| A_32_P46112 | | chr1: 245013047-245013106 | 0.789197 | 0.048288 |
| A_23_P355439 | NM_170745 | chr6: 25834601-25834542 | 0.778722 | 0.003445 |
| A_24_P278167 | NM_006007 | chr9: 74161676-74160788 | 1.650125 | 0.041739 |
| A_23_P137830 | XM_060887 | chr1: 45226866-45226807 | 1.75224 | 0.021859 |
| A_32_P118578 | AI818152 | chr22: 34150173-34149302 | 0.771822 | 0.02569 |
| A_24_P349788 | NM_001130677 | chr17: 34083371-34083312 | 0.710098 | 0.034881 |
| A_24_P158065 | NM_001040057 | chr7: 92028597-92028538 | 1.475776 | 0.0447 |
| A_23_P167066 | NM_003359 | chr4: 39178216-39178157 | 1.560648 | 0.01389 |
| A_23_P166353 | NM_198157 | chr22: 20307666-20307725 | 0.604863 | 0.035482 |
| A_32_P183356 | THC2608750 | chr4: 99583317-99583302 | 0.596629 | 0.005427 |
| A_23_P150255 | NM_006328 | chr11: 66151269-66151328 | 0.606369 | 0.005911 |
| A_24_P321125 | NM_001006939 | chr10: 49791528-49791469 | 0.659756 | 0.012007 |
| A_23_P209995 | NM_173842 | chr2: 113607479-113607538 | 1.2949 | 0.04549 |
| A_23_P64560 | NM_014489 | chr11: 3803928-3803987 | 0.636608 | 0.000434 |
| A_23_P17307 | NM_018270 | chr20: 60901779-60901838 | 0.54452 | 0.013775 |
| A_23_P7783 | NM_016604 | chr5: 137800264-137800323 | 1.845426 | 0.019352 |
| A_23_P14886 | NM_032520 | chr16: 1352855-1353004 | 0.620703 | 0.008998 |
| A_24_P227831 | NM_019862 | chr16: 16143525-16143584 | 1.684358 | 0.02691 |
| A_23_P33465 | NM_024324 | chr22: 48706922-48706981 | 0.778192 | 0.049382 |
| A_23_P126540 | NM_020683 | chr1: 111832941-111832882 | 0.730611 | 0.008682 |
| A_23_P34968 | NM_024041 | chr1: 149408123-149408182 | 2.106646 | 0.015707 |
| A_23_P120572 | NM_018993 | chr20: 19929789-19929848 | 1.599477 | 0.011139 |
| A_23_P90220 | NM_152655 | chr19: 42334475-42334416 | 1.944279 | 0.020082 |
| A_23_P25187 | NM_002281 | chr12: 50966199-50966140 | 0.762804 | 0.008335 |
| A_24_P323805 | | chr4: 043106956-043106893 | 0.409903 | 0.000296 |
| A_32_P117697 | ENST00000378969 | chr15: 91056819-91056878 | 2.000064 | 0.017294 |
| A_23_P302386 | M38056 | chr6: 33084122-33084063 | 0.803464 | 0.013918 |
| A_23_P107801 | NM_032207 | chr19: 16493086-16493145 | 0.754712 | 0.016231 |
| A_23_P44768 | NM_013254 | chr12: 63181710-63181769 | 2.072356 | 0.025259 |
| A_24_P255543 | AL390214 | chr5: 14483194-14483253 | 1.772311 | 0.020142 |
| A_23_P88819 | NM_017458 | chr16: 29766743-29766802 | 0.625789 | 0.006304 |
| A_23_P169154 | NM_024896 | chr9: 5775753-5775694 | 0.61989 | 0.028242 |
| A_32_P28158 | | chr1: 000661777-000661836 | 0.5195 | 0.008167 |
| A_32_P90455 | ENST00000376615 | chr12: 131598488-131598547 | 0.780602 | 0.029401 |
| A_24_P222655 | NM_015991 | chr1: 22838382-22838441 | 0.720873 | 0.032971 |
| A_32_P188694 | THC2676656 | chr19: 16012146-16012205 | 0.835712 | 0.043899 |
| A_24_P222054 | AF271776 | chrM: 8902-8843 | 1.737426 | 0.00897 |
| A_24_P156113 | NM_014601 | chr19: 52938036-52938095 | 1.852213 | 0.008481 |
| A_32_P148914 | BC037255 | chr8: 12561140-12561081 | 0.790814 | 0.048948 |
| A_23_P76218 | NM_053283 | chr12: 53324771-53324712 | 0.796942 | 0.013373 |
| A_23_P500956 | NM_006577 | chr2: 62304735-62304794 | 1.562677 | 0.036133 |
| A_24_P915904 | BU164445 | chr20: 3859864-3859805 | 1.877069 | 0.022794 |
| A_23_P208340 | NM_020428 | chr19: 10615950-10616009 | 0.581846 | 0.026059 |
| A_32_P121334 | | chr9: 132354576-132345635 | 0.663044 | 0.002961 |
| A_32_P351277 | BC004968 | chr16: 668578-668519 | 0.798055 | 0.036923 |
| A_23_P23421 | NM_198236 | chr1: 155174859-155173879 | 0.616309 | 0.048831 |
| A_24_P388810 | NM_003135 | chr5: 112231013-112231072 | 2.327029 | 0.010602 |
| A_32_P36715 | CR627135 | chr19: 21431227-21431168 | 0.705976 | 0.01022 |
| A_24_P342572 | NM_138346 | chr1: 11905540-11905481 | 0.657577 | 0.001349 |
| A_23_P139547 | NM_006009 | chr12: 47865739-47865680 | 1.779839 | 0.021668 |
| A_32_P20997 | BU561469 | chr20: 47092755-47092734 | 0.805861 | 0.02883 |
| A_32_P113193 | NM_014630 | chr15: 83147453-83147512 | 0.646196 | 0.000316 |
| A_32_P222664 | ENST00000372899 | chr6: 42768660-42768719 | 0.468384 | 0.002247 |
| A_24_P924752 | M14087 | | 0.847958 | 0.043259 |
| A_24_P686956 | THC2542546 | chr1: 172099988-172099929 | 1.465298 | 0.038258 |
| A_23_P256898 | NM_005104 | chr6: 33056120-33056179 | 2.554708 | 0.009174 |
| A_23_P131111 | NM_198580 | chr19: 17476894-17476953 | 20.06193 | 0.021813 |
| A_24_P918044 | AK023572 | chr2: 46455107-46455166 | 0.720447 | 0.032047 |
| A_23_P130585 | NM_054113 | chr19: 16136614-16136555 | 0.747784 | 0.014901 |
| A_23_P215454 | NM_000501 | chr7: 73121535-73121594 | 1.389468 | 0.016044 |
| A_24_P524262 | THC2522342 | chr15: 30602568-30593602 | 0.607717 | 0.011153 |
| A_23_P22382 | NM_015527 | chr16: 30275997-30275938 | 0.582175 | 0.010512 |
| A_23_P131289 | NM_024536 | chr2: 220112685-220112626 | 0.860931 | 0.031815 |
| A_24_P360529 | NM_002604 | chr8: 66797273-66797214 | 1.536288 | 0.04126 |
| A_23_P11461 | NM_001032288 | chr20: 48134098-48132825 | 0.281902 | 0.000355 |
| A_23_P207154 | NM_022644 | chr17: 59303401-59303342 | 0.845277 | 0.012161 |
| A_24_P239183 | NM_018406 | chr3: 196999422-196999027 | 0.804471 | 0.039391 |
| A_23_P368645 | NM_003659 | chr2: 178111052-178111111 | 2.493197 | 0.000615 |
| A_23_P5995 | NM_006420 | chr20: 47082037-47082096 | 0.47731 | 0.001278 |
| A_23_P36183 | NM_005316 | chr11: 18344166-18344225 | 1.909523 | 0.04937 |
| A_32_P8251 | NM_020901 | chr11: 601931-601990 | 0.601609 | 0.008135 |
| A_23_P342709 | NM_152676 | chr18: 69891777-69891718 | 0.70605 | 0.000791 |
| A_32_P175979 | BG686022 | chr19: 47282261-47282202 | 0.72678 | 0.044386 |
| A_24_P920090 | NM_205860 | chr1: 198275459-198275518 | 0.831228 | 0.032827 |
| A_23_P71300 | NM_018246 | chr8: 27647122-27647063 | 0.694242 | 0.036552 |
| A_32_P38500 | | chr19: 052856683-052856742 | 0.763465 | 0.00077 |
| A_23_P161552 | NM_032389 | chr11: 47144413-47143589 | 2.019197 | 0.020213 |
| A_32_P49284 | THC2568453 | chr8: 047610082-047610023 | 1.490049 | 0.034682 |
| A_23_P76851 | NM_001039619 | chr14: 22460067-22460008 | 0.583874 | 0.017319 |
| A_24_P387158 | NM_005782 | chr17: 77441933-77441874 | 0.714998 | 0.024526 |
| A_23_P47135 | NM_005693 | chr11: 47246899-47246958 | 0.649815 | 0.034297 |
| A_23_P161386 | NM_003061 | chr10: 98750624-98750601 | 0.827708 | 0.031747 |
| A_23_P114374 | NM_020061 | chrX: 153071648-153071707 | 0.775332 | 0.034936 |
| A_24_P255798 | NM_030974 | chr8: 145230010-145229951 | 0.696252 | 0.013038 |
| A_23_P386942 | NM_145173 | chr19: 2665640-2665581 | 0.769442 | 0.034664 |
| A_23_P32500 | NM_015136 | chr3: 52532723-52532782 | 1.465452 | 0.038845 |
| A_24_P326957 | NM_025230 | chr14: 23662877-23662936 | 0.59526 | 0.010816 |
| A_24_P166407 | NM_003544 | chr6: 26135187-26135128 | 1.87955 | 0.010753 |
| A_32_P197720 | BX100171 | chr9: 046230446-046230504 | 0.751993 | 0.018602 |
| A_24_P614579 | BC000206 | chr1: 46541826-46541885 | 1.661736 | 0.031359 |
| A_24_P274640 | NM_001042490 | chr5: 70373168-70373109 | 1.493833 | 0.041816 |
| A_23_P169007 | NM_182795 | chr8: 21949981-21950151 | 0.831741 | 0.023119 |
| A_23_P306346 | NM_152420 | chr9: 76788496-76788437 | 1.873583 | 0.041305 |
| A_23_P398460 | NM_000189 | chr2: 74971786-74971845 | 1.501971 | 0.042839 |
| A_23_P168629 | NM_018077 | chr7: 127738104-127738045 | 0.666395 | 0.012803 |
| A_24_P295543 | NM_001001342 | chr10: 102024588-102024529 | 1.810064 | 0.021079 |
| A_32_P76811 | ENST00000383511 | chr6: 29802573-29802514 | 1.595358 | 0.017557 |
| A_23_P79043 | NM_032635 | chr19: 40729743-40729891 | 1.60831 | 0.034715 |
| A_24_P151920 | NM_014573 | chr17: 23677691-23677750 | 1.520319 | 0.000887 |
| A_24_P156566 | NM_001078171 | chrX: 133994849-133994908 | 0.769348 | 0.009951 |
| A_23_P110686 | NM_003714 | chr5: 172677032-172676973 | 0.811861 | 0.011801 |
| A_23_P75288 | NM_032810 | chr10: 89503038-89502979 | 2.186211 | 0.02094 |
| A_32_P226009 | NM_023073 | chr5: 37242102-37241301 | 1.858657 | 0.034717 |
| A_24_P764866 | | chr7: 000676993-000677052 | 0.772694 | 0.004581 |
| A_23_P85236 | NM_001078171 | chrX: 133995056-133995115 | 1.586438 | 0.039872 |
| A_24_P913178 | THC2515524 | chr22: 36501865-36501806 | 0.628836 | 0.000788 |
| A_32_P109057 | NM_173825 | chr3: 121932261-121911416 | 0.607093 | 0.018491 |
| A_23_P168818 | NM_005536 | chr8: 82735352-82734244 | 0.686742 | 0.034437 |
| A_23_P13222 | NM_002901 | chr11: 32082505-32082564 | 1.658045 | 0.020321 |
| A_23_P300826 | NM_145029 | chr6: 30727194-30728434 | 1.938399 | 0.041619 |
| A_24_P840868 | | chr16: 073204624-073204244 | 0.737356 | 0.003527 |
| A_23_P10135 | NM_001001349 | chr17: 37429725-37429784 | 0.673414 | 0.029946 |
| A_23_P381261 | NM_139247 | chr14: 23857567-23857508 | 0.740716 | 0.016973 |
| A_23_P159920 | NM_003639 | chrX: 153446058-153446117 | 1.666709 | 0.04234 |
| A_32_P79152 | THC2539923 | chr10: 13809705-13809764 | 0.716361 | 0.012379 |
| A_32_P34480 | THC2664581 | chr7: 151205680-151205621 | 0.75954 | 0.014379 |
| A_23_P326296 | NM_144987 | chr19: 40927136-40927077 | 1.673739 | 0.044445 |
| A_23_P167324 | NM_000297 | chr4: 89198171-89198230 | 1.528733 | 0.017852 |
| A_23_P217958 | NM_033500 | chr10: 70831164-70831223 | 1.818146 | 0.046807 |
| A_24_P259083 | NM_001040022 | chr20: 1867162-1867221 | 1.75291 | 0.01655 |
| A_23_P369471 | NM_178431 | chr1: 150861998-150861939 | 0.799876 | 0.024615 |
| A_32_P165823 | AI431972 | chr20: 48242278-48242219 | 1.610779 | 0.008944 |
| A_23_P118643 | NM_001968 | chr4: 100020603-100020544 | 1.820638 | 0.012796 |
| A_32_P145502 | BE710618 | chr7: 45919919-45920948 | 1.57142 | 0.02158 |
| A_23_P304287 | NM_002803 | chr7: 102795233-102795292 | 0.594166 | 0.038323 |
| A_23_P27873 | NM_018049 | chr19: 2184225-2184166 | 0.55608 | 0.036973 |
| A_24_P85365 | ENST00000398701 | chr10: 075241391-075241332 | 0.792231 | 0.034166 |
| A_23_P132718 | NM_004636 | chr3: 50289469-50289528 | 0.68528 | 0.018169 |
| A_24_P194068 | NM_024310 | chr19: 34857276-34857335 | 0.805306 | 0.01785 |
| A_24_P45446 | NM_052941 | chr1: 89420383-89420324 | 0.687353 | 0.019079 |
| A_23_P201357 | NM_018188 | chr1: 1459646-1459705 | 0.710808 | 0.00277 |
| A_23_P22487 | NM_013271 | chrX: 48574740-48574681 | 0.761933 | 0.010562 |
| A_32_P919718 | NM_178520 | chr17: 76899734-76899675 | 0.817556 | 0.04711 |
| A_23_P256172 | NM_199417 | chr3: 49035622-49035681 | 0.658527 | 0.014532 |
| A_24_P82106 | NM_004995 | chr14: 22385631-22385690 | 1.624931 | 0.01402 |
| A_23_P34606 | NM_004958 | chr1: 11090138-11090079 | 0.555963 | 0.005037 |
| A_23_P391764 | NM_198679 | chr9: 133444470-133444411 | 1.632388 | 0.018556 |
| A_24_P358857 | | chr15: 47052254-47052313 | 0.780843 | 0.032526 |
| A_23_P155079 | NM_013356 | chr22: 36804339-36804280 | 0.700792 | 0.001922 |
| A_24_P134195 | NM_001020818 | chr19: 59069709-59069768 | 1.844039 | 0.00033 |
| A_24_P396650 | NM_002953 | chr1: 26773456-26773515 | 0.693171 | 0.02802 |
| A_23_P427754 | NM_152219 | chr17: 35773541-35773482 | 0.826513 | 0.038198 |
| A_23_P164691 | NM_002162 | chr19: 10305713-10305654 | 0.657001 | 0.012242 |
| A_24_P345451 | NM_024843 | chr2: 172119360-172119419 | 1.710903 | 0.009256 |
| A_23_P158829 | NM_004313 | chr17: 4571277-4571336 | 0.588051 | 0.01289 |
| A_24_P281514 | | chr8: 977214-977155 | 0.746216 | 0.036548 |
| A_24_P384018 | NR_002171 | chr13: 63213041-63214539 | 0.660384 | 0.032227 |
| A_23_P169766 | NM_022740 | chr7: 138907963-138907904 | 1.526373 | 0.02403 |
| A_32_P192545 | NM_001006938 | chrX: 101282291-101282232 | 0.668162 | 0.027348 |
| A_24_P576591 | AL137705 | chr19: 47200304-47200363 | 0.800698 | 0.022177 |
| A_23_P111194 | NM_012391 | chr6: 34614144-34614085 | 0.765605 | 0.03412 |
| A_24_P418744 | | chr4: 25116901-25116960 | 0.708466 | 0.041497 |
| A_32_P227525 | AL522622 | chr19: 12773074-12773602 | 2.459634 | 0.001399 |
| A_24_P98783 | NM_031433 | chr11: 118717792-118717649 | 0.852577 | 0.036708 |
| A_23_P10605 | | chr10: 125759178-125758773 | 1.792582 | 0.022177 |
| A_23_P106814 | | chr16: 002829368-002829427 | 0.74314 | 0.018965 |
| A_23_P374288 | NM_007359 | chr17: 35581538-35581597 | 0.700793 | 0.008956 |
| A_24_P924920 | NM_007011 | chr15: 87544400-87544461 | 2.539821 | 0.005027 |
| A_23_P212968 | NM_001073 | chr4: 70108751-70108692 | 0.830103 | 0.048192 |
| A_24_P161853 | XM_001718037 | chr16: 032913565-032914075 | 0.760967 | 0.03607 |
| A_23_P157478 | NM_015941 | chr8: 54791009-54790950 | 1.491287 | 0.045653 |
| A_23_P31532 | NM_080660 | chr7: 138362032-138361826 | 0.788542 | 0.00237 |
| A_23_P501887 | NM_013407 | chr19: 12651482-12651344 | 0.6611 | 0.042718 |
| A_24_P552677 | | chr8: 143881799-143881740 | 0.806858 | 0.024078 |
| A_24_P873764 | NM_004327 | chr22: 21990120-21990179 | 0.720391 | 0.039766 |
| A_23_P163630 | NM_152287 | chr16: 88331901-88331960 | 0.697387 | 0.012936 |
| A_23_P86390 | NM_003873 | chr10: 33509104-33509045 | 1.640022 | 0.028959 |
| A_23_P153022 | NM_033184 | chr17: 36474895-36474846 | 0.750899 | 0.004846 |
| A_23_P208674 | NM_175063 | chr19: 55678242-55678301 | 1.868216 | 0.028454 |
| A_24_P290114 | | chr14: 103988579-103988638 | 0.745876 | 0.006694 |
| A_24_P384539 | | chrX: 135701082-135701141 | 1.591778 | 0.040925 |
| A_24_P295745 | NM_153201 | chr11: 122435688-122435629 | 0.579891 | 0.002146 |
| A_23_P4060 | AK026312 | chr17: 19063230-19063289 | 0.733434 | 0.00172 |
| A_32_P69456 | DB524775 | chr19: 56490401-56490460 | 0.821202 | 0.040145 |
| A_23_P156017 | NM_022130 | chr5: 32161093-32161034 | 1.668593 | 0.040753 |
| A_32_P57013 | BU540282 | chr2: 60634059-60634117 | 0.747115 | 0.030331 |
| A_24_P148521 | NM_022152 | chr2: 218852052-218851521 | 2.08355 | 0.007144 |
| A_24_P118874 | | chr8: 006744105-006744046 | 1.405351 | 0.025326 |
| A_32_P93187 | AL517609 | chr18: 028246271-028246330 | 0.500737 | 0.008173 |
| A_23_P164999 | NM_004359 | chr19: 492675-492734 | 1.573506 | 0.030072 |
| A_24_P120827 | NM_152419 | chr8: 43175304-43175363 | 1.427312 | 0.034639 |
| A_24_P659415 | BC062632 | chr17: 26386646-26386705 | 0.513961 | 0.008605 |
| A_23_P338134 | ENST00000333235 | chr15: 82744155-82744096 | 0.611698 | 0.017092 |
| A_32_P186450 | NR_024271 | chr7: 45742970-45742911 | 0.678389 | 0.008281 |
| A_23_P117662 | NM_002112 | chr15: 48321521-48321462 | 0.772703 | 0.025742 |
| A_23_P4782 | NM_031896 | chrl9: 59136594-59136653 | 0.78527 | 0.00986 |
| A_23_P157072 | NM_001037283 | chr7: 2386588-2386647 | 1.633341 | 0.028983 |
| A_23_P95810 | NM_007183 | chr11: 394785-394844 | 0.681573 | 0.009603 |
| A_24_P485742 | THC2689427 | chr2: 219643790-219643731 | 0.740288 | 0.033913 |
| A_24_P790909 | AI652176 | chr21: 43708921-43708980 | 0.752243 | 0.02417 |
| A_23_P330532 | NM_080861 | chr16: 1767800-1767438 | 0.67195 | 0.030219 |
| A_32_P191120 | THC2744687 | chr5: 42992782-42992841 | 1.609144 | 0.030933 |
| A_24_P365442 | NM_020062 | chr20: 61843846-61843978 | 0.719179 | 0.02405 |
| A_23_P252653 | NM_006374 | chr2: 242083694-242083635 | 0.499554 | 0.012243 |
| A_23_P17030 | NM_018226 | chr2: 241166303-241166362 | 0.762085 | 0.030255 |
| A_23_P434118 | NM_001024912 | chr19: 47708347-47705199 | 0.715092 | 0.001735 |
| A_23_P55319 | NM_004475 | chr17: 24230908-24230849 | 1.785094 | 0.000423 |
| A_23_P1594 | NM_003377 | chr11: 63762687-63762746 | 1.843174 | 0.03413 |
| A_23_P129103 | AK074778 | chr15_random: 664106-664165 | 0.537464 | 0.005063 |
| A_23_P373126 | BC035647 | chr6: 30004277-30004779 | 0.745113 | 0.027521 |
| A_32_P34926 | AL833696 | chr17: 75789934-75789875 | 0.630492 | 0.039085 |
| A_23_P373598 | NM_002360 | chr7: 1548755-1548814 | 0.669044 | 0.001605 |
| A_32_P527232 | NM_033200 | chr22: 49289695-49289636 | 0.812384 | 0.03376 |
| A_23_P69362 | NM_024661 | chr3: 48448903-48448844 | 0.786201 | 0.02117 |
| A_23_P207896 | NM_001893 | chr17: 77795992-77795933 | 0.572976 | 0.006567 |
| A_23_P119593 | NM_024794 | chr19: 15198870-15198811 | 0.817563 | 0.019777 |
| A_24_P14731 | NM_013271 | chrX: 48575465-48575406 | 0.825667 | 0.035891 |
| A_23_P81399 | NM_003900 | chr5: 179196315-179196374 | 1.524728 | 0.027748 |
| A_24_P88763 | NM_032603 | chr2: 74613930-74613871 | 0.650285 | 0.007097 |
| A_24_P87931 | NM_145343 | chr22: 34993036-34993095 | 0.779933 | 0.047556 |
| A_23_P28434 | NM_003761 | chr2: 85662543-85662602 | 0.624797 | 0.018942 |
| A_32_P167278 | AK056033 | chr21: 39636439-39636384 | 1.87851 | 0.019166 |
| A_24_P322847 | NM_138338 | chr22: 40251872-40251813 | 0.627759 | 0.003377 |
| A_24_P396340 | NM_138428 | chr1: 35093082-35093023 | 0.692771 | 0.021698 |
| A_24_P401150 | | chrX: 119751054-119751113 | 0.784049 | 0.026448 |
| A_32_P738377 | ENST00000400385 | chr21: 44050861-44050802 | 0.72971 | 0.028627 |
| A_24_P50567 | | chr6: 144563421-144563344 | 1.998991 | 0.018917 |
| A_23_P425224 | NM_020119 | chr7: 138381499-138381440 | 1.683804 | 0.040293 |
| A_23_P315320 | NM_145659 | chr16: 28418548-28418489 | 0.740051 | 0.01154 |
| A_24_P29260 | NM_054013 | chr5: 179158842-179158702 | 0.701013 | 0.015682 |
| A_32_P1869 | | chr10: 007367758-007367817 | 0.564491 | 0.000465 |
| A_24_P690983 | ENST00000398662 | chr22: 20991533-20991592 | 0.75006 | 0.045605 |
| A_23_P360209 | ENST00000361227 | chrM: 10200-10259 | 1.487745 | 0.021294 |
| A_32_P228869 | NM_006937 | chr17: 70675543-70675484 | 1.398088 | 0.039926 |
| A_24_P168494 | NM_001082 | chr19: 15850683-15850624 | 0.766442 | 0.012545 |
| A_32_P111072 | THC2781239 | chr8: 106546265-106546324 | 0.769756 | 0.034634 |
| A_23_P155453 | NM_033199 | chr3: 48575459-48575400 | 0.801765 | 0.011484 |
| A_32_P538017 | NM_005995 | chr11: 67155447-67155388 | 0.699853 | 0.000424 |
| A_24_P158587 | NM_021117 | chr11: 45859972-45860031 | 0.779865 | 0.029633 |
| A_23_P343900 | ENST00000390638 | chr14: 106330534-106330475 | 0.730262 | 0.000822 |
| A_24_P912300 | THC2498439 | chr1: 70489546-70489487 | 2.017327 | 0.009744 |
| A_23_P165295 | NM_002501 | chr19: 12997196-12997255 | 1.473636 | 0.044191 |
| A_23_P374104 | NM_012098 | chr9: 128891100-128891041 | 1.835073 | 0.013082 |
| A_23_P331895 | NM_025250 | chr7: 2670100-2670159 | 1.629169 | 0.028917 |
| A_32_P172917 | XR_038215 | chr2: 132241326-132241385 | 0.61857 | 0.004252 |
| A_24_P102726 | NM_000309 | chr1: 159403528-159403587 | 0.814606 | 0.048433 |
| A_23_P152818 | NM_007067 | chr17: 45260689-45260748 | 1.395269 | 0.041711 |
| A_23_P20832 | NM_003127 | chr9: 130435543-130435602 | 2.037597 | 0.00698 |
| A_32_P27240 | THC2665816 | chr19: 20974746-20974805 | 0.812646 | 0.040468 |
| A_23_P90470 | XR_037365 | chr19: 015556842-015556901 | 0.735872 | 0.008198 |
| A_23_P116091 | NM_130443 | chr11: 66028759-66028818 | 0.712999 | 0.044424 |
| A_23_P334751 | NM_152490 | chr1: 233680177-233680118 | 1.677826 | 0.02135 |
| A_24_P341897 | NM_004302 | chr12: 50674874-50674933 | 2.125911 | 0.004587 |
| A_23_P81180 | NM_022832 | chr4: 53157446-53157387 | 1.614026 | 0.042467 |
| A_32_P12504 | BC024745 | chr11: 43921760-43921701 | 1.404586 | 0.038383 |
| A_23_P40272 | NM_001501 | chr20: 2973170-2973339 | 0.801108 | 0.01852 |
| A_23_P160367 | NM_005119 | chr1: 36542554-36542613 | 1.610099 | 0.047639 |
| A_24_P290046 | | chr3: 075801154-075801213 | 0.799639 | 0.012536 |
| A_24_P269527 | NM_020427 | chr8: 143820264-143819679 | 0.751678 | 0.0101 |
| A_32_P156628 | BQ379494 | chr1: 180620346-180620405 | 1.852983 | 0.005621 |
| A_23_P45864 | NM_003285 | chr1: 173571497-173565963 | 0.794655 | 0.033751 |
| A_23_P4153 | NM_001113738 | chr17: 38545526-38545585 | 0.637539 | 0.042285 |
| A_23_P81241 | NM_030571 | chr5: 141512298-141512357 | 1.74678 | 0.031904 |
| A_23_P112666 | NM_012186 | chr1: 47656101-47656160 | 0.834136 | 0.038143 |
| A_24_P336848 | NM_138448 | chr2: 54219300-54219359 | 0.60039 | 0.01894 |
| A_23_P128084 | NM_002206 | chr12: 54364746-54364687 | 0.68604 | 0.016219 |
| A_32_P56618 | XM_001726460 | chr7: 62861124-62861117 | 0.745808 | 0.004401 |
| A_32_P73491 | | chr20: 020323475-020323534 | 0.746263 | 0.034085 |
| A_24_P182122 | ENST00000361390 | chrM: 3369-3428 | 1.524121 | 0.034686 |
| A_32_P77759 | | chr17: 076918586-076918537 | 0.754056 | 0.005343 |
| A_23_P6355 | NR_024591 | chr22: 17226105-17226164 | 0.727273 | 0.038556 |
| A_32_P227496 | BQ684512 | chr17_random: | 0.745369 | 0.020952 |
| | | 000554768-000554710 | | |
| A_24_P237231 | NM_173354 | chr21: 43661409-43661350 | 0.713887 | 0.012916 |
| A_32_P79115 | XR_039086 | chr1: 147940002-147939943 | 0.755594 | 0.016662 |
| A_32_P2354 | BG114486 | chr16: 29617003-29617062 | 0.65226 | 0.008409 |
| A_24_P178175 | NM_005265 | chr22: 23353871-23353930 | 0.688469 | 0.025098 |
| A_24_P127564 | NR_024330 | chr19: 56898301-56898360 | 0.703226 | 0.009484 |
| A_23_P200560 | NM_001039802 | chr1: 22291856-22291915 | 1.472702 | 0.039877 |
| A_32_P130962 | AF131777 | chr13: 110352016-110352075 | 1.721149 | 0.020873 |
| A_23_P166677 | NM_022736 | chr3: 160030005-160030064 | 1.727465 | 0.008953 |
| A_23_P31536 | NM_003143 | chr7: 141091792-141091851 | 1.852285 | 0.042023 |
| A_24_P310864 | NM_152499 | chr1: 44234715-44234774 | 0.652835 | 0.005731 |
| A_23_P40078 | NM_003400 | chr2: 61559399-61559340 | 1.673559 | 0.023194 |
| A_24_P928689 | | chr1: 242927874-242927815 | 0.753745 | 0.028628 |
| A_32_P111565 | NM_002032 | chr11: 61488920-61488861 | 1.830969 | 0.049528 |
| A_23_P143559 | NM_007098 | chr22: 17547335-17547276 | 0.738781 | 0.019885 |
| A_24_P237613 | NM_182488 | chr13: 26541088-26541029 | 2.251617 | 0.015584 |
| A_23_P122508 | NM_080870 | chr6: 31029261-31029320 | 0.855787 | 0.046748 |
| A_24_P499481 | | chr1: 004480750-004480809 | 0.805722 | 0.034872 |
| A_32_P202621 | | chr16: 087079898-087079848 | 0.757405 | 0.023263 |
| A_23_P361085 | NR_003038 | chr6: 86443878-86443587 | 1.710845 | 0.007895 |
| A_23_P58529 | NM_014473 | chr5: 61720364-61720305 | 0.75395 | 0.013539 |
| A_23_P316064 | NM_180990 | chr17: 71588975-71589034 | 1.737994 | 0.004329 |
| A_23_P116682 | NM_139067 | chr12: 54845744-54845685 | 0.678934 | 0.014089 |
| A_24_P405054 | NM_015609 | chr1: 16594123-16594182 | 0.715208 | 0.028296 |
| A_32_P74831 | THC2614421 | chr15: 25460642-25460701 | 0.822746 | 0.008571 |
| A_24_P415624 | NM_014938 | chr12: 121184407-121184466 | 0.742505 | 0.03877 |
| A_32_P212886 | NM_001130966 | chr7: 139133619-139133678 | 0.733876 | 0.005208 |
| A_24_P124662 | NM_139078 | chr12: 110815163-110815222 | 1.841789 | 0.043009 |
| A_23_P429340 | NM_021961 | chr11: 12908300-12908359 | 1.785435 | 0.044776 |
| A_23_P218937 | NM_032402 | chr5: 140837991-140838050 | 1.611973 | 0.039616 |
| A_24_P104538 | BC000924 | chr17: 41720014-41719955 | 0.63938 | 0.016507 |
| A_24_P390793 | NM_001416 | chr17: 7421746-7421925 | 1.950107 | 0.010344 |
| A_23_P16415 | NM_002333 | chr19: 38390176-38390235 | 0.739469 | 0.01551 |
| A_32_P118686 | BC021035 | chr17: 69770671-69770730 | 2.307322 | 0.019136 |
| A_23_P29575 | NM_033281 | chr5: 68560792-68560851 | 0.596546 | 0.006342 |
| A_23_P23443 | NM_024329 | chr1: 15629224-15629283 | 1.564378 | 0.021267 |
| A_24_P659202 | THC2527772 | chr6: 32077541-32077595 | 1.314037 | 0.025327 |
| A_24_P293089 | NM_152600 | chr19: 60781891-60781832 | 0.809286 | 0.044096 |
| A_23_P3663 | NM_138418 | chr16: 638364-638423 | 0.686795 | 0.00706 |
| A_23_P9152 | NM_005772 | chr9: 4850885-4850943 | 0.600644 | 0.00117 |
| A_32_P160398 | AA911065 | chr12: 111327461-111327520 | 0.642519 | 0.002349 |
| A_24_P314534 | NM_181621 | chr21: 30665941-30665882 | 0.851305 | 0.009516 |
| A_23_P365817 | NM_138689 | chr11: 63768626-63768567 | 1.509694 | 0.049224 |
| A_32_P88965 | BX537651 | chr1: 161021051-161021110 | 1.56779 | 0.03733 |
| A_23_P149470 | NM_004550 | chr1: 159446761-159447021 | 1.615954 | 0.039389 |
| A_23_P80353 | NM_031488 | chr22: 39956133-39956192 | 0.775858 | 0.003404 |
| A_23_P372386 | NM_173833 | chr8: 27879913-27879854 | 0.803731 | 0.020577 |
| A_23_P153540 | NM_017708 | chr19: 53804993-53798970 | 0.758274 | 0.00664 |
| A_24_P30389 | NM_152717 | chr11: 60297528-60297939 | 0.811369 | 0.033589 |
| A_24_P58187 | XM_001718684 | chr11: 110164223-110164164 | 0.713088 | 0.035328 |
| A_23_P30435 | NM_006058 | chr5: 150390018-150389959 | 0.585692 | 0.012443 |
| A_23_P52298 | NM_006993 | chr10: 103531268-103531209 | 0.67509 | 0.015754 |
| A_23_P11713 | NM_080392 | chr1: 32157158-32154117 | 2.004381 | 0.005277 |
| A_23_P171223 | NM_022101 | chrX: 118562437-118562378 | 0.653074 | 0.021452 |
| A_23_P392575 | NM_080671 | chr2: 223626768-223626827 | 0.625374 | 0.003933 |
| A_24_P929974 | | | 1.678479 | 0.038693 |
| A_23_P30884 | NM_001288 | chr6: 31808084-31808025 | 2.091714 | 0.002737 |
| A_24_P651736 | AI859463 | chr14: 21040395-21040454 | 0.57805 | 0.022414 |
| A_23_P108785 | NM_005721 | chr2: 114431973-114432032 | 1.898012 | 0.034477 |
| A_24_P384469 | | chr18: 074515771-074515830 | 0.849351 | 0.029233 |
| A_23_P93818 | AL137492 | chr7: 74857450-74857509 | 1.617337 | 0.039727 |
| A_24_P914095 | CB269608 | chr11: 066365944-066365885 | 1.616229 | 0.043693 |
| A_32_P204258 | THC2688486 | chr10: 79324402-79324343 | 0.54943 | 0.005577 |
| A_24_P126873 | NM_003170 | chr17: 24050992-24051330 | 1.644126 | 0.001251 |
| A_23_P210048 | NM_006037 | chr2: 239635672-239635613 | 1.500397 | 0.031263 |
| A_24_P303524 | NM_182924 | chr7: 1440600-1440541 | 1.521523 | 0.043368 |
| A_24_P336596 | NM_030663 | chr1: 151123583-151123642 | 0.815077 | 0.000928 |
| A_23_P28707 | NM_007346 | chr20: 60915658-60915717 | 0.676541 | 0.010119 |
| A_24_P38930 | NM_001331 | chr11: 57341186-57341245 | 1.961536 | 0.030042 |
| A_24_P410895 | AF119866 | chr9: 125067881-125067822 | 1.571149 | 0.025331 |
| A_24_P377489 | NM_015622 | chr7: 5930024-5930083 | 1.95629 | 0.019129 |
| A_23_P408341 | NR_002781 | chr20: 30241066-30241007 | 0.819988 | 0.011129 |
| A_23_P56380 | NM_018471 | chr2: 187081961-187082020 | 1.85158 | 0.009653 |
| A_24_P417984 | NM_138689 | chr11: 63769321-63769262 | 1.687321 | 0.045622 |
| A_23_P207680 | NM_001039083 | chr17: 41794825-41791505 | 0.467351 | 0.002017 |
| A_24_P401321 | | chr2: 61791518-61791459 | 0.762653 | 0.000907 |
| A_23_P18901 | NM_004707 | chr5: 115201321-115201262 | 1.915359 | 0.049917 |
| A_23_P84762 | NM_032789 | chr8: 145123577-145123518 | 0.820464 | 0.034354 |
| A_24_P353905 | NM_032348 | chr1: 1279460-1279315 | 1.474091 | 0.048213 |
| A_24_P519817 | AL834347 | chr16: 30904409-30904350 | 0.731844 | 0.005381 |
| A_24_P153363 | | chr15: 071849165-071849225 | 0.763642 | 0.026378 |
| A_24_P125993 | NM_004712 | chr17: 77278493-77278552 | 0.666241 | 0.029027 |
| A_32_P509169 | NM_207322 | chr15: 60147273-60147332 | 0.813716 | 0.005211 |
| A_24_P402222 | NM_022555 | | 0.706652 | 0.042571 |
| A_23_P168541 | NM_024067 | chr7: 6614508-6614567 | 0.673857 | 0.014283 |
| A_23_P42282 | NM_001002029 | chr6: 32077570-32077891 | 1.305862 | 0.035657 |
| A_24_P639679 | AK095831 | chr5: 9602609-9602668 | 1.557769 | 0.007228 |
| A_23_P88439 | NM_152332 | chr14: 91317428-91317369 | 1.367174 | 0.04384 |
| A_24_P577694 | NM_021116 | chr7: 45728853-45728910 | 0.754316 | 0.021738 |
| A_32_P109755 | AF462446 | chr14: 104128400-104128341 | 0.731888 | 0.021728 |
| A_23_P37111 | NM_177438 | chr14: 94626045-94625986 | 1.645578 | 0.038694 |
| A_24_P383019 | NM_181599 | chr21: 30690727-30690786 | 0.85746 | 0.020035 |
| A_23_P91015 | NM_006062 | chr2: 73307430-73307489 | 0.636325 | 0.03803 |
| A_32_P77178 | AA085955 | chr1: 152822085-152822144 | 1.503584 | 0.038676 |
| A_23_P213857 | NM_000587 | chr5: 41017658-41017717 | 0.833891 | 0.044383 |
| A_23_P44112 | NM_014387 | chr16: 28909233-28909292 | 0.725 | 0.039676 |
| A_24_P222860 | NM_002651 | chr1: 149533232-149533173 | 0.710672 | 0.02764 |
| A_23_P17769 | NM_001355 | chr22: 22643789-22643730 | 0.739168 | 0.023167 |
| A_23_P152115 | NM_002513 | chr16: 1760411-1760352 | 0.652119 | 0.000642 |
| A_24_P775659 | CR593500 | chr19: 3803247-3803188 | 0.802978 | 0.039854 |
| A_23_P88753 | NM_001001410 | chr16: 1340067-1339903 | 2.150441 | 0.003981 |
| A_24_P176409 | NM_133458 | chr16: 67156831-67156890 | 1.911686 | 0.030594 |
| A_24_P110062 | BC014048 | chr11: 47551402-47551461 | 2.348103 | 0.019428 |
| A_24_P135322 | NM_001024629 | chr10: 33542356-33536628 | 1.660504 | 0.019762 |
| A_23_P86242 | NM_018713 | chr1: 218155127-218155068 | 0.794917 | 0.005744 |
| A_23_P25935 | NM_007176 | chr14: 75187656-75187597 | 0.670457 | 0.02967 |
| A_23_P339225 | AK025264 | chr4: 48081812-48081871 | 1.718992 | 0.00813 |
| A_23_P107735 | NM_001783 | chr19: 47077196-47077255 | 0.802409 | 0.039097 |
| A_23_P88234 | NM_016049 | chr14: 23678419-23678197 | 0.65988 | 0.03303 |
| A_23_P329212 | NM_005238 | chr11: 127861222-127860166 | 0.650712 | 0.03374 |
| A_24_P143686 | NM_001080394 | chr8: 48804580-48810480 | 1.938406 | 0.009656 |
| A_24_P151959 | AK021860 | chr19: 44127908-44127849 | 0.749559 | 0.019489 |
| A_23_P203702 | THC2707284 | chr11: 74653186-74653127 | 0.666048 | 0.005282 |
| A_23_P354734 | NM_032088 | chr5: 140872578-140872637 | 1.531338 | 0.011589 |
| A_24_P190424 | NM_005370 | chr19: 16104038-16104097 | 0.711634 | 0.029074 |
| A_24_P392436 | | chr16: 002912909-002912850 | 1.350572 | 0.042277 |
| A_32_P98298 | NM_004420 | chr11: 1535970-1535717 | 0.748729 | 0.013101 |
| A_23_P168882 | NM_033285 | chr8: 96007929-96007870 | 0.725582 | 0.021605 |
| A_23_P203994 | NM_002813 | chr12: 120838216-120838614 | 0.737711 | 0.014199 |
| A_23_P55256 | NM_014897 | chr17: 44727663-44727604 | 1.297794 | 0.042704 |
| A_24_P828125 | | chr20: 061170510-061170569 | 0.768033 | 0.025978 |
| A_23_P23206 | NM_006341 | chr1: 11657809-11657750 | 0.741427 | 0.007239 |
| A_24_P919460 | AB065089 | chr16: 1952735-1952676 | 0.753059 | 0.01969 |
| A_23_P15647 | NM_016231 | chr17: 23545825-23545884 | 1.564451 | 0.00873 |
| A_23_P399112 | NM_001020818 | chr19: 59068804-59068863 | 2.037354 | 0.003484 |
| A_23_P67648 | NM_001749 | chr19: 41332649-41332708 | 1.805757 | 0.027041 |
| A_23_P251855 | NM_006598 | chr5: 1105147-1105088 | 1.549271 | 0.028642 |
| A_24_P930327 | | chr19: 044379827-044379768 | 0.823073 | 0.025189 |
| A_24_P930415 | NM_001012508 | chr10: 135085621-135085562 | 0.808522 | 0.020092 |
| A_23_P55076 | NM_130766 | chr17: 1344681-1344622 | 0.672555 | 0.025233 |
| A_23_P202683 | NM_021924 | chr11: 607509-607450 | 0.825114 | 0.049959 |
| A_24_P273423 | AK023542 | chr2: 39502439-39502380 | 2.069125 | 0.010677 |
| A_24_P269895 | NM_194247 | chr2: 177788575-177788634 | 2.543235 | 0.033132 |
| A_24_P380919 | NM_002140 | chr9: 85773320-85773261 | 2.182459 | 0.019304 |
| A_23_P34983 | NM_006694 | chr1: 152215845-152214999 | 1.836803 | 0.041942 |
| A_23_P12730 | NM_015235 | chr10: 53127442-53127383 | 1.660372 | 0.021102 |
| A_23_P38712 | NM_001099733 | chr18: 900632-900691 | 0.802165 | 0.004626 |
| A_23_P27473 | NM_032488 | chr19: 47583085-47583026 | 0.693536 | 0.005277 |
| A_23_P62021 | L12350 | chr6: 169358199-169358136 | 1.402605 | 0.044105 |
| A_23_P57227 | NM_003098 | chr20: 31459519-31459460 | 0.733469 | 0.034644 |
| A_24_P828054 | | chr6: 27287611-27287670 | 0.616305 | 0.048639 |
| A_23_P27840 | NM_003169 | chr19: 44658591-44658650 | 1.718733 | 0.022579 |
| A_32_P190294 | AK302866 | chr5: 34211760-34211701 | 1.75933 | 0.028266 |
| A_23_P64247 | BC033000 | chr11: 43422465-43422524 | 1.996471 | 0.026372 |
| A_24_P824578 | | chr5: 33538193-33538252 | 0.79357 | 0.026762 |
| A_32_P92922 | | | 0.734289 | 0.011712 |
| A_23_P55251 | NM_002204 | chr17: 45522364-45522423 | 1.398795 | 0.010428 |
| A_24_P478153 | DA572835 | chr17: 19790635-19790694 | 0.609788 | 0.000353 |
| A_24_P353794 | NM_004481 | chr1: 228483885-228483944 | 1.841595 | 0.008901 |
| A_23_P408768 | NM_032482 | chr19: 2178105-2180821 | 0.77677 | 0.009766 |
| A_23_P66473 | NM_181671 | chr17: 63102083-63113665 | 1.639653 | 0.006615 |
| A_23_P56949 | NM_014755 | chr2: 64716407-64716348 | 1.767511 | 0.01652 |
| A_24_P160466 | NM_052899 | chr5: 175955615-175955556 | 0.728298 | 0.018914 |
| A_23_P54840 | NM_005946 | chr16: 55230706-55231309 | 0.757511 | 0.031298 |
| A_23_P108574 | NM_005813 | chr2: 37333967-37333908 | 1.862101 | 0.03714 |
| A_23_P146084 | NM_000637 | chr8: 30656570-30656511 | 0.465652 | 0.002044 |
| A_32_P185229 | NM_020452 | chr1: 152590102-152590161 | 0.771476 | 0.030345 |
| A_24_P872967 | THC2662876 | chr18: 31082088-31082029 | 1.599877 | 0.031562 |
| A_24_P46159 | NM_016479 | chr3: 48485833-48485774 | 0.767944 | 0.046465 |
| A_23_P133279 | NM_199133 | chr5: 10279717-10279658 | 0.697508 | 0.001081 |
| A_23_P127995 | NM_001293 | chr11: 77005043-77004984 | 0.722614 | 0.04807 |
| A_23_P154345 | NM_014362 | chr2: 190778046-190777987 | 0.584022 | 0.000611 |
| A_23_P4754 | NM_006012 | chr19: 6319801-6319860 | 0.572185 | 0.015147 |
| A_24_P924185 | S81524 | | 1.459766 | 0.044313 |
| A_32_P65022 | BC144530 | chr22: 15775318-15775259 | 0.718471 | 0.00364 |
| A_23_P102994 | NM_002650 | chr22: 19395051-19394992 | 0.563781 | 0.012733 |
| A_32_P194072 | NM_015395 | chr7: 97684201-97684142 | 0.765872 | 0.020564 |
| A_32_P213637 | AK092942 | chr4: 120546277-120546218 | 1.804727 | 0.011219 |
| A_24_P221206 | NM_174894 | chr19: 7653604-7653663 | 0.743825 | 0.024492 |
| A_23_P156087 | NM_000163 | chr5: 42755379-42755438 | 0.708507 | 0.004524 |
| A_23_P106299 | NM_001018108 | chr15: 41873224-41873283 | 1.732693 | 0.043417 |
| A_23_P218203 | NM_001114632 | chr15: 39916967-39917026 | 0.586464 | 0.018456 |
| A_24_P103803 | NM_001497 | chr9: 33100782-33100723 | 1.888212 | 0.014661 |
| A_32_P134290 | NM_017742 | chr18: 58396505-58396564 | 1.576204 | 0.025556 |
| A_32_P84289 | NM_152371 | chr1: 2512337-2512396 | 0.651356 | 0.006745 |
| A_24_P734892 | | chr5: 001386540-001386486 | 1.567678 | 0.011665 |
| A_24_P510116 | AK057970 | chr13: 110346303-110346244 | 1.81279 | 0.018203 |
| A_23_P206806 | NM_002209 | chr16: 30441409-30441468 | 0.756912 | 0.048631 |
| A_23_P354175 | NM_138385 | chr4: 1687570-1687511 | 1.715454 | 0.014545 |
| A_32_P76112 | | chr12: 67322848-67322789 | 1.774569 | 0.008603 |
| A_23_P26745 | AK026675 | chr17: 17388803-17388744 | 0.750479 | 0.004737 |
| A_24_P19544 | NM_013233 | chr2: 168519167-168519108 | 1.4162 | 0.041129 |
| A_23_P31335 | NM_016265 | chr7: 6696883-6696824 | 1.840177 | 0.035644 |
| A_23_P154025 | NM_003142 | chr2: 170376560-170376619 | 1.665845 | 0.048929 |
| A_24_P33552 | XM_001133763 | chr6: 133360475-133360416 | 1.583745 | 0.030399 |
| A_23_P257672 | NM_177458 | chr8: 143842833-143842774 | 0.825493 | 0.040573 |
| A_24_P848662 | CR594528 | chr16: 645331-645272 | 0.81886 | 0.037643 |
| A_23_P127367 | NM_021173 | chr11: 66875773-66875714 | 0.48353 | 0.000906 |
| A_23_P28869 | NM_002836 | chr20: 2966788-2966847 | 0.694377 | 0.028787 |
| A_23_P106532 | NM_130468 | chr15: 38552502-38552561 | 0.715039 | 0.014348 |
| A_24_P238016 | U63828 | chr20: 61945392-61945333 | 0.793387 | 0.035389 |
| A_24_P417546 | BC004936 | chr4: 83845462-83845403 | 0.80392 | 0.039956 |
| A_23_P51572 | NM_005999 | chr1: 229767022-229767081 | 1.886385 | 0.033974 |
| A_32_P11372 | NR_003674 | chr9: 46585969-46586028 | 0.779693 | 0.048612 |
| A_23_P381347 | NM_022640 | chr17: 59326294-59326235 | 0.841982 | 0.013342 |
| A_24_P358321 | ENST00000390267 | chr2: 89624196-89624255 | 0.661046 | 0.000126 |
| A_23_P90601 | NM_182915 | chr2: 119739468-119739527 | 0.734565 | 0.005996 |
| A_24_P24183 | NR_024416 | chr7: 44047281-44047340 | 0.695713 | 0.002568 |
| A_23_P160103 | NM_022902 | chr5: 68454816-68454875 | 1.857934 | 0.041135 |
| A_23_P27515 | NM_012268 | chr19: 45575592-45575751 | 1.889715 | 0.009001 |
| A_24_P366390 | NM_198181 | chr15_random: 637901-638997 | 0.713543 | 0.019008 |
| A_23_P218434 | NM_001032374 | chr19: 49371127-49371186 | 0.785133 | 0.030347 |
| A_23_P151106 | NM_007273 | chr12: 6944919-6944860 | 0.443039 | 0.00782 |
| A_24_P394569 | AK056855 | chr6: 52856647-52856706 | 0.479529 | 0.020206 |
| A_32_P234543 | THC2719015 | chr10: 22055702-22055643 | 0.717476 | 0.049142 |
| A_24_P207907 | NM_014925 | chr12: 55934717-55934658 | 1.967566 | 0.02728 |
| A_23_P312415 | NM_052866 | chr9: 18674771-18674830 | 0.858784 | 0.024243 |
| A_23_P148785 | NM_199344 | chr1: 166472583-166475002 | 1.554619 | 0.048843 |
| A_32_P49959 | BX111592 | chrX: 152314410-152314351 | 0.760868 | 0.0377 |
| A_24_P34545 | NM_032329 | chr2: 242299491-242299550 | 0.817642 | 0.037147 |
| A_24_P106145 | NM_017491 | chr4: 9688522-9688159 | 1.84784 | 0.04131 |
| A_32_P203362 | AL561405 | chr3: 125939505-125939446 | 0.65427 | 0.005511 |
| A_32_P187975 | AI698357 | chr6: 31915358-31915299 | 2.03003 | 0.044942 |
| A_23_P56249 | NM_001281 | chr19: 41308258-41308461 | 1.665113 | 0.049492 |
| A_32_P77416 | BF368414 | chr13: 98801902-98801843 | 1.55959 | 0.012085 |
| A_23_P162336 | NM_014515 | chr12: 69026358-69033921 | 1.915879 | 0.016854 |
| A_32_P65793 | XM_001714405 | chr13: 98537685-98537744 | 0.823569 | 0.042705 |
| A_23_P161074 | NM_015602 | chr1: 178154288-178154347 | 1.39301 | 0.031908 |
| A_23_P366455 | NM_133264 | chr17: 35689355-35689414 | 0.662929 | 0.019166 |
| A_23_P73559 | NM_004463 | chrX: 54488965-54488906 | 0.675906 | 0.021772 |
| A_23_P127579 | NM_000317 | chr11: 111609468-111609527 | 1.679069 | 0.036456 |
| A_23_P67521 | NM_019612 | chr19: 48915642-48915701 | 0.776532 | 0.005888 |
| A_32_P91633 | AA725389 | chr3: 23935882-23935825 | 0.533156 | 0.022747 |
| A_24_P24244 | NM_001007026 | chr12: 6916260-6916319 | 0.702237 | 0.024583 |
| A_32_P211253 | ENST00000331096 | chr12: 109859550-109859609 | 0.847591 | 0.035981 |
| A_24_P401521 | | chr7: 006724586-006724534 | 1.51723 | 0.036043 |
| A_23_P27584 | NM_001020818 | chr19: 59071373-59071432 | 1.573065 | 0.018594 |
| A_24_P50157 | XR_017483 | chr6: 151588395-151588356 | 0.579027 | 0.007797 |
| A_32_P49844 | NM_012249 | chr2: 46656883-46657251 | 1.94753 | 0.027065 |
| A_24_P40055 | NM_052925 | chr19: 59664946-59665005 | 2.085775 | 0.005284 |
| A_32_P159820 | THC2677326 | chr12: 123433886-123433945 | 0.750007 | 0.015432 |
| A_23_P79889 | NM_020673 | chr20: 56368866-56368925 | 0.605885 | 0.025466 |
| A_23_P164089 | NM_057178 | chr17: 30360383-30360324 | 1.506548 | 0.019038 |
| A_23_P16078 | NM_002573 | chr19: 47496053-47495994 | 0.642901 | 0.01577 |
| A_23_P119418 | NM_003796 | chr19: 35195068-35195127 | 1.468543 | 0.036247 |
| A_32_P820503 | NM_002032 | chr11: 61488347-61488317 | 1.444066 | 0.042041 |
| A_24_P683861 | ENST00000390594 | chr14: 105523996-105523937 | 0.804079 | 0.041227 |
| A_23_P167067 | NM_003359 | chr4: 39178256-39178197 | 1.619394 | 0.009149 |
| A_23_P152655 | NM_000873 | chr17: 59433751-59433692 | 0.671557 | 0.040313 |
| A_24_P362432 | NM_002393 | chr1: 202778643-202780292 | 1.769722 | 0.030887 |
| A_32_P3113 | AF070529 | chr19: 8409714-8409655 | 0.445298 | 0.01757 |
| A_23_P89310 | NM_014964 | chr17: 19180507-19180566 | 0.422224 | 0.000441 |
| A_24_P113521 | NM_022720 | chr22: 18454131-18454190 | 0.589372 | 0.01747 |
| A_23_P71889 | NM_153437 | chr9: 130294867-130296630 | 0.741531 | 0.034588 |
| A_23_P53976 | NM_206949 | chr14: 93638655-93638714 | 0.537129 | 0.001066 |
| A_32_P220625 | THC2654007 | chr2: 64734902-64734843 | 0.771601 | 0.048082 |
| A_32_P116219 | | chr11: 000497468-000497409 | 0.694594 | 0.018837 |
| A_23_P430442 | NM_020772 | chr17: 24613046-24612987 | 1.337862 | 0.044318 |
| A_23_P56314 | NM_006830 | chr19: 1549153-1549094 | 0.744203 | 0.013904 |
| A_24_P821988 | H62159 | chr10: 48546115-48546170 | 0.755667 | 0.036312 |
| A_23_P159325 | NM_139314 | chr19: 8345169-8345228 | 1.408508 | 0.008295 |
| A_23_P110445 | NM_006051 | chr5: 139918331-139918272 | 0.498454 | 0.022171 |
| A_24_P350252 | | chr11: 000970519-000970460 | 0.782674 | 0.024698 |
| A_32_P133464 | AI885257 | chr5: 1371327-1371386 | 2.097782 | 0.030132 |
| A_23_P164674 | NM_013362 | chr19: 49328223-49328282 | 0.593186 | 0.000277 |
| A_23_P343382 | AB011086 | chr10: 46425532-46425591 | 0.746718 | 0.017323 |
| A_24_P367247 | NM_181607 | chr21: 30774393-30774334 | 0.78165 | 0.005491 |
| A_23_P215461 | NM_002314 | chr7: 73174599-73174658 | 1.798458 | 0.030826 |
| A_23_P391506 | NM_006469 | chr1: 183532792-183532733 | 1.820165 | 0.029618 |
| A_24_P270890 | NM_001007226 | chr17: 45032058-45031999 | 1.589423 | 0.0118 |
| A_23_P9293 | NM_004817 | chr9: 71059264-71059323 | 0.676537 | 0.017439 |
| A_23_P340338 | NM_178435 | chr1: 150805288-150805229 | 0.741866 | 0.009764 |
| A_24_P392833 | | chr1: 149143099-149143158 | 0.715582 | 0.046453 |
| A_23_P99891 | NM_022566 | chr15: 79083223-79083282 | 1.627852 | 0.041003 |
| A_23_P252825 | NM_012235 | chr3: 47430537-47430478 | 0.655871 | 0.017061 |
| A_23_P85853 | NM_024587 | chr1: 44892936-44892877 | 0.708167 | 0.017014 |
| A_23_P66073 | AF113013 | chr16: 80065900-80065959 | 0.768709 | 0.00709 |
| A_23_P158627 | NM_024078 | chr12: 131202605-131202664 | 0.836921 | 0.039518 |
| A_24_P316532 | BC040881 | chr6: 44385206-44385265 | 0.641532 | 0.008707 |
| A_24_P14531 | NM_014742 | chr20: 30218555-30218614 | 1.660587 | 0.029945 |
| A_23_P46170 | NM_052877 | chr1: 43622444-43622385 | 1.706827 | 0.027109 |
| A_23_P213458 | NM_001037637 | chr5: 72836942-72837001 | 0.618978 | 0.015604 |
| A_23_P405129 | NM_000428 | chr14: 74035264-74035205 | 1.603293 | 0.03924 |
| A_24_P764598 | NM_080392 | chr1: 32157839-32157780 | 1.688737 | 0.029045 |
| A_32_P97513 | THC2679050 | chr20: 015711101-015711160 | 0.699441 | 0.002273 |
| A_24_P99152 | NM_004926 | chr14: 68326087-68326028 | 1.742469 | 0.008939 |
| A_23_P23572 | NM_003557 | chr1: 149481666-149486974 | 1.59364 | 0.047201 |
| A_23_P11262 | NM_012151 | chrX: 153769414-153769473 | 1.598362 | 0.030687 |
| A_23_P46412 | NM_002978 | chr1: 1216176-1216346 | 0.775452 | 0.009781 |
| A_23_P204696 | NM_004064 | chr12: 12762386-12762445 | 0.733086 | 0.04309 |
| A_32_P104619 | AK094156 | chr1: 143228185-143228126 | 1.501386 | 0.022457 |
| A_24_P914376 | CR610088 | chr19: 11353383-11352946 | 0.782607 | 0.007248 |
| A_23_P30464 | NM_030567 | chr5: 176815750-176815809 | 0.84019 | 0.029131 |
| A_23_P382775 | NM_014417 | chr19: 52416073-52416014 | 0.611818 | 0.003612 |
| A_23_P45496 | NM_001493 | chrX: 153324796-153324855 | 1.983347 | 0.025052 |
| A_24_P182182 | NM_001152 | chrX: 118488101-118488385 | 0.515503 | 0.002471 |
| A_24_P106454 | NM_025263 | chr6: 30638147-30638206 | 0.685983 | 0.007895 |
| A_24_P405621 | NM_007184 | chr3: 52501418-52501477 | 0.800236 | 0.049274 |
| A_23_P163347 | NM_016166 | chr15: 66267161-66267220 | 0.680077 | 0.002098 |
| A_23_P154585 | NM_001042633 | chr20: 43903697-43903756 | 0.586485 | 0.024027 |
| A_23_P301925 | ENST00000361624 | chr1: 557430-557489 | 1.677235 | 0.032737 |
| A_24_P376309 | NM_032595 | chr17: 45566188-45566129 | 1.487071 | 0.036001 |
| A_32_P179615 | THC2614521 | chr16: 5241397-5241456 | 0.683772 | 0.010835 |
| A_32_P74243 | BF366211 | chr18: 044731599-044731658 | 1.767291 | 0.017371 |
| A_23_P100022 | NM_014848 | chr15: 89639333-89639392 | 0.805696 | 0.049892 |
| A_23_P358555 | NM_032356 | chr17: 7700841-7700782 | 0.58962 | 0.038802 |
| A_24_P818010 | AK097080 | chr1: 124783-124724 | 2.160989 | 0.004497 |
| A_24_P861622 | AA782850 | chr16: 19420838-19420896 | 1.425145 | 0.047093 |
| A_23_P3368 | NM_002569 | chr15: 89227001-89227060 | 1.771172 | 0.01279 |
| A_24_P765784 | AK057670 | chr7: 73811291-73811350 | 1.737696 | 0.039929 |
| A_24_P32203 | ENST00000397321 | chr12: 131559725-131559666 | 0.845234 | 0.049895 |
| A_23_P361841 | NM_020734 | chr12: 8820586-8820645 | 1.719603 | 0.031174 |
| A_23_P419602 | NM_004599 | chr22: 40632145-40632204 | 0.626161 | 0.007491 |
| A_23_P64058 | NM_153819 | chr11: 64251049-64250990 | 0.799517 | 0.03396 |
| A_32_P103060 | AI150964 | chr3: 130369697-130369756 | 0.576221 | 0.011497 |
| A_24_P231513 | NM_005721 | chr2: 114431570-114431629 | 2.047084 | 0.039766 |
| A_32_P190568 | AK094885 | chr3: 162629838-162629897 | 1.623932 | 0.004781 |
| A_24_P125977 | NM_033184 | chr17: 36475303-36475244 | 0.765714 | 0.019108 |
| A_23_P349321 | NM_022166 | chr16: 17109197-17109138 | 1.895605 | 0.007701 |
| A_23_P115683 | NM_024747 | chr10: 103817665-103817724 | 0.553043 | 0.006663 |
| A_23_P166548 | NM_025204 | chr22: 48979115-48979174 | 0.718068 | 0.003659 |
| A_23_P204702 | NM_003217 | chr12: 48438529-48438790 | 2.178006 | 0.007058 |
| A_24_P618476 | THC2654949 | chr16: 85863090-85863031 | 0.780705 | 0.008187 |
| A_23_P57733 | NM_033663 | chr3: 115373284-115361370 | 0.705769 | 0.008189 |
| A_23_P119377 | NM_004228 | chr19: 53667405-53667464 | 1.612532 | 0.046459 |
| A_24_P846988 | XM_374705 | chr7: 156927307-156927248 | 0.787892 | 0.01519 |
| A_24_P264211 | NM_002823 | chr2: 232281702-232284357 | 1.710542 | 0.029439 |
| A_24_P333357 | AK090463 | chr16: 596449-596508 | 0.509095 | 0.007526 |
| A_23_P210496 | NM_022104 | chr20: 44009424-44009483 | 0.746051 | 0.008521 |
| A_23_P75220 | NM_031212 | chr10: 101360723-101360664 | 0.631716 | 0.000784 |
| A_24_P365327 | NM_015578 | chr19: 39410605-39410664 | 1.892327 | 0.006354 |
| A_23_P351232 | NM_173680 | chr7: 149726423-149726482 | 0.795294 | 0.047188 |
| A_23_P17345 | NM_005461 | chr20: 38748283-38748224 | 0.701313 | 0.02807 |
| A_32_P88415 | NM_133371 | chr5: 150038984-150039043 | 0.75241 | 0.012775 |
| A_24_P313334 | NM_001018108 | chr15: 41875244-41875303 | 1.578196 | 0.035384 |
| A_24_P340679 | NM_021130 | chr7: 44805546-44805826 | 1.901676 | 0.031209 |
| A_23_P487 | NM_012474 | chr1: 164143746-164143805 | 1.491552 | 0.040373 |
| A_24_P144134 | | chr17: 025420565-025420624 | 1.766489 | 0.023242 |
| A_24_P59485 | AK095300 | chr2: 242032343-242032402 | 0.564768 | 0.020442 |
| A_23_P64010 | NM_014342 | chr11: 47597047-47596988 | 2.557233 | 0.003241 |
| A_24_P290999 | U60873 | chr11: 64948580-64948638 | 1.419458 | 0.031276 |
| A_32_P86150 | NM_001025200 | chr16: 73795560-73795501 | 0.762665 | 0.013152 |
| A_23_P92754 | NM_213647 | chr5: 176455727-176455786 | 0.777059 | 0.047413 |
| A_24_P585004 | THC2570492 | chr11: 117740-117681 | 0.659938 | 0.011785 |
| A_24_P198844 | NM_004870 | chr17: 7431735-7431794 | 2.10837 | 0.017121 |
| A_24_P79153 | NM_079834 | chr19: 1876547-1876606 | 1.508146 | 0.023437 |
| A_23_P152651 | NM_007372 | chr17: 59249724-59249783 | 1.562627 | 0.028372 |
| A_24_P706901 | BF895757 | chr19: 63752181-63752122 | 0.780508 | 0.033086 |
| A_32_P234405 | CK570316 | chr1: 205168681-205168740 | 0.773043 | 0.003329 |
| A_23_P65357 | NM_018373 | chr14: 69909035-69908976 | 1.719427 | 0.01662 |
| A_24_P341853 | NM_031454 | chr22: 48997978-48998037 | 0.6943 | 0.005878 |
| A_23_P116797 | NM_021640 | chr12: 51986779-51986838 | 0.68098 | 0.030606 |
| A_23_P398275 | AF316855 | chr15_random: 664627-664686 | 0.562619 | 0.004806 |
| A_24_P245767 | NM_006936 | chr21: 45051097-45051038 | 1.889643 | 0.019319 |
| A_24_P316363 | NM_001521 | chr2: 27413898-27413701 | 2.474468 | 0.002909 |
| A_23_P134953 | NM_001122 | chr9: 19106429-19106370 | 1.645587 | 0.004864 |
| A_24_P937931 | | chr17: 034667924-034667983 | 0.750506 | 0.023369 |
| A_23_P33436 | NM_002383 | chr16: 29727512-29728906 | 0.712306 | 0.036905 |
| A_32_P187599 | NM_001018067 | chr1: 67658403-67658344 | 1.717883 | 0.040252 |
| A_23_P67669 | NM_052890 | chr19: 15443754-15441735 | 0.809061 | 0.014005 |
| A_23_P369983 | NM_174905 | chr19: 43591400-43591459 | 1.767332 | 0.033382 |
| A_23_P24755 | NM_003164 | chr11: 62331173-62331114 | 1.870845 | 0.042542 |
| A_23_P312181 | NM_207013 | chr16: 2762063-2762004 | 0.720212 | 0.001689 |
| A_32_P9816 | AI857589 | chr16: 1691696-1691637 | 0.691755 | 0.00195 |
| A_23_P144453 | NM_033296 | chr4: 6694652-6694711 | 2.212837 | 0.0104 |
| A_23_P106773 | NM_177528 | chr16: 28510827-28510768 | 0.584927 | 0.008304 |
| A_23_P125533 | NM_033215 | chrX: 49029842-49029901 | 0.803944 | 0.029964 |
| A_23_P395493 | BC031631 | chr10: 89594388-89594447 | 2.721491 | 0.002263 |
| A_24_P279704 | NM_080600 | chr19: 40496421-40496480 | 0.803193 | 0.030398 |
| A_23_P156880 | NM_006208 | chr6: 132253564-132253623 | 1.738051 | 0.004347 |
| A_23_P132294 | NM_001001560 | chr22: 36359426-36359485 | 0.51767 | 0.042943 |
| A_32_P38286 | NM_174910 | chr6: 169893448-169893389 | 0.806183 | 0.032563 |
| A_24_P679984 | | chr1: 159455581-159455522 | 1.710268 | 0.003265 |
| A_23_P344594 | NM_153217 | chr5: 72505922-72505981 | 0.770745 | 0.003784 |
| A_24_P333106 | CR595167 | chr14: 34025181-34025240 | 1.541592 | 0.008919 |

The predictive algorithm will be used for a validation study with a predefined cut-off on the remaining cases from the B31 study (N=1000).

HER2 was not the main determinant of the degree of benefit from trastuzumab added to adjuvant chemotherapy. Without being held to any specific mechanism of action, Applicants believe that this could be due to the fact that HER2 positive tumors are sensitive to the legacy treatment (chemohormonal therapy) and disseminated tumor cells may express different levels of HER2 in the index tumor (see, e.g., Gangnus, et al., Clin. Cancer Res. 10:3457-3463, 2004, Schardt, et al., Cancer Cell 8:227-239, 2005, and Ignatiadis, et al., Clin. Cancer Res. 14:2593-2600, 2008). Prediction of the degree of benefit from trastuzumab added to chemotherapy may be quite different from predicting tumor response in a metastatic or neoadjuvant setting where direct tumor shrinkage is measured. This data has broad implications on development of targeted therapies in an adjuvant setting.

Having now fully described the present disclosure in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference in their entirety to the same extent as if each individual publication, patent or patent application was specifically and individually indicated to be incorporated by reference. Citation of the above patents, patent applications, publications and documents is not an admission that any of the foregoing is pertinent prior art, nor does it constitute any admission as to the contents or date of these publications or documents.

Unless defined otherwise, all technical and scientific terms used herein have the same meanings as commonly understood by one of ordinary skill in the art to which this invention belongs. Although any methods and systems similar or equivalent to those described herein can be used in the practice or testing of the present invention, the methods, devices, and materials are now described. All publications mentioned herein are incorporated herein by reference for the purpose of describing and disclosing the processes, systems and methodologies which are reported in the publications which might be used in connection with the invention. Nothing herein is to be construed as an admission that the invention is not entitled to antedate such disclosure by virtue of prior invention.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A method of treating a patient having breast cancer comprising:
a) measuring the expression level or amplification of one or more of the genes listed in Tables 5-14 in a patient having breast cancer; and
b) providing a patient having increased expression level or amplification of one or more of the genes listed in Tables 5-14 with a therapeutically effective amount of a combination of one or more adjuvant chemotherapeutic compounds and at least one compound that inhibits the activity, amount, or signaling of HER2.

2. The method of claim 1, wherein the expression level is measured by an enzyme-linked immunosorbent assay, a radioimmunoassay, or flow cytometry.

3. The method of claim 1, wherein the expression level is measured via a real time quantitative polymerase chain reaction assay.

4. The method of claim 1, wherein the amplification is measured via fluorescent in situ hybridization.

5. The method of claim 1, wherein the expression level or amplification of one or more of the genes listed in Tables 6, 9, or 11 is measured.

6. The method of claim 1, wherein the expression level or amplification of one or more of the genes listed in Table II is measured.

7. The method of claim 1, wherein the at least one compound that inhibits the activity, amount, or signaling of HER2 is an anti-HER2 antibody.

8. The method of claim 7, wherein the anti-HER2 antibody is a monoclonal antibody.

9. The method of claim 8, wherein the anti-HER2 antibody is trastuzumab.

10. The method of claim 1, wherein the one or more adjuvant chemotherapeutic compounds are doxorubicin, cyclophosphamide, and paclitaxel.

11. The method of claim 1, further comprising screening said patient for increased expression or amplification of the HER2 gene.

12. The method of claim 11, wherein said patient is screened for increased expression of the HER2 gene.

13. The method of claim 11, wherein said patient is screened for amplification of the HER2 gene.

14. The method of claim 11, wherein said patient has increased expression or amplification of the HER2 gene.

15. The method of claim 11, wherein said patient does not have increased expression or amplification of the HER2 gene.

16. A method of diagnosing a patient with breast cancer undergoing adjuvant therapy to determine the benefit of administering a compound that inhibits the activity, amount, or signaling of HER2, comprising measuring the expression level or amplification of one or more of the genes listed in Tables 5-14 in said patient, wherein an increased expression level or amplification of one or more of the genes listed in Tables 5-14 is indicative of a patient with breast cancer undergoing adjuvant therapy who would benefit from administering a compound that inhibits the activity, amount, or signaling of HER2.

17. The method of claim 16, further comprising screening the patient with breast cancer for increased expression or amplification of the HER2 gene.

18. The method of claim 16, wherein the compound that inhibits the activity, amount, or signaling of HER2 is an anti-HER2 antibody.

19. The method of claim 18, wherein the anti-HER2 antibody is a monoclonal antibody.

20. The method of claim 19, wherein the anti-HER2 antibody is trastuzumab.
